(19) United States
(12) Patent Application Publication
(10) Pub. No.: US 2005/0059021 A1
(43) Pub. Date: Mar. 17, 2005

(54) Insulin-like growth factor-1 receptor (IGF-1R) polymorphic alleles and use of the same to identify DNA markers for reproductive longevity

(75) Inventors: Abdol Hossain Farid, Truro (CA); Charles J. Otieno, Vancouver (CA)

Correspondence Address: MCKEE, VOORHEES & SEASE, P.L.C., 801 GRAND AVENUE, SUITE 3200, DES MOINES, IA, 50309-2721, US

(73) Assignee: PERFORMANCE GENOMICS, INC., Truro (CA)

(21) Appl. No.: 10/662,613

(22) Filed: Sep. 15, 2003

(51) Int. Cl. C12Q 1/68

(52) U.S. Cl. 435/6

(57) ABSTRACT

Disclosed herein are embodiments for genotyping an animal for the presence of polymorphic alleles in the IGF-1R gene that are associated with reproductive longevity and/or ability to better sustain stress, and preferably selecting those animals for future breeding purposes.

### BACKGROUND OF THE INVENTION

Genetic mutations are the basis of evolution and genetic diversity. Genetic markers represent specific loci in the genome of a species, population or closely related species, and sampling of different genotypes at these marker loci reveals genetic variation. The genetic variation at marker loci can then be described and applied to genetic studies, commercial breeding, diagnostics, and cladistics. Genetic markers have the greatest utility when they are codominant, highly heritable, multi-allelic, and numerous. Most genetic markers are heritable because their alleles are determined by the nucleotide sequence of DNA which is highly conserved from one generation to the next, and the detection of their alleles is unaffected by the natural environment. Markers have multiple alleles because, in the evolutionary process, rare, genetically-stable mutations in DNA sequences defining marker loci arose and were disseminated through the generations along with other existing alleles. The highly conserved nature of DNA combined with rare occurrences of stable mutations allows genetic markers to be both predictable and discerning of different genotypes. The repertoire of genetic-marker technologies today allows multiple technologies to be used simultaneously in the same project. The invention of each new genetic-marker technology and each new DNA polymorphism adds additional utility to genetic markers. Many genetic-marker technologies exist. Some examples are restriction-fragment-length polymorphism (RFLP) Bostein et al (1980) Am J Hum Genet 32:314-331; single-strand conformation polymorphism (SSCP) Fischer et al. (1983) Proc Natl Acad Sci USA 80:1579-1583, Orita et al. (1989) Genomics 5:874-879; amplified fragment-length polymorphism (AFLP) Vos et al. (1995) Nucleic Acids Res 23:4407-4414; microsatellite or single-sequence repeat (SSR) Weber J L and May P E (1989) Am J Hum Genet 44:388-396; random-amplified polymorphic DNA (RAPD) Williams et al (1990) Nucleic Acids Res 18:6531-6535; sequence tagged site (STS) Olson et al. (1989) Science 245:1434-1435; genetic-bit analysis (GBA) Nikiforov et al (1994) Nucleic Acids Res 22:4167-4175; allele-specific polymerase chain reaction (ASPCR) Gibbs et al. (1989) Nucleic Acids Res 17:2437-2448, Newton et al. (1989) Nucleic Acids Res 17:2503-2516; nick-translation PCR (e.g., TAQMAN™) Lee et al. (1993) Nucleic Acids Res 21:3761-3766; and allele-specific hybridization (ASH) Wallace et al. (1979) Nucleic Acids Res 6:3543-3557, (Sheldon et al. (1993) Clinical Chemistry 39(4):718-719) among others. Each technology has its own particular basis for detecting polymorphisms in DNA sequence.

The ability to follow a specific favorable genetic allele involves a novel and lengthy process of the identification of a DNA molecular marker for a major effect gene. The marker may be linked to a single gene with a major effect or linked to a number of genes with additive effects. DNA markers have several advantages; segregation is easy to measure and is unambiguous, and DNA markers are co-dominant, i.e., heterozygous and homozygous animals can be distinctively identified. Once a marker system is established selection decisions could be made very easily, since DNA markers can be assayed any time after a tissue or blood sample can be collected from the individual infant animal, or even an embryo.

Poor reproductive performance is one of the major causes for culling in dairy (Beaudeau et al. 1995; Durr et al. 1997; Kulak et al. 1997; Bascom and Young 1998) and beef cattle (Tanida et al. 1988), and leads to a decrease in profitability (Tanida et al. 1988; Beaudeau et al. 1995; Kulak et al. 1997; Bascom and Young 1998). The highest level of profitability in a dairy herd is achieved when high yielding cows are maintained in the herd for several lactations (Gill and Allaire 1976; Allaire and Gibson 1992; Kulak et al. 1997). An increase in length of production from 3 to 4 lactations increases milk yield per lactation and profit per year by 11 and 13% respectively (Strandberg 1996). Reproductive longevity is even more important in beef cattle, sheep, swine and fur bearing animals, where replacement cost is, after nutrition, the second highest source of expenditure. Clearly, improving reproductive longevity offers one of the greatest opportunities for increasing productive efficiency and economic return in the multi-billion dollar livestock industry in the world. This is illustrated by the fact that reproductive longevity is included in the national dairy genetic evaluation systems in Canada (herd life) and the U.S. (production life).

Moderate variation exists for reproductive longevity within and among different breeds of cattle (Silva et al. 1986; Smith and Quass 1984; Bailey 1991; Arthur et al. 1993), suggesting the possibility for genetic improvement in this trait. However, despite its obvious economic importance, it is difficult to improve reproductive longevity through conventional breeding methods because of the low heritability of this trait (Smith and Quass 1984; Tanida et al. 1988; Boldman et al. 1992; VanRaden and Klaaskate 1993) and the long time necessary to obtain information on reproductive longevity in livestock. Attempts to improve reproductive longevity of dairy cattle through indirect selection, such as the use of ‘type traits’ that are measured early in life, has been ineffective (Smith and Quass 1984; Boldman et al. 1992; VanRaden and Klasskate 1993).

The above limitations make reproductive longevity an ideal candidate trait for the use of DNA markers (Lande and Thompson 1990), which would provide a means of identification of animals with superior breeding value at an early age on the basis of a simple laboratory test. Developing DNA markers for reproductive longevity is, however, a difficult and time-consuming task in long-lived livestock resources. A logical strategy would involve identification of candidate genes in a mammalian model with a short generation interval and later validating them in livestock (Copeland et al. 1993). This is especially true in the case of genes that control reproductive longevity and life span (Rose and Nusbaum 1994), since direct selection for prolonged reproductive age in large mammals is very time consuming and prohibitively expensive. The genes identified in animals will be putative candidates for the development of DNA markers for reproductive longevity in other species.

Although there are several reports on the quantitative genetics aspects of reproductive longevity in livestock (VanRaden and Klaaskate 1993; Smith and Quass 1984; Kulak et al. 1997; Bascom and Young 1998), little information is available on the genetic control of this trait in any mammalian species. Most of the available information on the genetic control of reproductive longevity and life span has been obtained on simple organisms, such as Drosophila and Caenorhabditis elegans (C. elegans). In C. elegans, for example, the daf genes (daf-2, -12, -16, -18 -23), which are components of the IGF-1R signaling cascade, have been shown to control the regulation of metabolism, development, reproduction and life span (Lakowski and Hekimi 1996; Apfeld and Kenyon 1998; Hekimi et al. 1998). Also, there is a positive relationship between life span and reproduction in C. elegans (Hsin and Kenyon 1999) and among mammals (Packer et al. 1998; Tissenbaum and Ruvkun 1998). Although information on lower organisms is useful, their usefulness in mammals should be assessed in an appropriate mammalian model that exhibits widely contrasting reproductive longevity phenotypes.

The use of DNA markers will facilitate the identification of animals that are genetically prone to a) reproduce longer than the average and, separately b) those that have a higher likelihood, compared with the average, of conceiving during lactation (sustained lactation and pregnancy stress). The marker may be directly involved in prolonging reproductive life, or may be linked to a single gene with a major effect, or may be linked to a number of genes with additive effects on animals' phenotype. Their segregation is easy to measure and is unambiguous, and DNA markers are co-dominant, i.e., heterozygous and homozygous animals can be distinctively identified. Once a marker system is established, selection decisions can be made easily, since DNA markers can be assayed any time after a tissue or blood sample can be collected from the individual infant animal, or even an embryo.

For the foregoing reasons, there is a need for a method of selecting animals with improved reproductive longevity and/or ability to better sustain stress factors. More particularly, a need for identifying markers which may be used to improve economically beneficial characteristics in animals by identifying and selecting animals with these favorable characteristics at the genetic level.

Therefore, an object of the present invention is to provide a method of identifying polymorphisms in the IGF-1R gene which are indicative of reproductive longevity in mammals and their ability to sustain performance in combination with stress factors such as lactation, pregnancy, and health status.

Another object of the invention is to provide assays for determining the presence of these genetic markers.

A further object of the invention is to provide methods for screening animals to determine those more likely to exhibit favorable traits associated with reproductive longevity and the ability to sustain performance under stress, which increases the accuracy of selection and breeding methods.

Yet another object of the invention is to provide PCR amplification and detection tests which will greatly expedite the determination of presence of the markers.

A still further object of the invention is to provide a method for determining the haplotype of the IGF-1R gene indicative of reproductive longevity and the ability to sustain performance under stress.

Additional objects and advantages of the invention will be set forth in part in the description that follows, and in part will be obvious from the description, or may be learned by the practice of the invention. The objects and advantages of the invention will be attained by means of the instrumentalities and combinations particularly pointed out in the appended claims.

### BRIEF SUMMARY OF THE INVENTION

This invention relates to the discovery of alternate forms of the insulin-like growth factor-1 receptor (IGF-1R) gene which are useful as a genetic markers associated with reproductive longevity and the ability to better sustain stress factors in animals such as lactation and pregnancy in animals.

According to an embodiment of the present invention there are provided methods for identifying a polymorphism in an animal. One embodiment includes a method for genetically identifying an animal comprising obtaining a sample of genetic material from an animal and assaying for the presence of a polymorphism in the insulin-like growth factor 1 receptor gene (IGF-1R), wherein said polymorphism is associated with reproductive longevity and/or ability to better sustain stress factors such as lactation and pregnancy stress.

A further embodiment includes a method for screening animals to determine those more likely to exhibit favorable traits associated with reproductive longevity and ability to sustain stress factors such as lactation and pregnancy stress. These methods include obtaining a genetic sample from the animal. The methods can further include assaying for the presence or absence of a polymorphism in the IGF-1R gene associated with reproductive longevity and/or the ability to sustain stress factors in animals such as lactation and pregnancy.

Further embodiments of the invention can include amplifying the gene or a region of the gene, which contains at least one polymorphism. Since one of the polymorphisms may involve changes in the amino acid composition of the IGF-1R protein, assay methods may even involve ascertaining the amino acid composition of these proteins. Methods for this type or purification and analysis typically involve isolation of the protein through means including fluorescence tagging with antibodies, separation and purification of the protein (i.e., through reverse phase HPLC system), and use of an automated protein sequencer to identify the amino acid sequence present. Protocols for this assay are standard and known in the art and are disclosed in Ausubel et al. (eds.), Short Protocols in Molecular Biology 4^{th} ed. (John Wiley and Sons 1999).

Another embodiment includes a method for determining the haplotype of the IGF-1R gene of an animal wherein the haplotype is indicative of reproductive longevity and/or ability to sustain stress factors.

In a preferred embodiment, a sample of genetic material is obtained from an animal and the sample is analyzed to determine the presence or absence of a polymorphism in the IGF-1R gene, which is correlated with reproductive longevity and/or ability to sustain stress factors such as lactation and pregnancy stress.

As is well known to those of skill in this art, a variety of techniques may be utilized when comparing nucleic acid molecules for sequence differences. These include by way of example, restriction fragment length polymorphism analysis, heteroduplex analysis, single-strand conformation polymorphism analysis, denaturing gradient electrophoresis and temperature gradient electrophoresis.

In a preferred embodiment the polymorphism is a 12-bp deletion and two restriction fragment length polymorphism and the assay comprises identifying the animal's IGF-1R gene from isolated genetic material; exposing the gene to a restriction enzyme that yields restriction fragments of the gene of varying length; separating the restriction fragments to form a restriction pattern, such as by electrophoresis or HPLC separation; and comparing the resulting restriction fragment pattern from a IGF-1R gene that is either known to have or not to have the desired marker.

In a most preferred embodiment the gene is isolated by the use of primers and DNA polymerase to amplify a specific region of the gene which contains the polymorphism. Next the amplified region is digested with a restriction enzyme and fragments are again separated. Visualization of the RFLP pattern is by simple staining of the fragments, or by labeling the primers or the nucleoside triphosphates used in amplification.

It expected that with no more than routine testing as described herein this marker can be applied to different animal species to select for reproductive longevity and/or sustained performance in a situation with stress caused by lactation, pregnancy, or health status based on the teachings herein. Female animals of the same breed or breed cross or similar genetic lineage are bred, and the reproductive longevity and/or sustained lactation and pregnancy stress shown by each animal is determined and correlated. For other species in which sequences are available a BLAST comparison of the IGF-1R may be used to ascertain whether the particular allele disclosed herein is present.

The term “analogous polymorphism” shall be a polymorphism which is the same as any of those disclosed herein as determined by BLAST comparisons.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.
(a) As used herein, “reference sequence” is a defined sequence used as a basis for sequence comparison. In this case the Reference is the IGF-1R sequence. A reference sequence may be a subset or the entirety of a specified sequence; for example, as a segment of a full-length cDNA or gene sequence, or the complete cDNA or gene sequence.
(b) As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (http://www.ncbi.nlm.nih.gov/).

This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.
(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).
(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.
(e) The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%.

These programs and algorithms can ascertain the analogy of a particular polymorphism in a target gene to those disclosed herein. It is expected that this polymorphism will exist in other animals and use of the same in other animals than disclosed herein involved no more than routine optimization of parameters using the teachings herein.

It is also possible to establish linkage between specific alleles of alternative DNA markers and alleles of DNA markers known to be associated with a particular gene (e.g. the IGF-1R gene discussed herein), which have previously been shown to be associated with a particular trait. Thus, in the present situation, taking the IGF-1R gene, it would be possible, at least in the short term, to select for animals likely to produce one or more of the traits of reproductive longevity and/or the ability to better sustain stress caused by lactation and pregnancy, or alternatively against animals less likely to exhibit the traits of reproductive longevity and/or the ability to better sustain stress caused by lactation and pregnancy, indirectly, by selecting for certain alleles of a IGF-1R associated marker through the selection of specific alleles of alternative chromosome markers. As used herein the term “genetic marker” shall include not only the polymorphism disclosed by any means of assaying for the protein changes associated with the polymorphism, be they linked markers, use of microsatellites, or even other means of assaying for the causative protein changes indicated by the marker and the use of the same to influence the traits of reproductive longevity and/or the ability to sustain stress in an animal.

As used herein, often the designation of a particular polymorphism is made by the name of a particular restriction enzyme. This is not intended to imply that the only way that the site can be identified is by the use of that restriction enzyme. There are numerous databases and resources available to those of skill in the art to identify other restriction enzymes which can be used to identify a particular polymorphism. Two examples are: http://www.geneseo.edu/˜bio/ and http://www.firstmarket.com/cutter/cut2.html. In fact, as disclosed in the teachings herein there are numerous ways of identifying a particular polymorphism or allele with alternate methods which may not even include a restriction enzyme, but which assay for the same genetic or proteomic alternative form.

The invention is intended to include these sequences as well as all conservatively modified variants thereof as well as those sequences which will hybridize under conditions of high stringency to the sequences disclosed. The term IGF-1R is used herein shall be interpreted to include these conservatively modified variants as well as those hybridized sequences.

The term “conservatively modified variants” applies to both amino acid and nucleic acid sequences. With respect to particular nucleic acid sequences, conservatively modified variants refer to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W). See also, Creighton, Proteins, W.H. Freeman and Company (1984).

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

The term “stringent conditions” or “stringent hybridization conditions” includes reference to conditions under which a probe will hybridize to its target sequence, to a detectably greater degree than to other sequences (e.g., at least 2-fold over background). Stringent conditions are sequence-dependent and be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. One of ordinary skill is apprised in knowing that the time of the hybridization is dependent on the concentration of the probe. Exemplary low stringency conditions include hybridization with a buffer solution of 30 to 35% formamide, 1 M NaCl, 1% SDS (sodium dodecyl sulphate) at 37° C., and a wash in 1× to 2× SSC (20× SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5X to 1× SSC at 55 to 50° C. Exemplary high stringency conditions include hybridization in 50% formamide, 1 M NaCl, 1% SDS at 37° C., and a wash in 0.1× SSC at 60 to 65° C. for at least 15 minutes.

Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-284 (1984): Tₘ=81.5° C.+16.6 (log M)+0.41 (% GC) −0.61 (% form) −500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of the complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acids Probes, Part I, Chapter 2, Ausubel, et al., Eds., Greene Publishing and Wiley-Interscience, New York (1995).

These and other features, aspects, and advantages of the present invention will become better understood with regard to the following description, appended claims, and accompanying drawings.

### BRIEF DESCRIPTION OF THE DRAWINGS

FIG. 1 depicts the nucleotide sequence of the insulin-like growth factor-1 receptor in mice (SEQ ID NO:1)(GenBank accession number AF056187).

FIG. 2 depicts the amino acid sequence of the insulin-like growth factor-1 receptor in mice (SEQ ID NO:2)(GenBank protein id AAC12782.1).

FIG. 3 depicts the mRNA sequence of insulin-like growth factor I receptor in mice (SEQ ID NO:3) (Genbank accession number XM_{—}133508).

FIG. 4 depicts the alignment of exon 21 of the mouse IGF1-R sequences from Genbank accession number AF056187 (SEQ ID NO: 1) and Genbank accession number XM_{—}133508 (SEQ ID NO:3), and the amino acid sequence of this region (SEQ ID NO:4). The A to G substitution at position 3876 of the Genbank accession number AF056187 (HpaII site, locus B) is bolded and underlined. The 12 bp insertion/deletion is bolded and underlined. The junction of exon 20 and exon 21 is shown by “0”.

FIG. 5 depicts intron 16 (SEQ ID NO:5) of the mouse IGF1-R gene and the surrounding exons amplified by primers PSEQ16F (SEQ ID NO:12) and PSEQ16R (SEQ ID NO:13), and its alignment with the mouse IGF1-R gene (Genbank accession number AC101879; SEQ ID NO:6). This sequence contains 102 bp of exon 16 (nucleotides 1 to 102), 283 bp of intron 16 (nucleotides 103 to 385) and 101 bp of exon 17 (nucleotides 386 to 486) of the mouse IFG1-R gene. Exon-intron junctions are shown by 0. The ‘G’ insertion is at position 176 of SEQ ID NO:5 after nucleotide 56456 of SEQ ID NO:6 (Genbank accession number AC101879). This insertion is bolded and underlined. Note that SEQ ID NO:6 (Genbank accession number AC101879) is the reverse complement of other sequences of the IGF1-R in Genbank. The ‘G’ to ‘A’ substitution (DpnII site, locus A) is at position 331 of SEQ ID NO:5, corresponding to nucleotide 556303 of SEQ ID NO:6 (Genbank accession number AC101879). This nucleotide is bolded and underlined. The forward (PSEQ16F) and reverse (PSEQ16R) primers are underlined.

FIG. 6 depicts mouse clone RP23-378H21, complete sequence (SEQ ID NO:6) (Genbank accession number AC101879).

FIG. 7 depicts the nucleotide sequence of the insulin-like growth factor-1 receptor in pig (SEQ ID NO:7). cDNA sequence in lower case letters comes from Accession No. AB003362. Intron 9 sequence in lower case letters comes from Accession No. AJ491314. Intron sequence in upper case letters was derived from Applicants sequencing efforts.

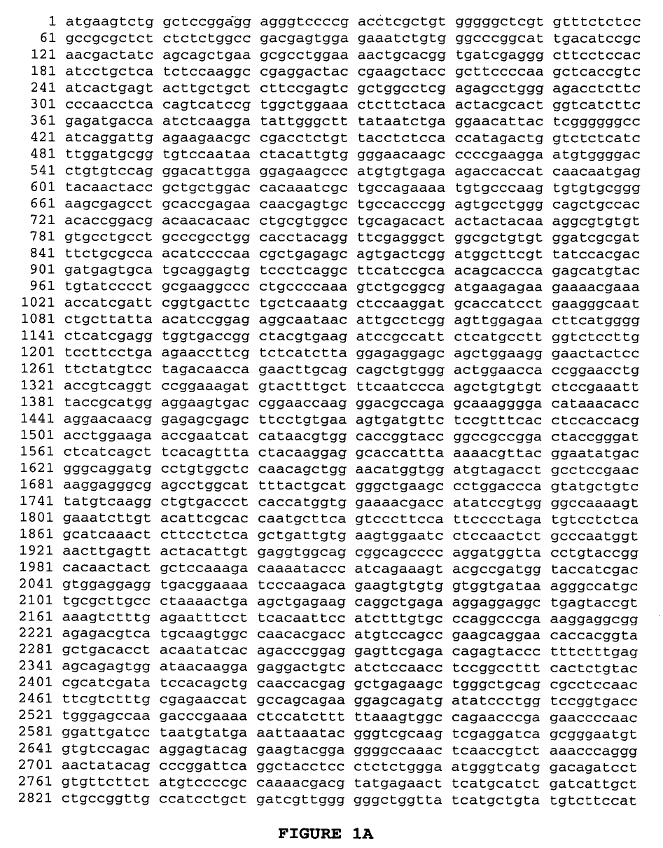

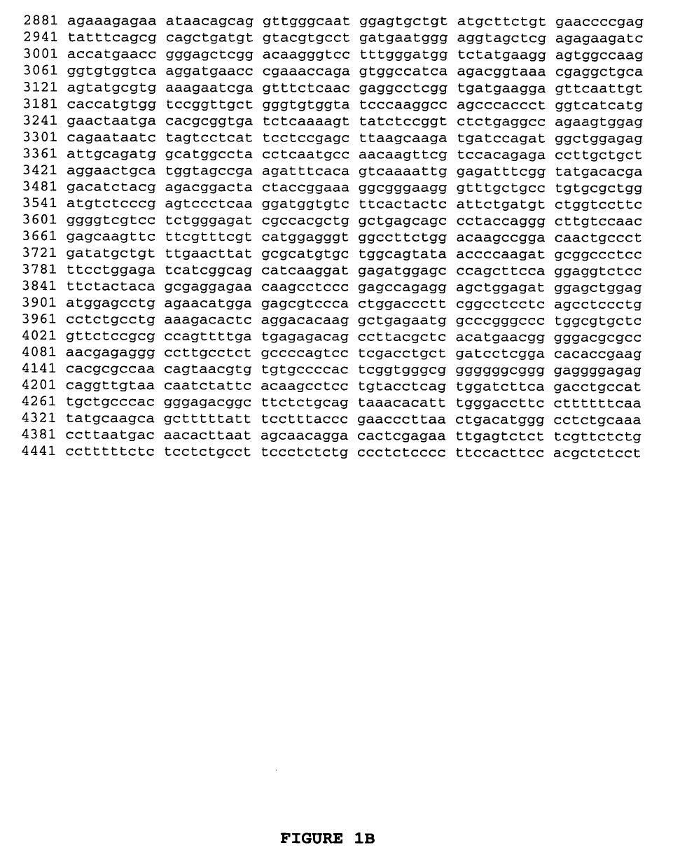

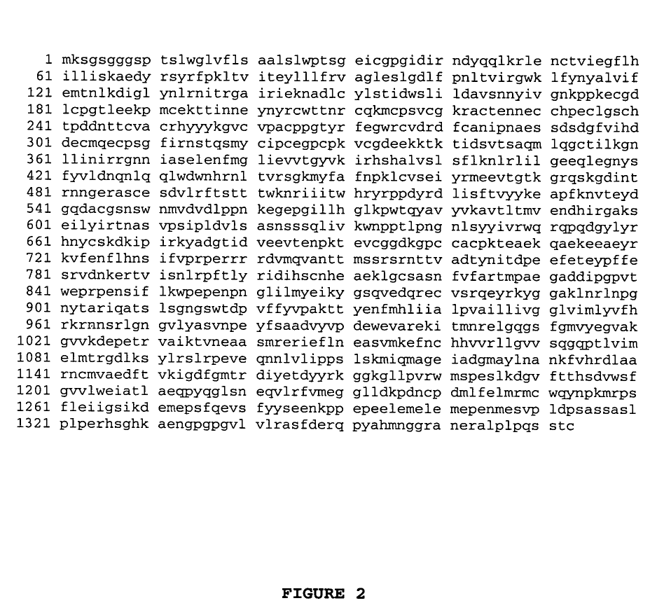

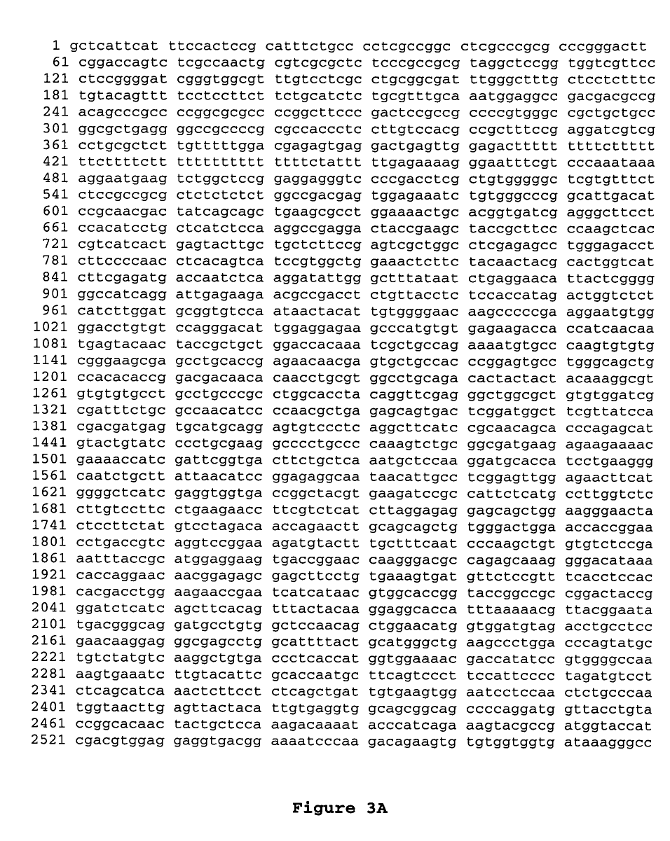

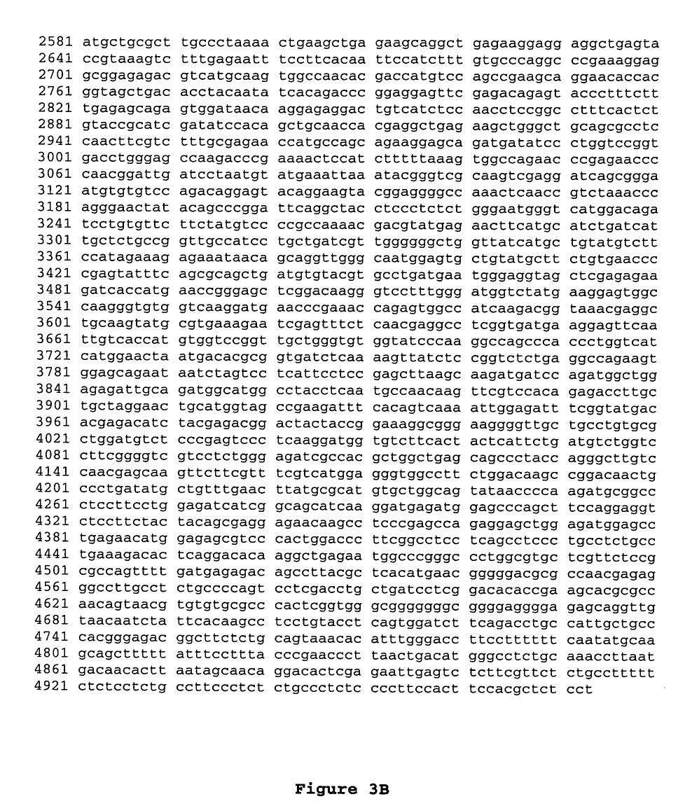

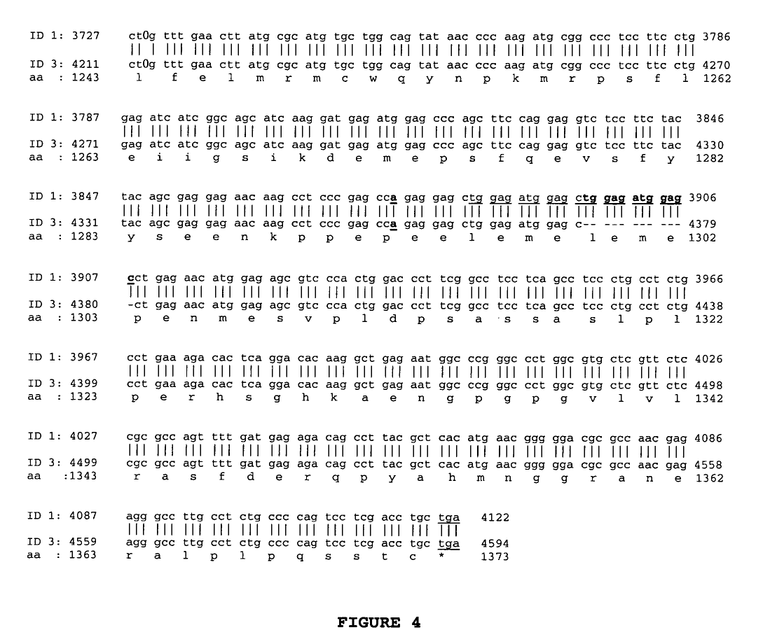

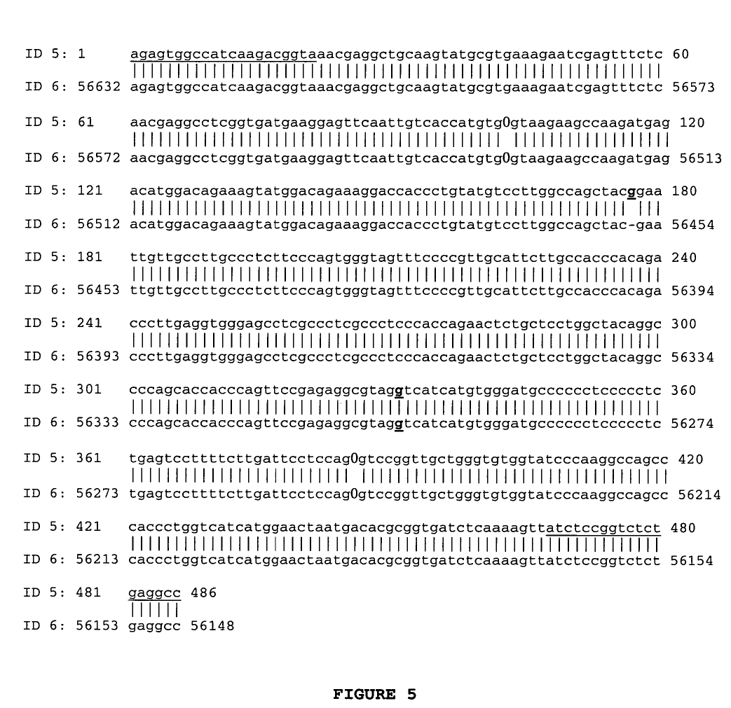

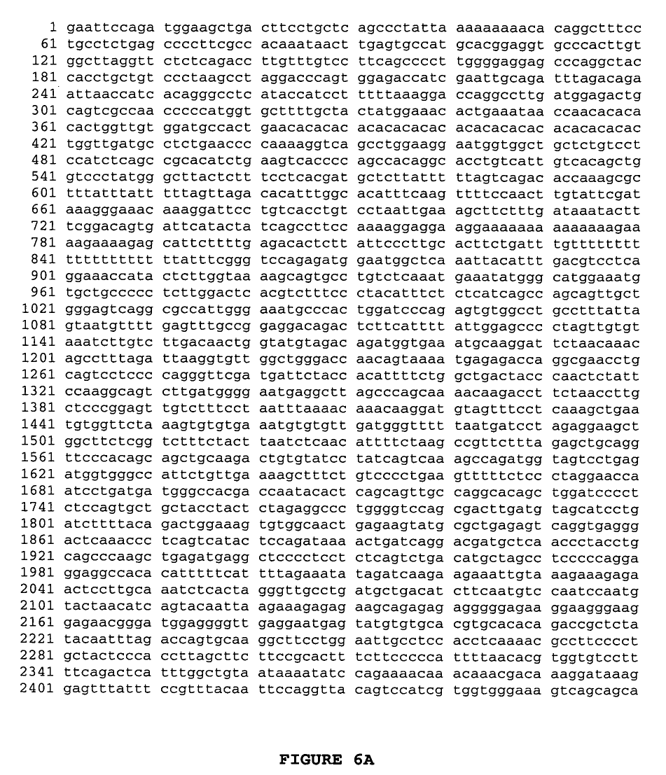

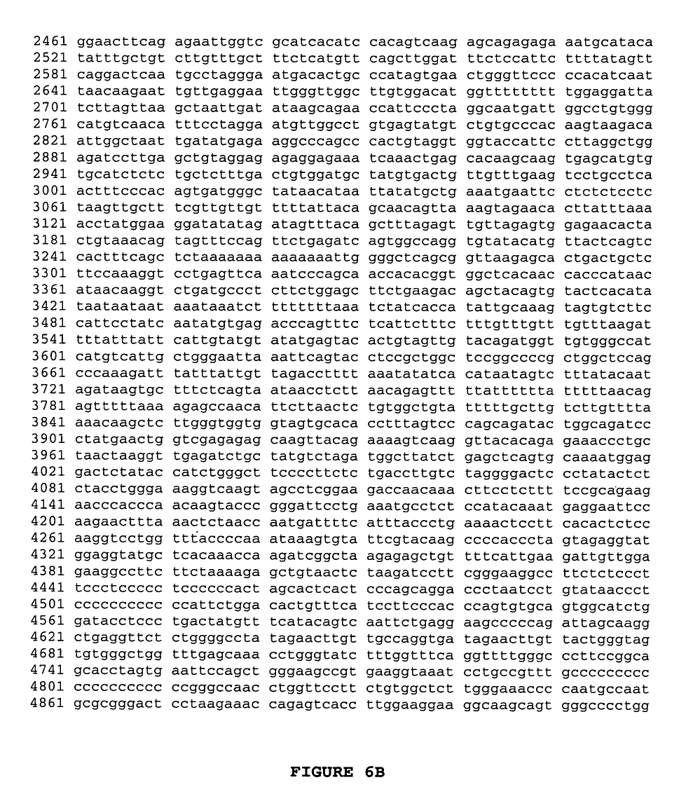

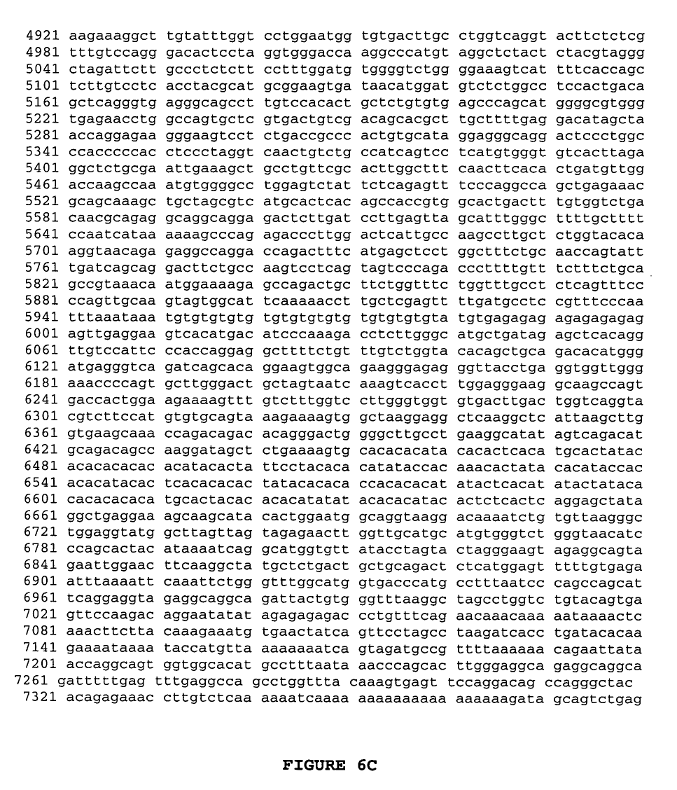

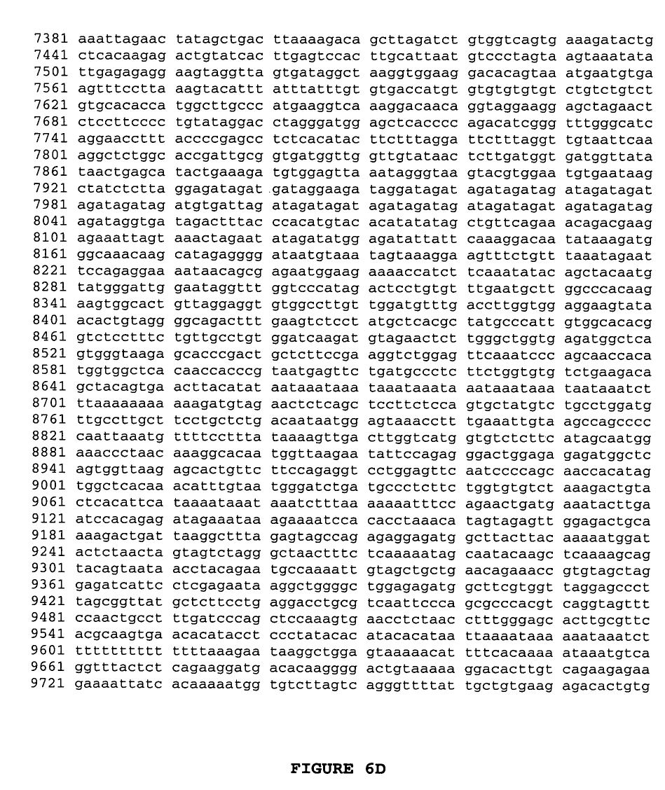

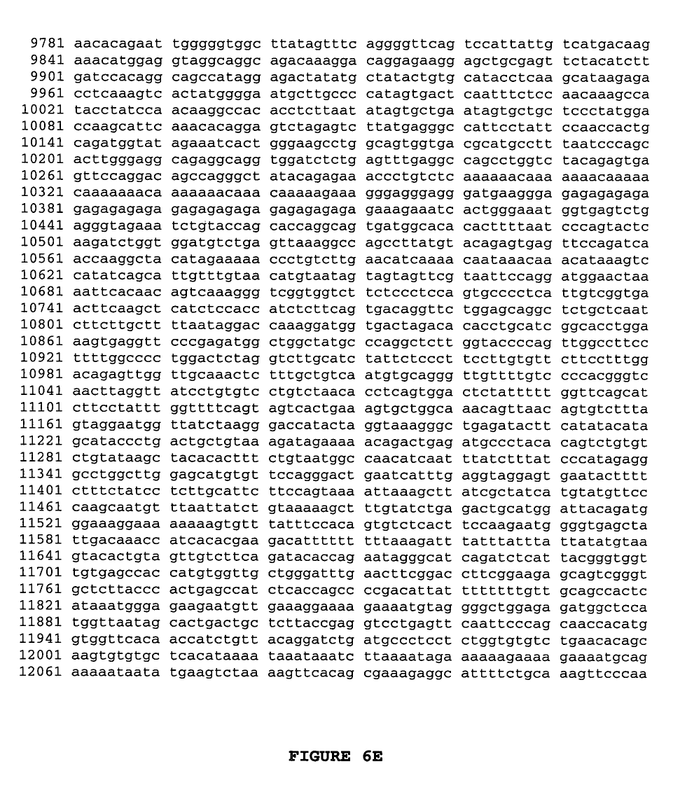

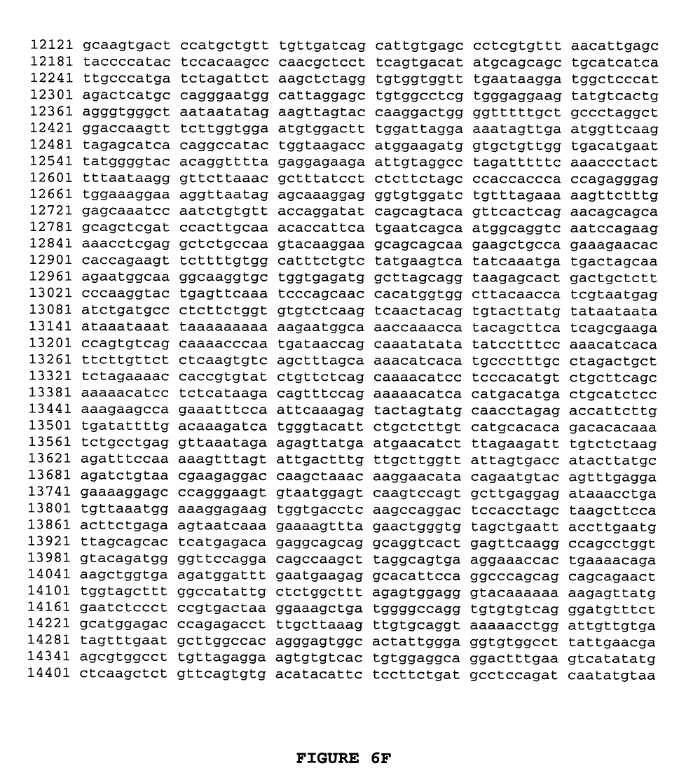

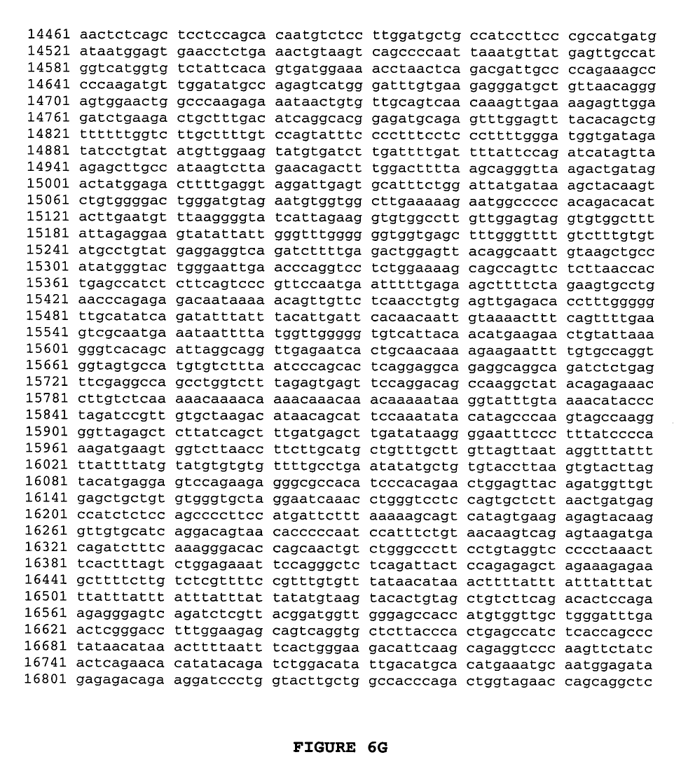

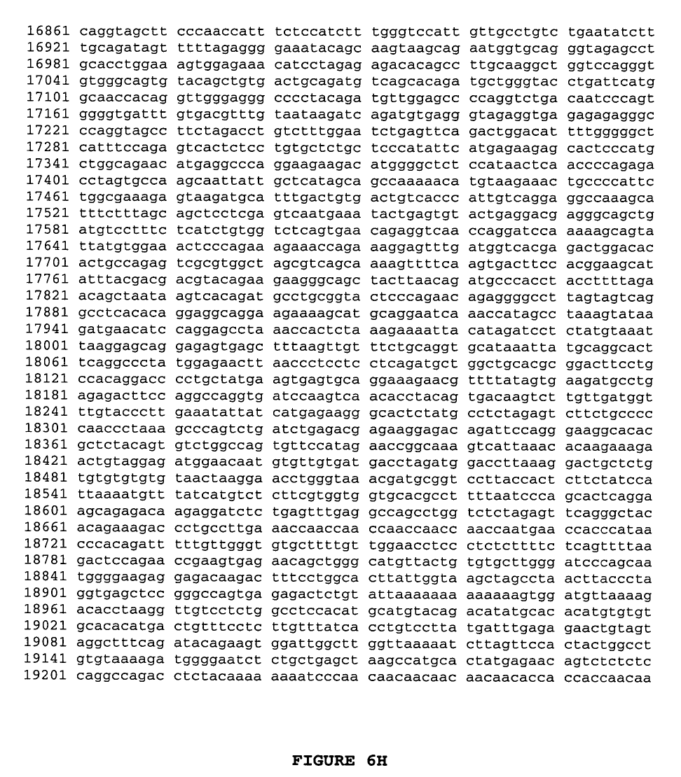

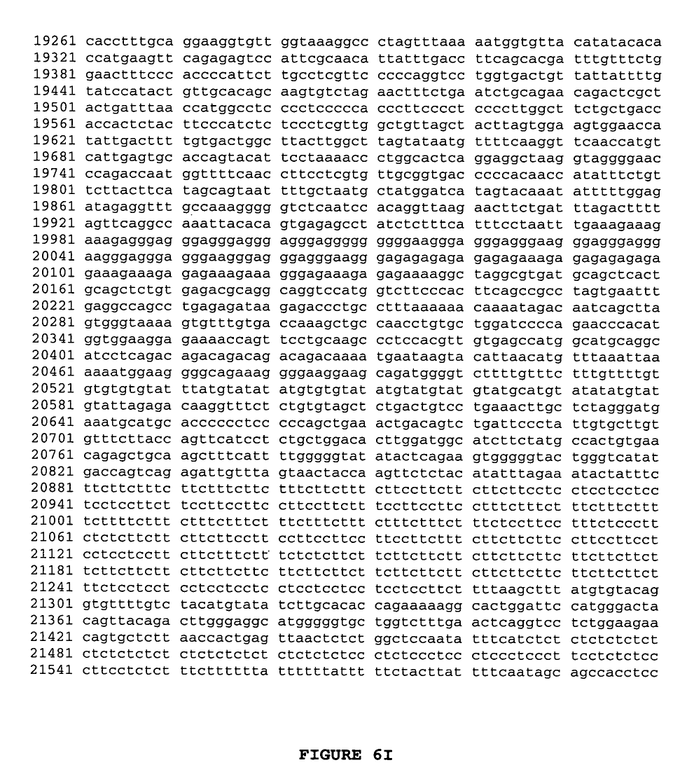

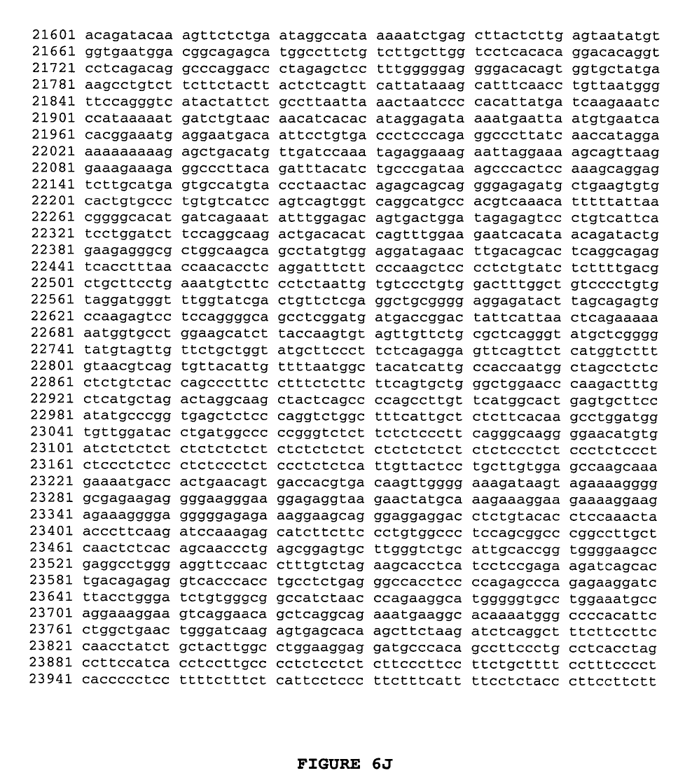

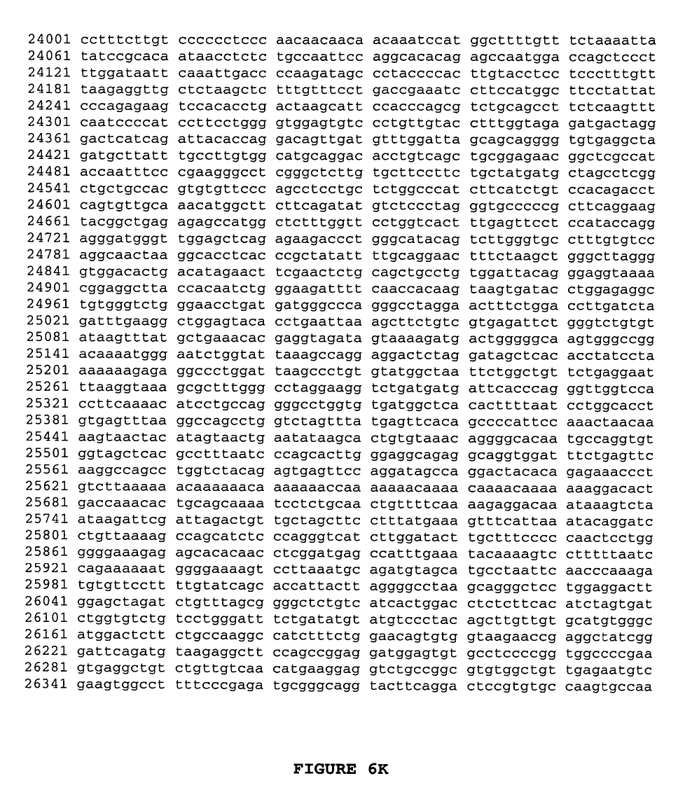

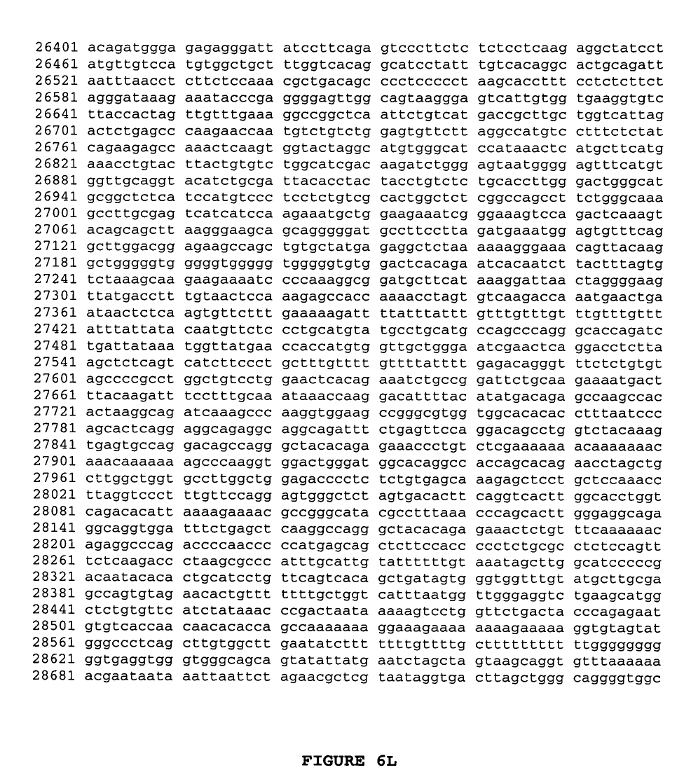

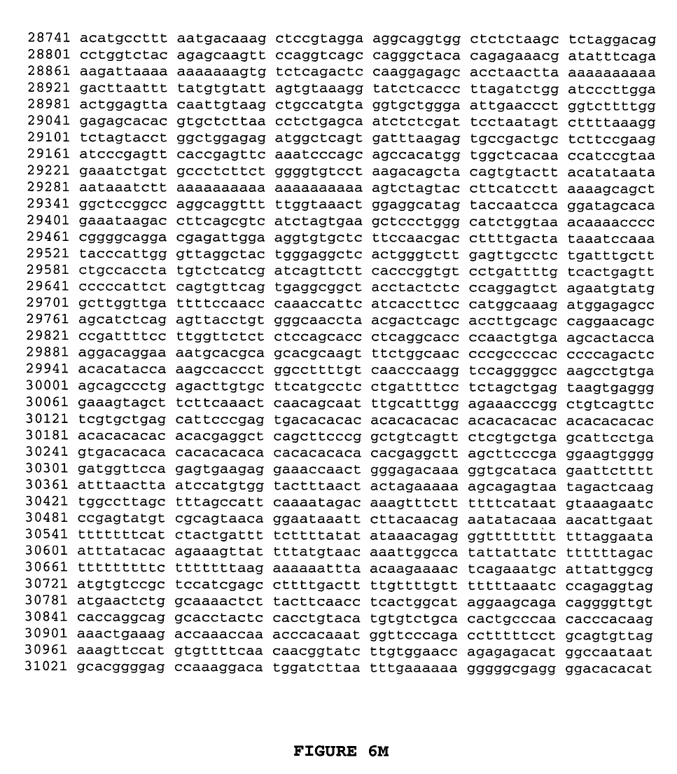

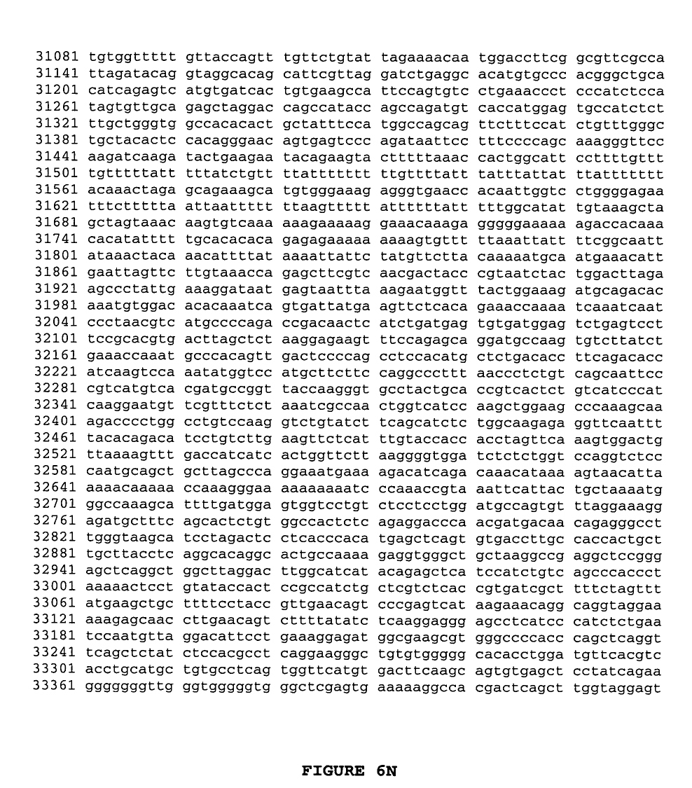

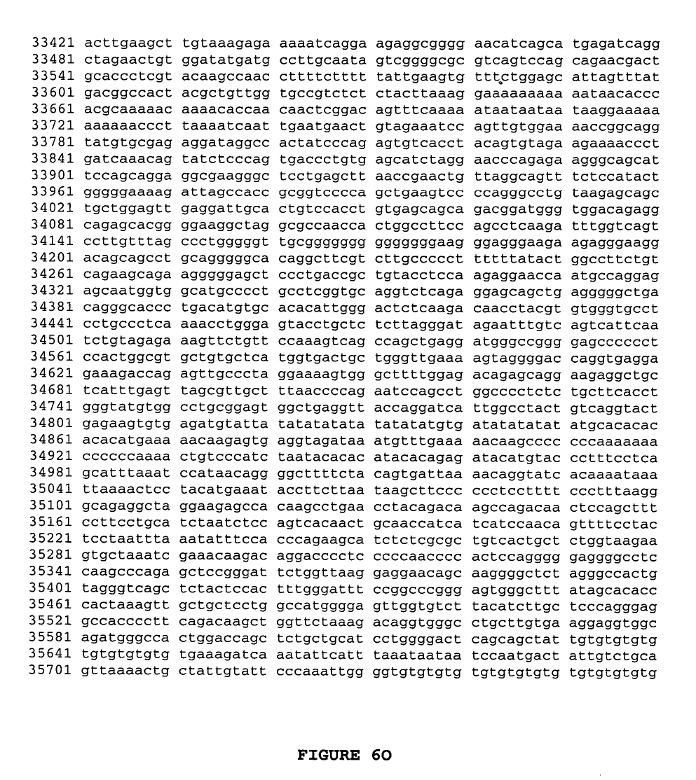

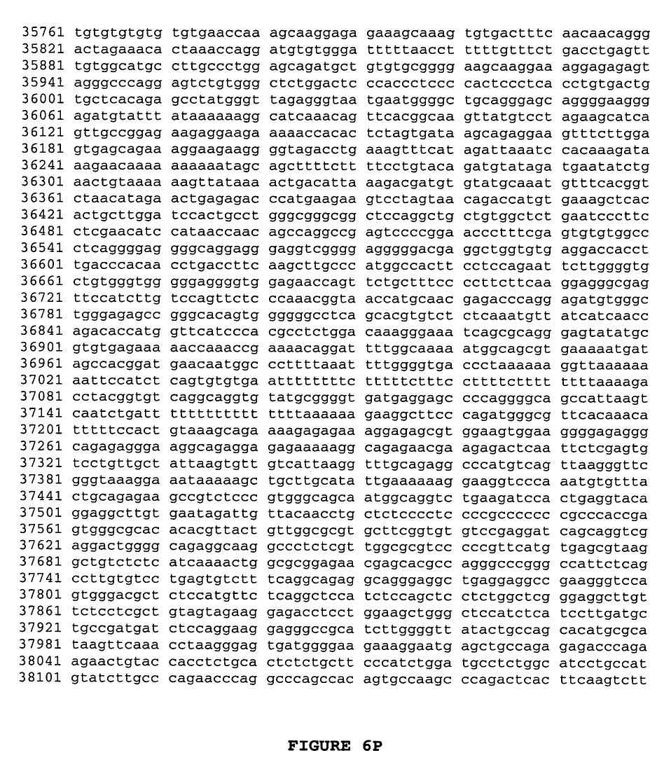

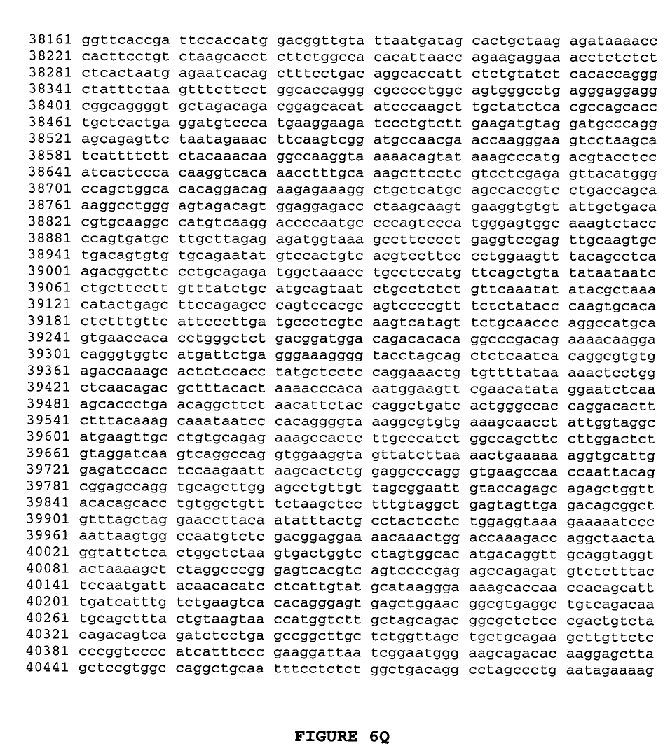

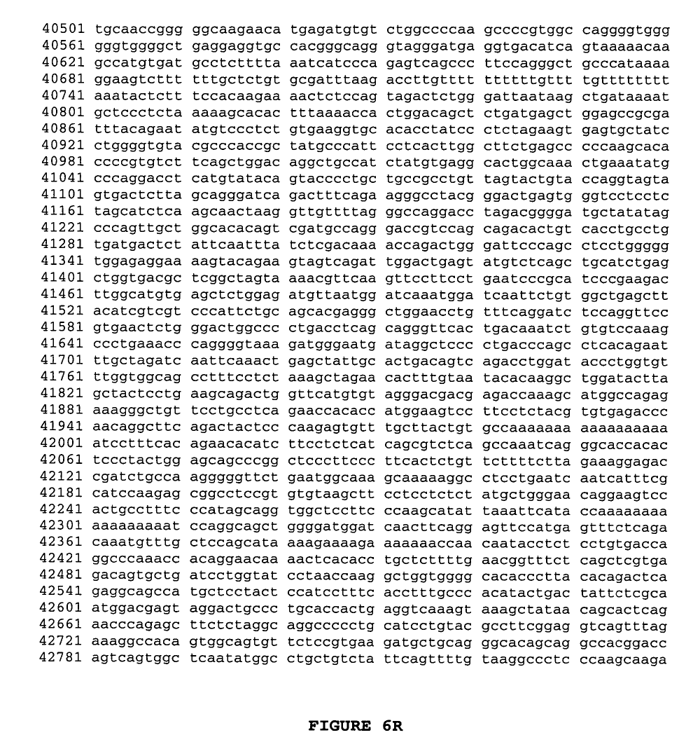

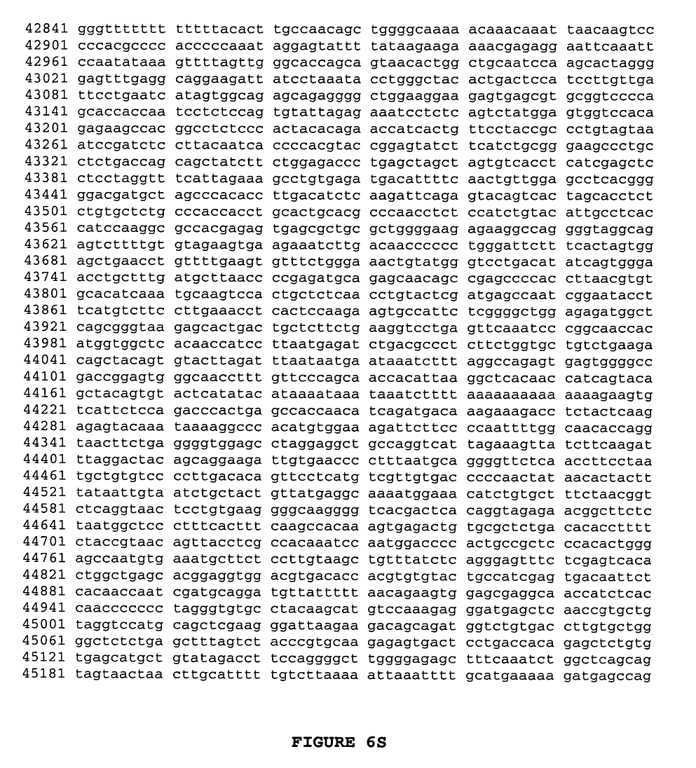

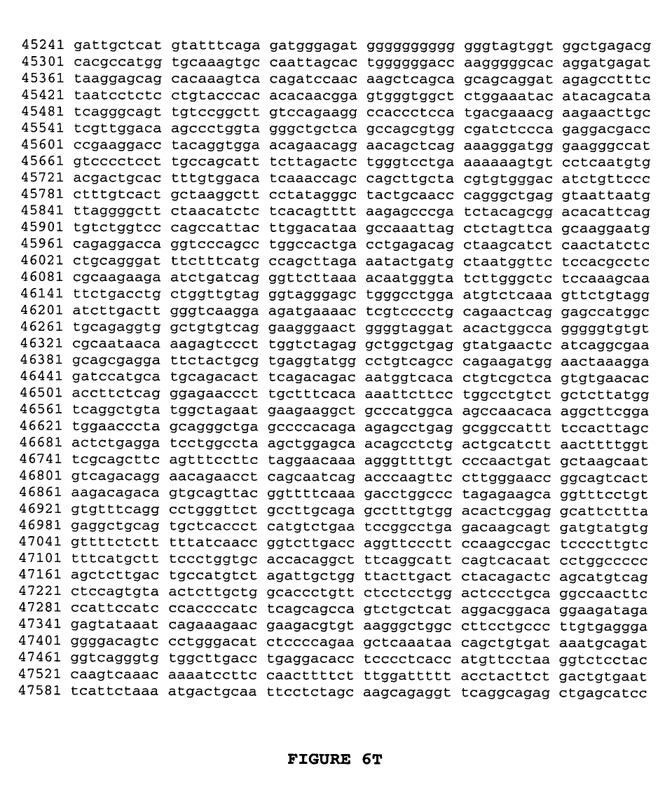

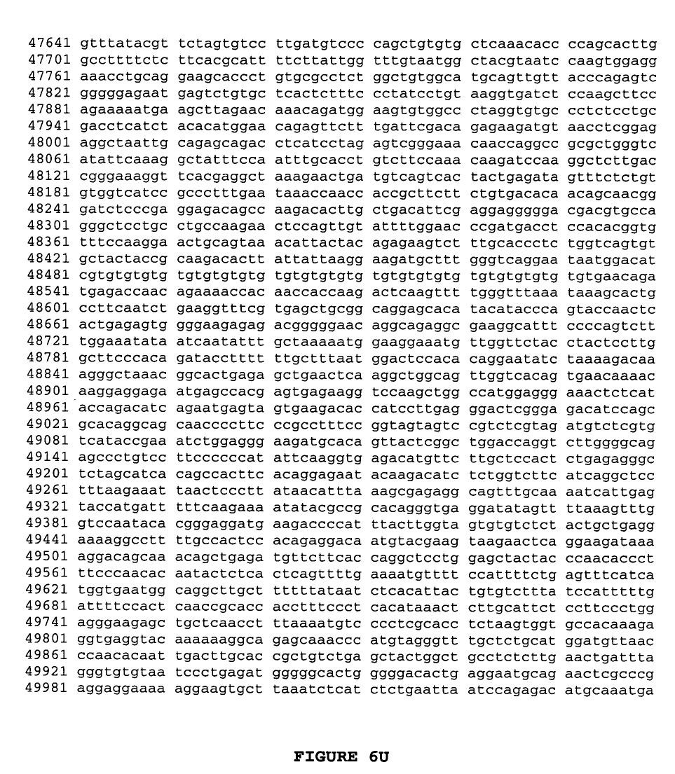

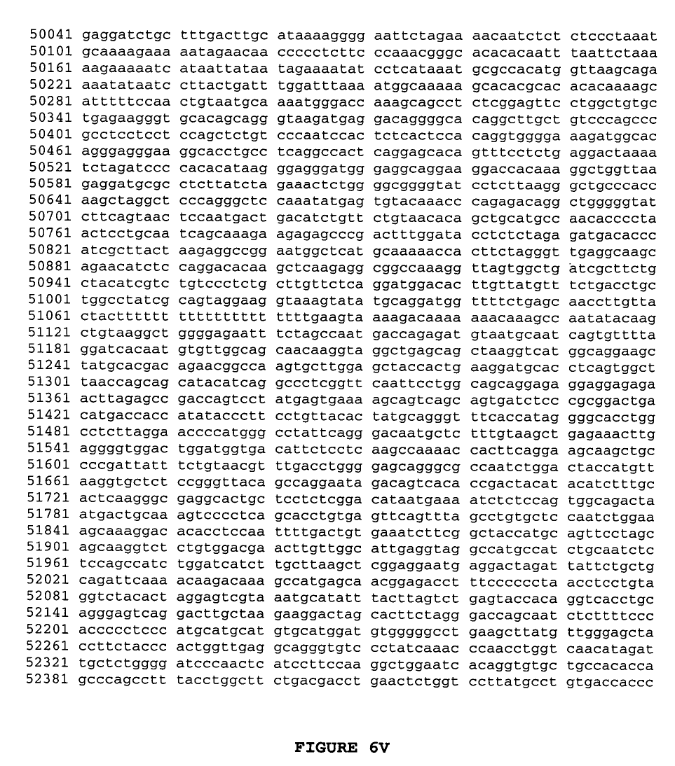

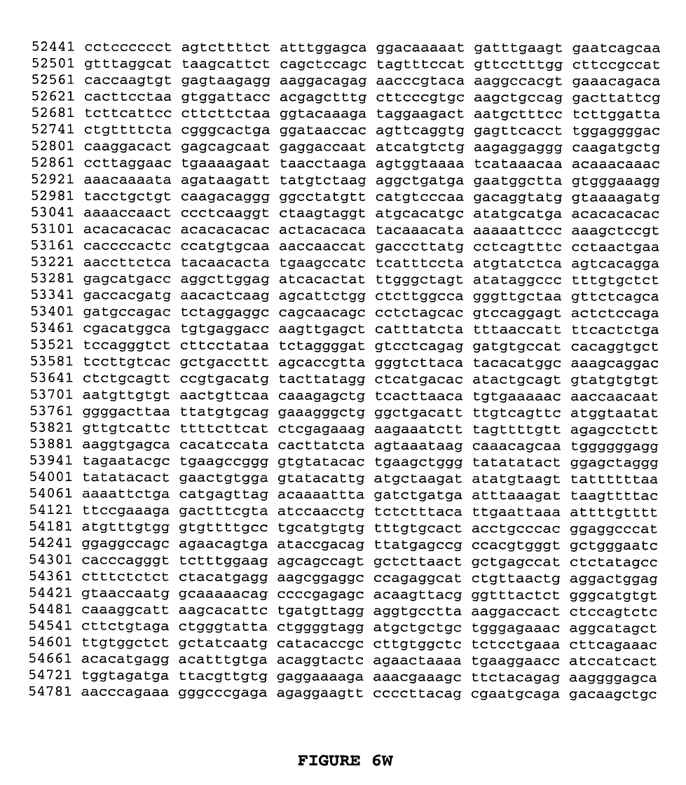

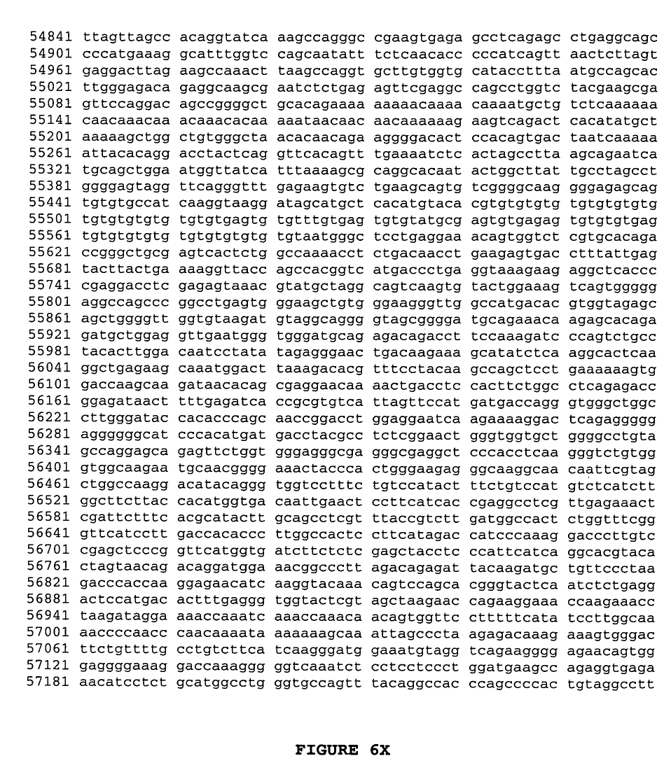

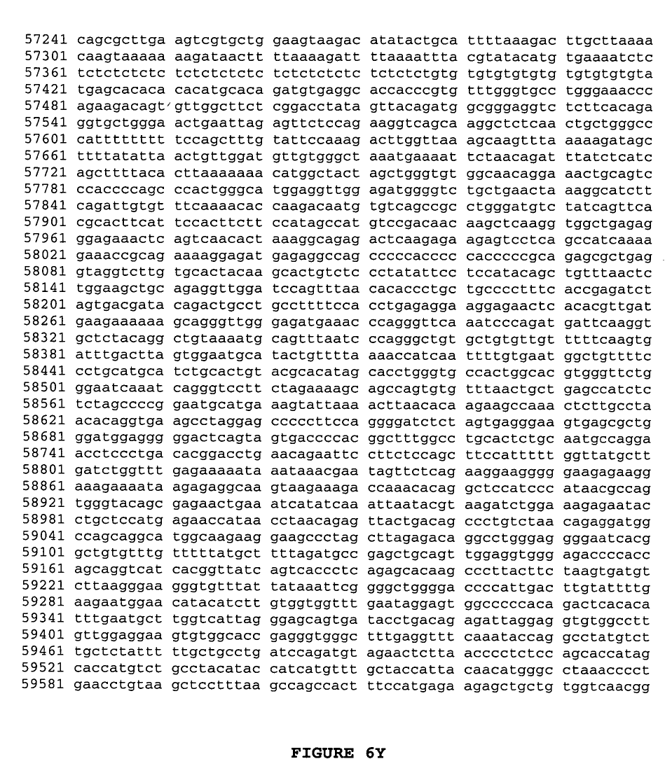

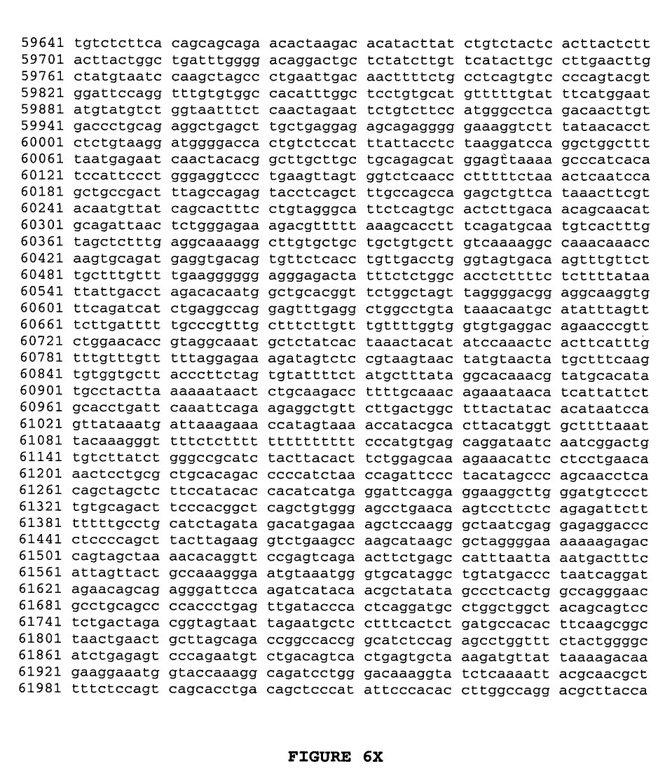

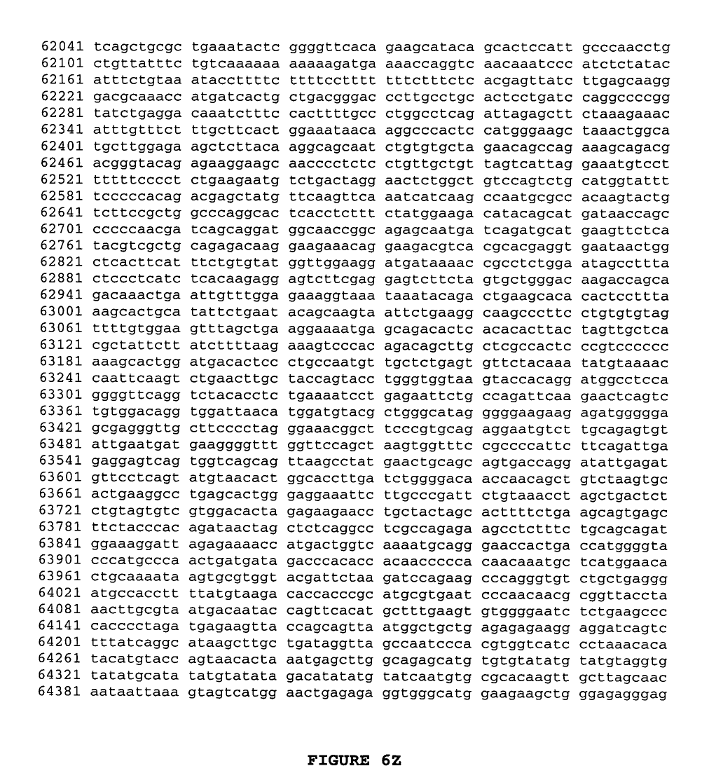

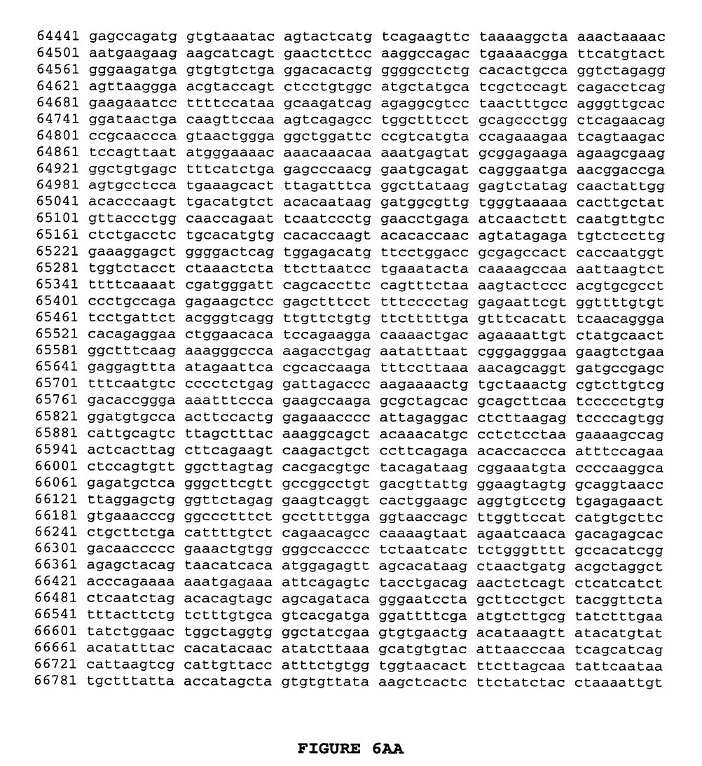

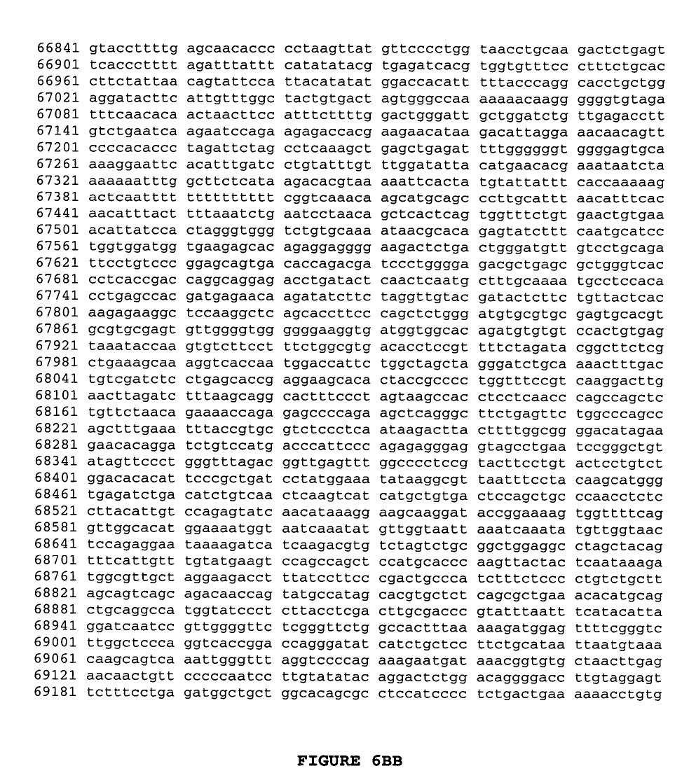

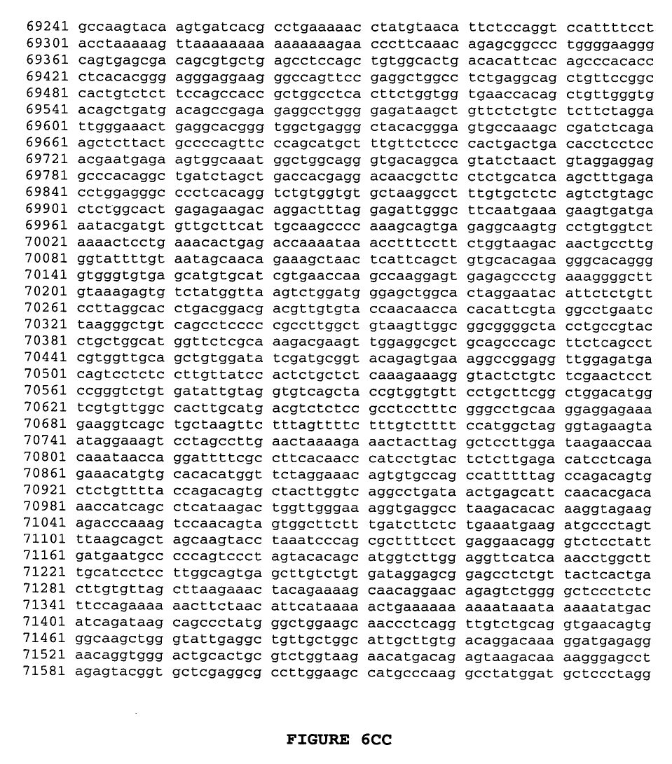

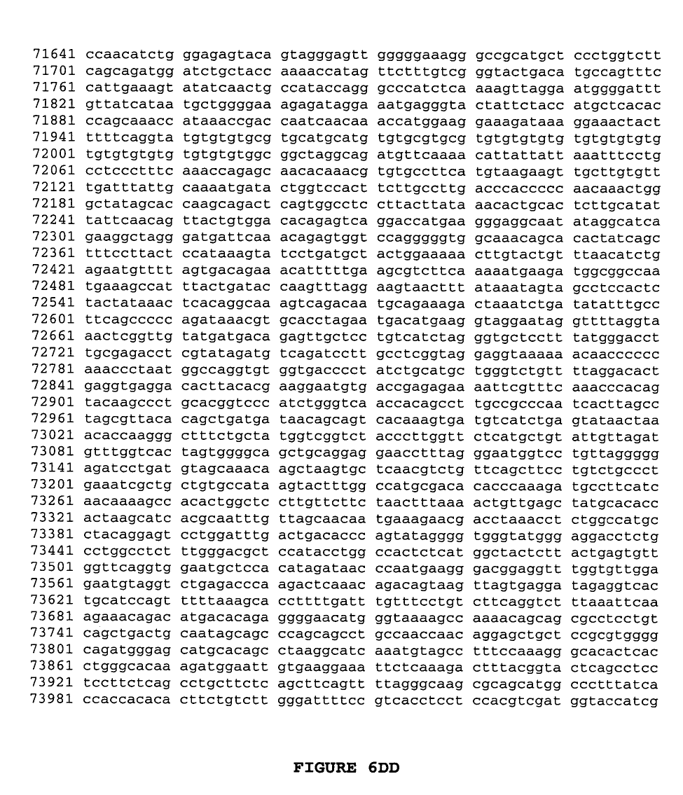

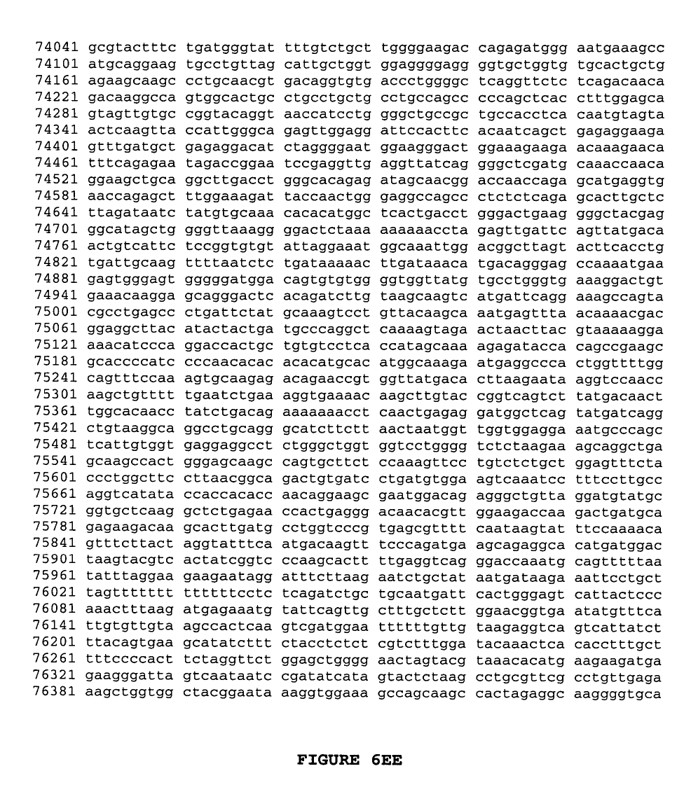

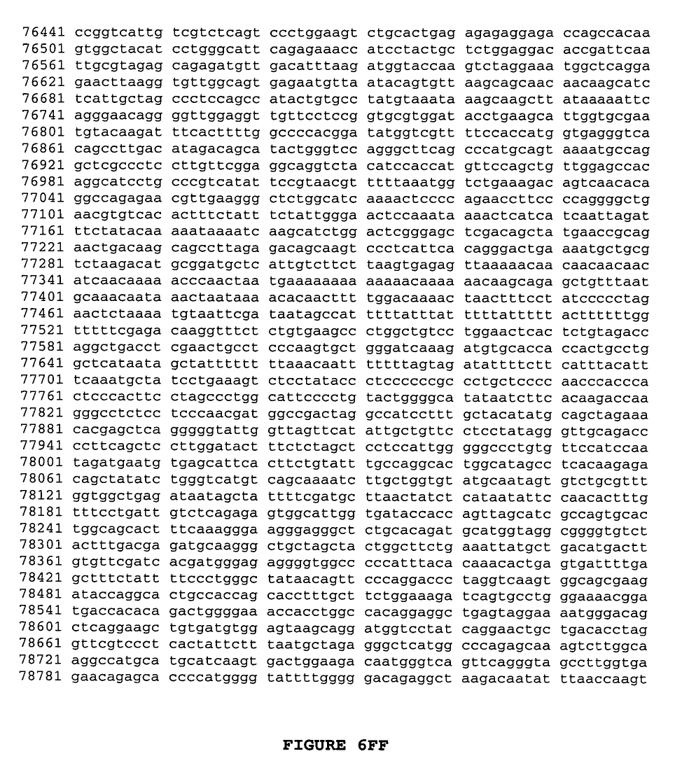

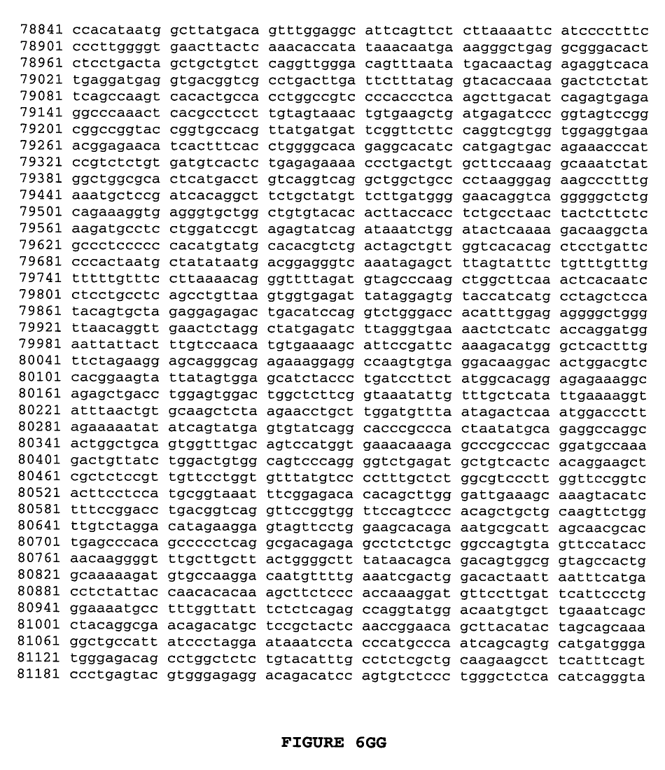

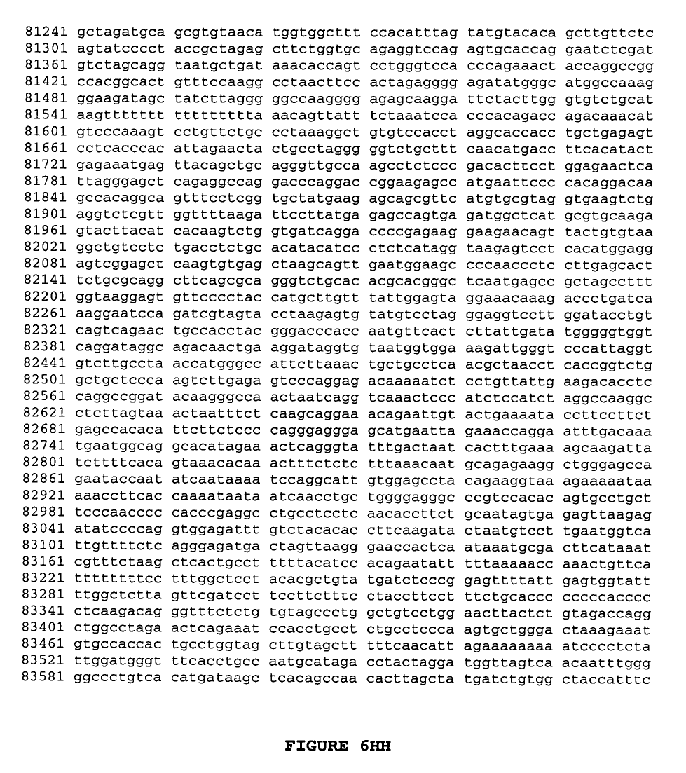

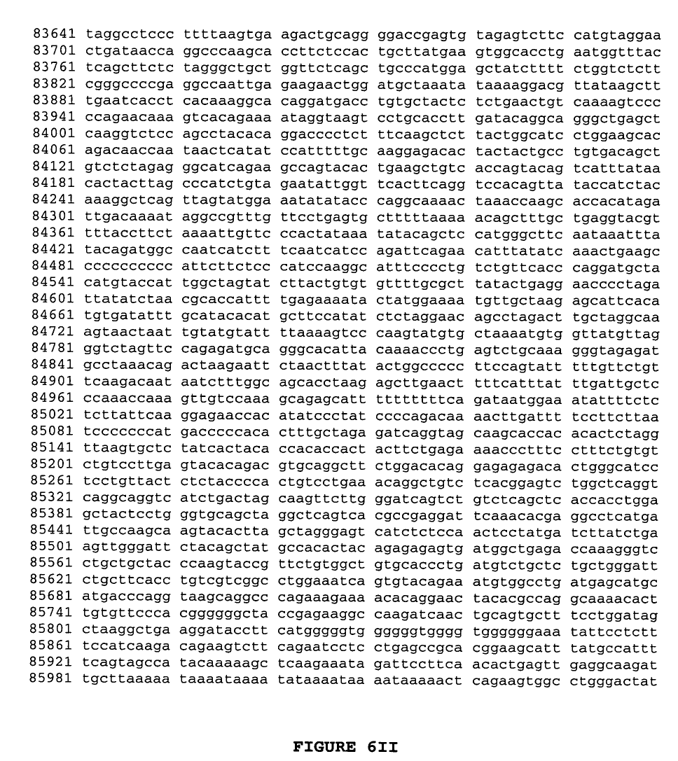

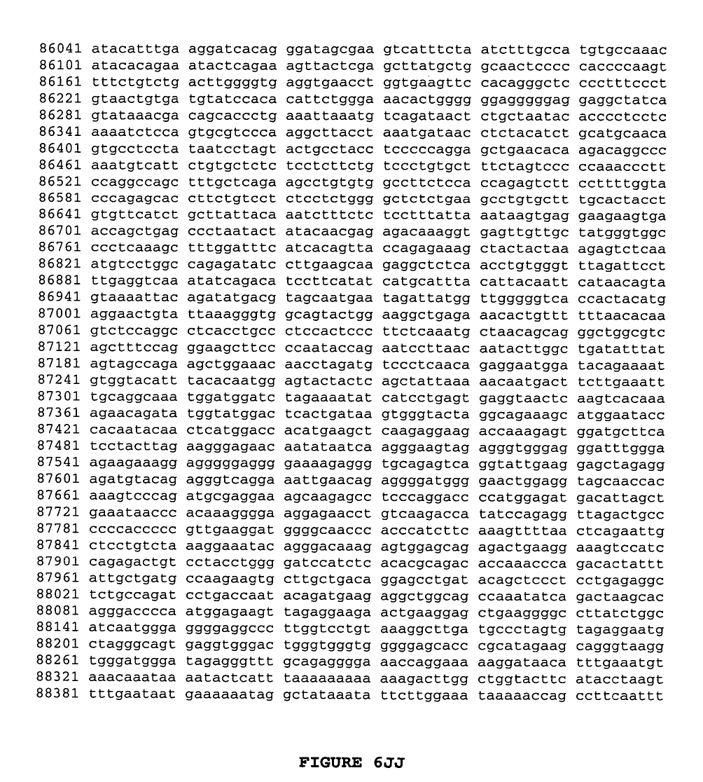

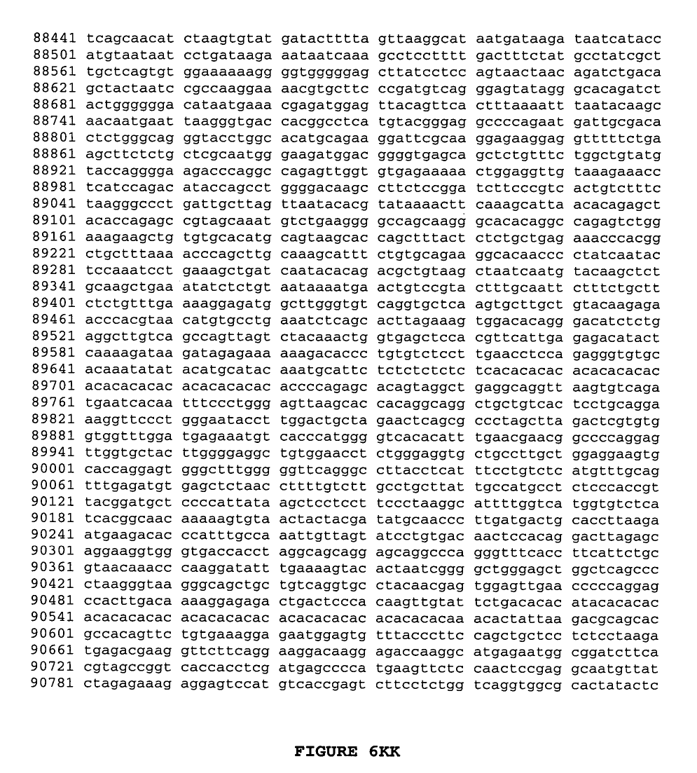

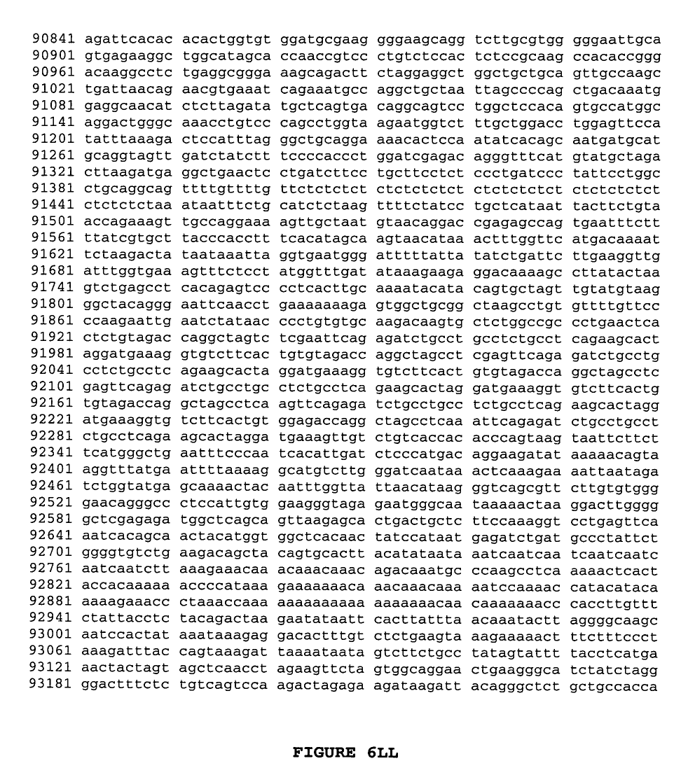

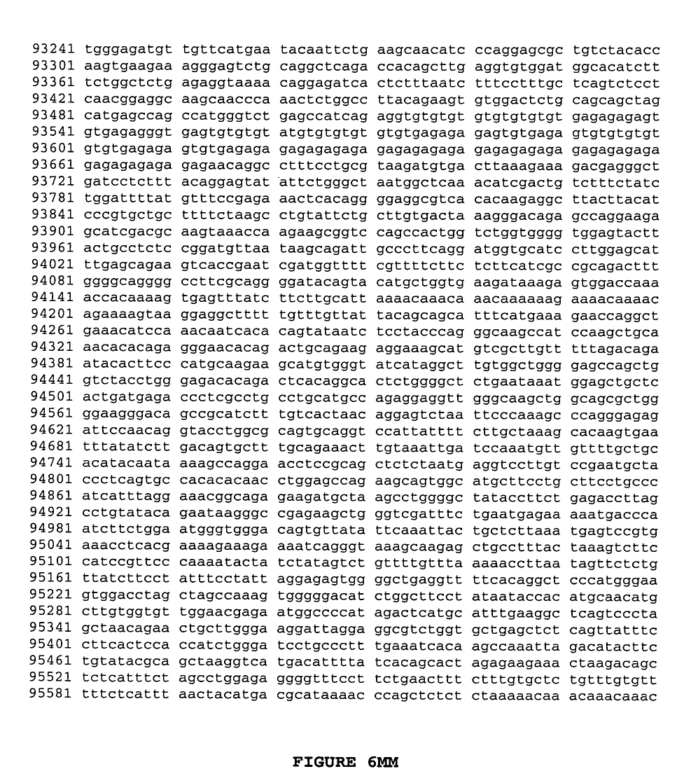

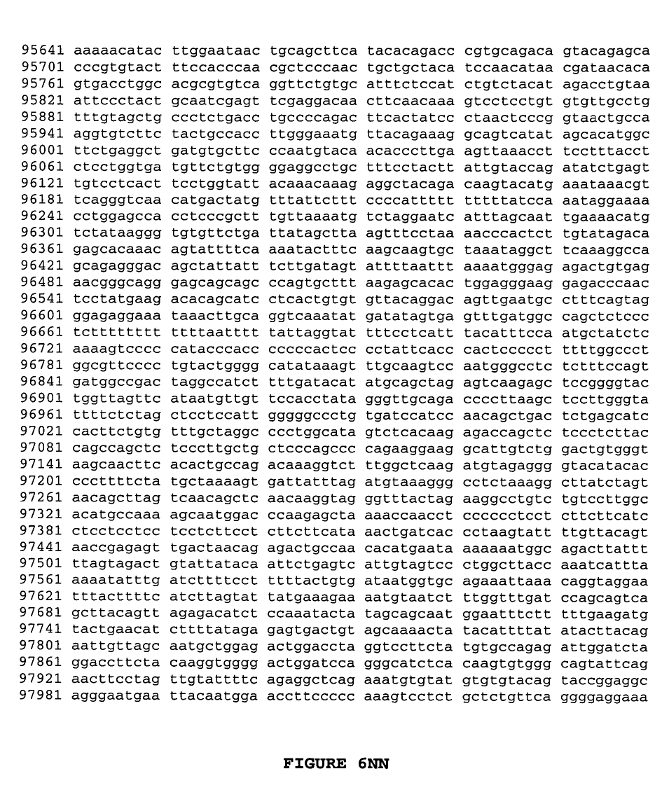

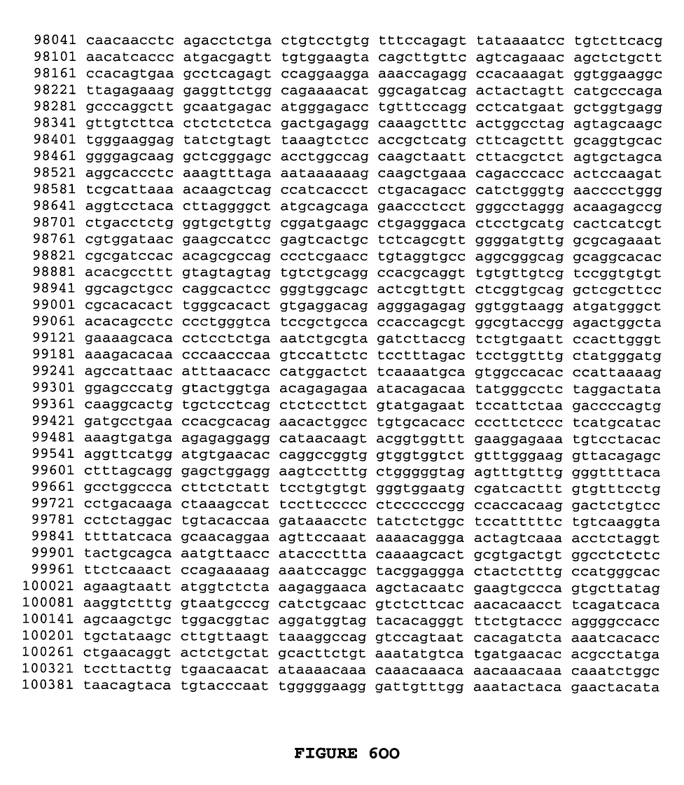

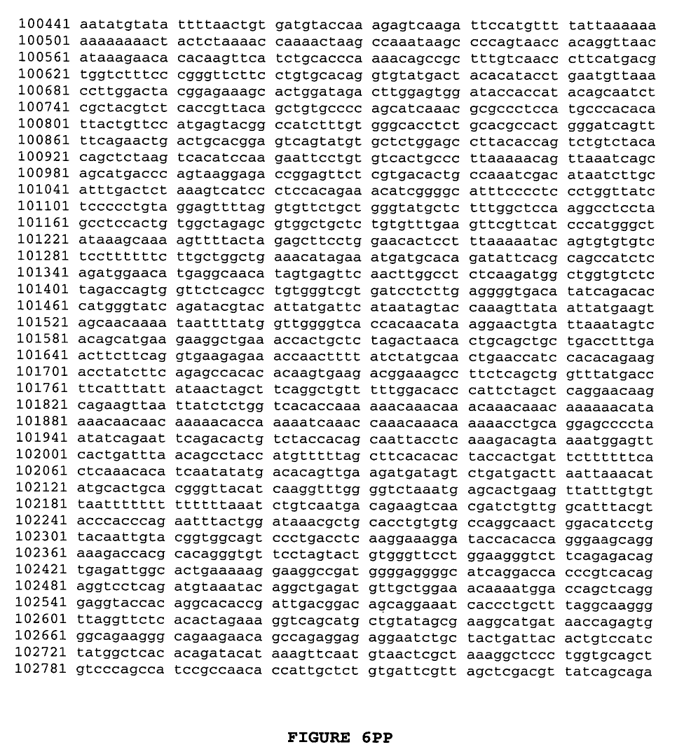

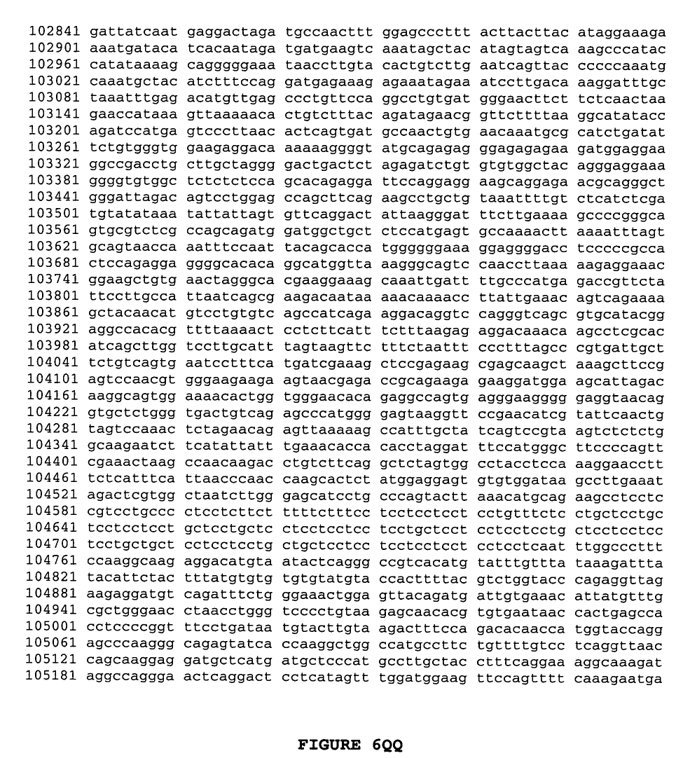

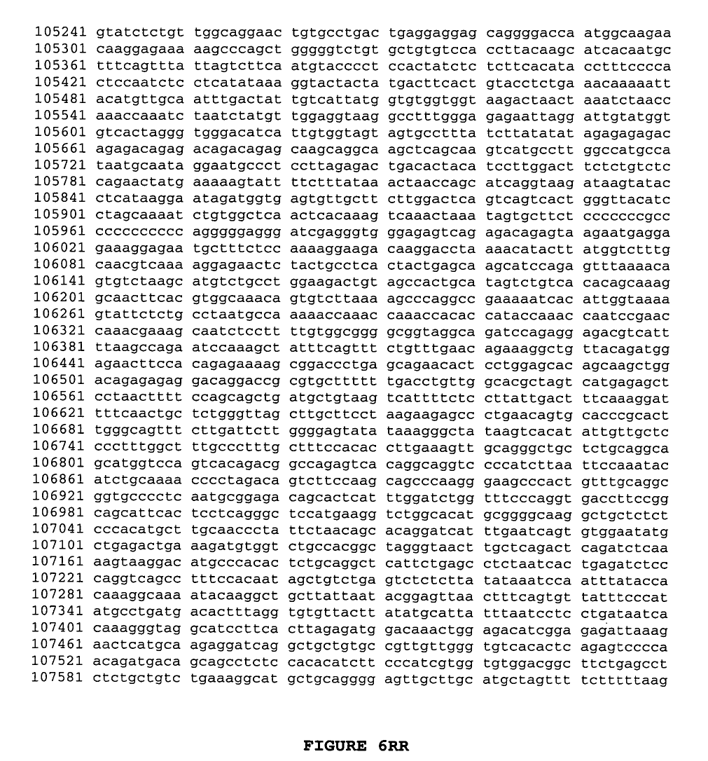

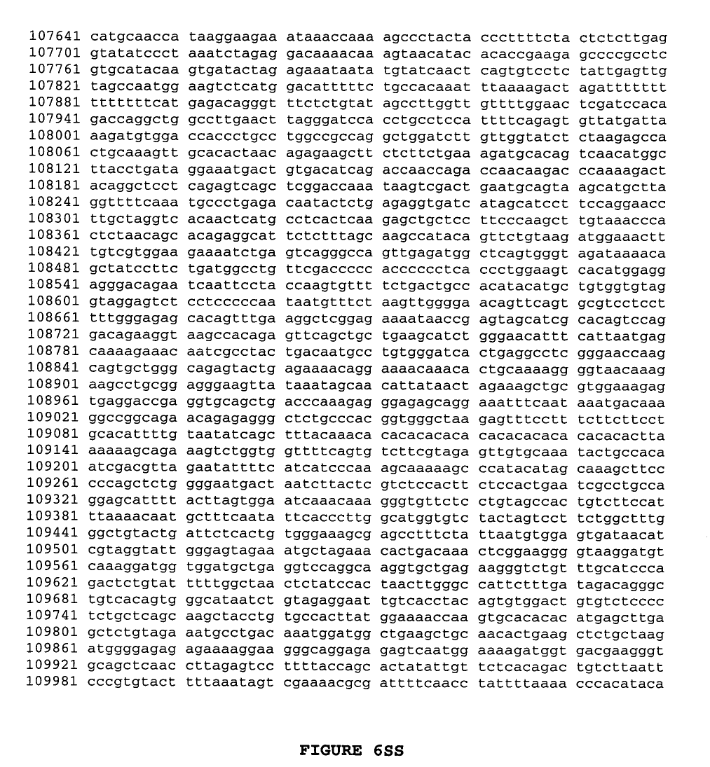

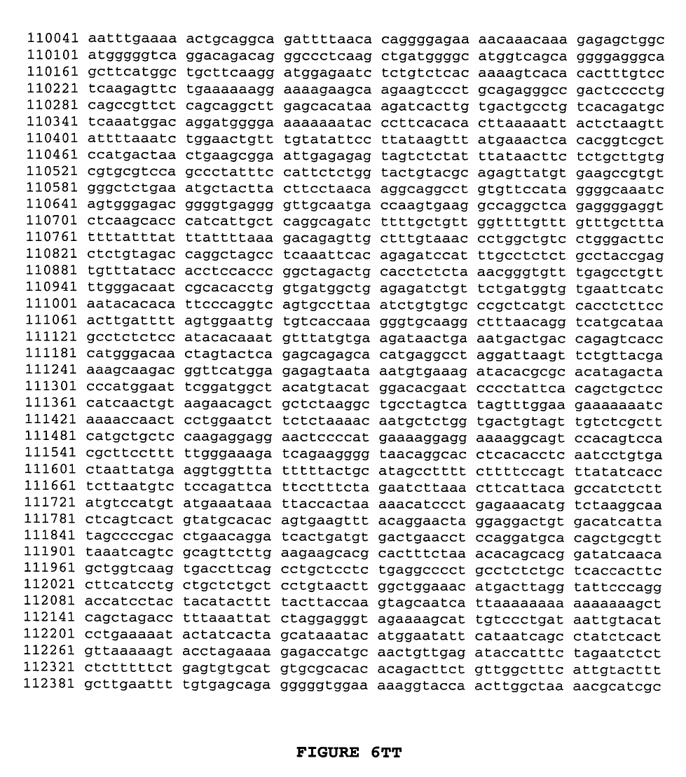

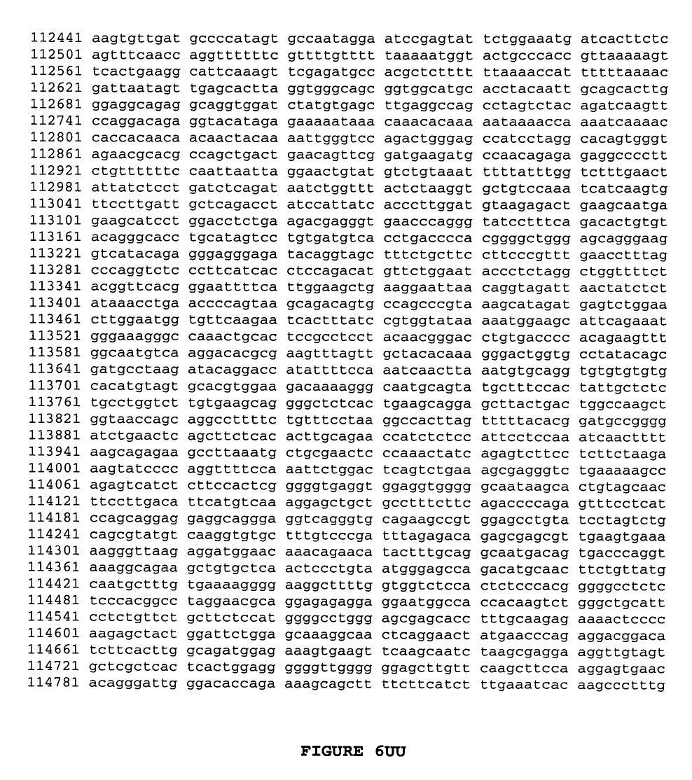

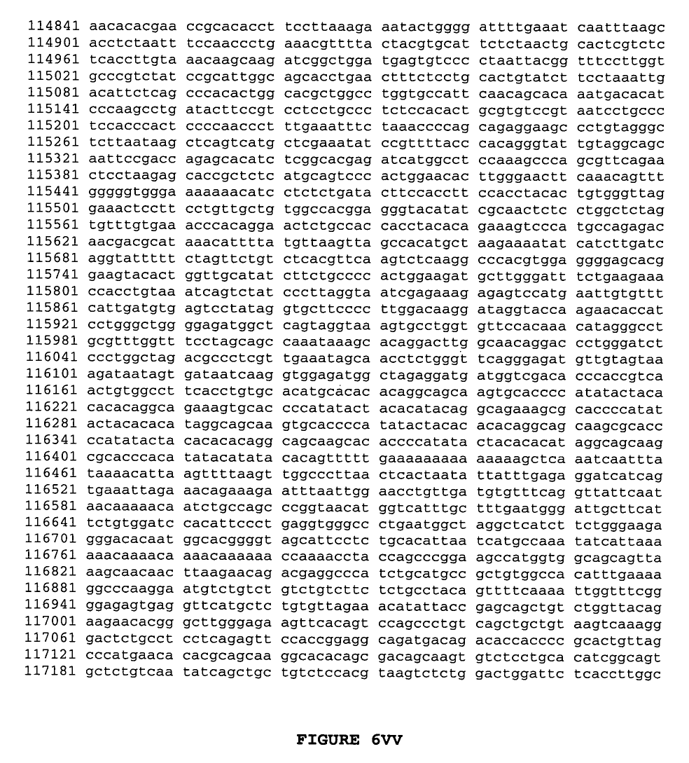

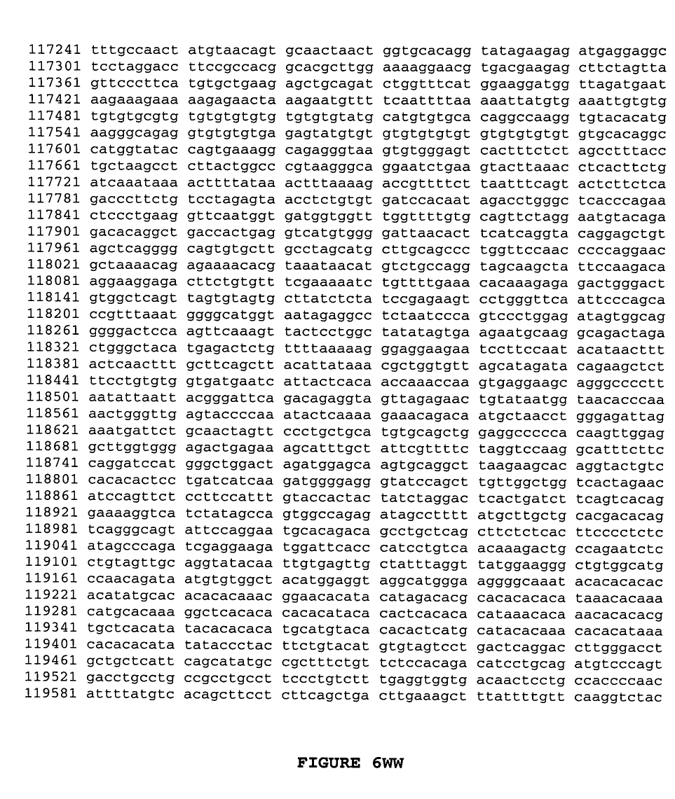

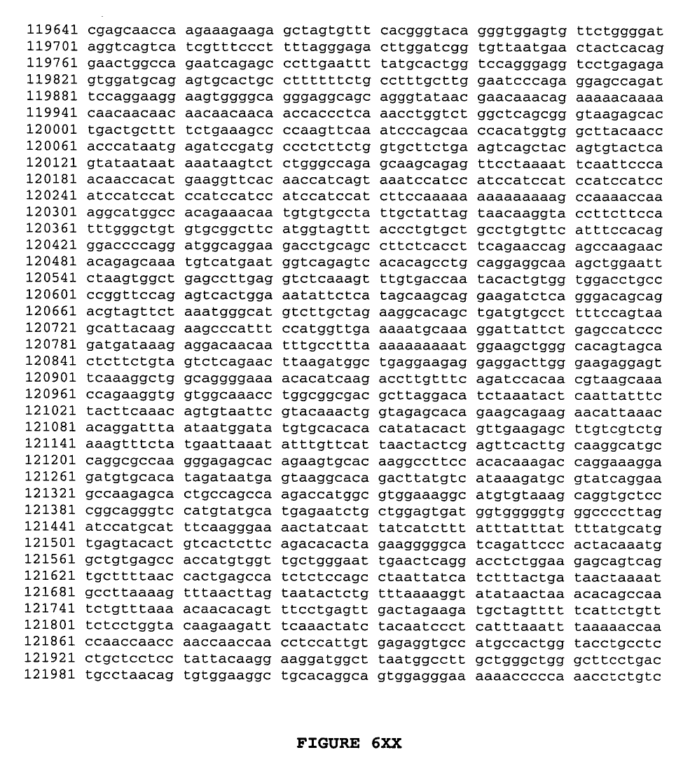

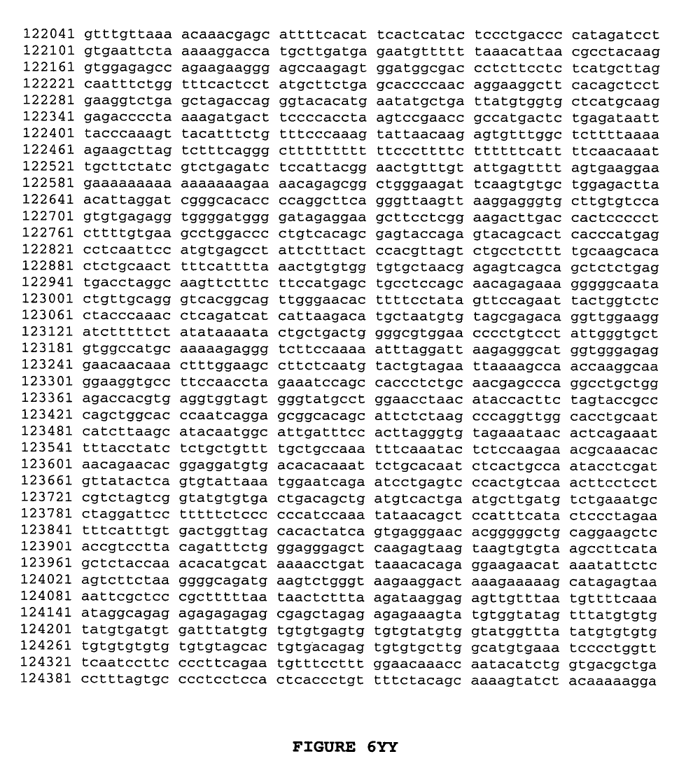

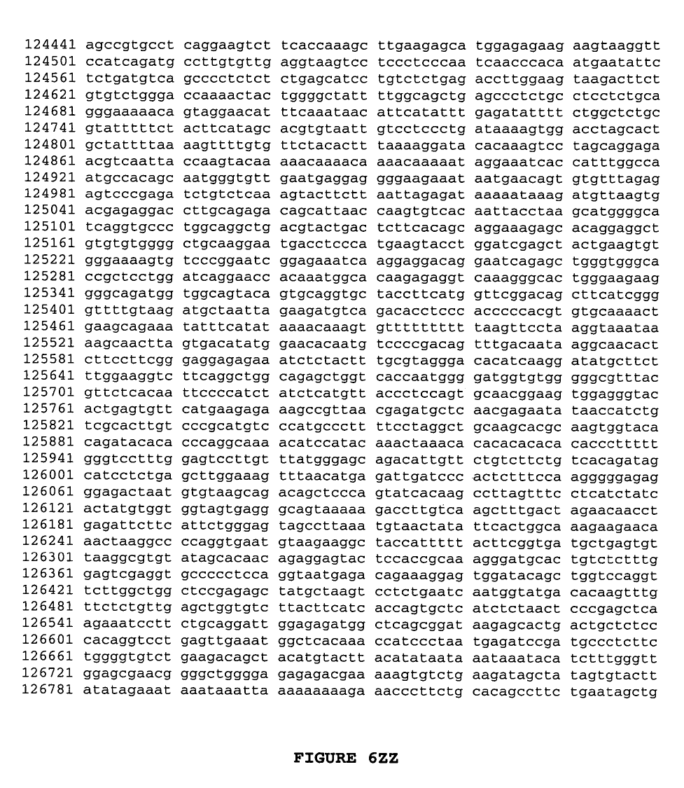

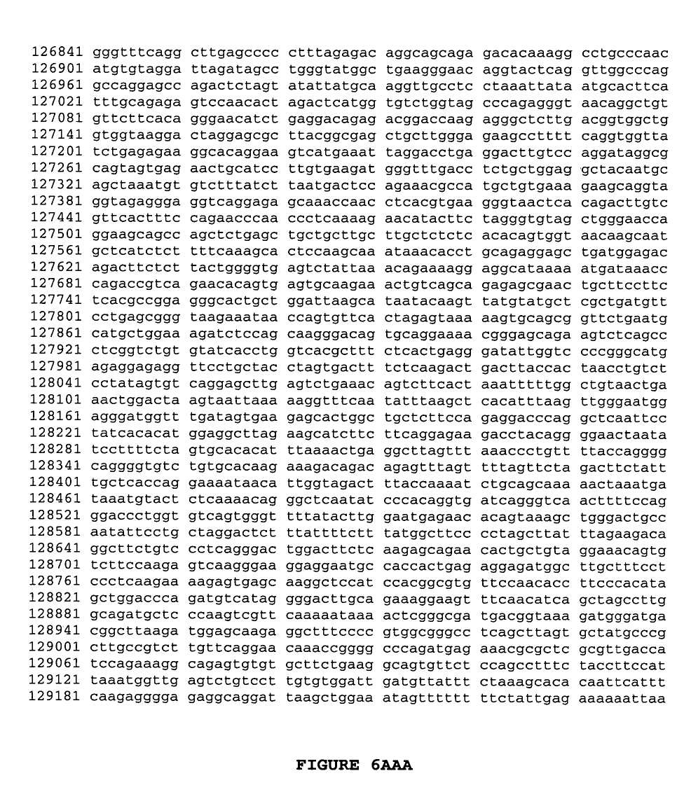

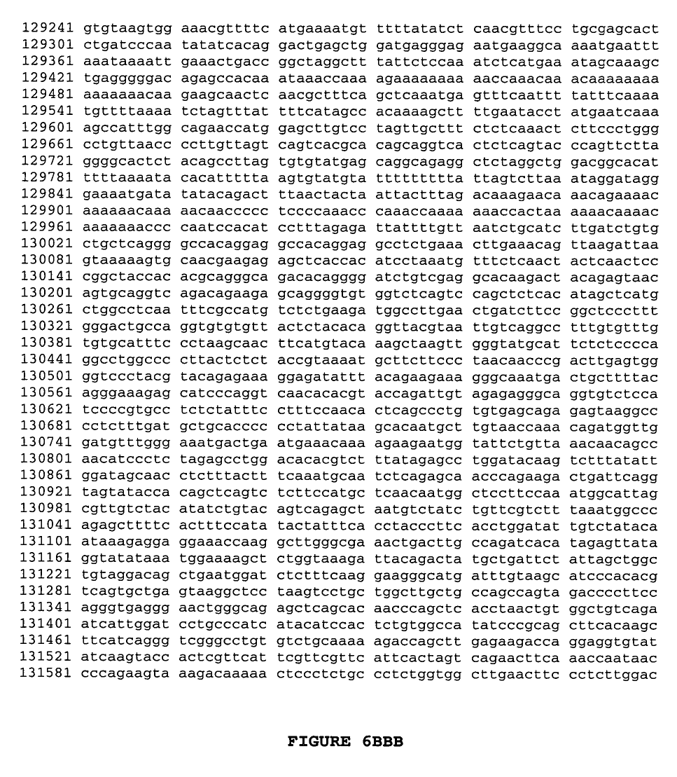

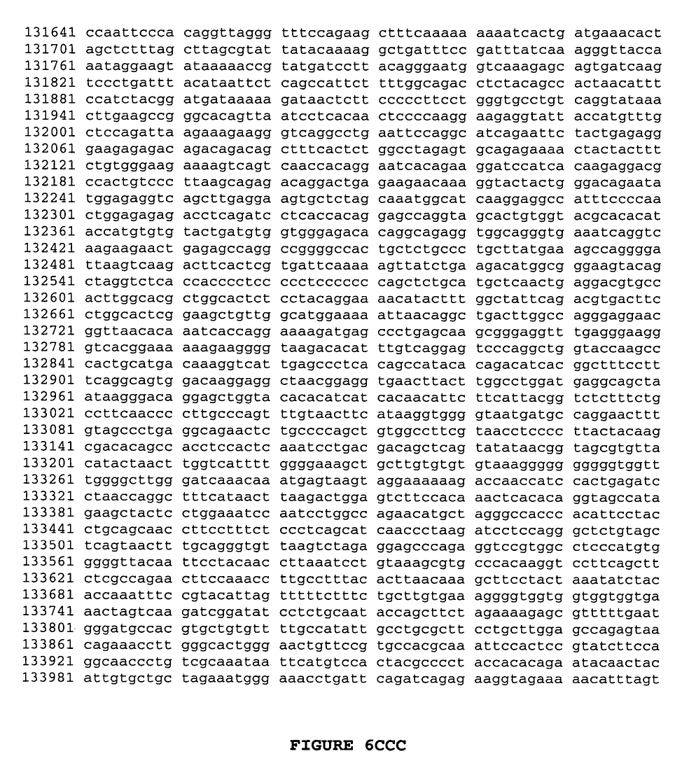

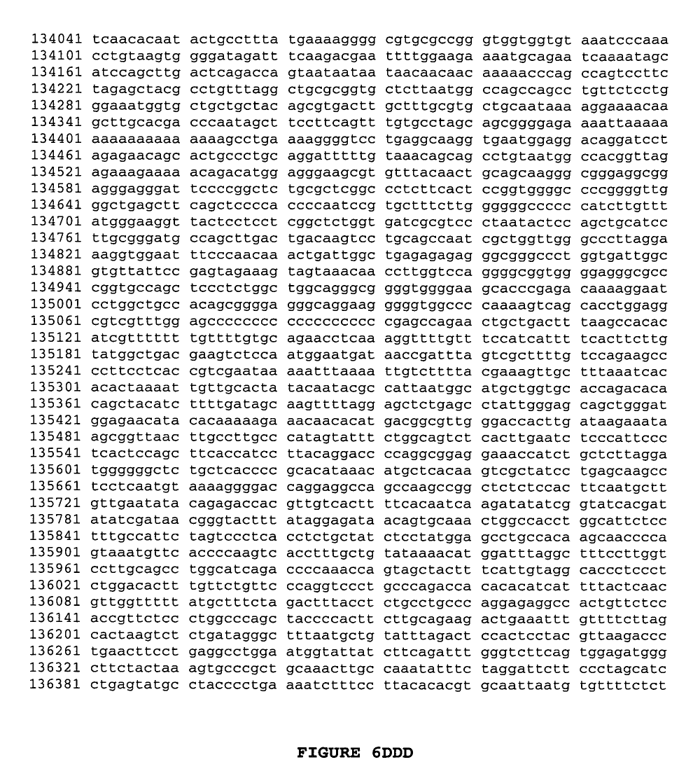

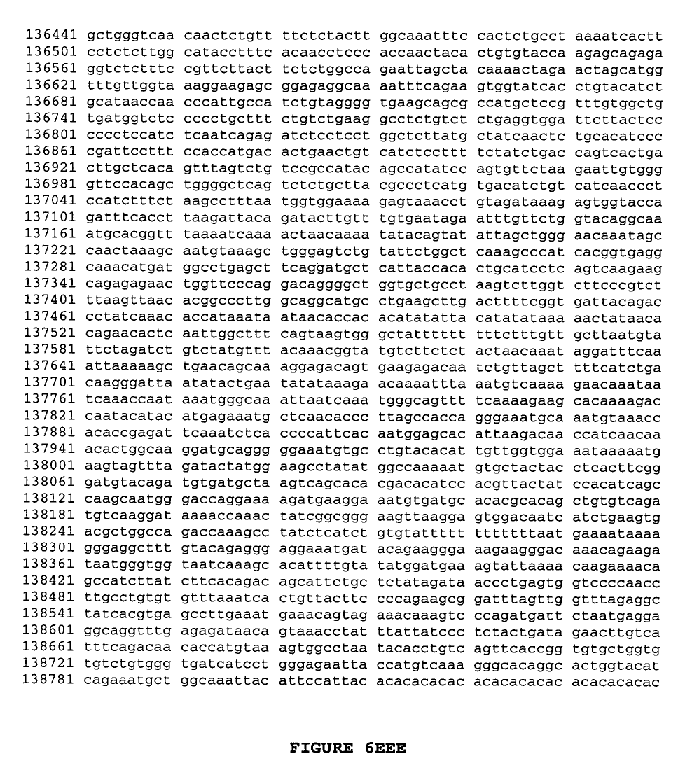

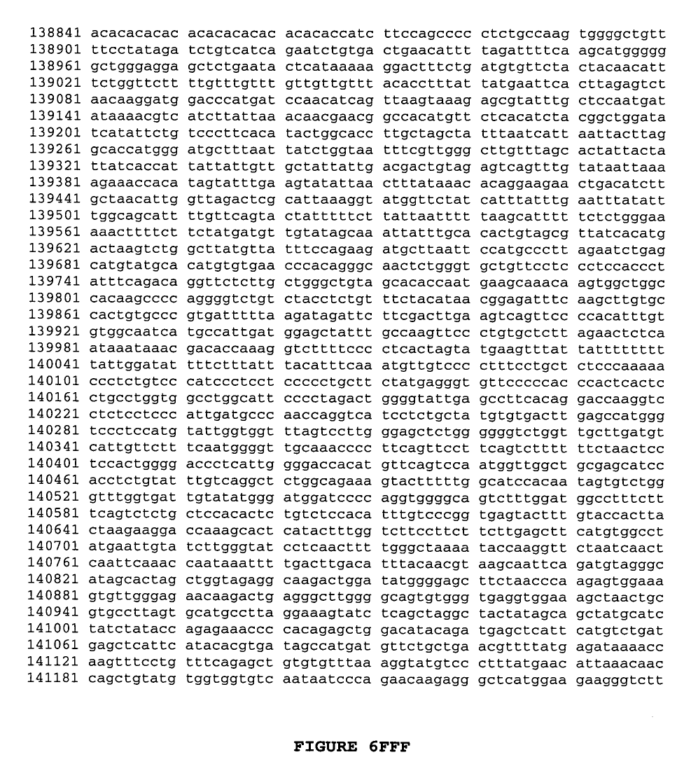

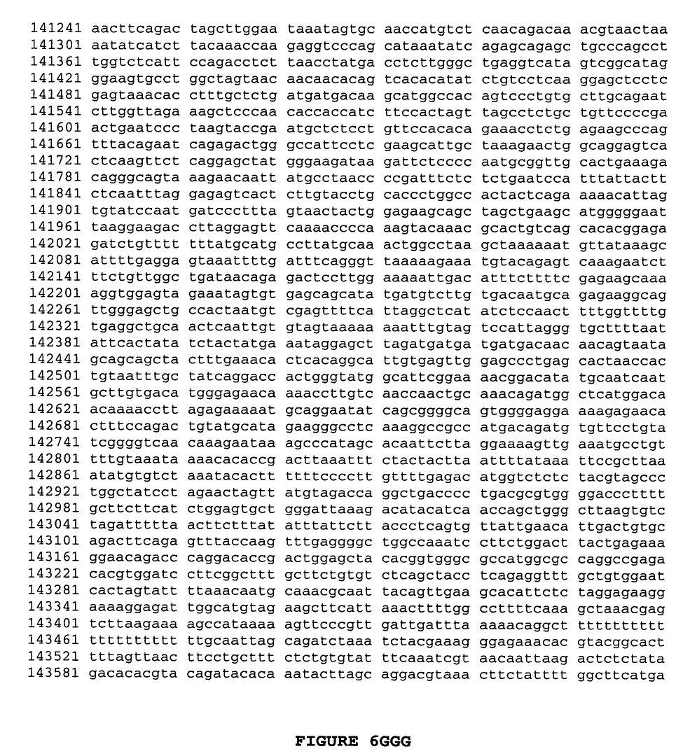

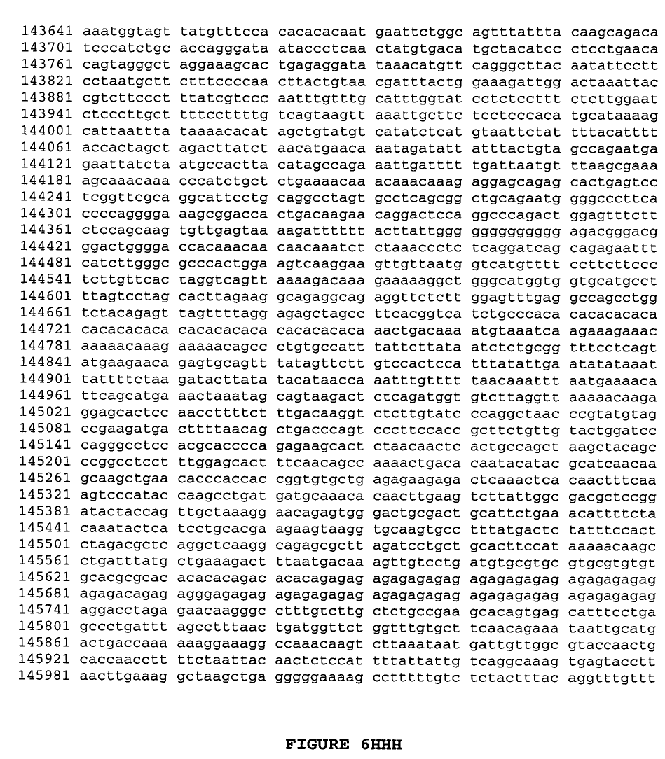

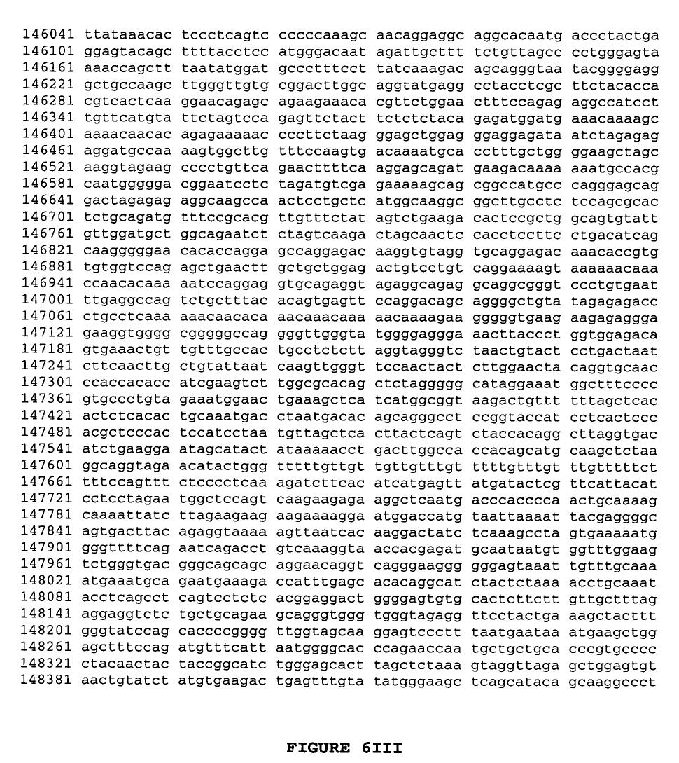

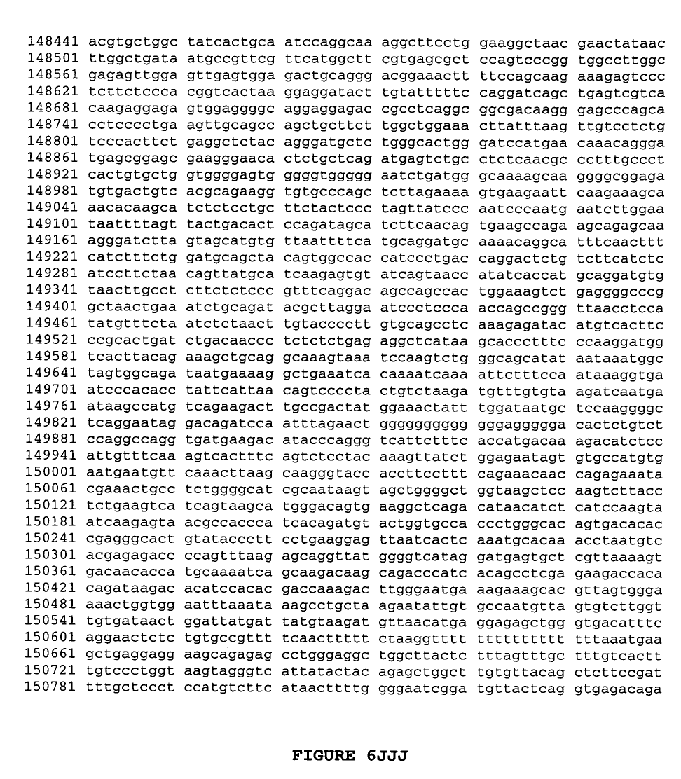

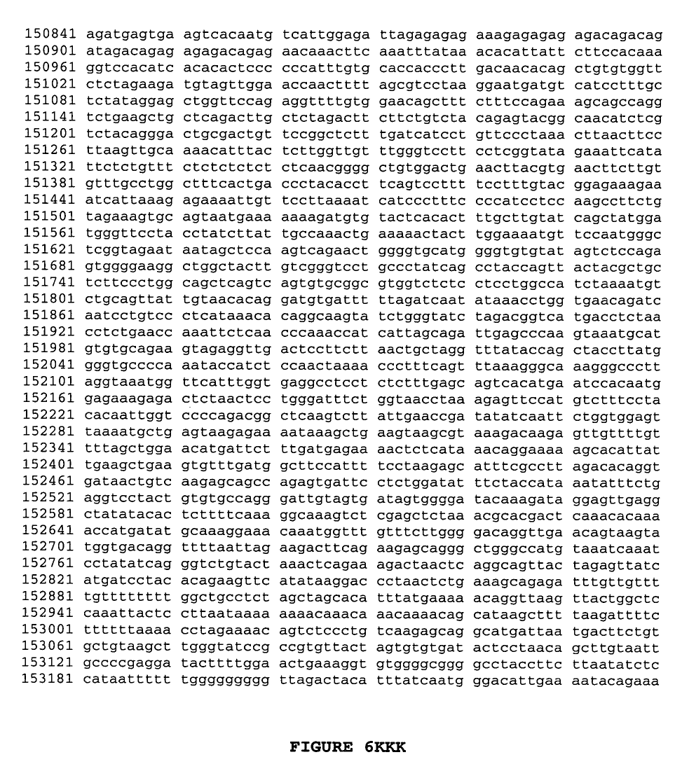

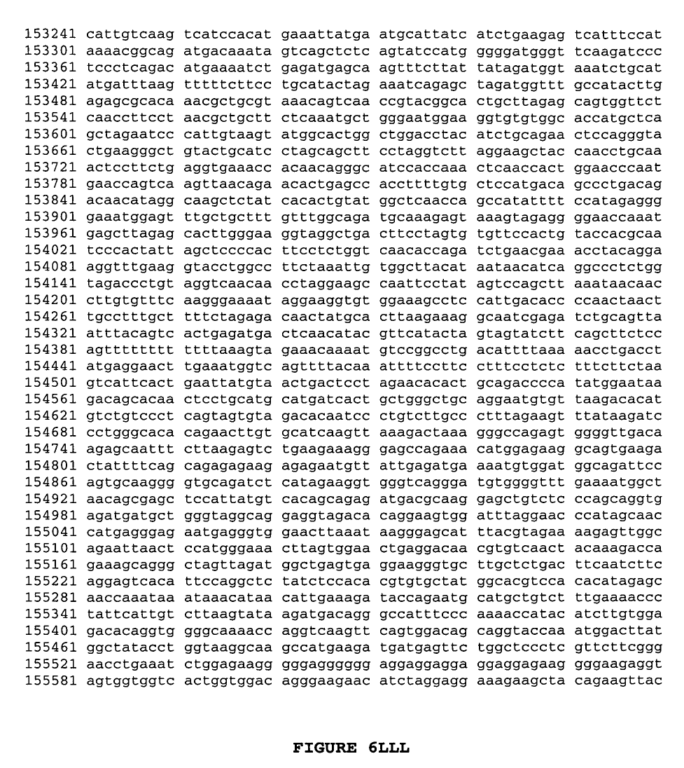

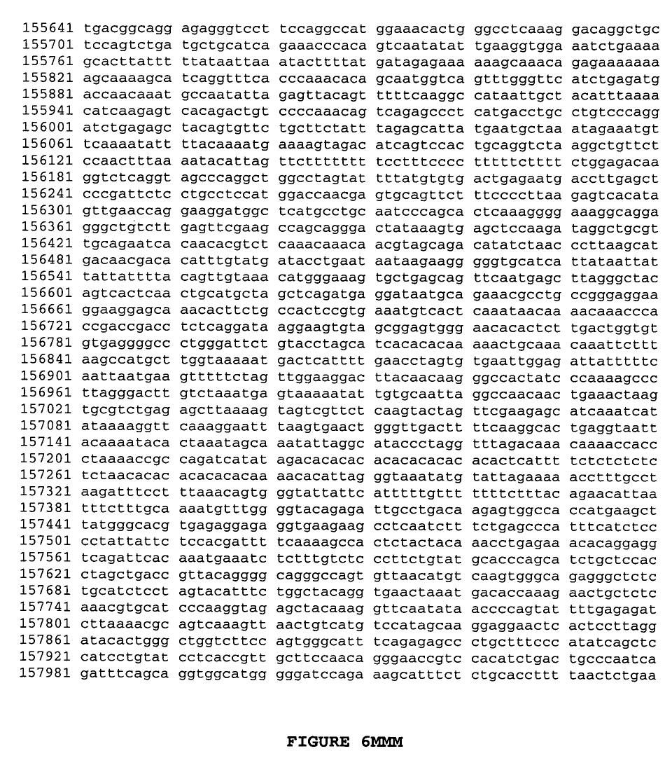

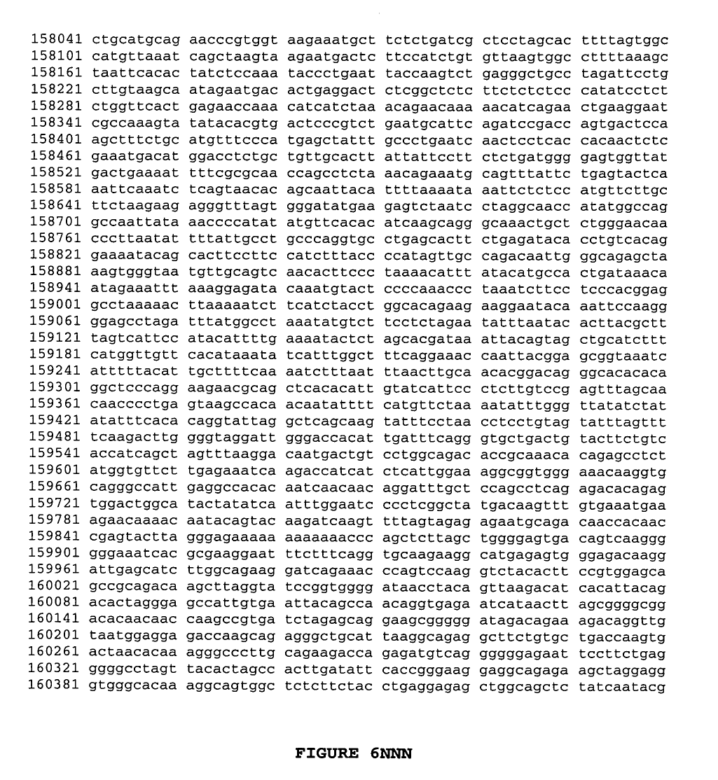

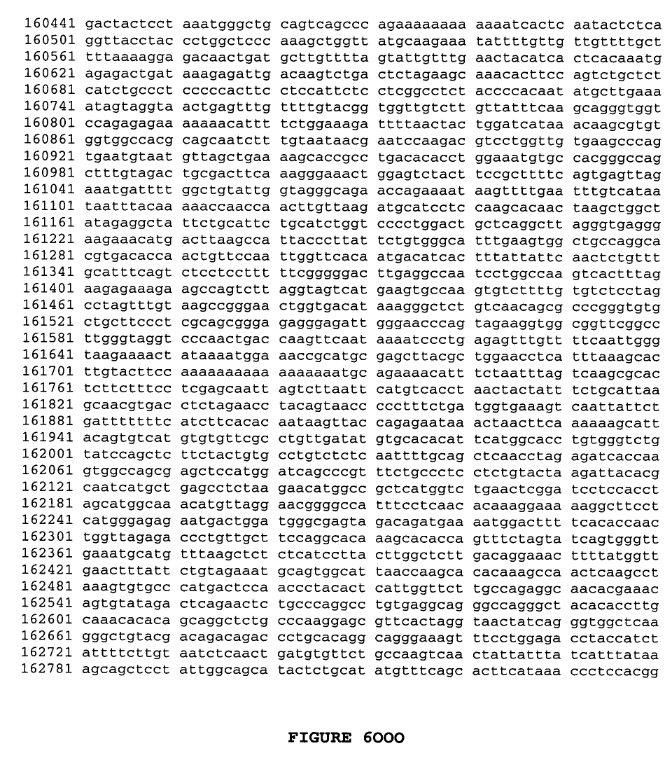

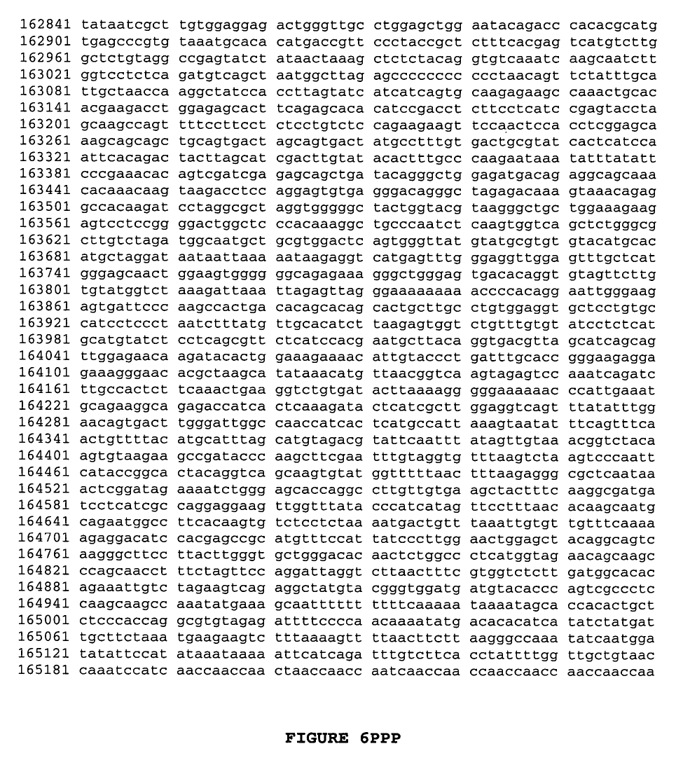

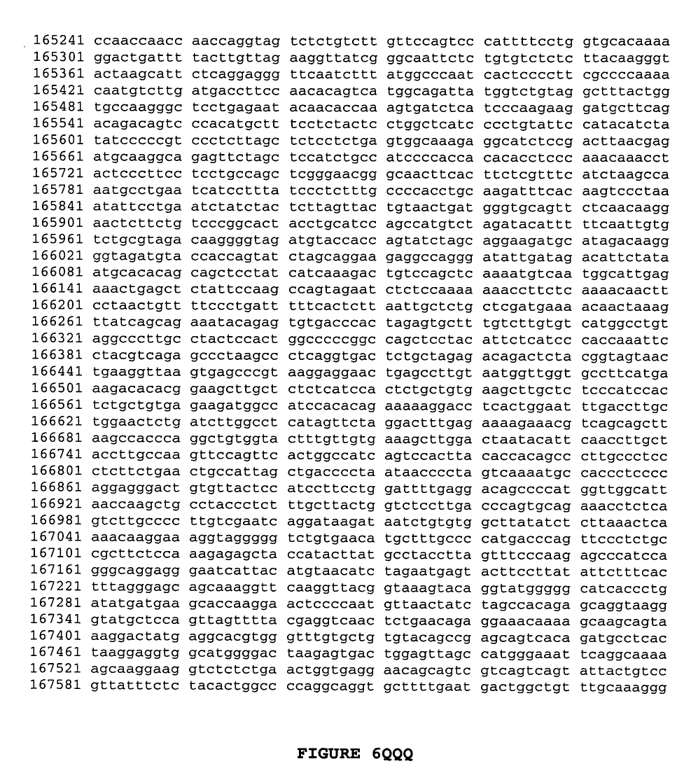

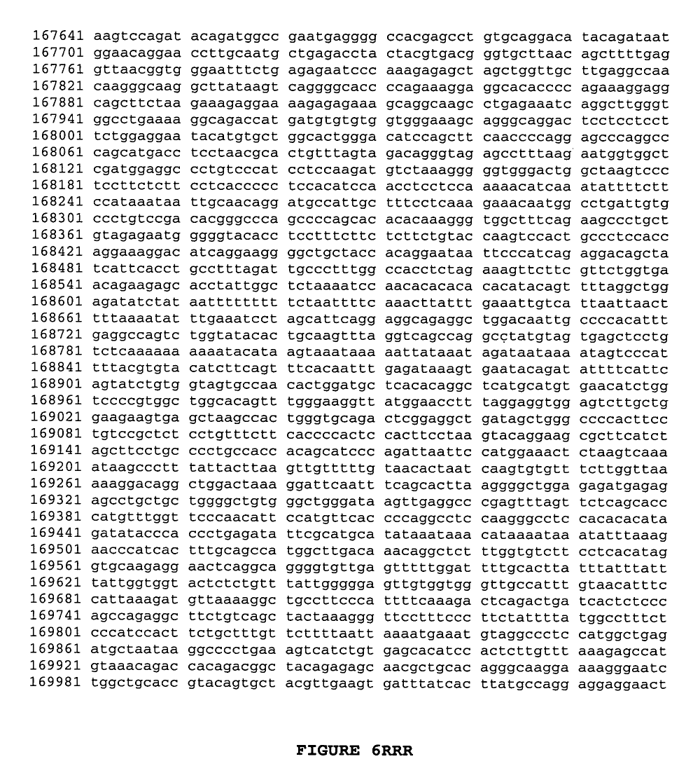

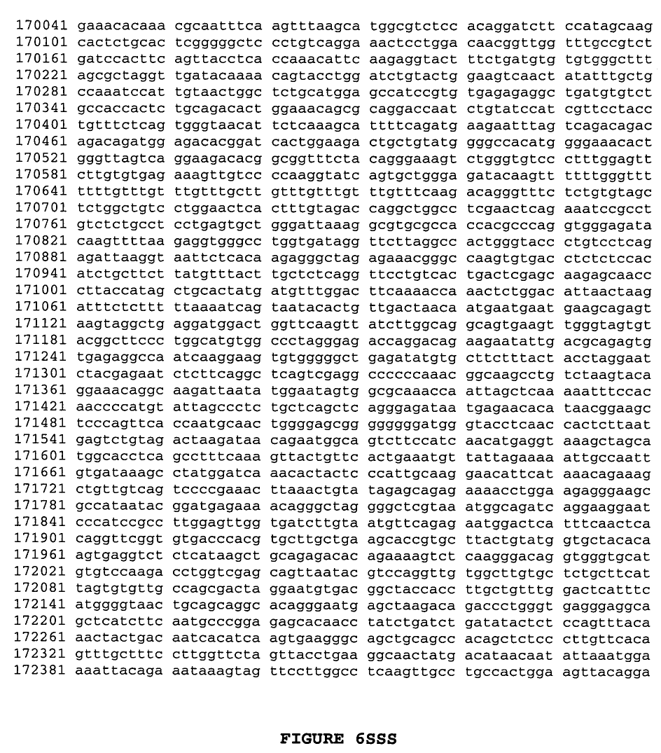

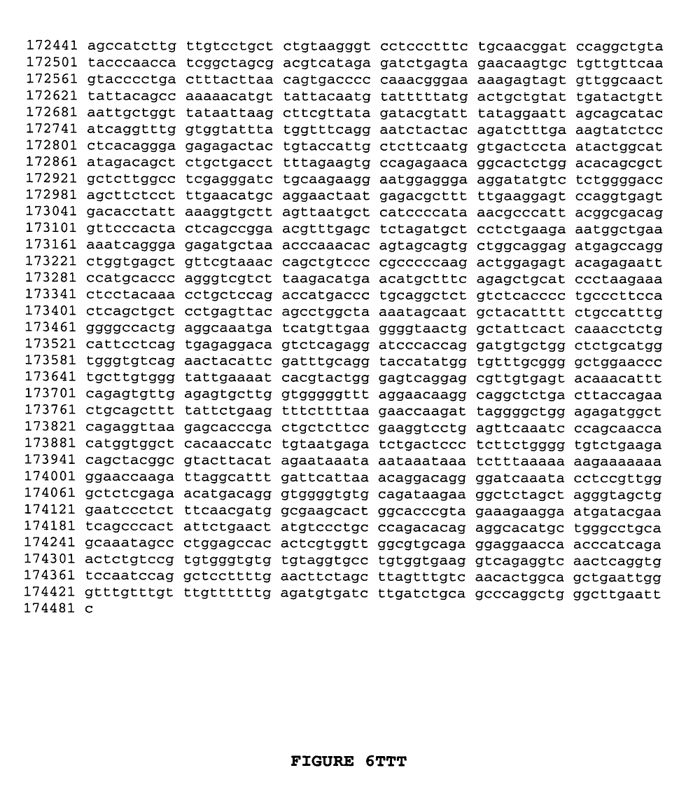

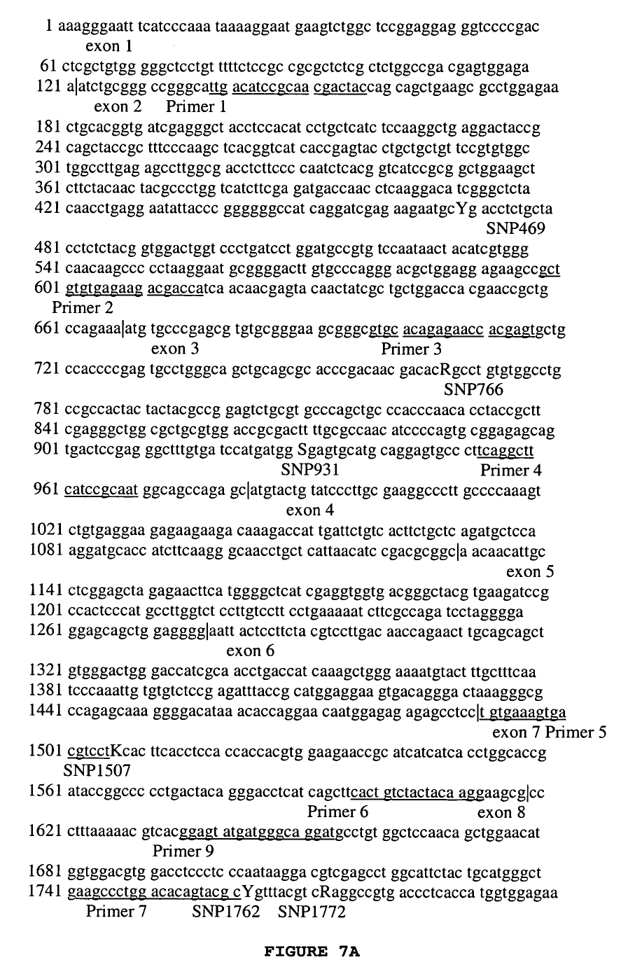

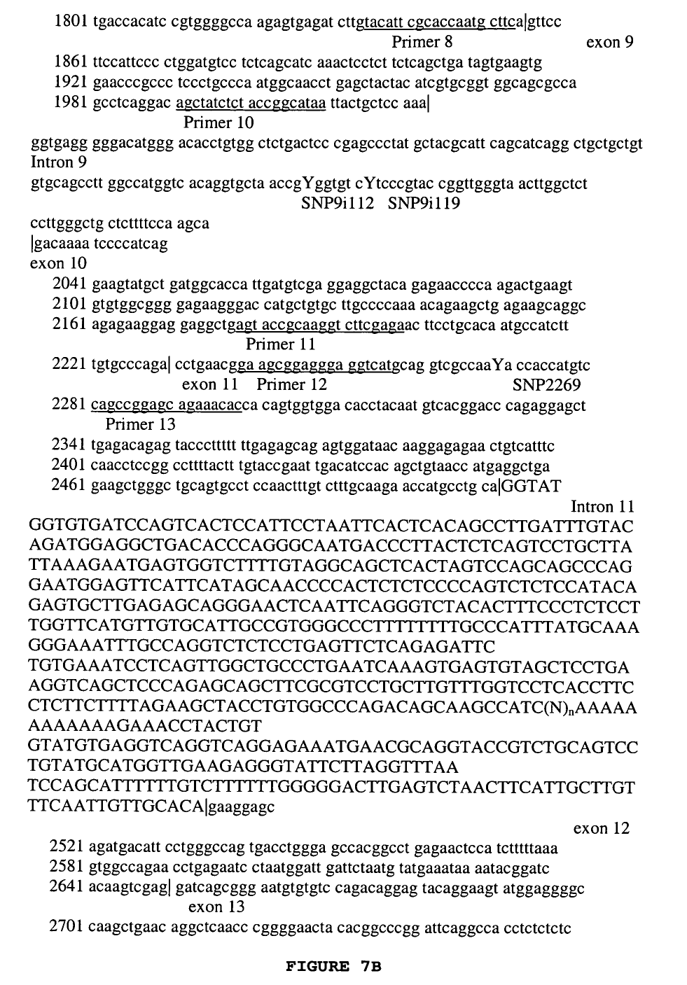

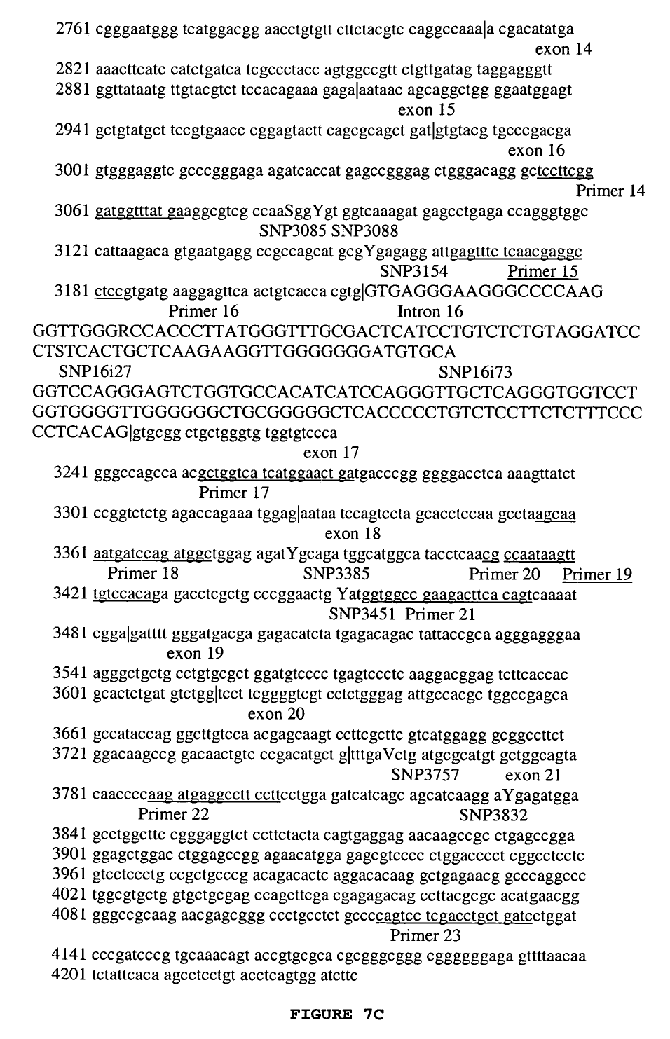

### DETAILED DESCRIPTION OF THE PREFERRED EMBODIMENT

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art.

As used herein, “reproductive longevity” means a biologically significant increase in the number of pregnancies and/or the duration of time an animal is capable of reproduction, relative to the mean of a given population, group or species.

As used herein, “the ability to sustain performance under stress” means a biologically significant increase in performance, in situations with stress, i.e., increase in the number of pregnancies and/or the duration of time while the animal is lactating and raising progeny, i.e., carrying a fetus while lactating at the same time, relative to the mean of a given population.

The insulin-like growth factor-I receptor (IGF-1R) gene is a plasma membrane-bound disulfide-bonded heterotetrameric glycoprotein composed of two extracellular α-subunits containing a ligand binding domain and two transmembrane β-subunits that include a cytoplasmic tyrosine kinase domain (Richards et al., 1998). The IGF-1R gene plays a vital role in growth and development in several different ways, such as mediating mitogenic and metabolic responses, maintaining transformed cell phenotype, protecting cells from apoptotic injuries, and inducing differentiation in certain cell types especially myoblasts, adipocytes, osteoblasts and cells of the central nervous system (Valentinis et al., 1999; Jin et al., 2000).

Binding of the ligand to IGF-1R leads to autophosphorylation of the (x-subunit and activation of the β-subunit tyrosine kinase domains resulting in phosphorylation of several intracellular proteins including insulin receptor substrates (IRS) and Shc with the subsequent trigger of multiple signaling cascades, for instance those of the Ras-Raf-MAP kinase network and phosphatidylinositol 3-kinase. The various effects may depend on specific domains of the receptor and the availability of different substrates (Peruzzi et al., 1999; Swantek et al., 1999; Valentinis et al., 1999; Xu et al., 1999; Soni et al.; 2000).

The IGF-1R gene also plays a role in certain functions of other growth factors and hormones. There is evidence that a signal generated by a functional IGF-1R is required for the mitogenic effects of other growth factors, such as epidermal growth factor (EGF) and platelet-derived growth factor (PDGF) (Swantek and Baserga, 1999). Furthermore, the estradiol-induced mitogenic effects in the mouse uterus and differentiation of rat adipocytes are dependent on the IGF-1R (Richards et al., 1998; Dieudonne et al., 2000).

According to an embodiment of the present invention variants or polymorphic sites in the IGF-1R gene have been located, and these genetic polymorphisms are associated with reproductive longevity and/or the ability the sustain stress factors such as lactation and pregnancy in mice. These four variants include an ‘A’ to ‘G’ substitution in intron 16, a ‘G’ nucleotide insertion in intron 16, an ‘A’ to ‘G’ substitution in exon 21, and a 12 bp-deletion in exon 21 which resulted in four fewer amino acids in the IGF-1R protein.

In another embodiment, assays are provided for detection of these different variants. The assays preferably involve amplifying the genomic DNA purified from blood, tissue, semen, or other convenient source of genetic material by the use of primers and standard techniques, such as polymerase chain reaction (PCR).

A 12 bp deletion, PCR product was identified in mice. The PCR product can be sized in a variety of ways, such as by agarose or polyacrylamide gel electrophoresis, use of an automated DNA sequencer, or mass spectrometry.

An ‘A’ to ‘G’ substitution, at position 3876 of SEQ ID NO:1 (Genbank accession number AF056187) was identified in mice. The PCR product was digested with a restriction enzyme (e.g., HpaII) so as to yield gene fragments of varying lengths, as separating at least some of the fragments from others using agarose or polyacrylamide gel electrophoresis. Since the ‘A’ to ‘G’ substitution is 20 base pairs upstream from the 12-base pair deletion, both polymorphisms may be detected by the digestion of PCR product with the enzyme HpaII.

A ‘G’ to ‘A’ substitution (GGTC to GATC) was detected in intron 16 of the gene in mice. The 486 bp PCR product, spanning exons 16 and 17 and intron 16, was cut into 454 and 32 bp fragments (A₁ allele) by the enzyme DpnII (↑GATC). This nucleotide substitution resulted in the creation of a new recognition site for this enzyme, which cleaved the 454 bp fragment into 328 and 125 bp fragments (A₂ allele). In addition, sequence information revealed a ‘G’ nucleotide insertion in intron 16, 153 bp 5′ to the above point mutation, but no restriction enzyme was found for discriminatory typing of this deletion.

In porcine, the following single nucleotide polymorphisms were found:

A ‘G’ to ‘A’ substitution, designated SNP16i27, at position 27 from the end of intron 16 was detected with an AvaII restriction site.

A ‘G’ to ‘C’ substitution, designated SNP16i73, was detected at position 73 from the end of intron 16. This nucleotide substitution resulted in a MnlI restriction site.

A ‘G’ to ‘A’ substitution, designated SNP1772, was detected in exon 8. This nucleotide substitution resulted in a TaqI restriction site.

The polymorphisms in animals may also be identified using a variety of methods such as direct sequencing, and hybridizing with nucleotide probes labeled with radioactive or chemiluminescence. The probes may be sequences containing all or a portion of the IGF-1R gene containing the polymorphisms, which will be hybridized to the separated digestion PCR products or digested genomic DNA. The polymorphism may also be detected by restriction fragment length polymorphism (RFLP) analysis, the single-stranded conformation polymorphism of the PCR product (SSCP-PCR), PCR amplification of specific alleles, the amplification of DNA target by PCR followed by single base extension which will be detected by fluorescent or radioactive substances or mass spectrometry, allelic discrimination during PCR, Genetic Bit Analysis, Pyrosequencing, oligonucleotide ligation assay, analysis of melting curves or other methods which detect differences in the length of a DNA fragment at this region or detect a single nucleotide substitution.

Another embodiment of the invention includes novel PCR primers comprising 4 to 30 contiguous bases on either side of the polymorphism to provide an amplification system allowing for detection of the polymorphism by PCR and identification of the fragments by standard methods. Any primers amplifying the region of the polymorphism may be used as taught herein and are also publically available.

The preferred primers for revealing the 12 bp deletion are PSEQDF: 5′-GGA GAT CAT CGG CAG CAT CAA G-3′ (SEQ ID NO:8), wherein the 5′ end is at position 3786 of the mouse IGF-1R gene and PSEQDR: 5′-GCC ATT CTC AGC CTT GTG TCC-3′ (SEQ ID NO:9), wherein the 5′ end is at the position 4002 of the mouse IGF-1R gene.

The preferred primers for revealing the A to G substitution in exon 21 of the IGF-1R gene are PSECAF: 5′-GCA TGT GCT GGC AGT ATA ACC-3′ (SEQ ID NO:10), wherein the 5′ end is at position 3743 of the IGF-IR gene and PSECAR: 5° CAG AGG CCC ATG TCA GTT AAG (SEQ ID NO:11), wherein the 5′ end is at position 4376 of the IGF-1R gene.

The preferred primers for revealing the G to A substitution in intron 16 of the IGF-1R gene are PSEQ16F: 5′ AGA GTG GCC ATC AAG ACG GTA 3′ (SEQ ID NO:12) and PSEQ16R: 5′ GGC CTC AGA GAC CGG AGA T 3′ (SEQ ID NO:13).

In porcine, the preferred primers for revealing SNP16i27 identified with an AvaII restriction site are Primer 16: 5′-CCT CCG TGA TGA AGG AGT TC-3′ (SEQ ID NO:14) and Primer 17: 5′-TCA GTT CCA TGA TGA CCA GC-3′ (SEQ ID NO: 15).

The preferred primers for revealing SNP16i73 identified with a MnII restriction site are Primer 16:5′-CCT CCG TGA TGA AGG AGT TC-3′ (SEQ ID NO:16) and Primer 17: 5′-TCA GTT CCA TGA TGA CCA GC-3′ (SEQ ID NO:17).

The preferred primers for revealing SNP1772 identified with a TaqI restriction site are designated as Primer 9: 5′-GGA GTA TGA TGG GCA GGA T-3′ (SEQ ID NO:18) and Primer 8: 5′-GAA GCA TTG GTG CGA ATG TA-3′ (SEQ ID NO:19). Computer programs available on the world wide web allows one of ordinary skill in the art to design other primers capable of amplifying polymorphic segments of the IGF-1R gene such as those shown above and depicted in Table 1. See Steve Rozen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386.

A further embodiment comprises a breeding method whereby assays of the above types are conducted on a plurality of gene sequences from different animals or animal embryos of various species to be selected from and, based on the results, certain animals are either selected or dropped out of the breeding program.

The following is a general overview of techniques which can be used to assay for the polymorphisms of the invention.

In the present invention, a sample of genetic material is obtained from an animal. Samples can be obtained from blood, tissue, semen, etc. Generally, peripheral blood cells are used as the source, and the genetic material is DNA. A sufficient amount of cells are obtained to provide a sufficient amount of DNA for analysis. This amount will be known or readily determinable by those skilled in the art. The DNA is isolated from the blood cells by techniques known to those skilled in the art.

Isolation and Amplification of Nucleic Acid

Samples of genomic DNA are isolated from any convenient source including saliva, buccal cells, hair roots, blood, cord blood, amniotic fluid, interstitial fluid, peritoneal fluid, chorionic villus, and any other suitable cell or tissue sample with intact nuclei. The cells can also be obtained from solid tissue as from a fresh or preserved organ or from a tissue sample or biopsy. The sample can contain compounds which are not naturally intermixed with the biological material such as preservatives, anticoagulants, buffers, fixatives, nutrients, antibiotics, or the like.

Methods for isolation of genomic DNA from these various sources are described in, for example, Kirby, DNA Fingerprinting, An Introduction, W.H. Freeman & Co. New York (1992). Genomic DNA can also be isolated from cultured primary or secondary cell cultures or from transformed cell lines derived from any of the aforementioned tissue samples.

Samples of animal RNA can also be used. RNA can be isolated from tissues expressing the IGF-1R gene as described in Sambrook et al., supra. RNA can be total cellular RNA, mRNA, poly A+ RNA, or any combination thereof. For best results, the RNA is purified, but can also be unpurified cytoplasmic RNA. RNA can be reverse transcribed to form DNA which is then used as the amplification template, such that the PCR indirectly amplifies a specific population of RNA transcripts. See, e.g., Sambrook, supra, Kawasaki et al., Chapter 8 in PCR Technology, (1992) supra, and Berg et al., Hum. Genet. 85:655-658 (1990).

PCR Amplification

The most common means for amplification is polymerase chain reaction (PCR), as described in U.S. Pat. Nos. 4,683,195, 4,683,202, 4,965,188 each of which is hereby incorporated by reference. If PCR is used to amplify the target regions in blood cells, heparinized whole blood should be drawn in a sealed vacuum tube kept separated from other samples and handled with clean gloves. For best results, blood should be processed immediately after collection; if this is impossible, it should be kept in a sealed container at 4° C. until use. Cells in other physiological fluids may also be assayed. When using any of these fluids, the cells in the fluid should be separated from the fluid component by centrifugation.

Tissues should be roughly minced using a sterile, disposable scalpel and a sterile needle (or two scalpels) in a 5 mm Petri dish. Procedures for removing paraffin from tissue sections are described in a variety of specialized handbooks well known to those skilled in the art.

To amplify a target nucleic acid sequence in a sample by PCR, the sequence must be accessible to the components of the amplification system. One method of isolating target DNA is crude extraction which is useful for relatively large samples. Briefly, mononuclear cells from samples of blood, amniocytes from amniotic fluid, cultured chorionic villus cells, or the like are isolated by layering on sterile Ficoll-Hypaque gradient by standard procedures. Interphase cells are collected and washed three times in sterile phosphate buffered saline before DNA extraction. If testing DNA from peripheral blood lymphocytes, an osmotic shock (treatment of the pellet for 10 sec with distilled water) is suggested, followed by two additional washings if residual red blood cells are visible following the initial washes. This will prevent the inhibitory effect of the heme group carried by hemoglobin on the PCR reaction. If PCR testing is not performed immediately after sample collection, aliquots of 10⁶ cells can be pelleted in sterile Eppendorf tubes and the dry pellet frozen at −20° C. until use.

The cells are resuspended (10⁶ nucleated cells per 100 μl) in a buffer of 50 mM Tris-HCl (pH 8.3), 50 mM KC1 1.5 mM MgC1₂, 0.5% Tween 20, 0.5% NP40 supplemented with 100 μg/ml of proteinase K. After incubating at 56° C. for 2 hr. the cells are heated to 95° C. for 10 min to inactivate the proteinase K and immediately moved to wet ice (snap-cool). If gross aggregates are present, another cycle of digestion in the same buffer should be undertaken. Ten μl of this extract is used for amplification.

When extracting DNA from tissues, e.g., chorionic villus cells or confluent cultured cells, the amount of the above mentioned buffer with proteinase K may vary according to the size of the tissue sample. The extract is incubated for 4-10 hrs at 50°-60° C. and then at 95° C. for 10 minutes to inactivate the proteinase. During longer incubations, fresh proteinase K should be added after about 4 hr at the original concentration.

When the sample contains a small number of cells, extraction may be accomplished by methods as described in Higuchi, “Simple and Rapid Preparation of Samples for PCR”, in PCR Technology, Ehrlich, H. A. (ed.), Stockton Press, New York, which is incorporated herein by reference. PCR can be employed to amplify target regions in very small numbers of cells (1000-5000) derived from individual colonies from bone marrow and peripheral blood cultures. The cells in the sample are suspended in 20 μl of PCR lysis buffer (10 mM Tris-HC1 (pH 8.3), 50 mM KC1, 2.5 mM MgC1₂, 0.1 mg/ml gelatin, 0.45% NP40, 0.45% Tween 20) and frozen until use. When PCR is to be performed, 0.6 μl of proteinase K (2 mg/ml) is added to the cells in the PCR lysis buffer. The sample is then heated to about 60° C. and incubated for 1 hr. Digestion is stopped through inactivation of the proteinase K by heating the samples to 95° C. for 10 min and then cooling on ice.

A relatively easy procedure for extracting DNA for PCR is a salting out procedure adapted from the method described by Miller et al., Nucleic Acids Res. 16:1215 (1988), which is incorporated herein by reference. Mononuclear cells are separated on a Ficoll-Hypaque gradient. The cells are resuspended in 3 ml of lysis buffer (10 mM Tris-HC1, 400 mM NaC1, 2 mM Na₂ EDTA, pH 8.2). Fifty μl of a 20 mg/ml solution of proteinase K and 150 μl of a 20% SDS solution are added to the cells and then incubated at 37° C. overnight. Rocking the tubes during incubation will improve the digestion of the sample. If the proteinase K digestion is incomplete after overnight incubation (fragments are still visible), an additional 50 μl of the 20 mg/ml proteinase K solution is mixed in the solution and incubated for another night at 37° C. on a gently rocking or rotating platform. Following adequate digestion, one ml of a 6 M NaCl solution is added to the sample and vigorously mixed. The resulting solution is centrifuged for 15 minutes at 3000 rpm. The pellet contains the precipitated cellular proteins, while the supernatant contains the DNA. The supernatant is removed to a 15 ml tube that contains 4 ml of isopropanol. The contents of the tube are mixed gently until the water and the alcohol phases have mixed and a white DNA precipitate has formed. The DNA precipitate is removed and dipped in a solution of 70% ethanol and gently mixed. The DNA precipitate is removed from the ethanol and air-dried. The precipitate is placed in distilled water and dissolved.

Kits for the extraction of high-molecular weight DNA for PCR include a Genomic Isolation Kit A.S.A.P. (Boehringer Mannheim, Indianapolis, Ind.), Genomic DNA Isolation System (GIBCO BRL, Gaithersburg, Md.), Elu-Quik DNA Purification Kit (Schleicher & Schuell, Keene, N. H.), DNA Extraction Kit (Stratagene, LaJolla, Calif.), TurboGen Isolation Kit (Invitrogen, San Diego, Calif.), and the like. Use of these kits according to the manufacturer's instructions is generally acceptable for purification of DNA prior to practicing the methods of the present invention.

The concentration and purity of the extracted DNA can be determined by spectrophotometric analysis of the absorbance of a diluted aliquot at 260 nm and 280 nm. After extraction of the DNA, PCR amplification may proceed. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In a particularly useful embodiment of PCR amplification, strand separation is achieved by heating the reaction to a sufficiently high temperature for a sufficient time to cause the denaturation of the duplex but not to cause an irreversible denaturation of the polymerase (see U.S. Pat. No. 4,965,188, incorporated herein by reference). Typical heat denaturation involves temperatures ranging from about 80° C. to 105° C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity. For example, the enzyme RecA has helicase activity in the presence of ATP. The reaction conditions suitable for strand separation by helicases are known in the art (see Kuhn Hoffman-Berling, 1978, CSH-Quantitative Biology, 43:63-67; and Radding, 1982, Ann. Rev. Genetics 16:405-436, each of which is incorporated herein by reference).

Template-dependent extension of primers in PCR is catalyzed by a polymerizing agent in the presence of adequate amounts of four deoxyribonucleotide triphosphates (typically dATP, dGTP, dCTP, and dTTP) in a reaction medium comprised of the appropriate salts, metal cations, and pH buffering systems. Suitable polymerizing agents are enzymes known to catalyze template-dependent DNA synthesis. In some cases, the target regions may encode at least a portion of a protein expressed by the cell. In this instance, mRNA may be used for amplification of the target region. Alternatively, PCR can be used to generate a cDNA library from RNA for further amplification, the initial template for primer extension is RNA. Polymerizing agents suitable for synthesizing a complementary, copy-DNA (cDNA) sequence from the RNA template are reverse transcriptase (RT), such as avian myeloblastosis virus RT, Moloney murine leukemia virus RT, or Thermus thermophilus (Tth) DNA polymerase, a thermostable DNA polymerase with reverse transcriptase activity marketed by Perkin Elmer Cetus, Inc. Typically, the genomic RNA template is heat degraded during the first denaturation step after the initial reverse transcription step leaving only DNA template. Suitable polymerases for use with a DNA template include, for example, E. coli DNA polymerase I or its Klenow fragment, T4 DNA polymerase, Tth polymerase, and Taq polymerase, a heat-stable DNA polymerase isolated from Thermus aquaticus and commercially available from Perkin Elmer Cetus, Inc. The latter enzyme is widely used in the amplification and sequencing of nucleic acids. The reaction conditions for using Taq polymerase are known in the art and are described in Gelfand, 1989, PCR Technology, supra.

Allele Specific PCR

Allele-specific PCR differentiates between target regions differing in the presence of a polymorphism. PCR amplification primers are chosen which bind only to certain alleles of the target sequence. This method is described by Gibbs, Nucleic Acid Res. 17:12427-2448 (1989).

Allele Specific Oligonucleotide Screening Methods

Further diagnostic screening methods employ the allele-specific oligonucleotide (ASO) screening methods, as described by Saiki et al., Nature 324:163-166 (1986). Oligonucleotides with one or more base pair mismatches are generated for any particular allele. ASO screening methods detect mismatches between variant target genomic or PCR amplified DNA and non-mutant oligonucleotides, showing decreased binding of the oligonucleotide relative to a mutant oligonucleotide. Oligonucleotide probes can be designed that under low stringency will bind to both polymorphic forms of the allele, but which at high stringency, bind to the allele to which they correspond. Alternatively, stringency conditions can be devised in which an essentially binary response is obtained, i.e., an ASO corresponding to a variant form of the target gene will hybridize to that allele, and not to the wild-type allele.

Ligase Mediated Allele Detection Method

Target regions of the DNA of a test subject can be compared with target regions in unaffected and affected family members by ligase-mediated allele detection. See Landegren et al., Science 241:107-1080 (1988). Ligase may also be used to detect point mutations in the ligation amplification reaction described in Wu et al., Genomics 4:560-569 (1989). The ligation amplification reaction (LAR) utilizes amplification of specific DNA sequence using sequential rounds of template dependent ligation as described in Wu, supra, and Barany, Proc. Nat. Acad. Sci. 88:189-193 (1990).

Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (Tₘ). Melting domains are at least 20 base pairs in length, and may be up to several hundred base pairs in length.

Differentiation between alleles based on sequence specific melting domain differences can be assessed using polyacrylamide gel electrophoresis, as described in Chapter 7 of Erlich, ed., PCR Technology, Principles and Applications for DNA Amplification, W.H. Freeman and Co., New York (1992), the contents of which are hereby incorporated by reference.

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527 (1986), and Myers et al., in Genomic Analysis, A Practical Approach, K. Davies Ed. IRL Press Limited, Oxford, pp. 95-139 (1988), the contents of which are hereby incorporated by reference. The electrophoresis system is maintained at a temperature slightly below the Tm of the melting domains of the target sequences.

In an alternative method of denaturing gradient gel electrophoresis, the target sequences may be initially attached to a stretch of GC nucleotides, termed a GC clamp, as described in Chapter 7 of Erlich, supra. Preferably, at least 80% of the nucleotides in the GC clamp are either guanine or cytosine. Preferably, the GC clamp is at least 30 bases long. This method is particularly suited to target sequences with high Tₘ's.

Generally, the target region is amplified by the polymerase chain reaction as described above. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions as described above. DNA fragments differing by a single base change will migrate through the gel to different positions, which may be visualized by ethidium bromide staining.

Temperature Gradient Gel Electrophoresis

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

Single-Strand Conformation Polymorphism Analysis Target sequences or alleles at the IGF-IR locus can be differentiated using single-strand conformation polymorphism analysis, which identifies base differences by alteration in electrophoretic migration of single stranded PCR products, as described in Orita et al., Proc. Nat. Acad. Sci. 85:2766-2770 (1989). Amplified PCR products can be generated as described above, and heated or otherwise denatured, to form single stranded amplification products. Single-stranded nucleic acids may refold or form secondary structures which are partially dependent on the base sequence. Thus, electrophoretic mobility of single-stranded amplification products can detect base-sequence difference between alleles or target sequences.

Chemical or Enzymatic Cleavage of Mismatches

Differences between target sequences can also be detected by differential chemical cleavage of mismatched base pairs, as described in Grompe et al., Am. J Hum. Genet. 48:212-222 (1991). In another method, differences between target sequences can be detected by enzymatic cleavage of mismatched base pairs, as described in Nelson et al., Nature Genetics 4:11-18 (1993). Briefly, genetic material from an animal and an affected family member may be used to generate mismatch free heterohybrid DNA duplexes. As used herein, “heterohybrid” means a DNA duplex strand comprising one strand of DNA from one animal, and a second DNA strand from another animal, usually an animal differing in the phenotype for the trait of interest. Positive selection for heterohybrids free of mismatches allows determination of small insertions, deletions or other polymorphisms that may be associated with IGF-1R polymorphisms.

Non-Gel Systems

Other possible techniques include non-gel systems such as TAQMAN™ (Perkin Elmer). In this system oligonucleotide PCR primers are designed that flank the mutation in question and allow PCR amplification of the region. A third oligonucleotide probe is then designed to hybridize to the region containing the base subject to change between different alleles of the gene. This probe is labeled with fluorescent dyes at both the 5′ and 3′ ends. These dyes are chosen such that while in this proximity to each other the fluorescence of one of them is quenched by the other and cannot be detected. Extension by Taq DNA polymerase from the PCR primer positioned 5′ on the template relative to the probe leads to the cleavage of the dye attached to the 5′ end of the annealed probe through the 5′ nuclease activity of the Taq DNA polymerase. This removes the quenching effect allowing detection of the fluorescence from the dye at the 3′ end of the probe. The discrimination between different DNA sequences arises through the fact that if the hybridization of the probe to the template molecule is not complete, i.e., there is a mismatch of some form, the cleavage of the dye does not take place. Thus only if the nucleotide sequence of the oligonucleotide probe is completely complimentary to the template molecule to which it is bound will quenching be removed. A reaction mix can contain two different probe sequences each designed against different alleles that might be present thus allowing the detection of both alleles in one reaction.

Yet another technique includes an Invader Assay which includes isothermic amplification that relies on a catalytic release of fluorescence.

Non-PCR Based DNA Diagnostics

The identification of a DNA sequence linked to IGF-1R can be made without an amplification step, based on polymorphisms including restriction fragment length polymorphisms in an animal and a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes are preferably labeled directly or indirectly, such that by assaying for the presence or absence of the probe, one can detect the presence or absence of the target sequence. Direct labeling methods include radioisotope labeling, such as with ³²p or ³⁵S. Indirect labeling methods include fluorescent tags, biotin complexes which may be bound to avidin or streptavidin, or peptide or protein tags. Visual detection methods include photoluminescents, Texas red, rhodamine and its derivatives, red leuco dye and 3,3′,5,5′-tetramethylbenzidine (TMB), fluorescein, and its derivatives, dansyl, umbelliferone and the like or with horse radish peroxidase, alkaline phosphatase and the like.

Hybridization probes include any nucleotide sequence capable of hybridizing to the mouse chromosome where IGF-1R resides, and thus defining a genetic marker linked to IGF-1R, including a restriction fragment length polymorphism, a hypervariable region, repetitive element, or a variable number tandem repeat. Hybridization probes can be any gene or a suitable analog. Further suitable hybridization probes include exon fragments or portions of cDNAs or genes known to map to the relevant region of the chromosome.

Preferred tandem repeat hybridization probes for use according to the present invention are those that recognize a small number of fragments at a specific locus at high stringency hybridization conditions, or that recognize a larger number of fragments at that locus when the stringency conditions are lowered.

One or more additional restriction enzymes and/or probes and/or primers can be used. Additional enzymes, constructed probes, and primers can be determined by routine experimentation by those of ordinary skill in the art and are intended to be within the scope of the invention.

Although the methods described herein may be in terms of the use of a single restriction enzyme and a single set of primers, the methods are not so limited. One or more additional restriction enzymes and/or probes and/or primers can be used, if desired. Indeed in some situations it may be preferable to use combinations of markers giving specific haplotypes. Additional enzymes, constructed probes and primers can be determined through routine experimentation, combined with the teachings provided and incorporated herein. Stand alone software as well as web-based software are avaible that allows the user to identify other restriction mapping sites in the DNA sequence, e.g., http://www.restrictionmapper.org/.

According to the invention, polymorphisms in the IGF-1R gene have been identified which have been associated with reproductive longevity and/or sustained performance under stress. The presence or absence of the markers, in one embodiment may be assayed by PCR-RFLP analysis using the restriction endonucleases and amplification primers may be designed using analogous human, mouse, or other IGF-1R sequences due to high homology in the region surrounding the polymorphisms, or may be designed using known IGF-1R gene sequence data as exemplified in Genbank or even designed from sequences obtained from linkage data from closely surrounding genes based upon the teachings and references herein. The sequences surrounding the polymorphism will facilitate the development of alternate PCR tests in which a primer of about 4-30 contiguous bases taken from the sequence immediately adjacent to the polymorphism is used in connection with a polymerase chain reaction to greatly amplify the region before treatment with the desired restriction enzyme. The primers need not be the exact complement; substantially equivalent sequences are acceptable. The design of primers for amplification by PCR is known to those of skill in the art and is discussed in detail in Ausubel (ed.), “Short Protocols in Molecular Biology, Fourth Edition” John Wiley and Sons 1999. The following is a brief description of primer design.

Primer Design Strategy

Increased use of polymerase chain reaction (PCR) methods has stimulated the development of many programs to aid in the design or selection of oligonucleotides used as primers for PCR. Four examples of such programs that are freely available via the Internet are: PRIMER by Mark Daly and Steve Lincoln of the Whitehead Institute (UNIX, VMS, DOS, and Macintosh), Oligonucleotide Selection Program (OSP) by Phil Green and LaDeana Hiller of Washington University in St. Louis (UNIX, VMS, DOS, and Macintosh), PGEN by Yoshi (DOS only), and Amplify by Bill Engels of the University of Wisconsin (Macintosh only). Generally these programs help in the design of PCR primers by searching for bits of known repeated-sequence elements and then optimizing the Tₘ by analyzing the length and GC content of a putative primer. Commercial software is also available and primer selection procedures are rapidly being included in most general sequence analysis packages.

Sequencing and PCR Primers

Designing oligonucleotides for use as either sequencing or PCR primers requires selection of an appropriate sequence that specifically recognizes the target, and then testing the sequence to eliminate the possibility that the oligonucleotide will have a stable secondary structure. Inverted repeats in the sequence can be identified using a repeat-identification or RNA-folding program such as those described above (see prediction of Nucleic Acid Structure). If a possible stem structure is observed, the sequence of the primer can be shifted a few nucleotides in either direction to minimize the predicted secondary structure. The sequence of the oligonucleotide should also be compared with the sequences of both strands of the appropriate vector and insert DNA. Obviously, a sequencing primer should only have a single match to the target DNA. It is also advisable to exclude primers that have only a single mismatch with an undesired target DNA sequence. For PCR primers used to amplify genomic DNA, the primer sequence should be compared to the sequences in the GenBank database to determine if any significant matches occur. If the oligonucleotide sequence is present in any known DNA sequence or, more importantly, in any known repetitive elements, the primer sequence should be changed. Depending on the desired test conditions, the sequences of the primers should be designed to provide for both efficient and faithful replication of the target nucleic acid. Methods of PCR primer design are common and well known in the art. (Rychlik, W. (1993) In White, B. A. (ed.), Methods in Molecular Biology, Vol. 15, pages 31-39, PCR Protocols: Current Methods and Applications. Humania Press, Inc., Totowa, N.J.).

The methods and materials of the invention may be used as the basis to search for polymorphisms in the IGF-1R gene of species that are associated with reproductive longevity and sustained performance under stress. This would allow uses to genetically type individual animals by detecting genetic differences in those animals. For instance, a sample of mouse genomic DNA may be evaluated by reference to one or more controls to determine if a polymorphism in the IGF-1R gene is present. Preferably, RFLP analysis is performed with respect to the mouse IGF-1R gene, and the results are compared with a control. The control is the result of a RFLP analysis of the mouse IGF-1R gene of a different mouse where the polymorphism of the mouse IGF-1R gene is known. Similarly, the IGF-1R genotype of a mouse may be determined by obtaining a sample of its genomic DNA, conducting RFLP analysis of the IGF-1R gene in the DNA, and comparing the results with a control. Again, the control is the result of RFLP analysis of the IGF-1R gene of a different mouse. The results genetically type the mouse by specifying the polymorphism(s) in its IGF-1R genes. Finally, genetic differences among mice can be detected by obtaining samples of the genomic DNA from at least two mice, identifying the presence a polymorphism in the IGF-1R gene, and comparing the results.

Such assays are useful for identifying the genetic markers relating reproductive longevity and the ability to sustained stress factors such as lactation and pregnancy, as discussed above and for the general scientific analysis of mouse genotypes' and phenotypes .

The examples and methods herein disclose certain genes which have been identified to have a polymorphism which is associated either positively or negatively with a beneficial trait that will have an effect on performance under stress in animals, such as cattle, birds, and aquatic species, such as shrimp carrying this polymorphism. The identification of the existence of a polymorphism within a gene is often made by a single base alternative that results in a restriction site in certain allelic forms. A certain allele, however, as demonstrated and discussed herein, may have a number of base changes associated with it that could be assayed for which are indicative of the same polymorphism (allele). Further, other genetic markers or genes may be linked to the polymorphisms disclosed herein so that assays may involve identification of other genes or gene fragments, but which ultimately rely upon genetic characterization of animals for the same polymorphism. Any assay which sorts and identifies animals based upon the allelic differences disclosed herein are intended to be included within the scope of this invention.

Linkage Analysis

Diagnostic screening may be performed for polymorphisms that are genetically linked to a phenotypic variant in IGF-1R activity or expression, particularly through the use of microsatellite markers or single nucleotide polymorphisms (SNP). The microsatellite or SNP polymorphism itself may not be phenotypically expressed, but is linked to sequences that result in altered activity or expression. Two polymorphic variants may be in linkage disequilibrium, i.e., where alleles show non-random associations between genes even though individual loci are in Hardy-Weinberg equilibrium.

Linkage analysis may be performed alone, or in combination with direct detection of phenotypically evident polymorphisms. The use of microsatellite markers for genotyping is well documented. For examples, see Mansfield et al. (1994) Genomics 24:225-233; and Ziegle et al. (1992) Genomics 14:1026-1031. The use of SNPs for genotyping is illustrated in Underhill et al. (1996) Proc. Natl. Acad. Sci. USA 93:196-200.

Genetic linkage maps show the relative locations of specific DNA markers along a chromosome. Any inherited physical or molecular characteristic that differs among animals and is easily detectable in the laboratory is a potential genetic marker. DNA sequence polymorphisms are useful markers because they are plentiful and easy to characterize precisely. Many such polymorphisms are located in non-coding regions and do not affect the phenotype of the organism, yet they are detectable at the DNA level and can be used as markers. Examples include restriction fragment length polymorphisms (RFLPs), which reflect sequence variations in DNA sites or differences in the length of the product, which can be cleaved by DNA restriction enzymes, microsatellite markers, which are short repeated sequences that vary in the number of repeated units, single nucleotide polymorphisms (SNPs), and the like.

The “linkage” aspect of the map is a measure of how frequently two markers are inherited together. The closer the markers are to each other physically, the less likely a recombination event will fall between and separate them. Recombination frequency thus provides an estimate of the distance between two markers. The value of the genetic map is that an inherited trait can be located on the map by following the inheritance of a DNA marker present in affected animals, but absent in unaffected animals, even though the molecular basis for the trait may not yet be understood.

SNPs are generally biallelic systems, that is, there are two alleles that a population may have for any particular marker. This means that the information content per SNP marker is relatively low when compared to microsatellite markers, which may have upwards of 10 alleles. SNPs also tend to be population-specific; a marker that is polymorphic in one population may not be very polymorphic in another. SNP markers offer a number of benefits that will make them an increasingly valuable tool. SNPs, found approximately every kilobase (see Wang et al. (1998) Science 280:1077-1082), offer the potential for generating high density genetic maps, which will be extremely useful for developing haplotyping systems for genes or regions of interest, and because of the nature of SNPs, they may in fact be the polymorphisms associated with the traits under study. The low mutation rate of SNPs also makes them excellent markers for studying complex genetic traits.

One of skill in the art, once a polymorphism has been identified and a correlation to a particular trait established, will understand that there are many ways to genotype animals for this polymorphism. The design of such alternative tests merely represents optimization of parameters known to those of skill in the art and is intended to be within the scope of this invention as fully described herein.

The following examples serves to better illustrate the invention described herein and are not intended to limit the invention in any way. Those skilled in the art will recognize that there are several different parameters which may be altered using routine experimentation and which are intended to be within the scope of this invention.

### EXAMPLE 1

### Identify Polymorphisms at the daf-2 (Insulin-Like Growth Factor-1 Receptor) Gene in Lines of Mice Selected for Reproductive Longevity and Evaluating this Gene as Putative Candidates for DNA Markers for Reproductive Longevity in Livestock.

Materials and Methods

The mouse population: The original mouse population, which was established by Agriculture and Agri-Food Canada in Ottawa in 1965, was a cross between two strains of mice (P and Q). The P strain was a cross between three inbred lines (C3H/HeJ, C57BL/6J, CBA/J, SWR/J) and the Q was Falconer's strain, which had a substantial heterogeneous background (Garnett and Falconer 1975). Ancestry of the Q strain goes back to 1948, with a large contribution from the ‘J’ stain (Falconer 1973). The ‘J’ strain was a heterogeneous population of mixed origin, which was made from crosses between Bateman's high-lactation line, Goodale's and MacArthur's large body weight selected lines, and four mutant stocks with the C57-BL inbred line as part of their ancestry (Brown and Falconer 1960). This population ‘was about as close as one could get with laboratory mice to a natural random-bred population’ (Brown and Falconer 1960). Several strains were derived from the J stock, including Falconer's control line (JC), an inbred line (JU), and a high litter size selected line (JH). The JC and JU lines constituted half of the ancestry of the Q strain. The other half was from crosses between Goodale's and MacArthur's large body weight selected lines (that had contributed to the J stock), MacArthur's small body weight selected line, JH, and a line that derived from the J stock and had been selected for high growth rate on a restricted diet (Falconer 1960). The four inbred lines and two of the lines that contributed to the Q strain (MacArthur's small body weight selected line (SM/J) and Goodale's large body weight selected line (LG/J)),are currently maintained at the Jackson Laboratories, Bar Harbor, Me. The contribution of so many strains to this colony, which is the only non-inbred mouse model in the world selected for reproductive longevity, was important for ensuring that the base population was heterozygous at many loci.

Prior to the implementation of the selection program for reproductive longevity, both the P and Q stocks were maintained by random mating for 23 generations (80 breeding pairs in P and 45 males and 90 females in Q) to achieve linkage equilibrium. Two lines from each of the P and Q strains were then established, each with 92 pairs of breeders. One line derived from each of the P and Q stocks was selected for nursing ability of the mother, and the other for body weight of progeny at 42 days of age. After 21 generations of selection, these four lines were crossed, and the synthetic stock was maintained by random mating for 12 generations to allow it to approach linkage equilibrium. One control (C1) and two selected lines, with (SA1) and without (SU1) standardizing litter size to 8, were established in 1982 and have been continuously selected for reproductive longevity since then (Nagai et al. 1995). Replications from each of the control and selected lines were established (C2, SA2, SU2) in 1993 using the existing lines (generation 18 of the SA1 and SU1 and generation 44 of the C1). Also, the high performing animals from the different selected lines were mated to generate a new line with a more diverse genetic background, and a sample from the control lines was used to generate a new control line. In each of the selected lines, one male and one female were caged at about eight weeks of age, and each pair was maintained in the same cage continuously until the next generation was established, using progeny from the latest parities. In the control lines, progenies from the first parity were used as breeders. The control and selected lines were maintained with 42 and 30 breeding pairs, respectively, avoiding full-sib mating (Nagai et al. 1995). Performance of the three original lines (SA1, SA2, C1) at generations 12 and 16 is reported by Nagai et al. (1995), and at generation 24 by Farid et al. (2002). The average number of days from mating to the last parturition in generation 12 was 236, 265 and 159 for lines SA1, SU1 and C1, respectively, showing that reproductive longevity was improved by 48% in the SA1 and 67% in the SU1. The corresponding values at generation 16 were 79% and 80%, and at generation 24 were 86% and 61% for the SA1 and SU1 lines, respectively. The number of parturitions during lifetime has not changed in the control line (5.34, 4.90, 5.30 at generations 12, 16 and 24, respectively), while the SA1 line showed a steady improvement: 8.63, 8.84 and 10.6 (61.6%, 80.4% and 100%). The corresponding values for the SU1 line were 79.9%, 93.0% and 83.0%.

Source of DNA: DNA was extracted from blood or tissue of 261 breeder males and females from the lines C1 (generation 69), C2 (generation 70), SA1 and SU1 (generation 24), and from one progeny from each of 153 families from lines C1, C2, SA1, SA2, SU1 and SU2. DNA samples from the four inbred lines that have contributed to the base population (C3H/HeJ, C57BL/6J, CBA/J, SWR/J) were obtained from the Jackson Laboratories, Bar Harbor, Me.

Laboratory procedures: There are two sequences of the mouse insulin-like growth factor-1 receptor cDNA in Genbank (accession numbers AF056187 (SEQ ID NO:1) and XM_{—}133508 (SEQ ID NO:3)), and sequences of most of this gene's exons and introns included in the clone RP23-378H21 (Genbank accession number AC101879) (SEQ ID NO:6). Several overlapping PCR primers were designed to cover the entire coding region of the IGF-1R gene and its 3′ UTR using the Oligo 6.0 primer analysis software (Molecular Biology Insight, cascade, CO, USA). Information on a few of these primers which amplified polymorphic regions is shown in Table 1.

TABLE 1

Information on the primers used to amplify polymorphic segments of
the IGF-1R gene in mice.

| | | | | | | |
|---|---|---|---|---|---|---|
| | | | | | Anneal. | |
| SEQ ID | Primer | | | MgCl2 | temp. | |
| NO | name | Sequence (5′-3′) | Location | (mM) | (° C.) | Size, bp |

| | | | | | | |
|---|---|---|---|---|---|---|
| 8 | PSEQDF | GGAGATCATCGGCAGCATCAAG | Exon 21 | 2.5 | 58.0 | 216 or 204 |
| 9 | PSEQDR | GCCATTCTCAGCCTTGTGTCC | Exon 21 | | | |
| 10 | PSECAF | GCATGTGCTGGCAGTATAACC | Exon 21 | 1.5 | 58.5 | 634 |
| 11 | PSECAR | CAGAGGCCCATGTCAGTTAAG | 3′ UTR | | | |
| 12 | PSEQ16F | AGAGTGGCCATCAAGACGGTA | Exon 16 | 2.0 | 58.5 | 486 |
| 13 | PSEQ16R | GGCCTCAGAGACCGGAGAT | Exon 17 | | | |

PCR amplifications were performed in 50 μL volumes containing (final concentration) 0.1% Tween 20, 1×PCR buffer, 1.5-2.0 mM MgCl₂, 0.2 mM each dNTP, 400 nM each primer, 2 units of Taq polymerase (Roche) and 100 ng template DNA. The thermal cycler was set at 95° C. for 2 min followed by 34 cycles at 94° C. for 1 min, 55-67° C. (depending on the primer) for 1 min, 72° C. for 1 min and a final 9 min extension at 72° C. Long fragments were amplified using PCR cocktails similar to those explained above, except using 0.35 mM of each dNTP and 2.5 units of Long-Range Taq polymerase (Roche). Thermal cycler was set at an initial 2 min denaturation at 95° C., followed by 10 cycles of 94° C. for 10 sec, 55-67° C. for 30 sec and 68° C. for 10 min. The next 20 cycles consisted of 94° C. for 10 sec, annealing at 55-67° C. for 30 sec, elongation at 68° C. for 10 min plus an additional 20 sec for each new cycle and a final 9 min extension at 68° C.

Genotyping for the 12 bp deletion in exon 21 was performed using the GenScan option of an ABI 377 automated DNA sequencer. Two primers flanking the deletion were designed. The Hex Amidite label was placed on the forward primer. Since the deletion was from 3896 to 3907, the PCR product was 216 bp in the wild type (4002-3786) or 204 bp for the deletion. The PCR cocktail contained 1.25 μL of a 10× buffer, 1.25 μL of a 25 mM MgCl₂, 1.0 μL of a 1.25 mM dNTPs, 5 pmol of each primer, 0.2 μL of a 5 U/μL Amplitaq gold polymerase, 25 ng of DNA and water to 12.5 μL total volume. Thermal cycler conditions were 95° C. for 8 minutes initial denaturation, followed by 30 cycles of 95° C. for 30 sec, 58° C. for 30 sec, 72° C. for 60 sec, and a final extension of 72° C. for 30 minutes. PCR products were maintained at 6° C. until processed. One μL of PCR products were loaded into the sequencer.

Data Analysis:

Conformation of genotype frequencies to Hardy-Weinberg equilibrium was tested using the GENEPOP computer package (http://wbiomed.curtin.edu.au/genepop) using the default options (1000 dememorisation, 100 batches and 1000 iterations). The program uses the Markov chain method to estimate the exact Hardy-Weinberg probability without bias (Guo and Thompson, 1992). The probability of rejecting Hₒ, i.e., genotype frequencies are in Hardy-Weinberg equilibrium and the standard error of this estimate were computed. When standard errors were larger than 0.01, the data were re-analysed using a larger number of batches. This program does not perform any test when a locus is monomorphic or quasi monomorphic (two alleles, but one is represented only once).

Pairwise tests for homogeneity of allele and genotype frequency distributions were also performed using the GENEPOP computer package which follows the Raymond and Rousset (1995) method. The hypotheses tested were that allele and genotype distributions were independent of lines (no difference between lines). An unbiased estimate of the Fisher's exact test on contingency tables is performed using the Markov chain method (1000 dememorisation, 100 batches and 1000 iterations). The program computes the probability of being wrong when Hₒ is rejected. Rare alleles (frequency of less than 5%) were not pooled together prior to the above tests. F_{IS} statistics, as the measures of inbreeding within each line (Wright, 1943, 1978), were computed for each polymorphic site in every line using the GENEPOP computer program.

Results

Polymorphism: A total of 4434 bp of the IGF-1R gene, consisting of exons 2, 3, 9, 10, 12, 13, 14, 15, 16, 17 and 21 (2344 bp) and introns 10, 12, 13, 14 and 16 (2090 bp) in five to seven individuals from each of the three main lines (C1, SA1, SU1) were sequenced. No polymorphism was detected in exons 2, 3, 9, 10, 12, 13, 14, 15, 16, 17 or in introns 10, 12, 13 and 14. The following polymorphic sites have been detected:

Site A: A ‘G’ to ‘A’ substitution (GGTC to GATC) was detected in intron 16 of the gene. The 486 bp PCR product, spanning exons 16 and 17 and intron 16, was cut into 454 and 32 bp fragments (Al allele) by the enzyme DpnII (↑GATC). This nucleotide substitution resulted in the creation of a new recognition site for this enzyme, which cleaved the 454 bp fragment into 328 and 125 bp fragments (A₂ allele). In addition, sequence information revealed a ‘G’ nucleotide insertion in intron 16, 153 bp 5′ to the above point mutation, but no restriction enzyme was found for discriminatory typing of this insertion.

Site B: An HpaII (C↑CGG) polymorphism was detected as a result of an ‘A’ to ‘G’ substitution at position 3876 in exon 21 (CCAG to CCGG). The enzyme had one recognition site in the PCR product (373 and 261 bp fragments, B₁ allele) and the nucleotide substitution resulted in an additional recognition site for the enzyme (373, 134 and 127 bp fragments, B₂ allele). This is a silent mutation, as both CCA and CCG code for the amino acid proline. The marker for coping with pregnancy and lactation stress in mice is the sequence containing the ‘A’ nucleotide at position 3876 of the mouse IGF-1R gene, identified by the 373/261 bp fragments (B₁ allele). Since the substitution is 20 base pairs upstream from the 12 base pair deletion, the 261 bp and 127 bp bands will shift by 12 base pairs when animals are homozygous or heterozygous for the deletion allele (D₂). As is known in the art, however, restriction patterns are not exact determinants of the sizes of fragments and are only approximate.

Site D: Site D: A 12 bp deletion was detected 20 bp 3′ to the site B in exon 21 (positions 3896-3907 of the IGF-1R gene cDNA, Genbank accession number AF056187, SEQ ID NO:1). This 12 bp fragment (tggagatggagc) (SEQ ID NO:20) appears twice in tandem (D₁ allele) in or only once (D₂ allele) in this region, resulting in the deletion of four amino acids (leucine, glutamic acid, methionine, and glutamic acid) from the IGF-1R protein. One IGF-1R sequence (Genbank accession number AF056187, SEQ ID NO:1) has two copies of this fragment while two others (Genbank accession numbers XM_{—}133508 (SEQ ID NO:3) and AC101879 (, SEQ ID NO:6) have one copy.

Allele and genotype frequency distributions: Although sites A and B are approximately 22 kb apart, all 153 juveniles and 261 breeders had exactly the same genotypes at these two sites, constituting only two alleles (A₁ and A₂). Replicate lines of juvenile mice were not different from the main lines for allele or genotype frequencies at site A. The frequency of Al allele in breeders from the SU1 line (0.84) was significantly greater than those in the other three lines (0.48, 0.62, 0.63, Tables 2 and 3). A similar pattern was observed in the juveniles, where frequencies of the A₁ allele in the SU1 and SU2 lines (0.83 and 0.89) were significantly greater than those in SA1 (0.55), SA2 (0.46), C1 (0.48) and C2 (0.61) lines (Tables 4 and 5). Frequencies of A₁ allele in the C1 line were similar in breeders and juveniles (0.48), and were smaller than those in the C2 line in breeders (0.67, P<0.01) and juveniles (0.61, NS). Frequency of Al allele in selected and control lines in which litter size was not standardized (SU1, SU2, C2) was greater than that in the lines in which litter size was standardized (SA1, SA2, C1) in both breeders and juveniles, suggesting that the Al allele was possibly selected for under high levels of maternal stress.

The frequency of the A₁A₁ genotype in breeders from SU1 line (0.71) was significantly greater than in other lines, which ranged between 0.23 (C₁) and 0.47 (C2) (Tables 2 and 3). Juveniles from the SU1 and SU2 lines had greater frequencies of the A₁A₁ genotype (0.75 and 0.77) and lower frequencies of the A₂A₂ genotype (0.10 and 0.0) than the other four lines, in which the frequencies of A₁A₁ ranged from 0.17 to 0.44 and frequencies of A₂A₂ ranged from 0.22 to 0.26 (Tables 4 and 5). Differences in genotype frequencies between SU1 and SU2 and the other lines were all significant, except for SU1 and C2 that approached significance (P=0.079). Genotype frequency distributions conformed to Hardy-Weinberg proportions in all the lines. All four inbred lines (C3H/HeJ, C57BL/6J, CBA/J, SWR/J) had the A₁A₁ genotype at site A and the B₁B₁ genotype at site B, indicating that the A₂ and B₂ alleles must have been introduced into the base population by the Q-strain.

No D₂ allele was detected in any of the control mice. The frequency of the D₂ allele (deletion) ranged from 0.10 to 0.19 in the selected lines in the juveniles and breeders. The selected lines within breeder and juvenile groups had comparable allele and genotype frequencies at site D. All selected lines had significantly different allele and genotype frequency distributions compared with the control lines in which the D₁ allele was fixed (Tables 6, 7, 8, 9). Replicate lines of juvenile mice were not different from the main lines for allele or genotype frequencies at site D. Genotype frequency distributions conformed to Hardy-Weinberg proportions in all the lines, except in juveniles from the SA1 line, which was deficient in heterozygotes (F_{IS}=+0.449, Table 4). High proportions of the D₂ allele appeared in the heterozygous state (0.179 to 0.385), and low proportions (0.0 to 0.107) were in homozygous form in all the selected lines, which is expected from a population in Hardy-Weinberg equilibrium in which one allele has a low frequency. The C57BL/6J had the D₂D₂ genotype, but the other three inbred lines had the D₁D₁ genotype.

Only six of the 10 possible genotypes were present in the population when the joint distribution of A and D sites was considered (Tables 10, 12), indicating the presence of three of the four possible haplotypes (A₁D₁, A₁D₂, A₂D₁). Haplotype and genotype frequency distributions were significantly different among all the lines within breeder and juvenile groups, except those between replicate lines (Tables 10, 11, 12, 13). Haplotype frequency differences between selected and control lines were largely due to the absence of the A₁D₂ haplotype in the latter. Differences among non-replicate selected lines for haplotype frequency distributions were mainly the result of higher frequencies of A₁D₁ (0.69 to 0.74) and lower frequencies of A₂D₁ (0.12 to 0.18) in selected non-standardized lines compared with those in standardized selected lines, which had lower frequencies of A₁D₁ (0.28 to 0.46) and higher frequencies of A₂D₁ (0.38 to 0.52). Genotype frequencies conformed to Hardy-Weinberg proportions in all the lines in both breeders and juveniles, except in the SA1 line in juveniles, which was deficient in heterozygotes (F_{IS}=+0.341, Table 12). There was no difference between male and female breeders for allele or genotype frequencies at any of the sites (data not shown).

Discussion

Similarities between the replicate lines for allele (haplotype) and genotype frequencies at all sites may indicate that the observed differences among non-replicate lines had happened before divergence of replicate lines from the main lines. These findings also imply that the size of the lines was great enough to make genetic drift a negligible force in changing the genetic profile of the lines in the last 8 generations of the selected lines (generations 18 to 24) and 26 generations of the controls (generations 44 to 69). The observed differences among the lines for allele frequency distributions can thus be largely attributed to the selection pressure applied to each line.

The finding that the A₁ allele had a significantly greater frequency in breeder animals in which litter size was not standardized to 8 (selected and control lines) may suggest that although this gene has not been under selection pressure for reproductive longevity, the A₁ allele may be linked to a QTL that has a favorable effect on maternal stress. Most female mice conceive while still nursing, which imposes a great pressure on them, and the effect will be more pronounced when litter size is large. It seems that the A₁ allele is associated with animals that may be able to better cope with such a stress. This finding has some ramifications in the livestock industry, such as swine and dairy cattle, where lactation and pregnancy often coincide. This is the first evidence showing that such a characteristic is genetically controlled.

The results from site D provide a different picture than of site A. The absence of the D₂ allele (deletion) in the control lines, and the similarity between all the selected lines for the allele and genotype frequencies within breeders and juveniles may suggest that the D₂ allele (or an allele which is linked to D₂) had a negative effect on early reproduction, and has therefore been eliminated from the control lines. This conclusion is based on three notions. First, the frequency of the D₂ allele in the original population was expected to be at least 0.125, because C57BL/6J with the D₂D₂ genotype provided ⅛ of the genes to the original population, and this line had also contributed to the Q-strain. The effects of 21 generations of selection for nursing ability of the mother and body weight of progeny that was applied to the original population before the establishment of the base population for this experiment is not known. Assuming, however, that the frequency of the D₂ allele was not drastically changed, it is unlikely that the D₂ allele with such a frequency had not been included in the first generation of the control line merely by chance. Second, absence of the D₂ allele in the control lines was not because of the small number of mice that were genotyped. The probability (a) that an allele with the frequency of Y or less in a population falls into a sample of size n (i.e., 2n alleles) is log (1−α)=2n log (1−Y). Setting n=25, which was the smallest sample size of the control lines in juveniles, and Y=0. 10 (the smallest estimate of the D₂ frequency in any line) will result in α=0.994, i.e., there is at least 99% probability that the D₂ allele with a frequency of 0.10 would be included in a sample of size 25. Combining the two control lines of juveniles will increase this probability to 99.99%. The total number of control mice tested juveniles and breeders) was 217, suggesting that the D₂ allele certainly does not exist in the control lines. Third, in the control lines, the male is removed from the cage 14 to 17 days following pairing. Replacement mice in the control lines are thus selected from females that conceived within the first 14 to 17 days after exposure to a male. The control lines, therefore, have been under mild selection for early reproduction. Although more studies are needed, it seems logical to believe that deletion of four amino acids from the IGF-1R would have some negative effect on the function of this polypeptide. The only explanation for the D₂ allele to have a frequency of 0.10 to 0.20 in the selected lines is that this allele, or one which is linked to it, had a positive effect on reproduction at a later age. In addition, the D₂ allele was largely in the heterozygous state, which will mask any negative effect of the allele.

The D and A sites are only 20 bp apart, and thus the likelihood of a crossing-over between them is very slim. Differences between lines for allele frequencies at sites A and D should be sought in the origin of the haplotypes. Since the only source of the A₂ allele was the Q strain, and there was no A₂D₂ haplotype in the population, it seems logical to assume that the A₂D₁ haplotype originated from the Q-strain and the A₁D₂ haplotype originated from C57BL/6J (the only inbred line carrying the A₁D₂ haplotype). Line C57BL/6J had a minor contribution to the Q strain, indicating that the Q strain might have carried the A₁D₂ haplotype as well. The A₁D₁ haplotype originated from the other three inbred lines as well as from the Q-strain. It seems reasonable to conclude that the A₁D₂ haplotype, which originated from the C57BL/6J line and has been eliminated from the control lines, is a QTL with a negative effect on early reproduction and a positive effect on reproductive longevity. The A₂D₁ haplotype that originated from the Q strain and had high frequencies in non-standardized lines (SU1, SU2, C2) may be a QTL that has been selected for under maternal pressure (large litter size, high milk production, pregnancy).

F_{IS} is a measure of the inbreeding coefficient of individuals in a subdivided population due to nonrandom mating, or inbreeding of an individual relative to the sub-population to which it belongs (Wright, 1943, 1978; Nei, 1973; Hartl and Clark, 1989). When mating is at random in a sub-population, F_{IS} is equal to zero. Positive F_{IS} values indicate within sub-populations inbreeding (more homozygosity than expected) due to mating between relatives. Negative F_{IS} values show less homozygosity than expected from a population at Hardy-Weinberg equilibrium. Conformation of genotype frequency distributions to Hardy-Weinberg values and small F_{IS} estimates indicate that mating between animals with respect to sites A and D and their joint distribution has been at random in all the lines except SA1 in juveniles. This is expected in view of the fact that the effect of individual alleles on phenotype (reproductive longevity) has not been visible.

Many lines of mice contributed to the base population, making it a heterogeneous stock with many segregating loci upon which selection pressure has been applied for 24 generations. The fact that allele frequencies at sites A and D in the entire sample were 0.63 and 0.89 in juveniles and 0.63 and 0.94 in breeders, respectively, point to the heterogeneity of the population at the present time. The observed genetic variability makes this colony unique.

TABLE 2
Distribution of allele and genotype frequencies at site A¹
at the IGF-1R locus in breeder mice, test for Hardy-Weinberg
equilibrium and F_{IS} estimates by line and sex.

| | | Allele | | | | | | | |
|---|---|---|---|---|---|---|---|---|---|
| | | frequency | | Genotype frequency | | | No. of | H-W | |
| Line | Sex | A₁ | A₂ | A₁A₁ | A₂A₂ | A₁A₂ | mice | prob | F_{IS} |
| SA1 | F | 0.648 | 0.352 | 0.407 | 0.111 | 0.481 | 27 | | |
| Selected | M | 0.603 | 0.397 | 0.379 | 0.172 | 0.448 | 29 | | |
| (standardized) | Total | 0.625 | 0.375 | 0.393 | 0.143 | 0.464 | 56 | 1.00 | 0.019 |
| SU1 | F | 0.881 | 0.119 | 0.809 | 0.048 | 0.143 | 21 | | |
| Selected | M | 0.810 | 0.190 | 0.619 | 0.000 | 0.381 | 21 | | |
| (Non-standardized) | Total | 0.845 | 0.155 | 0.714 | 0.024 | 0.262 | 42 | 1.00 | 0.011 |
| C1 | F | 0.488 | 0.512 | 0.190 | 0.214 | 0.595 | 42 | | |
| Control | M | 0.476 | 0.523 | 0.262 | 0.310 | 0.429 | 42 | | |
| (standardized) | Total | 0.482 | 0.518 | 0.226 | 0.262 | 0.512 | 84 | 1.00 | −0.019 |
| C2 | F | 0.700 | 0.300 | 0.500 | 0.100 | 0.400 | 40 | | |
| Selected | M | 0.641 | 0.359 | 0.436 | 0.154 | 0.410 | 39 | | |
| (non-standardized) | Total | 0.671 | 0.329 | 0.468 | 0.127 | 0.405 | 79 | 0.45 | 0.089 |
| Total | | 0.628 | 0.372 | 0.414 | 0.157 | 0.429 | 261 | 0.99 | |

¹Site A is a ‘G’ to ‘A’ substitution in intron 16, which is in linkage disequilibrium with an ‘A’ to ‘G’ substitution in exon 21 (site B).

TABLE 3
Pairwise comparison of the lines for
allele frequency (above diagonal) and
genotype frequency (below diagonal) for site A in breeder mice.

| Line | SA1 | SU1 | C1 | C2 |
|---|---|---|---|---|
| SA1 | — | 0.001 | 0.020 | 0.436 |
| SU1 | 0.001 | — | 0.000 | 0.003 |
| C1 | 0.020 | 0.000 | — | 0.002 |
| C2 | 0.446 | 0.006 | 0.001 | — |

TABLE 4
Distribution of allele and genotype frequencies at site A
of the IGF-1R locus in juveniles, test for Hardy-Weinberg
equilibrium and F_{IS} estimates by line.

| | | Allele | | | | | No. | | |
|---|---|---|---|---|---|---|---|---|---|
| | | frequency | | Genotype frequency | | | of | H-W | |
| Line | | A₁ | A₂ | A₁A₁ | A₂A₂ | A₁A₂ | mice | prob. | F_{IS} |
| Selected | SA1 | 0.552 | 0.448 | 0.345 | 0.241 | 0.414 | 29 | 0.45 | 0.180 |
| (Standardized) | SA2 | 0.458 | 0.542 | 0.167 | 0.250 | 0.583 | 24 | 0.68 | −0.154 |
| Selected | SU1 | 0.825 | 0.175 | 0.750 | 0.100 | 0.150 | 20 | 0.07 | 0.500 |
| (Non-standardized) | SU2 | 0.885 | 0.115 | 0.769 | 0.000 | 0.231 | 26 | 1.00 | −0.111 |
| Control | C1 | 0.481 | 0.519 | 0.222 | 0.259 | 0.519 | 27 | 1.00 | −0.020 |
| (standardized) | | | | | | | | | |
| Control (non-standardized) | C2 | 0.611 | 0.389 | 0.444 | 0.222 | 0.333 | 27 | 0.13 | 0.316 |
| Total | | 0.627 | 0.373 | 0.438 | 0.183 | 0.379 | 153 | 0.46 | 0.109 |

TABLE 5
Pairwise comparison of the lines for allele frequency
(above diagonal) and genotype frequency (below diagonal)
for site A in juveniles.

| Line | SA1 | SA2 | SU1 | SU2 | C1 | C2 |
|---|---|---|---|---|---|---|
| SA1 | — | 0.428 | 0.005 | 0.000 | 0.570 | 0.567 |
| SA2 | 0.451 | — | 0.000 | 0.000 | 0.845 | 0.160 |
| SU1 | 0.014 | 0.001 | — | 0.545 | 0.001 | 0.038 |
| SU2 | 0.000 | 0.000 | 0.592 | — | 0.000 | 0.001 |
| C1 | 0.588 | 0.836 | 0.002 | 0.000 | — | 0.246 |
| C2 | 0.617 | 0.189 | 0.079 | 0.004 | 0.279 | — |

TABLE 6
Distribution of allele and genotype frequencies of the deletion¹
at the IGF-1R locus in breeder mice, test for Hardy-Weinberg
equilibrium and F_{IS} estimates by line and sex.

| | | Allele | | | | | | |
|---|---|---|---|---|---|---|---|---|
| | | frequency | | Genotype frequency | | | H-W | |
| Line | Sex | D₁ | D₂ | D₁D₁ | D₂D₂ | D₁D₂ | Prob | F_{IS} |
| SA1 Selected (standardized) | F | 0.796 | 0.204 | 0.593 | 0.000 | 0.407 | | |
| | M | 0.862 | 0.138 | 0.758 | 0.034 | 0.207 | | |
| | Total | 0.830 | 0.170 | 0.679 | 0.018 | 0.304 | 1.00 | −0.069 |
| SU1 Selected | F | 0.929 | 0.071 | 0.857 | 0.000 | 0.143 | | |
| (Non-standardized) | M | 0.857 | 0.143 | 0.714 | 0.000 | 0.286 | | |
| | Total | 0.893 | 0.107 | 0.786 | 0.000 | 0.214 | 1.00 | −0.108 |
| C1 Control (standardized) | F | 1.000 | 0.000 | 1.000 | 0.000 | 0.000 | | |
| | M | 1.000 | 0.000 | 1.000 | 0.000 | 0.000 | | |
| | Total | 1.000 | 0.000 | 1.000 | 0.000 | 0.000 | — | — |
| C2 Selected | F | 1.000 | 0.000 | 1.000 | 0.000 | 0.000 | | |
| (non-standardized) | M | 1.000 | 0.000 | 1.000 | 0.000 | 0.000 | | |
| | Total | 1.000 | 0.000 | 1.000 | 0.000 | 0.000 | — | — |
| Total | | 0.946 | 0.054 | 0.897 | 0.003 | 0.100 | 1.00 | |

¹A 12 bp deletion (D₂ allele) in exon 21 of the IGF-1R gene.

TABLE 7
Pairwise comparison of the lines for
allele frequency (above diagonal) and
genotype frequency (below diagonal) for site D in breeder mice.

| Line | SA1 | SU1 | C1 | C2 |
|---|---|---|---|---|
| SA1 | — | 0.305 | 0.000 | 0.000 |
| SU1 | 0.218 | — | 0.000 | 0.000 |
| C1 | 0.000 | 0.000 | — | 1.000 |
| C2 | 0.000 | 0.000 | 1.000 | — |

TABLE 8
Distribution of allele and genotype frequencies of the deletion¹
at the IGF-1R locus (site D) in juveniles, test for
Hardy-Weinberg equilibrium and F_{IS} estimates by line.

| | Allele | | | | | | | |
|---|---|---|---|---|---|---|---|---|
| | frequency | | Genotype frequency | | | No. of | H-W | |
| Line | D₁ | D₂ | D₁D₁ | D₂D₂ | D₁D₂ | mice | prob. | F_{IS} |
| SA1 | 0.804 | 0.196 | 0.714 | 0.107 | 0.179 | 28 | 0.04 | 0.449 |
| SA2 | 0.804 | 0.196 | 0.652 | 0.044 | 0.304 | 23 | 1.00 | 0.055 |
| SU1 | 0.900 | 0.100 | 0.800 | 0.000 | 0.200 | 20 | 1.00 | −0.086 |
| SU2 | 0.808 | 0.192 | 0.615 | 0.000 | 0.385 | 26 | 0.54 | −0.220 |
| C1 | 1.000 | 0.000 | 1.000 | 0.000 | 0.000 | 27 | — | — |
| C2 | 1.000 | 0.000 | 1.000 | 0.000 | 0.000 | 25 | — | — |
| | 0.886 | 0.114 | 0.799 | 0.027 | 0.174 | 149 | 0.46 | 0.083 |

TABLE 9
Pairwise comparison of the lines for allele frequency
(above diagonal) and genotype frequency (below diagonal)
for site D in juvenile mice.

| Line | SA1 | SA2 | SU1 | SU2 | C1 | C2 |
|---|---|---|---|---|---|---|
| SA1 | — | 1.000 | 0.257 | 1.000 | 0.000 | 0.001 |
| SA2 | 1.000 | — | 0.261 | 1.000 | 0.001 | 0.001 |
| SU1 | 0.333 | 0.250 | — | 0.261 | 0.031 | 0.036 |
| SU2 | 1.000 | 1.000 | 0.211 | — | 0.001 | 0.001 |
| C1 | 0.005 | 0.001 | 0.029 | 0.000 | — | 1.000 |
| C2 | 0.005 | 0.001 | 0.032 | 0.000 | 1.000 | — |

TABLE 10
Distribution of haplotype and genotype frequencies for the
joint A and D sites in breeder mice, test for Hardy-Weinberg
equilibrium and F_{IS} estimates by line and sex.

| | | Haplotype | | | | | | | | | | |
|---|---|---|---|---|---|---|---|---|---|---|---|---|
| | | frequency | | | Genotype frequency | | | | | | | |
| | | | | A₂ | A₁A₁ | A₁A₁ | A₁A₁ | A₂A₂ | A₁A₂ | A₁A₂ | H-W | |
| Line | Sex | A₁ D₁ | A₁ D₂ | D₁ | D₁D₁ | D₂D₂ | D₁D₂ | D₁D₁ | D₁D₁ | D₁D₂ | prob | F_{IS} |
| SA1 | F | 0.444 | 0.204 | 0.352 | 0.185 | 0.000 | 0.222 | 0.111 | 0.296 | 0.185 | | |
| | M | 0.466 | 0.138 | 0.396 | 0.172 | 0.034 | 0.172 | 0.172 | 0.414 | 0.034 | | |
| | Total | 0.455 | 0.169 | 0.375 | 0.179 | 0.018 | 0.196 | 0.143 | 0.357 | 0.107 | 0.82 | −0.051 |
| SU1 | F | 0.810 | 0.071 | 0.119 | 0.667 | 0.000 | 0.143 | 0.047 | 0.143 | 0.000 | | |
| | M | 0.667 | 0.143 | 0.190 | 0.429 | 0.000 | 0.190 | 0.000 | 0.286 | 0.095 | | |
| | Total | 0.738 | 0.107 | 0.155 | 0.547 | 0.000 | 0.167 | 0.024 | 0.214 | 0.048 | 0.90 | −0.009 |
| C1 | F | 0.488 | 0.000 | 0.512 | 0.190 | 0.000 | 0.000 | 0.214 | 0.595 | 0.000 | | |
| | M | 0.476 | 0.000 | 0.524 | 0.262 | 0.000 | 0.000 | 0.310 | 0.428 | 0.000 | | |
| | Total | 0.482 | 0.000 | 0.518 | 0.226 | 0.000 | 0.000 | 0.262 | 0.512 | 0.000 | 1.00 | −0.019 |
| C2 | F | 0.700 | 0.000 | 0.300 | 0.500 | 0.000 | 0.000 | 0.100 | 0.500 | 0.000 | | |
| | M | 0.641 | 0.000 | 0.357 | 0.436 | 0.000 | 0.000 | 0.154 | 0.410 | 0.000 | | |
| | Total | 0.671 | 0.000 | 0.329 | 0.468 | 0.000 | 0.000 | 0.127 | 0.405 | 0.000 | 0.45 | 0.089 |
| Total | | 0.575 | 0.054 | 0.371 | 0.341 | 0.004 | 0.069 | 0.157 | 0.399 | 0.031 | 0.98 | |

TABLE 11
Pairwise comparison of the lines for
haplotype frequency (above diagonal) and
genotype frequency (below diagonal) for
joint A and D sites in breeder mice.

| Line | SA1 | SU1 | C1 | C2 |
|---|---|---|---|---|
| SA1 | — | 0.000 | 0.000 | 0.000 |
| SU1 | 0.000 | — | 0.000 | 0.000 |
| C1 | 0.000 | 0.000 | — | 0.000 |
| C2 | 0.000 | 0.000 | 0.001 | — |

TABLE 12
Distribution of haplotype and genotype frequencies for the
joint A and D sites in juveniles, test for Hardy-Weinberg
equilibrium and F_{IS} estimates by line.

| | Haplotype | | | Genotype frequency | | | | | | | |
|---|---|---|---|---|---|---|---|---|---|---|---|
| | frequency | | | A₁A₁ | A₁A₁ | A₁A₁ | A₂A₂ | A₁A₂ | A₁A₂ | H-W | |
| Lines | A₁ D₁ | A₁ D₂ | A₂ D₁ | D₁D₁ | D₂D₂ | D₁D₂ | D₁D₁ | D₁D₁ | D₁D₂ | prob. | F_{IS} |
| SA1 | 0.357 | 0.196 | 0.446 | 0.21 | 0.11 | 0.00 | 0.25 | 0.25 | 0.14 | 0.04 | 0.341 |
| SA2 | 0.283 | 0.196 | 0.522 | 0.09 | 0.00 | 0.00 | 0.22 | 0.35 | 0.26 | 0.63 | −0.048 |
| SU1 | 0.725 | 0.100 | 0.175 | 0.55 | 0.00 | 0.20 | 0.10 | 0.15 | 0.00 | 0.17 | 0.218 |
| SU2 | 0.692 | 0.192 | 0.115 | 0.46 | 0.00 | 0.31 | 0.00 | 0.15 | 0.08 | 0.68 | −0.125 |
| C1 | 0.481 | 0.000 | 0.519 | 0.22 | 0.00 | 0.00 | 0.26 | 0.52 | 0.00 | 1.00 | −0.022 |
| C2 | 0.620 | 0.000 | 0.380 | 0.48 | 0.00 | 0.00 | 0.24 | 0.28 | 0.00 | 0.08 | 0.423 |
| Total | 0.520 | 0.114 | 0.366 | 0.33 | 0.00 | 0.09 | 0.18 | 0.29 | 0.08 | 0.16 | 0.135 |

TABLE 13
Pairwise comparison of the lines for haplotype frequency
(above diagonl) and genotype frequency (below diagonal)
for joint A and D sites in juveniles.

| Line | SA1 | SA2 | SU1 | SU2 | C1 | C2 |
|---|---|---|---|---|---|---|
| SA1 | — | 0.696 | 0.001 | 0.000 | 0.001 | 0.001 |
| SA2 | 0.749 | — | 0.000 | 0.000 | 0.001 | 0.000 |
| SU1 | 0.008 | 0.001 | — | 0.415 | 0.000 | 0.010 |
| SU2 | 0.001 | 0.000 | 0.442 | — | 0.000 | 0.000 |
| C1 | 0.005 | 0.001 | 0.000 | 0.000 | — | 0.171 |
| C2 | 0.003 | 0.000 | 0.018 | 0.000 | 0.217 | — |

### EXAMPLE 2

### Identification of Polymorphisms in the IGF-1R Gene in a Line of Pigs for the Development of DNA

Animals from a single commercial operation were used to find polymorphisms in candidate genes for reproductive longevity in pigs. Sourcing all animals from a single farm should ensure a similar environment for both high and low reproductive longevity groups. Five living sows with very high parity numbers were chosen as representing high reproductive longevity and five animals culled for reproductive reasons at low parity numbers were chosen as representing low reproductive longevity.

DNA was extracted from tissue samples from these 10 animals and the DNA used to amplify regions of candidate genes using PCR. PCR primers were designed from pig DNA sequence, or from exonic sequence of the homologous gene in other species such as mouse or human. The DNA sequence of these PCR products was then determined and the sequences compared to identify any polymorphisms. Each polymorphism was then assayed over a larger sample of animals from the same commercial population to look for evidence of association with increased reproductive longevity.

Five polymorphisms were found. Of these five, 2 were in intron 16 (SNP16i27 and SNP16i73); one in exon 8 (SNP1772); one in exon 16 (SNP3085); and one in exon 21 (SNP3757).

The polymorphism designated SNP1772, was characterized as a G/A SNP. It is a TaqI RFLP. Polymorphism SNP16i27 (position 27 from the end of exon 16) is a G/A SNP. It is an AvaII RFLP. SNP16i73 (position 73 from the end of exon 16) is a G/C SNP. It is a MnlI RFLP.

### PCR-RFLP Protocol for SNP16i27

Primers used in RLFP Analysis were as Follow:

| | |
|---|---|
| Primer 16 | |
| 5′-CCT CCG TGA TGA AGG AGT TC-3′ | (SEQ ID NO: 14) |
| Primer 17 | |
| 5′-TCA GTT CCA TGA TGA CCA GC -3′ | (SEQ ID NO: 15) |

PCR was Carried out using the Following Conditions:

| | |
|---|---|
| 10X PCR Buffer | 1.0 ul |
| 2 mM dNTPs | 1.0 ul |
| 25 mM MgCl₂ | 1.0 ul |
| 5 uM Primer 16 | 1.0 ul |
| 5 uM Primer 17 | 1.0 ul |
| Amplitaq Gold | 0.1 ul |
| QH₂O | 3.9 ul |
| DNA | 1.0 ul |

Thermal Cycling conditions on the PE9700 were as follow:
94° C.-12 min
94° C.-30 sec
58° C.-30 sec
72° C.-30 sec
(repeated for 39 additional cycles)
72° C.-7 min
4° C.-hold

Digested with AvaII restriction endonuclease.

The expected product sizes were: allele 1: 141, 122, 44; allele 2: 122, 81, 60, 44.

### PCR-RFLP Protocol for SNP16i73

Primers used in RLFP Analysis were as Follow:

| | |
|---|---|
| Primer 16 | |
| 5′-CCT CCG TGA TGA AGG AGT TC-3′ | (SEQ ID NO: 16) |
| Primer 17 | |
| 5′-TCA GTT CCA TGA TGA CCA GC-3′ | (SEQ ID NO: 17) |

PCR was Carried out sing the Following Conditions:

| | |
|---|---|
| 10X PCR Buffer | 1.0 ul |
| 2 mM dNTPs | 1.0 ul |
| 25 mM MgCl₂ | 1.0 ul |
| 5 uM Primer 16 | 1.0 ul |
| 5 uM Primer 17 | 1.0 ul |
| Amplitaq Gold | 0.1 ul |
| QH₂O | 3.9 ul |
| DNA | 1.0 ul |

Thermal Cycling conditions on the PE9700
94° C.-12 min
94° C.-30 sec
58° C.-30 sec
72° C.-30 sec
(repeat for 39 additional cycles)
72° C.-7 min
4° C.-hold

Digested with MnlI restriction endonuclease.

The expected product sizes were: allele 1: 241, 55, 11; allele 2: 137, 104, 55, 11.

### PCR-RFLP Protocol for SNP1772

Primers used in RLFP Analysis were as Follow:

| | |
|---|---|
| Primer 9 | |
| 5′-GGA GTA TGA TGG GCA GGA T-3′ | (SEQ ID NO: 18) |
| Primer 8 | |
| 5′-GAA GCA TTG GTG CGA ATG TA-3′ | (SEQ ID NO: 19) |

PCR was Carried out using the Following Conditions:

| | |
|---|---|
| 10X PCR Buffer | 1.0 ul |
| 2 mM dNTPs | 1.0 ul |
| 25 mM MgCl₂ | 0.6 ul |
| 5 uM Primer 9 | 1.0 ul |
| 5 uM Primer 8 | 1.0 ul |
| Amplitaq Gold | 0.1 ul |
| QH₂O | 4.3 ul |
| DNA | 1.0 ul |

Thermal Cycling conditions on the PE9700
94° C.-12 min
94° C.-30 sec
56° C.-30 sec
72° C.-30 sec
(repeat for 39 additional cycles)
72° C.-7 min
4° C.-hold

Digested with TaqI restriction endonuclease.

The expected product sizes were: allele 1: 219; allele 2: 135, 84.

### EXAMPLE 3

### SNP 3832

Samples from old surviving sows and from young sows culled during the first 4 parities.

996 sows from four different farms were genotyped and tested for the effect of SNP 3832 on the number of parities. Allele “2” was found to be positively associated with longevity. In average sows of the 22, 12 and 11 genotypes were culled after 7.4, 6.7 and 5.1 parities, respectively. The additive effect of SNP 3832 was estimated to be 1.11/parities/allele (P=0.004) with no dominance effect. The effect is significant, but over estimated due to the data structure.

Germany (GER): Longevity (reproduction) data from sows with known pedigree with DNA samples from their sires.

Data of over 19,000 sows, daughters of 179 sires were used in the analysis. Each sire had at least 50 daughters. There are 76 litter farms represented and the litters were from 1996 to 2001. Phenotypic performance of each sire was estimated based on the daughters' performances, and genotypic data was collected for the sires. Allele “2” found to be positively associated with longevity. SNP 3832estimated additive effect represent a contrast between homozygous sows of 38 days to culling (P=0.062).

A large number of animals were genotyped for the SNP 3832 marker. Animals carrying two copies of the “2” allele (homozygous) are expected to produce more parities and stay in the herd longer.

PCR for SNP 3832

| | |
|---|---|
| Primer 22 | |
| 5′-AAG ATG AGG CCT TCC TT-3′ | (SEQ ID NO: 21) |
| Primer 23 | |
| 5′-GAT CAG CAG GTC GAG GAC TG-3′ | (SEQ ID NO: 22) |

PCR Conditions:

| | | |
|---|---|---|
| 10X PCR Buffer | | 1.0 ul |
| 2 mM dNTPs | | 1.0 ul |
| 25 mM MgCl2 | | 0.6 ul |
| 5 uM Primer 22 | | 1.0 ul |
| 5 uM Primer 23 | | 1.0 ul |
| Amplitaq Gold | seem to | 0.1 ul |
| QH2O | | 4.3 ul |
| DNA | | 1.0 ul |

Thermal Cycling conditions on the PE9700
94° C.-12 min
94° C.-30 sec
58° C.-30 sec
72° C.-1 min
(repeat for 34 additional cycles)
72° C.-7 min
4° C.-hold

Digest with FokI

Expected product sizes: allele 1: 347; allele 2: 292, 55.

### References

Agriculture and Agri-Food Canada. 2000b. Canadian Export Trade Highlights-1999. Agriculture and Agri-Food Canada Argi-Food Trade Service, May 2000. http://atn-riae.agr.ca/public/htmldocs/e2854.pdf
Allaire, F. R. and J. P. Gibson. 1992. Genetic value of herd life adjusted for milk production. J. Dairy Science. 75:1349-1356.
Apfeld, J. and C. Kenyon. 1998. Cell nonautonomy of C. elegans daf-2 function in theregulation of diapause and life span. Cell, 95:199-210.
Arthur, P. F., M. Makarechian, R. T. Berg and R. Weingardt. 1993. Longevity and lifetime productivity of cows in a purebred Hereford and two multibreed synthetic groups under range conditions. J. Anim. Sci. 71:1142-1147.
Bailey, C. M. 1991. Life span of beef-type Bos taurus and Bos indicus×Bos taurus females in a dry, temperate climate. J. Anim. Sci. 2379-2386.
Bascom, S. S. and A. J. Young. 1998. A summary of the reasons why farmers cull cows. J. Dairy Sci. 81:2299-2305.
Beaudeau, F., V. Ducrocq, C. Fourichon and H. Seegers. 1995. Effect of disease on length of productive life of French Holstein dairy cows by survival analysis. J. Dairy Sci. 78:103-117.
Boldman, K. G., A. E. Freeman, B. L. Harris and A. L. Kuck. 1992. Prediction of sire transmitting abilities for herd life from transmitting abilities linear type traits. J. Dairy Sci. 75:552-563.
Brown, J. C. and D. S. Falconer, 1960. Inbreeding depression and heterosis of litter size in mice. Genet. Res., camb. 1:262-274.
Copeland, N. G., N. A. Jenkins, D. J. Gilbert, J. T. Eppig, L. J. Malthus, J. C. Miller, W. F. Dietrich, A. Weaver, S. E. Lincoln, R. G. Steen. 1993. A genetic linkage map of mouse: current applications and future prospects. Science, 262:57-66.
Dieudonne, M. N., R. Pecquery, M. C. Leneveu and Y. Giudicelli. 2000. “Opposite effects of androgens and estrogens on adipogenesis in rat preadipocytes: Evidence for sex and site-related specificities and possible involvement of insulin-like growth factor-1 receptor and peroxisome proliferator-activated receptor γ2”. Endocrinol. 141(2): 649-656.
Durr, J. W., H. G. Monardes, R. I. Cue and J. C. Philpot. 1997. Culling in Quebec Holstein herds: 1. Study of phenotypic trends in herd life. Can. J. Anim. Sci. 77:593-600.
Falconer, D. S. 1960. Selection of mice for growth on high and low planes of nutrition. Genet. Res., camb. 1:91-113.
Falconer, D. S. 1973. Replicated selection for body weight in mice. Genet. Res., camb. 22:291-321.
Farid, A., D. C. Crober, H. van der Steen, D. L. Patterson and M. P. Sabour. 2002. Reproductive performance of a mice selected for reproductive longevity. 7^{th} World Cong. Genet. Appl. Live. Prod. Aug. 19021, Montpellier, France, Paper No. 08-13.
Garnett, I. and D. S. Falconer. 1975. Protein variation in strains of mice differing in body size. Genet. Ress., camb. 25:45-57.
Gill, G. S. and F. R.Allaire. 1976. Relationship of age at first calving, days open, days dry, and herdlife to a profit function for dairy cattle. J. Dairy Sci. 59:1131-1139.
Guo, S. W. and Thompson, E. A. 1992. Performing the exact test of Hardy-Weinberg proportions for multiple alleles. Biometrics 48:361-372.
Hartl, D. L., and A. G. Clark. 1989. Principles of population genetics. Sinauer Associates, Sunderland, Mass., USA, P 682.
Hekimi, S., B. Lakowski, T. M. Barnes and J. J. Ewbank. 1998. Molecular genetics of life span in C. elegans: how much does it teach us? TIG, 14 (1):14-20.
Hsin, H. and C. Kenyon. 1999. Signals from the reproductive system regulate the lifespan of C. elegans. Nature, 399: 362-366.
Jin, S., B. Zhai, Z. Qui, J. Wu, M. D. Lane and K. Liao. 2000. “c-Crk, a substrate of the insulin-like growth factor-1 receptor tyrosine kinase, functions as an early signal mediator in the adipocyte differentiation process”. J. Bio. Chem. 275(44): 34344-34352.
Kulak, K. K., J. C. M. Dekkers, A. J. McAllister and A. J. Lee. 1977. Lifetime profitability measures for dairy cows and their relationships to lifetime performance traits. Can. J. Anim. Sci. 77:609-616.
Lakowski, B. and S. Hekimi. 1996. Determination of life-span in Caenorhabditis elegans by four clock genes. Science, 272:1010-1013.
Lande, R. and Thopmson, R. 1990. Efficiency of Marker-assisted selection in the improvement of quantitative traits. Genetics 124:743-756.
Liu, J. P., J. Baker, A. S. Perkins, E. J. Robertson and A. Efstratiadis. 1993. “Mice carrying null mutations of the genes encoding insulin-like growth factor-1 (IGF-1) and type 1 IGF receptor (IGF-1R)”. Cell, 75: 59-72.
Nagai, J., C. Y. Lin and M. P. Sabour. 1995. Lines of mice selected for reproductive longevity. Growth, Development & Aging 57:79-91.
Nei, M. 1973. Analysis of gene diversity in subdivided populations. Proc. Natl. Acad. Sci. USA 70:3321-3323.
Packer, C, M. Tatar and A. Collins. 1998. Reproductive cessation in female mammals. Nature, 392:807-811.
Peruzzi, F., M. Prisco, M. Dews, P. Salomoni, E. Grassili, G. Romano, B. Calabretta and R. Baserga. 1999. “Multiple signaling pathways of the insulin-like growth factor-1 receptor in protection from apoptosis”. Mol. Cell. Biol., 19(10): 7203-7215.
Raymond, M. and Rousset, F. 1995. An exact test for population differentiation. Evolution 76:153.
Richards, R. G., M. P. Walker, J. Sebastian and R. P. DiAugustine. 1998. “Insulin-like growth factor-1 (IGF-1) receptor-insulin receptor substrate complexes in the uterus”. J. Biol. Chem., 273(19): 11962-11969.
Rose, M. P. and T. J. Nusbaum. 1994. Prospect for postponing human aging. FASEB J. 8:925-928.
Sell, C., G. Dumenil, C. Deveraud, M. Miura, D. Coppola, T. DeAngelis, R. Rubin, A.Efstratiadis and R. Baserga. 1994. “Effect of a null mutation of the insulin-like growth factor-1 receptor gene on growth and transformation of mouse embryo fibroblasts”. Mol. Cell. Biol., 14: 3604-3612.
Sell, C. M. Rubini, R. Rubin, J. P. Liu, A. Efstratiadis and R. Baserga. 1993. “Simian virus 40 large tumor antigen is unable to transform mouse embryonic fibroblasts lacking type-linsulin-like growth factor receptor”. Proc. Natl. Acad. Sci. USA 90: 11217-11221.
Silva, H. M., C. J. Wilcox, A. H. Spurlock, F. G. martin and R. B. Becker. 1986. Factors affecting age at first parturition, lifespan and vital statistics of Florida dairy cows. J. Dairy Sci. 69:470-476.
Smith, S. P. and R. L. Quass. 1984. Productive lifespan of bull progeny groups: failure time analysis. J. Dairy Sci. 67:2999-3007.
Soni, P., M. Lakkis, M. N. Poy, M. A. Fernstrom and S. M. Najjar. 2000. “The differential effects of pp120 on the mitogenic action of insulin and insulin-like growth factor-1 are regulated by the non-conserved tyrosine 1316 in the insulin receptor”. Mol. Cell. Biol., 20(11): 3896-3905.
Strandberg, E. 1996. Breeding for longevity in dairy cows. In ‘Progress in Dairy Science’ CAB Int., Wallingford, Oxon, United Kingdom, p 125.
Swantek, J. L. and R. Baserga. 1999. “Prolonged activation of ERK2 by epidermal growth factor and other growth factors requires a functional insulin-like growth factor-1 receptor”. Endocrinol., 140(7): 3163-3169.
Tanida, H., W. D. Hohenboken and S. K. DeNise. 1988. Genetic aspects of longevity in Angus and Hereford cows. J. Anim. Sci. 66:640-647.
Tissenbaum, H. A. and G. Ruvkun. 1998 An insulin-like signalling pathway affects both longevity and reproduction in Caenorhabditis elegans. Genetics 148:703-717.
Valentinis, B., G. Romano, F. Peruzzi, A. Morrione, M. Prisco, S. Soddu, B. Cristofanelli, A. Sacchi and R. Baserga. 1999. “Growth and differentiation signals by the insulin-like growth factor-1 receptor in hemopoietic cells are mediated through different pathways”. J. Biol. Chem. 274(18): 12423-12430.
VanRaden, P. M. and E. J. H. Klaaskate. 1993. Genetic evaluation of productive life including predicted longevity of live cows. J. Dairy Sci. 76:2758-2764.
Wright, S. 1943. Isolation by distance. Genetics 28:114-138.
Wright, S. 1978. Evolution and the genetics of populations. Vol. 4, Variability within and among natural populations. University of Chicago Press, Chicago, Ill., USA, p. 580.
Xu, P., A. R. Jacobs and S. I. Taylor. 1999. “Interaction of insulin receptor substrate 3 with insulin receptor, insulin receptor-related receptor, insulin-like growth factor-1 receptor and downstream signaling proteins”. J. Biol. Chem. 274(21): 15262-15270.

1. A method for genetically identifying an animal with respect to its potential to
reproductive longevity comprising:
obtaining a sample of genetic material from an animal; and
assaying for the presence of a polymorphism in the insulin-like growth factor 1 receptor gene (IGF-1R), wherein the polymorphism is associated with reproductive longevity.

2. The method of claim 1 wherein said polymorphism is selected from the group consisting of: a single nucleotide polymorphism (SNP), a deletion, and an insertion.

3. The method of claim 1 wherein the animal is selected from a group consisting of: a mouse, a pig, and a cow.

4. The method of claim 1 wherein a step of assaying the polymorphism is selected from the group consisting of: direct sequencing, restriction fragment length polymorphism (RFLP) analysis, single-stranded conformation polymorphism (SSCP), PCR amplification of specific alleles, amplification of DNA target by PCR followed by a mini-sequencing assay, allelic discrimination during PCR, Genetic Bit Analysis, Pyrosequencing, oligonucleotide ligation assay, and analysis of melting curves.

5. The method of claim 4 wherein the step of assaying the polymorphism is RFLP.

6. The method of claim 4 wherein the step of assaying the polymorphism is SSCP.

7. The method of claim 1 wherein the step of assaying for the presence of the polymorphism comprises the steps of:
digesting the genetic material with a restriction endonuclease that cleaves the gene in at least one place, wherein a particular restriction endonuclease pattern indicates the presence or absence of a polymorphism;
separating the fragments obtained from the digestion;
detecting a restriction pattern generated by the fragments; and
comparing the pattern with a second restriction pattern for the gene obtained by using the restriction endonuclease, wherein the second restriction pattern is associated with reproductive longevity.

8. The method of claim 7 wherein said separation is by gel electrophoresis.

9. The method of claim 7 further comprising:
amplifying the gene or a portion thereof which contains at least one polymorphism, prior to digestion.

10. The method of claim 9 wherein the amplification includes selecting a forward and a reverse sequence primer capable of amplifying a region of the gene which contains a polymorphism.

11. The method of claim 1 wherein the polymorphism is identified as an A to G nucleotide substitution at position 3876 of the gene.

12. The method of claim 1 wherein the polymorphism is identified as a G to A nucleotide substitution at position 331 of the gene.

13. The method of claim 1 wherein the polymorphism is a 12 base pair deletion at positions 3896-3907 of the gene.

14. The method of claim 7 wherein the restriction endonuclease is HpaII.

15. The method of claim 7 wherein the restriction endonuclease DpnII.

16. The method of claim 7 wherein the restriction endonuclease is TaqI.

17. The method of claim 7 wherein the restriction endonuclease is MnlI.

18. The method of claim 7 wherein the restriction endonuclease is AvaII.

19. The method of claim 10 wherein the forward primer is SEQ ID NO:8 and wherein the reverse primer is SEQ ID NO:9.

20. The method of claim 10 wherein the forward primer is SEQ ID NO:10 and wherein the reverse primer is SEQ ID NO:11.

21. The method of claim 10 wherein the forward primer is SEQ ID NO:12 and wherein the reverse primer is SEQ ID NO:13.

22. The method of claim 10 wherein the forward primer is SEQ ID NO:14 and wherein the reverse primer is SEQ ID NO:15.

23. The method of claim 10 wherein the forward primer is SEQ ID NO:16 and wherein the reverse primer is SEQ ID NO:17.

24. The method of claim 10 wherein the forward primer is SEQ ID NO:18 and wherein the reverse primer is SEQ ID NO:19.

25. A method of screening animals to determine those more likely to have reproductive longevity, the method comprising:
obtaining a biological sample from an animal; and
assaying for the presence of a genotype in the IGF-1R gene, wherein the genotype is associated with reproductive longevity and characterized by a restriction fragment pattern, wherein said pattern when compared to a second restriction pattern is known to have or not have a desired polymorphic marker, the presence of said marker being indicative of an animal more likely to have reproductive longevity.

26. The method of claim 25 wherein the assaying step comprises amplifying the gene or a region thereof containing the marker with a forward and a reverse sequence primer.

27. The method of claim 26 wherein the forward primer is SEQ ID NO:8 and the reverse primer is SEQ ID NO:9.

28. The method of claim 26 wherein the forward primer is SEQ ID NO:10 and the reverse primer is SEQ ID NO:11.

29. The method of claim 26 wherein the forward primer is SEQ ID NO:12 and said reverse primer is SEQ ID NO:13.

30. The method of claim 26 wherein the forward primer is SEQ ID NO:14 and the reverse primer is SEQ ID NO:15.

31. The method of claim 26 wherein the forward primer is SEQ ID NO:16 and the reverse primer is SEQ ID NO:17.

32. The method of claim 26 wherein the forward primer is SEQ ID NO:18 and the reverse primer is SEQ ID NO:19.

33. The method of claim 25 wherein the marker is DpnII.

34. The method of claim 25 wherein the marker is HpaII.

35. The method of claim 25 wherein the marker is Taql.

36. The method of claim 25 wherein the marker is MnlI.

37. The method of claim 25 wherein the marker is AvaII.

38. The method of claim 33 wherein a G to A nucleotide substitution results in a restriction pattern characterized by a 328 nucleotide fragment, a 125 nucleotide fragment, and a 32 nucleotide fragment.

39. The method of claim 34 wherein an A to G nucleotide substitution results in a restriction pattern characterized by a 373 nucleotide fragment, a 134 nucleotide fragment, and a 127 nucleotide fragment.

40. The method of claim 34 wherein the deletion is characterized by a 12 bp fragment having SEQ ID NO:20 appearing once in the IGF-1R gene.

41. The method of claim 35 wherein a G to A nucleotide substitution results in a restriction pattern characterized by a 135 nucleotide fragment and an 84 nucleotide fragment.

42. The method of claim 36 wherein an G to C nucleotide substitution results in a restriction pattern characterized by a 137 nucleotide fragment, a 104 nucleotide fragment, a 55 nucleotide fragment, and an 11 nucleotide fragment.

43. The method of claim 37 wherein an G to A nucleotide substitution results in a restriction pattern characterized by a 122 nucleotide fragment, an 81 nucleotide fragment, a 60 nucleotide fragment, and a 44 nucleotide fragment.

44. The method of claim 25 wherein said animal is selected from the group consisting of: a pig and a mouse.

45. A method for screening animals to determine those more likely to exhibit favorable traits associated with reproductive longevity, said method comprising:
obtaining a genetic sample from an animal; and
detecting the presence or absence of at least one allele in the IGF-1R gene wherein the presence of the allele is predictive of the animal having reproductive longevity.

46. The method of claim 45 wherein the allele is defined in intron 16 of the gene.

47. The method of claim 45 wherein the allele is defined in exon 21 at position 3876 of the gene.

48. The method of claim 45 wherein the allele is defined in exon 21 at positions 3896-3907 of the gene.

49. The method of claim 45 wherein the allele is defined at position 27 at the end of intron 16 of the gene.

50. The method of claim 45 wherein the allele is defined at position 73 at the end of intron 16 of the gene.

51. The method of claim 45 wherein the animal is selected from a group consisting of: a pig and a mouse.

52. A method for determining the haplotype of the IGF-1R gene of an animal comprising:
obtaining a genetic sample from an animal; and
analyzing the genetic sample for the presence of an IGF-1R gene A₁D₁, A₁D₂, or A₂D₁ haplotype allele, wherein the haplotype effects reproductive performance or the ability to sustain stress factors.

53. The method of claim 52 wherein the A₁D₁ allele is indicative of having a favorable effect on lactation and pregnancy stress.

54. The method of claim 52 wherein the A₁D₂ allele is indicative of having a negative effect on reproductive performance.

55. The method of claim 52 wherein the A₂D₁ allele is indicative of reproductive longevity.

56. The method of claim 52 wherein the animal is a mouse.

57. A method for genotyping an animal for reproductive longevity, the method comprising:
obtaining a sample of genetic material from an animal;
detecting a polymorphism in the IGF-1R gene of the animal;
determining whether the animal possesses a marker, wherein the marker is indicative of the animal having two copies of allele 2.

58. The method of claim 57 wherein the step of detecting the polymorphism comprises: digesting amplified nucleic acid with a restriction enzyme; and
separating the nucleic acid fragments according to size such that a restriction fragment pattern is generated,
wherein the restriction fragment pattern generated is indicative of an animal reproductive longevity.

59. The method of claim 57 wherein prior to digesting the nucleic acid with a restriction enzyme, amplifying the nucleic acid with a forward primer and a reverse primer.

60. The method of claim 59 wherein the forward and reverse primer is SEQ ID NO:21 and SEQ ID NO:22.

61. The method of claim 57 wherein the restriction enzyme is FokI.

62. The method of claim 58 wherein the restriction pattern characterized by a 295 nucleotide fragment, and a 55 nucleotide fragment.

63. The method of claim 57 wherein the marker is positively associated with longevity.

64. The method of claim 57 wherein the animal is a pig.

65. A method for genetically identifying an animal comprising:
obtaining a sample of genetic material from an animal; and
assaying for the presence of a genotype in the IGF-1R gene sequence as set forth in SEQ ID NO:1 or a region thereof in the sample,
wherein the animal possesses a nucleic acid sequence having at least 95% sequence identity to SEQ ID NO:1 or a fragment thereof.

66. The method of claim 65 wherein the polymorphism is identified by a G to A nucleotide substitution in intron 16.

67. The method of claim 65 wherein the polymorphism is identified by an A to G nucleotide substitution in exon 21.

68. The method of claim 65 wherein the polymorphism is identified as a 12 bp deletion in exon 21.

69. The method of claim 65 wherein the polymorphism is identified as an insertion of a G nucleotide in intron 16 at position 176.

70. The method of claim 65 wherein the animal is a mouse.

71. A method for genetically identifying an animal comprising:
obtaining a sample of genetic material from an animal; and
assaying for the presence of a genotype in the IGF-1R gene sequence as set forth in SEQ ID NO:7 or a region thereof in the sample,
wherein the animal posses a nucleic acid sequence having at least 95% sequence identity to SEQ ID NO:7 or a fragment thereof.

72. The method of claim 71 wherein said polymorphism is identified as a G to A nucleotide substitution in intron 16.

73. The method of claim 71 wherein said polymorphism is identified as a G to C nucleotide substitution in intron 16.

74. The method of claim 71 wherein said polymorphism is identified as a G to A nucleotide substitution in exon 8.

75. The method of claim 71 wherein the animal is a pig.

76. The method of claim 65 wherein the polymorphism is an A to G nucleotide substitution in exon 21 at position 3876.

77. The method of claim 65 wherein the polymorphism is a 12 bp deletion in exon 21 at positions 3896-3907.

78. The method of claim 71 wherein said polymorphism is a G to A nucleotide substitution at position 27 from the end of intron 16.

79. The method of claim 71 wherein said polymorphism is a G to C nucleotide substitution at position 73 from the end of intron 16.

80. A method for genetically identifying cattle with respect to its potential to reproductive longevity comprising:
obtaining a sample of genetic material from a cow; and
assaying for the presence of a polymorphism in the insulin-like growth factor 1 receptor gene (IGF-1R), wherein the polymorphism is associated with reproductive longevity.


SEQUENCE LISTING (22 sequences)

SEQ ID NO: 1 - DNA, 4500 bp, Mus musculus
atgaagtctggctccggaggagggtccccgacctcgctgtgggggctcgtgtttctctccgccgcgctctctctctggccgacgagtggagaaatctgtgggcccggcattgacatccgcaacgactatcagcagctgaagcgcctggaaaactgcacggtgatcgagggcttcctccacatcctgctcatctccaaggccgaggactaccgaagctaccgcttccccaagctcaccgtcatcactgagtacttgctgctcttccgagtcgctggcctcgagagcctgggagacctcttccccaacctcacagtcatccgtggctggaaactcttctacaactacgcactggtcatcttcgagatgaccaatctcaaggatattgggctttataatctgaggaacattactcggggggccatcaggattgagaagaacgccgacctctgttacctctccaccatagactggtctctcatcttggatgcggtgtccaataactacattgtggggaacaagcccccgaaggaatgtggggacctgtgtccagggacattggaggagaagcccatgtgtgagaagaccaccatcaacaatgagtacaactaccgctgctggaccacaaatcgctgccagaaaatgtgcccaagtgtgtgcgggaagcgagcctgcaccgagaacaacgagtgctgccacccggagtgcctgggcagctgccacacaccggacgacaacacaacctgcgtggcctgcagacactactactacaaaggcgtgtgtgtgcctgcctgcccgcctggcacctacaggttcgagggctggcgctgtgtggatcgcgatttctgcgccaacatccccaacgctgagagcagtgactcggatggcttcgttatccacgacgatgagtgcatgcaggagtgtccctcaggcttcatccgcaacagcacccagagcatgtactgtatcccctgcgaaggcccctgccccaaagtctgcggcgatgaagagaagaaaacgaaaaccatcgattcggtgacttctgctcaaatgctccaaggatgcaccatcctgaagggcaatctgcttattaacatccggagaggcaataacattgcctcggagttggagaacttcatggggctcatcgaggtggtgaccggctacgtgaagatccgccattctcatgccttggtctccttgtccttcctgaagaaccttcgtctcatcttaggagaggagcagctggaagggaactactccttctatgtcctagacaaccagaacttgcagcagctgtgggactggaaccaccggaacctgaccgtcaggtccggaaagatgtactttgctttcaatcccaagctgtgtgtctccgaaatttaccgcatggaggaagtgaccggaaccaagggacgccagagcaaaggggacataaacaccaggaacaacggagagcgagcttcctgtgaaagtgatgttctccgtttcacctccaccacgacctggaagaaccgaatcatcataacgtggcaccggtaccggccgccggactaccgggatctcatcagcttcacagtttactacaaggaggcaccatttaaaaacgttacggaatatgacgggcaggatgcctgtggctccaacagctggaacatggtggatgtagacctgcctccgaacaaggagggcgagcctggcattttactgcatgggctgaagccctggacccagtatgctgtctatgtcaaggctgtgaccctcaccatggtggaaaacgaccatatccgtggggccaaaagtgaaatcttgtacattcgcaccaatgcttcagtcccttccattcccctagatgtcctctcagcatcaaactcttcctctcagctgattgtgaagtggaatcctccaactctgcccaatggtaacttgagttactacattgtgaggtggcagcggcagccccaggatggttacctgtaccggcacaactactgctccaaagacaaaatacccatcagaaagtacgccgatggtaccatcgacgtggaggaggtgacggaaaatcccaagacagaagtgtgtggtggtgataaagggccatgctgcgcttgccctaaaactgaagctgagaagcaggctgagaaggaggaggctgagtaccgtaaagtctttgagaatttccttcacaattccatctttgtgcccaggcccgaaaggaggcggagagacgtcatgcaagtggccaacacgaccatgtccagccgaagcaggaacaccacggtagctgacacctacaatatcacagacccggaggagttcgagacagagtaccctttctttgagagcagagtggataacaaggagaggactgtcatctccaacctccggcctttcactctgtaccgcatcgatatccacagctgcaaccacgaggctgagaagctgggctgcagcgcctccaacttcgtctttgcgagaaccatgccagcagaaggagcagatgatatccctggtccggtgacctgggagccaagacccgaaaactccatctttttaaagtggccagaacccgagaaccccaacggattgatcctaatgtatgaaattaaatacgggtcgcaagtcgaggatcagcgggaatgtgtgtccagacaggagtacaggaagtacggaggggccaaactcaaccgtctaaacccagggaactatacagcccggattcaggctacctccctctctgggaatgggtcatggacagatcctgtgttcttctatgtccccgccaaaacgacgtatgagaacttcatgcatctgatcattgctctgccggttgccatcctgctgatcgttggggggctggttatcatgctgtatgtcttccatagaaagagaaataacagcaggttgggcaatggagtgctgtatgcttctgtgaaccccgagtatttcagcgcagctgatgtgtacgtgcctgatgaatgggaggtagctcgagagaagatcaccatgaaccgggagctcggacaagggtcctttgggatggtctatgaaggagtggccaagggtgtggtcaaggatgaacccgaaaccagagtggccatcaagacggtaaacgaggctgcaagtatgcgtgaaagaatcgagtttctcaacgaggcctcggtgatgaaggagttcaattgtcaccatgtggtccggttgctgggtgtggtatcccaaggccagcccaccctggtcatcatggaactaatgacacgcggtgatctcaaaagttatctccggtctctgaggccagaagtggagcagaataatctagtcctcattcctccgagcttaagcaagatgatccagatggctggagagattgcagatggcatggcctacctcaatgccaacaagttcgtccacagagaccttgctgctaggaactgcatggtagccgaagatttcacagtcaaaattggagatttcggtatgacacgagacatctacgagacggactactaccggaaaggcgggaagggtttgctgcctgtgcgctggatgtctcccgagtccctcaaggatggtgtcttcactactcattctgatgtctggtccttcggggtcgtcctctgggagatcgccacgctggctgagcagccctaccagggcttgtccaacgagcaagttcttcgtttcgtcatggagggtggccttctggacaagccggacaactgccctgatatgctgtttgaacttatgcgcatgtgctggcagtataaccccaagatgcggccctccttcctggagatcatcggcagcatcaaggatgagatggagcccagcttccaggaggtctccttctactacagcgaggagaacaagcctcccgagccagaggagctggagatggagctggagatggagcctgagaacatggagagcgtcccactggacccttcggcctcctcagcctccctgcctctgcctgaaagacactcaggacacaaggctgagaatggcccgggccctggcgtgctcgttctccgcgccagttttgatgagagacagccttacgctcacatgaacgggggacgcgccaacgagagggccttgcctctgccccagtcctcgacctgctgatcctcggacacaccgaagcacgcgccaacagtaacgtgtgtgccccactcggtgggcgggggggcggggaggggagagcaggttgtaacaatctattcacaagcctcctgtacctcagtggatcttcagacctgccattgctgcccacgggagacggcttctctgcagtaaacacatttgggaccttccttttttcaatatgcaagcagctttttatttcctttacccgaacccttaactgacatgggcctctgcaaaccttaatgacaacacttaatagcaacaggacactcgagaattgagtctcttcgttctctgcctttttctctcctctgccttccctctctgccctctccccttccacttccacgctctcct

SEQ ID NO: 2 - protein, 1373 aa, Mus musculus
MKSGSGGGSPTSLWGLVFLSAALSLWPTSGEICGPGIDIRNDYQQLKRLENCTVIEGFLHILLISKAEDYRSYRFPKLTVITEYLLLFRVAGLESLGDLFPNLTVIRGWKLFYNYALVIFEMTNLKDIGLYNLRNITRGAIRIEKNADLCYLSTIDWSLILDAVSNNYIVGNKPPKECGDLCPGTLEEKPMCEKTTINNEYNYRCWTTNRCQKMCPSVCGKRACTENNECCHPECLGSCHTPDDNTTCVACRHYYYKGVCVPACPPGTYRFEGWRCVDRDFCANIPNAESSDSDGFVIHDDECMQECPSGFIRNSTQSMYCIPCEGPCPKVCGDEEKKTKTIDSVTSAQMLQGCTILKGNLLINIRRGNNIASELENFMGLIEVVTGYVKIRHSHALVSLSFLKNLRLILGEEQLEGNYSFYVLDNQNLQQLWDWNHRNLTVRSGKMYFAFNPKLCVSEIYRMEEVTGTKGRQSKGDINTRNNGERASCESDVLRFTSTTTWKNRIIITWHRYRPPDYRDLISFTVYYKEAPFKNVTEYDGQDACGSNSWNMVDVDLPPNKEGEPGILLHGLKPWTQYAVYVKAVTLTMVENDHIRGAKSEILYIRTNASVPSIPLDVLSASNSSSQLIVKWNPPTLPNGNLSYYIVRWQRQPQDGYLYRHNYCSKDKIPIRKYADGTIDVEEVTENPKTEVCGGDKGPCCACPKTEAEKQAEKEEAEYRKVFENFLHNSIFVPRPERRRRDVMQVANTTMSSRSRNTTVADTYNITDPEEFETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVPAKTTYENFMHLIIALPVAILLIVGGLVIMLYVFHRKRNNSRLGNGVLYASVNPEYFSAADVYVPDEWEVAREKITMNRELGQGSFGMVYEGVAKGVVKDEPETRVAIKTVNEAASMRERIEFLNEASVMKEFNCHHVVRLLGVVSQGQPTLVIMELMTRGDLKSYLRSLRPEVEQNNLVLIPPSLSKMIQMAGEIADGMAYLNANKFVHRDLAARNCMVAEDFTVKIGDFGMTRDIYETDYYRKGGKGLLPVRWMSPESLKDGVFTTHSDVWSFGVVLWEIATLAEQPYQGLSNEQVLRFVMEGGLLDKPDNCPDMLFELMRMCWQYNPKMRPSFLEIIGSIKDEMEPSFQEVSFYYSEENKPPEPEELEMELEMEPENMESVPLDPSASSASLPLPERHSGHKAENGPGPGVLVLRASFDERQPYAHMNGGRANERALPLPQSSTC

SEQ ID NO: 3 - DNA, 4973 bp, Mus musculus
gctcattcatttccactccgcatttctgcccctcgccggcctcgcccgcgcccgggacttcggaccagtctcgccaactgcgtcgcgctctcccgccgcgtaggctccggtggtcgttccctccggggatcgggtggcgtttgtcctcgcctgcggcgatttgggctttgctcctctttctgtacagttttcctccttcttctgcatctctgcgtttgcaaatggaggccgacgacgccgacagcccgccccggcgcgccccggcttcccgactccgccgccccgtgggccgctgctgccggcgctgaggggccgccccgcgccaccctccttgtccacgccgctttccgaggatcgtcgcctgcgctcttgtttttggacgagagtgaggactgagttggagactttttttttctttttttcttttcttttttttttttttttctatttttgagaaaagggaatttcgtcccaaataaaaggaatgaagtctggctccggaggagggtccccgacctcgctgtgggggctcgtgtttctctccgccgcgctctctctctggccgacgagtggagaaatctgtgggcccggcattgacatccgcaacgactatcagcagctgaagcgcctggaaaactgcacggtgatcgagggcttcctccacatcctgctcatctccaaggccgaggactaccgaagctaccgcttccccaagctcaccgtcatcactgagtacttgctgctcttccgagtcgctggcctcgagagcctgggagacctcttccccaacctcacagtcatccgtggctggaaactcttctacaactacgcactggtcatcttcgagatgaccaatctcaaggatattgggctttataatctgaggaacattactcggggggccatcaggattgagaagaacgccgacctctgttacctctccaccatagactggtctctcatcttggatgcggtgtccaataactacattgtggggaacaagcccccgaaggaatgtggggacctgtgtccagggacattggaggagaagcccatgtgtgagaagaccaccatcaacaatgagtacaactaccgctgctggaccacaaatcgctgccagaaaatgtgcccaagtgtgtgcgggaagcgagcctgcaccgagaacaacgagtgctgccacccggagtgcctgggcagctgccacacaccggacgacaacacaacctgcgtggcctgcagacactactactacaaaggcgtgtgtgtgcctgcctgcccgcctggcacctacaggttcgagggctggcgctgtgtggatcgcgatttctgcgccaacatccccaacgctgagagcagtgactcggatggcttcgttatccacgacgatgagtgcatgcaggagtgtccctcaggcttcatccgcaacagcacccagagcatgtactgtatcccctgcgaaggcccctgccccaaagtctgcggcgatgaagagaagaaaacgaaaaccatcgattcggtgacttctgctcaaatgctccaaggatgcaccatcctgaagggcaatctgcttattaacatccggagaggcaataacattgcctcggagttggagaacttcatggggctcatcgaggtggtgaccggctacgtgaagatccgccattctcatgccttggtctccttgtccttcctgaagaaccttcgtctcatcttaggagaggagcagctggaagggaactactccttctatgtcctagacaaccagaacttgcagcagctgtgggactggaaccaccggaacctgaccgtcaggtccggaaagatgtactttgctttcaatcccaagctgtgtgtctccgaaatttaccgcatggaggaagtgaccggaaccaagggacgccagagcaaaggggacataaacaccaggaacaacggagagcgagcttcctgtgaaagtgatgttctccgtttcacctccaccacgacctggaagaaccgaatcatcataacgtggcaccggtaccggccgccggactaccgggatctcatcagcttcacagtttactacaaggaggcaccatttaaaaacgttacggaatatgacgggcaggatgcctgtggctccaacagctggaacatggtggatgtagacctgcctccgaacaaggagggcgagcctggcattttactgcatgggctgaagccctggacccagtatgctgtctatgtcaaggctgtgaccctcaccatggtggaaaacgaccatatccgtggggccaaaagtgaaatcttgtacattcgcaccaatgcttcagtcccttccattcccctagatgtcctctcagcatcaaactcttcctctcagctgattgtgaagtggaatcctccaactctgcccaatggtaacttgagttactacattgtgaggtggcagcggcagccccaggatggttacctgtaccggcacaactactgctccaaagacaaaatacccatcagaaagtacgccgatggtaccatcgacgtggaggaggtgacggaaaatcccaagacagaagtgtgtggtggtgataaagggccatgctgcgcttgccctaaaactgaagctgagaagcaggctgagaaggaggaggctgagtaccgtaaagtctttgagaatttccttcacaattccatctttgtgcccaggcccgaaaggaggcggagagacgtcatgcaagtggccaacacgaccatgtccagccgaagcaggaacaccacggtagctgacacctacaatatcacagacccggaggagttcgagacagagtaccctttctttgagagcagagtggataacaaggagaggactgtcatctccaacctccggcctttcactctgtaccgcatcgatatccacagctgcaaccacgaggctgagaagctgggctgcagcgcctccaacttcgtctttgcgagaaccatgccagcagaaggagcagatgatatccctggtccggtgacctgggagccaagacccgaaaactccatctttttaaagtggccagaacccgagaaccccaacggattgatcctaatgtatgaaattaaatacgggtcgcaagtcgaggatcagcgggaatgtgtgtccagacaggagtacaggaagtacggaggggccaaactcaaccgtctaaacccagggaactatacagcccggattcaggctacctccctctctgggaatgggtcatggacagatcctgtgttcttctatgtccccgccaaaacgacgtatgagaacttcatgcatctgatcattgctctgccggttgccatcctgctgatcgttggggggctggttatcatgctgtatgtcttccatagaaagagaaataacagcaggttgggcaatggagtgctgtatgcttctgtgaaccccgagtatttcagcgcagctgatgtgtacgtgcctgatgaatgggaggtagctcgagagaagatcaccatgaaccgggagctcggacaagggtcctttgggatggtctatgaaggagtggccaagggtgtggtcaaggatgaacccgaaaccagagtggccatcaagacggtaaacgaggctgcaagtatgcgtgaaagaatcgagtttctcaacgaggcctcggtgatgaaggagttcaattgtcaccatgtggtccggttgctgggtgtggtatcccaaggccagcccaccctggtcatcatggaactaatgacacgcggtgatctcaaaagttatctccggtctctgaggccagaagtggagcagaataatctagtcctcattcctccgagcttaagcaagatgatccagatggctggagagattgcagatggcatggcctacctcaatgccaacaagttcgtccacagagaccttgctgctaggaactgcatggtagccgaagatttcacagtcaaaattggagatttcggtatgacacgagacatctacgagacggactactaccggaaaggcgggaaggggttgctgcctgtgcgctggatgtctcccgagtccctcaaggatggtgtcttcactactcattctgatgtctggtccttcggggtcgtcctctgggagatcgccacgctggctgagcagccctaccagggcttgtccaacgagcaagttcttcgtttcgtcatggagggtggccttctggacaagccggacaactgccctgatatgctgtttgaacttatgcgcatgtgctggcagtataaccccaagatgcggccctccttcctggagatcatcggcagcatcaaggatgagatggagcccagcttccaggaggtctccttctactacagcgaggagaacaagcctcccgagccagaggagctggagatggagcctgagaacatggagagcgtcccactggacccttcggcctcctcagcctccctgcctctgcctgaaagacactcaggacacaaggctgagaatggcccgggccctggcgtgctcgttctccgcgccagttttgatgagagacagccttacgctcacatgaacgggggacgcgccaacgagagggccttgcctctgccccagtcctcgacctgctgatcctcggacacaccgaagcacgcgccaacagtaacgtgtgtgcgcccactcggtgggcgggggggcggggaggggagagcaggttgtaacaatctattcacaagcctcctgtacctcagtggatcttcagacctgccattgctgcccacgggagacggcttctctgcagtaaacacatttgggaccttccttttttcaatatgcaagcagctttttatttcctttacccgaacccttaactgacatgggcctctgcaaaccttaatgacaacacttaatagcaacaggacactcgagaattgagtctcttcgttctctgcctttttctctcctctgccttccctctctgccctctccccttccacttccacgctctcct

SEQ ID NO: 4 - protein, 131 aa, Mus musculus
LFELMRMCWQYNPKMRPSFLEIIGSIKDEMEPSFQEVSFYYSEENKPPEPEELEMELEMEPENMESVPLDPSASSASLPLPERHSGHKAENGPGPGVLVLRASFDERQPYAHMNGGRANERALPLPQSSTC

SEQ ID NO: 5 - DNA, 446 bp, Mus musculus
aacgaggctgcaagtatgcgtgaaagaatcgagtttctcaacgaggcctcggtgatgaaggagttcaattgtcaccatgtggtaagaagccaagatgagacatggacagaaagtatggacagaaaggaccaccctgtatgtccttggccagctacggaattgttgccttgccctcttcccagtgggtagtttccccgttgcattcttgccacccacagacccttgaggtgggagcctcgccctcgccctcccaccagaactctgctcctggctacaggccccagcaccacccagttccgagaggcgtaggtcatcatgtgggatgcccccctccccctctgagtccttttcttgattcctccaggtccggttgctgggtgtggtatcccaaggccagcccaccctggtcatcatggaactaatgacacgcggtgatctcaaaagtt

SEQ ID NO: 6 - DNA, 174481 bp, Mus musculus
gaattccagatggaagctgacttcctgctcagccctattaaaaaaaaacacaggctttcctgcctctgagccccttcgccacaaataacttgagtgccatgcacggaggtgcccacttgtggcttaggttctctcagaccttgtttgtccttcagccccttggggaggagcccaggctaccacctgctgtccctaagcctaggacccagtggagaccatcgaattgcagatttagacagaattaaccatcacagggcctcataccatcctttttaaaggaccaggccttgatggagactgcagtcgccaacccccatggtgcttttgctactatggaaacactgaaataaccaacacacacactggttgtggatgccactgaacacacacacacacacacacacacacacacacacacactggttgatgcctctgaaccccaaaaggtcagcctggaaggaatggtggctgctctgtcctccatctcagccgcacatctgaagtcaccccagccacaggcacctgtcattgtcacagctggtccctatgggcttactctttcctcacgatgctcttatttttagtcagacaccaaagcgctttatttatttttagttagacacatttggcacatttcaagttttccaacttgtattcgataaagggaaacaaaggattcctgtcacctgtcctaattgaaagcttctttgataaatactttcggacagtgattcatactatcagccttccaaaaggaggaaggaaaaaaaaaaaaaagaaaagaaaagagcattcttttgagacactcttattcccttgcacttctgatttgttttttttttttttttttttatttcgggtccagagatggaatggctcaaattacatttgacgtcctcaggaaaccatactcttggtaaaagcagtgcctgtctcaaatgaaatatgggcatggaaatgtgctgccccctcttggactcacgtctttccctacatttctctcatcagccagcagttgctgggagtcaggcgccattgggaaatgcccactggatcccagagtgtggcctgcctttattagtaatgttttgagtttgccggaggacagactcttcattttattggagcccctagttgtgtaaatcttgtcttgacaactggtatgtagacagatggtgaaatgcaaggattctaacaaacagcctttagattaaggtgttggctgggaccaacagtaaaatgagagaccaggcgaacctgcagtcctccccagggttcgatgattctaccacattttctggctgactacccaactctattccaaggcagtcttgatggggaatgaggcttagcccagcaaaacaagaccttctaaccttgctcccggagttgtctttcctaatttaaaacaaacaaggatgtagtttcctcaaagctgaatgtggttctaaagtgtgtgaaatgtgtgttgatgggtttttaatgatcctagaggaagctggcttctcggtctttctacttaatctcaacattttctaagccgttctttagagctgcaggttcccacagcagctgcaagactgtgtatcctatcagtcaaagccagatggtagtcctgagatggtgggccattctgttgaaaagctttctgtcccctgaagtttttctccctaggaaccaatcctgatgatgggccacgaccaatacactcagcagttgccaggcacagctggatcccctctccagtgctgctacctactctagaggccctggggtccagcgacttgatgtagcatcctgatcttttacagactggaaagtgtggcaactgagaagtatgcgctgagagtcaggtgagggactcaaaccctcagtcatactccagataaaactgatcaggacgatgctcaaccctacctgcagcccaagctgagatgaggctcccctcctctcagtctgacatgctagcctcccccaggaggaggccacacatttttcattttagaaatatagatcaagaagaaattgtaaagaaagagaactccttgcaaatctcactagggttgcctgatgctgacatcttcaatgtccaatccaatgtactaacatcagtacaattaagaaagagagaagcagagagagggggagaaggaagggaaggagaacgggatggaggggttgaggaatgagtatgtgtgcacgtgcacacagaccgctctatacaatttagaccagtgcaaggcttcctggaattgcctccacctcaaaacgccttcccctgctactcccaccttagcttcttccgcactttcttcccccattttaacacgtggtgtccttttcagactcatttggctgtaataaaatatccagaaaacaaacaaacgacaaaggataaaggagtttatttccgtttacaattccaggttacagtccatcgtggtgggaaagtcagcagcaggaacttcagagaattggtcgcatcacatccacagtcaagagcagagagaaatgcatacatatttgctgtcttgtttgctttctcatgttcagcttggatttctccattcttttatagttcaggactcaatgcctagggaatgacactgcccatagtgaactgggttcccccacatcaattaacaagaattgttgaggaattgggttggcttgtggacatggtttttttttggaggattatcttagttaagctaattgatataagcagaaccattccctaggcaatgattggcctgtgggcatgtcaacatttcctaggaatgttggcctgtgagtatgtctgtgcccacaagtaagacaattggctaattgatatgagaaggcccagcccactgtaggtggtaccattccttaggctggagatccttgagctgtaggagagaggagaaatcaaactgagcacaagcaagtgagcatgtgtgcatctctctgctctttgactgtggatgctatgtgactgttgtttgaagtcctgcctcaactttcccacagtgatgggctataacataattatatgctgaaatgaattcctctctcctctaagttgctttcgttgttgtttttattacagcaacagttaaagtagaacacttatttaaaacctatggaaggatatatagatagtttacagctttagagttgttagagtggagaacactactgtaaacagtagtttccagttctgagatcagtggccaggtgtatacatgttactcagtccactttcagctctaaaaaaaaaaaaaattggggctcagcggttaagagcactgactgctcttccaaaggtcctgagttcaaatcccagcaaccacacggtggctcacaaccacccataacataacaaggtctgatgccctcttctggagcttctgaagacagctacagtgtactcacatataataataataaataaatcttttttttaaatctatcaccatattgcaaagtagtgtcttccattcctatcaatatgtgagacccagtttctcattctttctttgtttgtttgtttaagattttatttattcattgtatgtatatgagtacactgtagttgtacagatggttgtgggccatcatgtcattgctgggaattaaattcagtacctccgctggctccggccccgctggctccagcccaaagatttatttattgttagaccttttaaatatatcacataatagtctttatacaatagataagtgctttctcagtaataacctcttaacagagtttttattttttatttttaacagagtttttaaaagagccaacattcttaactctgtggctgtatttttgcttgtcttgttttaaaacaagctcttgggtggtggtagtgcacacctttagtcccagcagatactggcagatccctatgaactggtcgagagagcaagttacagaaaagtcaaggttacacagagaaaccctgctaactaaggttgagatctgctatgtctagatggcttatctgagctcagtgcaaaatggaggactctataccatctgggcttccccttctctgaccttgtctaggggactccctatactctctacctgggaaaggtcaagtagcctcggaagaccaacaaacttcctcttttccgcagaagaacccacccaacaagtacccgggattcctgaaatgcctctccatacaaatgaggaattccaagaactttaaactctaaccaatgattttcatttaccctgaaaactccttcacactctccaaggtcctggtttaccccaaataaagtgtattcgtacaagccccaccctagtagaggtatggaggtatgctcacaaaccaagatcggctaagagagctgttttcattgaagattgttggagaaggccttcttctaaaagagctgtaactctaagatccttcgggaaggccttctctcccttccctccccctccccccactagcactcactcccagcaggaccctaatcctgtataaccctccccccccccccattctggacactgtttcatccttcccacccagtgtgcagtggcatctggatacctccctgactatgtttcatacagtcaattctgaggaagcccccagattagcaaggctgaggttctctggggcctatagaacttgttgccaggtgatagaacttgttactgggtagtgtgggctggtttgagcaaacctgggtatctttggtttcaggttttgggcccttccggcagcacctagtgaattccagctgggaagccgtgaaggtaaatcctgccgtttgcccccccccccccccccccccgggccaacctggttccttctgtggctcttgggaaaccccaatgccaatgcgcgggactcctaagaaaccagagtcaccttggaaggaaggcaagcagtgggcccctggaagaaaggcttgtatttggtcctggaatggtgtgacttgcctggtcaggtacttctctcgtttgtccagggacactcctaggtgggaccaaggcccatgtaggctctactctacgtagggctagattcttgccctctcttcctttggatgtggggtctggggaaagtcattttcaccagctcttgtcctcacctacgcatgcggaagtgataacatggatgtctctggcctccactgacagctcagggtgagggcagccttgtccacactgctctgtgtgagcccagcatggggcgtgggtgagaacctggccagtgctcgtgactgtcgacagcacgcttgcttttgaggacatagctaaccaggagaagggaagtcctctgaccgcccactgtgcataggagggcaggactccctggcccacccccacctccctaggtcaactgtctgccatcagtcctcatgtgggtgtcacttagaggctctgcgaattgaaagctgcctgttcgcacttggctttcaacttcacactgatgttggaccaagccaaatgtggggcctggagtctattctcagagtttcccaggccagctgagaaacgcagcaaagctgctagcgtcatgcactcacagccaccgtggcactgactttgtggtctgacaacgcagaggcaggcaggagactcttgatccttgagttagcatttgggcttttgcttttccaatcataaaaaagcccagagacccttggactcattgccaagccttgctctggtacacaaggtaacagagaggccaggaccagactttcatgagctcctggctttctgcaaccagtatttgatcagcaggacttctgccaagtcctcagtagtcccagacccttttgtttctttctgcagccgtaaacaatggaaaagagccagactgcttctggtttctggtttgcctctcagtttccccagttgcaagtagtggcattcaaaaaccttgctcgagttttgatgcctccgtttcccaatttaaataaatgtgtgtgtgtgtgtgtgtgtgtgtgtgtatgtgagagagagagagagagagttgaggaagtcacatgacatcccaaagacctcttgggcatgctgatagagctcacaggttgtccattcccaccaggaggcttttctgtttgtctggtacacagctgcagacacatgggatgagggtcagatcagcacaggaagtggcagaagggagagggttacctgaggtggttgggaaaccccagtgcttgggactgctagtaatcaaagtcaccttggagggaaggcaagccagtgaccactggaagaaaagtttgtctttggtccttgggtggtgtgacttgactggtcaggtacgtcttccatgtgtgcagtaaagaaaagtggctaaggaggctcaaggctcattaagcttggtgaagcaaaccagacagacacagggactggggcttgcctgaaggcatatagtcagacatgcagacagccaaggatagctctgaaaagtgcacacacatacacactcacatgcactatacacacacacacacatacactattcctacacacatataccacaaacactatacacataccacacacatacactcacacacactatacacacaccacacacatatactcacatatactatacacacacacacatgcactacacacacatatatacacacatacactctcactcaggagctataggctgaggaaagcaagcatacactggaatggcaggtaaggacaaaatctgtgttaagggctggaggtatggcttagttagtagagaacttggttgcatgcatgtgggtctgggtaacatcccagcactacataaaatcaggcatggtgttatacctagtactagggaagtagaggcagtagaattggaacttcaaggctatgctctgactgctgcagactctcatggagtttttgtgagaatttaaaattcaaattctgggtttggcatggtgacccatgcctttaatcccagccagcattcaggaggtagaggcaggcagattactgtgggtttaaggctagcctggtctgtacagtgagttccaagacaggaatatatagagagagaccctgtttcagaacaaacaaaaataaaactcaaacttcttacaaagaaatgtgaactatcagttcctagcctaagatcacctgatacacaagaaaataaaataccatgttaaaaaaaatcagtagatgccgttttaaaaaacagaattataaccaggcagtggtggcacatgcctttaataaacccagcacttgggaggcagaggcaggcagatttttgagtttgaggccagcctggtttacaaagtgagttccaggacagccagggctacacagagaaaccttgtctcaaaaaatcaaaaaaaaaaaaaaaaaaaagatagcagtctgagaaattagaactatagctgacttaaaagacagcttagatctgtggtcagtgaaagatactgctcacaagagactgtatcacttgagtccacttgcattaatgtccctagtaagtaaatatattgagagaggaagtaggttagtgataggctaaggtggaaggacacagtaaatgaatgtgaagtttccttaaagtacatttatttatttgtgtgaccatgtgtgtgtgtgtctgtctgtctgtgcacaccatggcttgcccatgaaggtcaaaggacaacaggtaggaaggagctagaactctccttcccctgtataggacctagggatggagctcaccccagacatcgggtttgggcatcaggaacctttaccccgagcctctcacatacttctttaggattctttaggttgtaattcaaaggctctggcaccgattgcggtgatggttggttgtataactcttgatggtgatggttatataactgagcatactgaaagatgtggagttaaatagggtaagtacgtggaatgtgaataagctatctcttaggagatagatgataggaagataggatagatagatagatagatagatagatagatagatagatgtgattagatagatagatagatagatagatagatagatagatagatagagataggtgatagactttacccacatgtacacatatatagctgttcagaaacagacgaagagaaattagtaaactagaatatagatatggagatattattcaaaggacaatataaagatgggcaaacaagcatagaggggataatgtaaatagtaaaggaagtttctgtttaaatagaattccagaggaaaataacagcgagaatggaagaaaaccatcttcaaatatacagctacaatgtatgggattggaataggtttggtcccatagactcctgtgtttgaatgcttggcccacaagaagtggcactgttaggaggtgtggccttgttggatgtttgaccttggtggaggaagtataacactgtaggggcagactttgaagtctcctatgctcacgctatgcccattgtggcacacggtctcctttctgttgcctgtggatcaagatgtagaactcttgggctggtgagatggctcagtgggtaagagcacccgactgctcttccgaaggtctggagttcaaatcccagcaaccacatggtggctcacaaccacccgtaatgagttctgatgccctcttctggtgtgtctgaagacagctacagtgaacttacatataataaataaataaataaataaataaataaataataaatctttaaaaaaaaaaagatgtagaactctcagctccttctccagtgctatgtctgcctggatgttgccttgcttcctgctctgacaataatggagtaaacctttgaaattgtaagccagcccccaattaaatgttttcctttataaaagttgacttggtcatggtgtctcttcatagcaatggaaaccctaacaaaggcacaatggttaagaatattccagagggactggagagagatggctcagtggttaagagcactgttcttccagaggtcctggagttcaatccccagcaaccacatagtggctcacaaacatttgtaatgggatctgatgccctcttctggtgtgtctaaagactgtactcacattcataaaataaataaatctttaaaaaaatttccagaactgatgaaatacttgaatccacagagatagaaataaagaaaatccacacctaaacatagtagagttggagactgcaaaagactgattaaggctttagagtagccagagaggagatggcttacttacaaaaatggatactctaactagtagtctagggctaactttctcaaaaatagcaatacaagctcaaaagcagtacagtaataacctacagaatgccaaaattgtagctgctgaacagaaaccgtgtagctaggagatcattcctcgagaataaggctggggctggagagatggcttcgtggttaggagcccttagcggttatgctcttcctgaggacctgcgtcaattcccagcgcccacgtcaggtagtttccaactgcctttgatcccagctccaaagtgaacctctaacctttgggagcacttgcgttcacgcaagtgaacacatacctccctatacacatacacataattaaaataaaaaataaatctttttttttttttttaaagaataaggctggagtaaaaacattttcacaaaaataaatgtcaggtttactctcagaaggatgacacaaggggactgtaaaaaggacacttgtcagaagagaagaaaattatcacaaaaatggtgtcttagtcagggttttattgctgtgaagagacactgtgaacacagaattgggggtggcttatagtttcaggggttcagtccattattgtcatgacaagaaacatggaggtaggcaggcagacaaaggacaggagaaggagctgcgagttctacatcttgatccacaggcagccataggagactatatgctatactgtgcatacctcaagcataagagacctcaaagtcactatggggaatgcttgccccatagtgactcaatttctccaacaaagccatacctatccaacaaggccacacctcttaatatagtgctgaatagtgctgctccctatggaccaagcattcaaacacaggagtctagagtcttatgagggccattcctattccaaccactgcagatggtatagaaatcactgggaagcctggcagtggtgacgcatgcctttaatcccagcacttgggaggcagaggcaggtggatctctgagtttgaggccagcctggtctacagagtgagttccaggacagccagggctatacagagaaaccctgtctcaaaaaacaaaaaaacaaaaacaaaaaaacaaaaaaacaaacaaaaagaaagggagggagggatgaagggagagagagagagagagagagagagagagagagagagagagagaaagaaatcactgggaaatggtgagtctgagggtagaaatctgtaccagcaccaggcagtgatggcacacacttttaatcccagtactcaagatctggtggatgtctgagttaaaggccagccttatgtacagagtgagttccagatcaaccaaggctacatagaaaaaccctgtcttgaacatcaaaacaataaacaaacataaagtccatatcagcattgtttgtaacatgtaatagtagtagttcgtaattccaggatggaactaaaattcacaacagtcaaagggtcggtggtcttctccctccagtgcccctcattgtcggtgaacttcaagctcatctccaccatctcttcagtgacaggttctggagcaggctctgctcaatcttcttgcttttaataggaccaaaggatggtgactagacacacctgcatcggcacctggaaagtgaggttcccgagatggctggctatgcccaggctcttggtaccccagttggccttccttttggcccctggactctaggtcttgcatctattctcccttccttgtgttcttcctttggacagagttggttgcaaactctttgctgtcaatgtgcagggttgttttgtccccacgggtcaacttaggttatcctgtgtcctgtctaacacctcagtggactctatttttggttcagcatcttcctatttggttttcagtagtcactgaaagtgctggcaaacagttaacagtgtctttagtaggaatggttatctaagggaccatactaggtaaagggctgagatacttcatatacatagcataccctgactgctgtaaagatagaaaaacagactgagatgccctacacagtctgtgtctgtataagctacacactttctgtaatggccaacatcaatttatctttatcccatagagggcctggcttggagcatgtgttccagggactgaatcatttgaggtaggagtgaatacttttctttctatcctcttgcattcttccagtaaaattaaagcttatcgctatcatgtatgttcccaagcaatgtttaattatctgtaaaaagctttgtatctgagactgcatggattacagatgggaaaggaaaaaaaagtgtttatttccacagtgtctcacttccaagaatggggtgagctattgacaaaccatcacacgaagacatttttttttaaagatttatttatttattatatgtaagtacactgtagttgtcttcagatacaccagaatagggcatcagatctcattacgggtggttgtgagccaccatgtggttgctgggatttgaacttcggaccttcggaagagcagtcgggtgctcttacccactgagccatctcaccagccccgacattattttttttgttgcagccactcataaatgggagaagaatgttgaaaggaaaagaaaatgtaggggctggagagatggctccatggttaatagcactgactgctcttaccgaggtcctgagttcaattcccagcaaccacatggtggttcacaaccatctgttacaggatctgatgccctcctctggtgtgtctgaacacagcaagtgtgtgctcacataaaataaataaatcttaaaatagaaaaaagaaaagaaaatgcagaaaaataatatgaagtctaaaagttcacagcgaaagaggcattttctgcaaagttcccaagcaagtgactccatgctgtttgttgatcagcattgtgagccctcgtgtttaacattgagctaccccatactccacaagcccaacgctccttcagtgacatatgcagcagctgcatcatcattgcccatgatctagattctaagctctaggtgtggtggtttgaataaggatggctcccatagactcatgccagggaatggcattaggagctgtggcctcgtgggaggaagtatgtcactgagggtgggctaataatatagaagttagtaccaaggactggggtttttgctgccctaggctggaccaagtttcttggtggaatgtggactttggattaggaaaatagttgaatggttcaagtagagcatcacaggccatactggtaagaccatggaagatggtgctgttggtgacatgaattatggggtacacaggttttagaggagaagaattgtaggcctagatttttcaaaccctacttttaataagggttcttaaacgctttatcctctcttctagcccaccacccaccagagggagtggaaaggaaaggttaatagagcaaaggagggtgtggatctgtttagaaaaagttctttggagcaaatccaatctgtgttaccaggatatcagcagtacagttcactcagaacagcagcagcagctcgatccacttgcaaacaccattcatgaatcagcaatggcaggtcaatccagaagaaacctcgaggctctgccaagtacaaggaagcagcagcaagaagctgccagaaagaacaccaccagaagttcttttgtggcatttctgtctatgaagtcatatcaaatgatgactagcaaagaatggcaaggcaaggtgctggtgagatggcttagcaggtaagagcactgactgctcttcccaaggtactgagttcaaatcccagcaaccacatggtggcttacaaccatcgtaatgagatctgatgccctcttctggtgtgtctcaagtcaactacagtgtacttatgtataataataataaataaattaaaaaaaaaaagaatggcaaaccaaaccatacagcttcatcagcgaagaccagtgtcagcaaaacccaatgataaccagcaaatatatatatcctttccaaacatcacattcttgttctctcaagtgtcagctttagcaaaacatcacatgccctttgcctagactgcttctagaaaaccaccgtgtatctgttctcagcaaaacatcctcccacatgtctgcttcagcaaaaacatcctctcataagacagtttccagaaaaacatcacatgacatgactgcatctccaaagaagccagaaatttccaattcaaagagtactagtatgcaacctagagaccattcttgtgatattttgacaaagatcatgggtacattctgctcttgtcatgcacacagacacacaaatctgcctgaggttaaatagaagagttatgaatgaacatctttagaagatttgtctctaagagatttccaaaaagtttagtattgactttgttgcttggttattagtgaccatacttatgcagatctgtaacgaagaggaccaagctaaacaaggaacatacagaatgtacagtttgaggagaaaaggagcccagggaagtgtaatggagtcaagtccagtgcttgaggagataaacctgatgttaaatggaaaggagaagtggtgacctcaagccaggactccacctagctaagcttccaacttctgagaagtaatcaaagaaaagtttagaactgggtgtagctgaattaccttgaatgttagcagcactcatgagacagaggcagcaggcaggtcactgagttcaaggccagcctggtgtacagatggggttccaggacagccaagcttaggcagtgaaggaaaccactgaaaacagaaagctggtgaagatggatttgaatgaagaggcacattccaggcccagcagcagcagaacttggtagctttggccatattgctctggctttagagtggagggtacaaaaaaaagagttatggaatctccctccgtgactaaggaaagctgatggggccaggtgtgtgtcagggatgtttctgcatggagacccagagacctttgcttaaagttgtgcaggtaaaaacctggattgttgtgatagtttgaatgcttggccacagggagtggcactattgggaggtgtggccttattgaacgaagcgtggccttgttagaggaagtgtgtcactgtggaggcaggactttgaagtcatatatgctcaagctctgttcagtgtgacatacattctccttctgatgcctccagatcaatatgtaaaactctcagctcctccagcacaatgtctccttggatgctgccatccttcccgccatgatgataatggagtgaacctctgaaactgtaagtcagccccaattaaatgttatgagttgccatggtcatggtgtctattcacagtgatggaaaacctaactcagacgattgccccagaaagcccccaagatgttggatatgccagagtcatgggatttgtgaagagggatgctgttaacagggagtggaactggcccaagagaaataactgtgttgcagtcaacaaagttgaaaagagttggagatctgaagactgctttgacatcaggcacggagatgcagagtttggagtttacacagctgttttttggtcttgcttttgtccagtatttcccctttcctcccttttgggatggtgatagatatcctgtatatgttggaagtatgtgatcttgattttgattttattccagatcatagttaagagcttgccataagtcttagaacagactttggacttttaagcagggttaagactgatagactatggagacttttgaggtaggattgagtgcatttctggattatgataaagctacaagtctgtggggactgggatgtagaatgtggtggcttgaaaaagaatggcccccacagacacatacttgaatgtttaaggggtatcattagaaggtgtggccttgttggagtaggtgtggctttattagaggaagtatattattgggtttggggggtggtgagctttgggttttgtctttgtgtatgcctgtatgaggaggtcagatcttttgagactggagttacaggcaattgtaagctgccatatgggtactgggaattgaacccaggtcctctggaaaagcagccagttctcttaaccactgagccatctcttcagtcccgttccaatgaatttttgagaagcttttctagaagtgcctgaacccagagagacaataaaaacagttgttctcaacctgtgagttgagacacctttgggggttgcatatcagatatttatttacattgattcacaacaattgtaaaactttcagttttgaagtcgcaatgaaataattttatggttgggggtgtcattacaacatgaagaactgtattaaagggtcacagcattaggcaggttgagaatcactgcaacaaaagaagaattttgtgccaggtggtagtgccatgtgtctttaatcccagcactcaggaggcagaggcaggcagatctctgagttcgaggccagcctggtctttagagtgagttccaggacagccaaggctatacagagaaaccttgtctcaaaaacaaaacaaaacaaacaaacaaaaataaggtatttgtaaaacataccctagatccgttgtgctaagacataacagcattccaaatatacatagcccaagtagccaaggggttagagctcttatcagctttgatgagcttgatataaggggaatttccctttatccccaaagatgaagtggtcttaaccttcttgcatgctgtttgcttgttagttaataggtttatttttattttatgtatgtgtgtgttttgcctgaatatatgctgtgtaccttaagtgtacttagtacatgaggagtccagaagagggcgccacatcccacagaactggagttacagatggttgtgagctgctgtgtgggtgctaggaatcaaacctgggtcctccagtgctcttaactgatgagccatctctccagccccttccatgattctttaaaaagcagtcatagtgaagagagtacaaggttgtgcatcaggacagtaacacccccaatccatttctgtaacaagtcagagtaagatgacagatctttcaaagggacaccagcaactgtctgggcccttcctgtaggtccccctaaacttcactttagtctggagaaattccagggctctcagattactccagagagctagaaagagaagcttttcttgtctcgttttccgtttgtgtttataacataaacttttatttatttatttatttatttatttatttatttattatatgtaagtacactgtagctgtcttcagacactccagaagagggagtcagatctcgttacggatggttgggagccaccatgtggttgctgggatttgaactcgggacctttggaagagcagtcaggtgctcttacccactgagccatctcaccagccctataacataaacttttaatttcactgggaagacattcaagcagaggtcccaagttctatcactcagaacacatatacagatctggacatattgacatgcacatgaaatgcaatggagatagagagacagaaggatccctggtacttgctggccacccagactggtagaaccagcaggctccaggtagcttcccaaccatttctccatctttgggtccattgttgcctgtctgaatatctttgcagatagtttttagaggggaaatacagcaagtaagcagaatggtgcagggtagagcctgcacctggaaagtggagaaacatcctagagagacacagccttgcaaggctggtccagggtgtgggcagtgtacagctgtgactgcagatgtcagcacagatgctgggtacctgattcatggcaaccacaggttgggagggcccctacagatgttggagccccaggtctgacaatcccagtggggtgatttgtgacgtttgtaataagatcagatgtgagggtagaggtgagagagagggcccaggtagccttctagacctgtctttggaatctgagttcagactggacattttgggggctcatttccagagtcactctcctgtgctctgctcccatattcatgagaagagcactcccatgctggcagaacatgaggcccaggaagaagacatggggctctccataactcaaccccagagacctagtgccaagcaattattgctcatagcagccaaaaacatgtaagaaactgccccattctggcgaaagagtaagatgcatttgactgtgactgtcacccattgtcaggaggccaaagcatttctttagcagctcctcgagtcaatgaaatactgagtgtactgaggacgagggcagctgatgtcctttctcatctgtggtctcagtgaacagaggtcaaccaggatccaaaaagcagtattatgtggaaactcccagaaagaaaccagaaaggagtttgatggtcacgagactggacacactgccagagtcgcgtggctagcgtcagcaaaagttttcaagtgacttccacggaagcatatttacgacgacgtacagaagaagggcagctacttaacagatgcccacctaccttttagaacagctaataagtcacagatgcctgcggtactcccagaacagaggggccttagtagtcaggcctcacacaggaggcaggaagaaaagcatgcaggaatcaaaccatagcctaaagtataagatgaacatccaggagcctaaaccactctaaagaaaattacatagatcctctatgtaaattaaggagcaggagagtgagctttaagttgtttctgcaggtgcataaattatgcaggcacttcaggccctatggagaacttaaccctcctcctcagatgctggctgcacgcggacttcctgccacaggacccctgctatgaagtgagtgcaggaaagaacgttttatagtgaagatgcctgagagacttccaggccaggtgatccaagtcaacacctacagtgacaagtcttgttgatggtttgtacccttgaaatattatcatgagaagggcactctatgcctctagagtcttctgcccccaaccctaaagcccagtctgatctgagacgagaaggagacagattccagggaaggcacacgctctacagtgtctggccagtgttccatagaaccggcaaagtcattaaacacaagaaagaactgtaggagatggaacaatgtgttgtgatgacctagatggaccttaaaggactgctctgtgtgtgtgtgtaactaaggaacctgggtaaacgatgcggtccttaccactcttctatccattaaaatgtttatcatgtctcttcgtggtggtgcacgccttttaatcccagcactcaggaagcagagacaagaggatctctgagtttgaggccagcctggtctctagagttcagggctacacagaaagaccctgccttgaaaccaaccaaccaaccaaccaaccaatgaaccacccataacccacagatttttgttgggtgtgcttttgttggaacctccctctcttttctcagttttaagactccagaaccgaagtgagaacagctgggcatgttactgtgtgcttgggatcccagcaatggggaagaggagacaagactttcctggcacttattggtaagctagcctaacttaccctaggtgagctccgggccagtgagagactctgtattaaaaaaaaaaaaagtggatgttaaaagacacctaaggttgtcctctggcctccacatgcatgtacagacatatgcacacatgtgtgtgcacacatgactgtttcctcttgtttatcacctgtccttatgatttgagagaactgtagtaggctttcagatacagaagtggattggcttggttaaaaatcttagttccactactggcctgtgtaaaagatggggaatctctgctgagctaagccatgcactatgagaacagtctctctccaggccagacctctacaaaaaaaatcccaacaacaacaacaacaacaccaccaccaacaacacctttgcaggaaggtgttggtaaaggccctagtttaaaaatggtgttacatatacacaccatgaagttcagagagtccattcgcaacattatttgaccttcagcacgatttgtttctggaactttcccaccccattcttgcctcgttcccccaggtcctggtgactgttattattttgtatccatactgttgcacagcaagtgtctagaactttctgaatctgcagaacagactcgctactgatttaaccatggcctcccctcccccacccttcccctccccttggcttctgctgaccaccactctacttcccatctctccctcgttggctgttagctacttagtggaagtggaaccatattgacttttgtgactggcttacttggcttagtataatgttttcaaggttcaaccatgtcattgagtgcaccagtacattcctaaaaccctggcactcaggaggctaaggtaggggaacccagaccaatggttttcaaccttcctcgtgttgcggtgacccccacaaccatatttctgttcttacttcatagcagtaattttgctaatgctatggatcatagtacaaatatttttggagatagaggtttgccaaagggggtctcaatccacaggttaagaacttctgatttagacttttagttcaggccaaattacacagtgagagcctatctctttcatttcctaatttgaaagaaagaaagagggagggagggagggagggagggggggggaagggagggagggaagggagggagggaagggagggagggaagggagggagggaagggagagagagagagagaaagagagagagagagaaagaaagagagaaagaaagggagaaagagagaaaaggctaggcgtgatgcagctcactgcagctctgtgagacgcaggcaggtccatggtcttcccacttcagccgcctagtgaatttgaggccagcctgagagataagagaccctgcctttaaaaaacaaaatagacaatcagcttagtgggtaaaagtgtttgtgaccaaagctgccaacctgtgctggatccccagaacccacatggtggaaggagaaaaccagttcctgcaagccctccacgttgtgagccatggcatgcaggcatcctcagacagacagacagacagacaaaatgaataagtacattaacatgtttaaattaaaaaatggaaggggcagaaaggggaaggaagcagatggggtcttttgtttctttgttttgtgtgtgtgtatttatgtatatatgtgtgtatatgtatgtatgtatgcatgtatatatgtatgtattagagacaaggtttctctgtgtagctctgactgtcctgaaacttgctctagggatgaaatgcatgcacccccctcccccagctgaaactgacagtctgattccctattgtgcttgtgtttcttaccagttcatcctctgctggacacttggatggcatcttctatgccactgtgaacagagctgcaagctttcattttgggggtatatactcagaagtgggggtactgggtcatatgaccagtcagagattgtttagtaactaccaagttctctacatatttagaaatactatttcttcttctttcttctttcttctttcttctttcttccttcttcttcttcctcctcctcctcctcctccttcttccttccttccttccttctttccttccttcctttctttctttctttcttttcttttctttctttctttctttctttctttctttctttctttctccttcctttctcccttctctcttcttcttcttccttccttccttccttccttctttcttcttcttccttccttcctcctcctccttcttctttctttctctcttcttcttcttcttcttcttcttcttcttcttcttcttcttcttcttcttcttcttcttcttcttcttcttcttcttcttcttcttcttcttctttctcctcctcctcctcctcctcctcctcctcctccttcttttaagctttatgtgtacaggtgttttgtctacatgtatatcttgcacaccagaaaaaggcactggattccatgggactacagttacagacttgggaggcatgggggtgctggtctttgaactcaggtcctctggaagaacagtgctcttaaccactgagttaactctctggctccaatatttcatctctctctctctctctctctctctctctctctctctctctctccctctccctccctccctcccttcctctctcccttcctctctttcttttttattttttatttttctacttattttcaatagcagccacctccacagatacaaagttctctgaataggccataaaaatctgagcttactcttgagtaatatgtggtgaatggacggcagagcatggccttctgtcttgcttggtcctcacacaggacacaggtcctcagacaggcccaggaccctagagctcctttgggggaggggacacagtggtgctatgaaagcctgtcttcttctacttactctcagttcattataaagcatttcaacctgttaatgggttccagggtcatactattctgccttaattaaactaatccccacattatgatcaagaaatcccataaaaatgatctgtaacaacatcacacataggagataaaatgaattaatgtgaatcacacggaaatgaggaatgacaattcctgtgaccctcccagaggcccttatcaaccataggaaaaaaaaaagagctgacatgttgatccaaatagaggaaagaattaggaaaagcagttaaggaaagaaagaggcccttacagatttacatctgcccgataaagcccactccaaagcaggagtcttgcatgagtgccatgtaccctaactacagagcagcaggggagagatgctgaagtgtgcactgtgccctgtgtcatccagtcagtggtcaggcatgccacgtcaaacatttttattaacggggcacatgatcagaaatatttggagacagtgactggatagagagtccctgtcattcatcctggatcttccaggcaagactgacacatcagtttggaagaatcacataacagatactggaagagggcgctggcaagcagcctatgtggaggatagaacttgacagcactcaggcagagtcacctttaaccaacacctcaggatttcttcccaagctcccctctgtatctcttttgacgctgcttcctgaaatgtcttccctctaattgtgtccctgtggactttggctgtcccctgtgtaggatgggtttggtatcgactgttctcgaggctgcggggaggagatacttagcagagtgccaagagtcctccaggggcagcctcggatgatgaccggactattcattaactcagaaaaaaatggtgcctggaagcatcttaccaagtgtagttgttctgcgctcagggtatgctcggggtatgtagttgttctgctggtatgcttcccttctcagaggagttcagttctcatggtctttgtaacgtcagtgttacattgttttaatggctacatcattgccaccaatggctagcctctcctctgtctaccagccctttcctttctcttcttcagtgctgggctggaacccaagactttgctcatgctagactaggcaagctactcagccccagccttgttcatggcactgagtgcttccatatgcccggtgagctctcccaggtctggctttcattgctctcttcacaagcctggatggtgttggatacctgatggcccccgggtctcttctctcccttcagggcaaggggaacatgtgatctctctctctctctctctctctctctctctctctctctctctccctctccctctccctctccctctccctctccctctccctctctcattgttactcctgcttgtggagccaagcaaagaaaatgaccactgaacagtgaccacgtgacaagttggggaaagataagtagaaaagggggcgagaagaggggaagggaaggagaggtaagaactatgcaaagaaaggaagaaaaggaagagaaaggggagggggagagaaaggaagcagggaggaggacctctgtacacctccaaactaacccttcaagatccaaagagcatcttcttccctgtggccctccagcggcccggccttgctcaactctcacagcaaccctgagcggagtgcttgggtctgcattgcaccggtggggaagccgaggcctgggaggttccaacctttgtctagaagcacctcatcctccgagaagatcagcactgacagagaggtcacccacctgcctctgagggccacctccccagagcccagagaaggatcttacctgggatctgtgggcggccatctaacccagaaggcatgggggtgcctggaaatgccaggaaaggaagtcaggaacagctcaggcagaaatgaaggcacaaaatgggccccacattcctggctgaactgggatcaagagtgagcacaagcttctaagatctcaggctttcttccttccaacctatctgctacttggcctggaaggaggatgcccacagccttccctgcctcacctagccttccatcacctccttgcccctctcctctcttcccttccttctgcttttcctttcccctcaccccctccttttctttctcattcctcccttctttcattttcctctacccttccttcttcctttcttgtcccccctcccaacaacaacaacaaatccatggcttttgtttctaaaattatatccgcacaataacctctctgccaattccaggcacacagagccaatggaccagctccctttggataattcaaattgaccccaagatagccctaccccacttgtacctcctccctttgtttaagaggttgctctaagctctttgtttcctgaccgaaatccttccatggcttcctattatcccagagaagtccacacctgactaagcattccacccagcgtctgcagccttctcaagtttcaatccccatccttcctggggtggagtgtccctgttgtacctttggtagagatgactagggactcatcagattacaccaggacagttgatgtttggattagcagcaggggtgtgaggctagatgcttatttgccttgtggcatgcaggacacctgtcagctgcggagaacggctcgccataccaatttcccgaagggcctcgggctcttgtgcttccttctgctatgatgctagcctcggctgctgccacgtgtgttcccagcctcctgctctggcccatcttcatctgtccacagacctcagtgttgcaaacatggcttcttcagatatgtctccctagggtgcccccgcttcaggaagtacggctgagagagccatggctctttggttcctggtcactttgagttcctccataccaggagggatgggttggagctcagagaagaccctgggcatacagtcttgggtgcctttgtgtccaggcaactaaggcacctcacccgctatattttgcaggaactttctaagctgggcttaggggtggacactgacatagaacttcgaactctgcagctgcctgtggattacagggaggtaaaacggaggcttaccacaatctgggaagattttcaaccacaagtaagtgatacctggagaggctgtgggtctgggaacctgatgatgggcccagggcctaggaactttctggaccttgatctagatttgaaggctggagtacacctgaattaaagcttctgtcgtgagattctgggtctgtgtataagtttatgctgaaacacgaggtagatagtaaaagatgactgggggcaagtgggccggacaaaatgggaatctggtattaaagccaggaggactctaggatagctcacacctatcctaaaaaaagagaggccctggattaagccctgtgtatggctaattctggctgttctgaggaatttaaggtaaagcgctttgggcctaggaaggtctgatgatgattcacccagggttggtccaccttcaaaacatcctgccaggggcctggtgtgatggctcacacttttaatcctggcacctgtgagtttaaggccagcctggtctagtttatgagttcacagccccattccaaactaacaaaagtaactacatagtaactgaatataagcactgtgtaaacaggggcacaatgccaggtgtggtagctcacgcctttaatcccagcacttgggaggcagaggcaggtggatttctgagttcaaggccagcctggtctacagagtgagttccaggatagccaggactacacagagaaaccctgtcttaaaaaacaaaaaacaaaaaaaccaaaaaaacaaaacaaaacaaaaaaaggacactgaccaaacactgcagcaaaatcctctgcaactgttttcaaaagaggacaaataaagtctaataagattcgattagactgttgctagcttcctttatgaaagtttcattaaatacaggatcctgttaaaagccagcatctcccagggtcatcttggatacttgctttcccccaactcctggggggaaagagagcacacaacctcggatgagccatttgaaatacaaaagtcctttttaatccagaaaaaatggggaaaagtccttaaatgcagatgtagcatgcctaattcaacccaaagatgtgttccttttgtatcagcaccattacttaggggcctaagcagggctcctggaggacttggagctagatctgtttagcggggctctgtcatcactggacctctcttcacatctagtgatctggtgtctgtcctgggatttctgatatgtatgtccctacagcttgttgtgcatgtgggcatggactcttctgccaaggccatctttctggaacagtgtggtaagaaccgaggctatcgggattcagatgtaagaggcttccagccggaggatggagtgtgcctccccggtggccccgaagtgaggctgtctgttgtcaacatgaaggaggtctgccggcgtgtggctgttgagaatgtcgaagtggccttttcccgagatgcgggcaggtacttcaggactccgtgtgccaagtgccaaacagatgggagagagggattatccttcagagtcccttctctctcctcaagaggctatcctatgttgtccatgtggctgctttggtcacaggcatcctatttgtcacaggcactgcagattaatttaacctcttctccaaacgctgacagcccctccccctaagcacctttcctctcttctagggataaagaaatacccgaggggagttggcagtaagggagtcattgtggtgaaggtgtcttaccactagttgtttgaaaggccggctcaattctgtcatgaccgcttgctggtcattagactctgagcccaagaaccaatgtctgtctggagtgttcttaggccatgtcctttctctatcagaagagccaaactcaagtggtactaggcatgtgggcatccataaactcatgcttcatgaaacctgtacttactgtgtctggcatcgacaagatctgggagtaatggggagtttcatgtggttgcaggtacatctgcgattacacctactacctgtctctgcaccttgggactgggcatgcggctctcatccatgtccctcctctgtcgcactggctctcggccagccttctgggcaaagccttgcgagtcatcatccaagaaatgctggaagaaatcgggaaagtccagactcaaagtacagcagcttaagggaagcagcagggggatgccttccttagatgaaatggagtgtttcaggcttggacggagaagccagctgtgctatgagaggctctaaaaaagggaaacagttacaaggctgggggtgggggtgggggtgggggtgtggactcacagaatcacaatcttactttagtgtctaaagcaagaagaaaatccccaaaggcggatgcttcataaaggattaactaggggaagttatgacctttgtaactccaaagagccaccaaaacctagtgtcaagaccaaatgaactgaataactctcaagtgttctttgaaaaagattttatttatttgtttgtttgtttgtttgtttatttattatacaatgttctccctgcatgtatgcctgcatgccagcccagggcaccagatctgattataaatggttatgaaccaccatgtggttgctgggaatcgaactcaggacctcttaagctctcagtcatcttccctgctttgttttgttttattttgagacagggtttctctgtgtagccccgcctggctgtcctggaactcacagaaatctgccggattctgcaagaaaatgactttacaagatttcctttgcaaataaaccaaggacattttacatatgacagagccaagccacactaaggcagatcaaagcccaaggtggaagccgggcgtggtggcacacacctttaatcccagcactcaggaggcagaggcaggcagatttctgagttccaggacagcctggtctacaaagtgagtgccaggacagccagggctacacagagaaaccctgtctcgaaaaaaacaaaaaaacaaacaaaaaaagcccaaggtggactgggatggcacaggccaccagcacagaacctagctgcttggctggtgccttggctggagacccctctctgtgagcaaagagctcctgctccaaaccttaggtccctttgttccaggagtgggctctagtgacacttcaggtcacttggcacctggtcagacacattaaaagaaaacgccgggcatacgcctttaaacccagcacttgggaggcagaggcaggtggatttctgagctcaaggccagggctacacagagaaactctgtttcaaaaaacagaggcccagaccccaacccccatgagcagctcttccaccccctctgcgcctctccagtttctcaagaccctaagcgcccatttgcattgtattttttgtaaatagcttggcatcccccgacaatacacactgcatcctgttcagtcacagctgatagtgggtggtttgtatgcttgcgagccagtgtagaacactgtttttttgctggtcatttaatggttgggaggtctgaagcatggctctgtgttcatctataaacccgactaataaaaagtcctggttctgactacccagagaatgtgtcaccaacaacacaccagccaaaaaaaggaaagaaaaaaaagaaaaaggtgtagtatgggccctcagcttgtggcttgaatatctttttttgttttgctttttttttttggggggggggtgaggtgggtgggcagcagtatattatgaatctagctagtaagcaggtgtttaaaaaaacgaataataaattaattctagaacgctcgtaataggtgacttagctgggcaggggtggcacatgcctttaatgacaaagctccgtaggaaggcaggtggctctctaagctctaggacagcctggtctacagagcaagttccaggtcagccagggctacacagagaaacgatatttcagaaagattaaaaaaaaaaagtgtctcagactccaaggagagcacctaacttaaaaaaaaaaagacttaattttatgtgtattagtgtaaaggtatctcacccttagatctggatcccttggaactggagttacaattgtaagctgccatgtaggtgctgggaattgaaccctggtcttttgggagagcacacgtgctcttaacctctgagcaatctctcgattcctaatagtcttttaaaggtctagtacctggctggagagatggctcagtgatttaagagtgccgactgctcttccgaagatcccgagttcaccgagttcaaatcccagcagccacatggtggctcacaaccatccgtaagaaatctgatgccctcttctggggtgtcctaagacagctacagtgtacttacatataataaataaatcttaaaaaaaaaaaaaaaaaaaaagtctagtaccttcatccttaaaagcagctggctccggccaggcaggtttttggtaaactggaggcatagtaccaatccaggatagcacagaaataagaccttcagcgtcatctagtgaagctccctgggcatctggtaaacaaaacccccggggcaggacgagattggaaggtgtgctcttccaacgaccttttgactataaatccaaatacccattgggttaggctactgggaggctcactgggtcttgagttgcctctgatttgcttctgccacctatgtctcatcgatcagttcttcacccggtgtcctgattttgtcactgagttcccccattctcagtgttcagtgaggcggctacctactctcccaggagtctagaatgtatggcttggttgattttccaacccaaaccattcatcaccttcccatggcaaagatggagagccagcatctcagagttacctgtgggcaacctaacgactcagcaccttgcagccaggaacagcccgattttccttggttctctctccagcaccctcaggcaccccaactgtgaagcactaccaaggacaggaaaatgcacgcagcacgcaagtttctggcaaccccgccccacccccagactcacacataccaaagccaccctggccttttgtcaacccaaggtccaggggccaagcctgtgaagcagccctgagacttgtgcttcatgcctcctgattttcctctagctgagtaagtgaggggaaagtagcttcttcaaactcaacagcaatttgcatttggagaaacccggctgtcagttctcgtgctgagcattcccgagtgacacacacacacacacacacacacacacacacacacacacacacacacacacgaggctcagcttcccggctgtcagttctcgtgctgagcattcctgagtgacacacacacacacacacacacacacacacgaggcttagcttcccgaggaagtgggggatggttccagagtgaagaggaaaccaactgggagacaaaggtgcatacagaattcttttatttaacttaatccatgtggtactttaactactagaaaaaagcagagtaatagactcaagtggccttagctttagccattcaaaatagacaaagtttcttttttcataatgtaaagaatcccgagtatgtcgcagtaacaggaataaattcttacaacagaatatacaaaaacattgaattttttttcatctactgattttcttttatatataaacagagggtttttttttttaggaataatttatacacagaaagttattttatgtaacaaattggccatattattatcttttttagactttttttttctttttttaagaaaaaatttaacaagaaaactcagaaatgcattattggcgatgtgtccgctccatcgagccttttgacttttgttttgtttttttaaatcccagaggtagatgaactctggcaaaactcttacttcaacctcactggcataggaagcagacaggggttgtcaccaggcaggcacctactccacctgtacatgtgtctgcacactgcccaacacccacaagaaactgaaagaccaaaccaaacccacaaatggttcccagacctttttcctgcagtgttagaaagttccatgtgttttcaacaacggtatcttgtggaaccagagagacatggccaataatgcacggggagccaaaggacatggatcttaatttgaaaaaagggggcgaggggacacacattgtggtttttgttaccagtttgttctgtattagaaaacaatggaccttcggcgttcgccattagatacaggtaggcacagcattcgttaggatctgaggcacatgtgcccacgggctgcacatcagagtcatgtgatcactgtgaagccattccagtgtcctgaaaccctcccatctccatagtgttgcagagctaggaccagccataccagccagatgtcaccatggagtgccatctctttgctgggtggccacacactgctatttccatggccagcagttctttccatctgtttgggctgctacactccacagggaacagtgagtcccagataattcctttccccagcaaagggttccaagatcaagatactgaagaatacagaagtactttttaaaccactggcattccttttgttttgtttttatttttatctgttttatttttttttgttttatttatttattatttatttttttacaaactagagcagaaagcatgtgggaaagagggtgaaccacaattggtcctggggagaatttctttttaattaatttttttaagtttttattttttatttttggcatattgtaaagctagctagtaaacaagtgtcaaaaaagaaaaaggaaacaaagagggggaaaaaagaccacaaacacatatttttgcacacacagagagaaaaaaaaagtgtttttaaattattttcggcaattataaactacaaacattttataaaattattctatgttcttacaaaaatgcaatgaaacattgaattagttcttgtaaaccagagcttcgtcaacgactacccgtaatctactggacttagaagccctattgaaaggataatgagtaatttaaagaatggtttactggaaagatgcagacacaaatgtggacacacaaatcagtgattatgaagttctcacagaaaccaaaatcaaatcaatccctaacgtcatgccccagaccgacaactcatctgatgagtgtgatggagtctgagtccttccgcacgtgacttagctctaaggagaagtttccagagcaggatgccaagtgtcttatctgaaaccaaatgcccacagttgactccccagcctccacatgctctgacaccttcagacaccatcaagtccaaatatggtccatgcttcttccaggccctttaaccctctgtcagcaattcccgtcatgtcacgatgccggttaccaagggtgcctactgcaccgtcactctgtcatcccatcaaggaatgttcgtttctctaaatcgccaactggtcatccaagctggaagcccaaagcaaagacccctggcctgtccaaggtctgtatcttcagcatctctggcaagagaggttcaattttacacagacatcctgtcttgaagttctcatttgtaccaccacctagttcaaagtggactgttaaaagtttgaccatcatcactggttcttaaggggtggatctctctggtccaggtctcccaatgcagctgcttagcccaggaaatgaaaagacatcagacaaacataaaagtaacattaaaaacaaaaaccaaagggaaaaaaaaaatcccaaaccgtaaattcattactgctaaaatgggccaaagcattttgatggagtggtcctgtctcctcctggatgccagtgtttaggaaaggagatgctttcagcactctgtggccactctcagaggacccaacgatgacaacagagggccttgggtaagcatcctagactcctcacccacatgagctcagtgtgaccttgccaccactgcttgcttacctcaggcacaggcactgccaaaagaggtgggctgctaaggccgaggctccgggagctcaggctggcttaggacttggcatcatacagagctcatccatctgtcagcccaccctaaaaactcctgtataccactccgccatctgctcgtctcaccgtgatcgcttttctagtttatgaagctgcttttcctaccgttgaacagtcccgagtcataagaaacaggcaggtaggaaaaagagcaaccttgaacagtcttttatatctcaaggagggagcctcatcccatctctgaatccaatgttaggacattcctgaaaggagatggcgaagcgtgggccccacccagctcaggttcagctctatctccacgcctcaggaagggctgtgtgggggcacacctggatgttcacgtcacctgcatgctgtgcctcagtggttcatgtgacttcaagcagtgtgagctcctatcagaagggggggttgggtgggggtgggctcgagtgaaaaaggccacgactcagcttggtaggagtacttgaagcttgtaaagagaaaaatcaggaagaggcggggaacatcagcatgagatcaggctagaactgtggatatgatgccttgcaatagtcggggcgcgtcagtccagcagaacgactgcaccctcgtacaagccaaccttttctttttattgaagtgtttctggagcattagtttatgacggccactacgctgttggtgccgtctctctacttaaaggaaaaaaaaaaataacacccacgcaaaaacaaaacaccaacaactcggacagtttcaaaaataataataataaggaaaaaaaaaaacccttaaaatcaattgaatgaactgtagaaatccagttgtggaaaaccggcaggtatgtgcgagaggataggccactatcccagagtgtcacctacagtgtagaagaaaaccctgatcaaacagtatctcccagtgaccctgtgagcatctaggaacccagagaagggcagcattccagcaggaggcgaagggctcctgagcttaaccgaactgttaggcagtttctccatactgggggaaaagattagccaccgcggtccccagctgaagtccccagggcctgtaagagcagctgctggagttgaggattgcactgtccacctgtgagcagcagacggatgggtggacagaggcagagcacggggaaggctaggcgccaaccactggccttccagcctcaagatttggtcagtccttgtttagccctgggggttgcggggggggggggggaagggagggaagaagagggaaggacagcagcctgcagggggcacaggcttcgtcttgcccccttttttatactggccttctgtcagaagcagaagggggagctccctgaccgctgtacctccaagaggaaccaatgccaggagagcaatggtggcatgcccctgcctcggtgcaggtctcagaggagcagctgagggggctgacagggcaccctgacatgtgcacacattgggactctcaagacaacctacgtgtgggtgcctcctgccctcaaaacctgggagtacctgctctcttagggatagaatttgtcagtcattcaatctgtagagaaagttctgttccaaagtcagccagctgaggatgggccggggagcccccctccactggcgtgctgtgctcatggtgactgctgggttgaaaagtaggggaccaggtgaggagaaagaccagagttgccctaggaaaagtgggcttttggagacagagcaggaagaggctgctcatttgagttagcgttgctttaaccccagaatccagcctggcccctctctgcttcacctgggtatgtggcctgcggagtggctgaggttaccaggatcattggcctactgtcaggtactgagaagtgtgagatgtattatatatatatatatatatgtgatatatatatatgcacacacacacatgaaaaacaagagtgaggtagataaatgtttgaaaaacaagcccccccaaaaaaaccccccaaaactgtcccatctaatacacacatacacagagatacatgtaccctttcctcagcatttaaatccataacaggggcttttctacagtgattaaaacaggtatcacaaaataaattaaaactcctacatgaaataccttcttaataagcttcccccctccttttccctttaagggcagaggctaggaagagccacaagcctgaacctacagacaagccagacaactccagctttccttcctgcatctaatctccagtcacaactgcaaccatcatcatccaacagttttcctactcctaatttaaatatttccacccagaagcatctctcgcgctgtcactgctctggtaagaagtgctaaatcgaaacaagacaggacccctcccccaaccccactccagggggaggggcctccaagcccagagctccgggattctggttaaggaggaacagcaaggggctctagggccactgtagggtcagctctactccactttgggatttccggcccgggagtgggctttatagcacacccactaaagttgctgctcctggccatggggagttggtgtcttacatcttgctcccagggaggccaccccttcagacaagctggttctaaagacaggtgggcctgcttgtgaaggaggtggcagatgggccactggaccagctctgctgcatcctggggactcagcagctattgtgtgtgtgtgtgtgtgtgtgaaagatcaaatattcatttaaataataatccaatgactattgtctgcagttaaaactgctattgtattcccaaattggggtgtgtgtgtgtgtgtgtgtgtgtgtgtgtgtgtgtgtgtgtgaaccaaagcaaggagagaaagcaaagtgtgactttcaacaacagggactagaaacactaaaccaggatgtgtgggatttttaacctttttgtttctgacctgagtttgtggcatgccttgccctggagcagatgctgtgtgcggggaagcaaggaaaggagagagtagggcccaggagtctgtgggctctggactcccaccctccccactccctcacctgtgactgtgctcacagagcctatgggttagagggtaatgaatggggctgcagggagcaggggaagggagatgtatttataaaaaaggcatcaaacagttcacggcaagttatgtcctagaagcatcagttgccggagaagaggaagaaaaaccacactctagtgataagcagaggaagtttcttggagtgagcagaaaggaagaaggggtagacctgaaagtttcatagattaaatccacaaagataaagaacaaaaaaaaaatagcagcttttcttttcctgtacagatgtatagatgaatatctgaactgtaaaaaagttataaaactgacattaaagacgatgtgtatgcaaatgtttcacggtctaacatagaactgagagacccatgaagaagtcctagtaacagaccatgtgaaagctcacactgcttggatccactgcctgggcgggcggctccaggctgctgtggctctgaatcccttcctcgaacatccataaccaacagccaggccgagtccccggaaccctttcgagtgtgtggccctcaggggaggggcaggagggaggtcggggagggggacgaggctggtgtgaggaccaccttgacccacaacctgaccttcaagcttgcccatggccacttcctccagaattcttggggtgctgtgggtgggggaggggtggagaaccagttctgctttccccttcttcaaggagggcgagttccatcttgtccagttctcccaaacggtaaccatgcaacgagacccaggagatgtgggctgggagagccgggcacagtggggggcctcagcacgtgtctctcaaatgttatcatcaaccagacaccatggttcatcccacgcctctggacaaagggaaatcagcgcagggagtatatgcgtgtgagaaaaaccaaaccgaaaacaggattttggcaaaaatggcagcgtgaaaaatgatagccacggatgaacaatggcccttttaaattttggggtgaccctaaaaaaggttaaaaaaaattccatctcagtgtgtgaattttttttctttttctttccttttcttttttttaaaagacctacggtgtcaggcaggtgtatgcggggtgatgaggagccccaggggcagccattaagtcaatctgattttttttttttttttaaaaaagaaggcttcccagatgggcgttcacaaacatttttccactgtaaagcagaaaagagagaaaggagagcgtggaagtggaaggggagagggcagagagggaaggcagaggagagaaaaaggcagagaacgaagagactcaattctcgagtgtcctgttgctattaagtgttgtcattaaggtttgcagaggcccatgtcagttaagggttcgggtaaaggaaataaaaagctgcttgcatattgaaaaaaggaaggtcccaaatgtgtttactgcagagaagccgtctcccgtgggcagcaatggcaggtctgaagatccactgaggtacaggaggcttgtgaatagattgttacaacctgctctcccctccccgcccccccgcccaccgagtgggcgcacacacgttactgttggcgcgtgcttcggtgtgtccgaggatcagcaggtcgaggactggggcagaggcaaggccctctcgttggcgcgtcccccgttcatgtgagcgtaaggctgtctctcatcaaaactggcgcggagaacgagcacgccagggcccgggccattctcagccttgtgtcctgagtgtctttcaggcagaggcagggaggctgaggaggccgaagggtccagtgggacgctctccatgttctcaggctccatctccagctcctctggctcgggaggcttgttctcctcgctgtagtagaaggagacctcctggaagctgggctccatctcatccttgatgctgccgatgatctccaggaaggagggccgcatcttggggttatactgccagcacatgcgcataagttcaaacctaagggagtgatggggaagaaaggaatgagctgccagagagacccagaagaactgtaccacctctgcactctctgcttcccatctggatgcctctggcatcctgccatgtatcttgcccagaacccaggcccagccacagtgccaagcccagactcacttcaagtcttggttcaccgattccaccatggacggttgtattaatgatagcactgctaagagataaaacccacttcctgtctaagcacctcttctggccacacattaaccagaagaggaaacctctctctctcactaatgagaatcacagctttcctgacaggcaccattctctgtatctcacaccagggctatttctaagtttcttcctggcaccagggcgcccctggcagtgggcctgagggaggaggcggcaggggtgctagacagacggagcacatatcccaagcttgctatctcacgccagcacctgctcactgaggatgtcccatgaaggaagatccctgtcttgaagatgtaggatgcccaggagcagagttctaatagaaacttcaagtcggatgccaacgaaccaagggaagtcctaagcatcattttcttctacaaacaaggccaaggtaaaaacagtataaagcccatgacgtacctccatcactcccacaaggtcacaaacctttgcaaagcttcctcgtcctcgagagttacatgggccagctggcacacaggacagaagagaaaggctgctcatgcagccaccgtcctgaccagcaaaggcctgggagtagacagtggaggagaccctaagcaagtgaaggtgtgtattgctgacacgtgcaaggccatgtcaaggaccccaatgccccagtcccatgggagtggcaaagtctaccccagtgatgcttgcttagagagatggtaaagccttcccctgaggtccgagttgcaagtgctgacagtgtgtgcagaatatgtccactgtcacgtccttcccctggaagtttacagcctcaagacggcttccctgcagagatggctaaacctgcctccatgttcagctgtatataataatcctgcttccttgtttatctgcatgcagtaatctgcctctctgttcaaatatatacgctaaacatactgagcttccagagcccagtccacgcagtccccgtttctctatacccaagtgcacactctttgttcattcccttgatgccctcgtcaagtcatagttctgcaacccaggccatgcagtgaaccacacctgggctctgacggatggacagacacacaggcccgacagaaaacaaggacagggtggtcatgattctgagggaaaggggtacctagcagctctcaatcacaggcgtgtgagaccaaagcactctccacctatgctcctccaggaaactgtgttttataaaaactcctggctcaacagacgctttacactaaaacccacaaatggaagttcgaacatataggaatctcaaagcaccctgaacaggcttctaacattctaccaggctgatcactgggccaccaggacacttctttacaaagcaaataatcccacaggggtaaaggcgtgtgaaagcaacctattggtaggcatgaagttgcctgtgcagagaaagccactcttgcccatctggccagcttccttggactctgtaggatcaagtcaggccaggtggaaggtagttatcttaaaactgaaaaaaggtgcattggagatccacctccaagaattaagcactctggaggcccagggtgaagccaaccaattacagcggagccaggtgcagcttggagcctgttgttagcggaattgtaccagagcagagctggttacacagcacctgtggctgtttctaagctcctttgtaggctgagtagttgagacagcggctgtttagctaggaaccttacaatatttactgcctactcctctggaggtaaagaaaaatcccaattaagtggccaatgtctcgacggaggaaaacaaactggaccaaagaccaggctaactaggtattctcactggctctaagtgactggtcctagtggcacatgacaggttgcaggtaggtactaaaagctctaggcccgggagtcacgtcagtccccgagagccagagatgtctctttactccaatgattacaacacatcctcattgtatgcataagggaaaagcaccaaccacagcatttgatcatttgtctgaagtcacacagggagtgagctggaacggcgtgaggctgtcagacaatgcagctttactgtaagtaaccatggtcttgctagcagacggcgctctcccgactgtctacagacagtcagatctcctgagccggcttgctctggttagctgctgcagaagcttgttctccccggtccccatcatttcccgaaggattaatcggaatgggaagcagacacaaggagcttagctccgtggccaggctgcaatttcctctctggctgacaggcctagccctgaatagaaaagtgcaaccgggggcaagaacatgagatgtgtctggccccaagccccgtggccaggggtggggggtggggctgaggaggtgccacgggcagggtagggatgaggtgacatcagtaaaaacaagccatgtgatgcctcttttaaatcatcccagagtcagcccttccagggctgcccataaaaggaagtcttttttgctctgtgcgatttaagaccttgttttttttttgttttgttttttttaaatactctttccacaagaaaactctccagtagactctgggattaataagctgataaaatgctccctctaaaaagcacactttaaaaccactggacagctctgatgagctggagccgcgatttacagaatatgtccctctgtgaaggtgcacacctatccctctagaagtgagtgctatcctggggtgtacgcccaccgctatgcccattcctcacttggcttctgagcccccaagcacaccccgtgtcttcagctggacaggctgccatctatgtgaggcactggcaaactgaaatatgcccaggacctcatgtatacagtacccctgctgccgcctgttagtactgtaccaggtagtagtgactcttagcagggatcagactttcagaagggcctacgggactgagtgggtcctcctctagcatctcaagcaactaaggttgttttagggccaggacctagacggggatgctatatagcccagttgctggcacacagtcgatgccagggaccgtccagcagacactgtcacctgcctgtgatgactctattcaatttatctcgacaaaaccagactgggattcccagcctcctgggggtggagaggaaaagtacagaagtagtcagattggactgagtatgtctcagctgcatctgagctggtgacgctcggctagtaaaacgttcaagttccttcctgaatcccgcatcccgaagacttggcatgtgagctctggagatgttaatggatcaaatggatcaattctgtggctgagcttacatcgtcgtcccattctgcagcacgagggctggaacctgtttcaggatctccaggttccgtgaactctgggactggcccctgacctcagcagggttcactgacaaatctgtgtccaaagccctgaaacccaggggtaaagatgggaatgataggctcccctgacccagcctcacagaatttgctagatcaattcaaactgagctattgcactgacagtcagacctggataccctggtgtttggtggcagcctttcctctaaagctagaacactttgtaatacacaaggctggatacttagctactcctgaagcagactggttcatgtgtagggacgacgagaccaaagcatggccagagaaagggctgttcctgcctcagaaccacaccatggaagtccttcctctacgtgtgagacccaacaggcttcagactactcccaagagtgtttgcttactgtgccaaaaaaaaaaaaaaaaaatcctttcacagaacacatcttcctctcatcagcgtctcagccaaatcagggcaccacactccctactggagcagcccggctcccttcccttcactctgttcttttcttagaaaggagaccgatctgccaagggggttctgaatggcaaagcaaaaaggcctcctgaatcaatcatttcgcatccaagagcggcctccgtgtgtaagcttcctcctctctatgctgggaacaggaagtccactgcctttcccatagcaggtggctccttcccaagcatattaaattcataccaaaaaaaaaaaaaaaaatccaggcagctggggatggatcaacttcaggagttccatgagtttctcagacaaatgtttgctccagcataaaagaaaagaaaaaaaccaacaatacctctcctgtgaccaggcccaaaccacaggaacaaaactcacacctgctcttttgaacggtttctcagctcgtgagacagtgctgatcctggtatcctaaccaaggctggtggggcacacccttacacagactcagaggcagccatgctcctactccatcctttcacctttgcccacatactgactattctcgcaatggacgagtaggactgccctgcaccactgaggtcaaagtaaagctataacagcactcagaacccagagcttctctaggcaggccccctgcatcctgtacgccttcggaggtcagtttagaaaggccacagtggcagtgttctccgtgaagatgctgcagggcacagcaggccacggaccagtcagtggctcaatatggcctgctgtctattcagttttgtaaggccctcccaagcaagagggttttttttttttacacttgccaacagctggggcaaaaacaaacaaattaacaagtcccccacgccccacccccaaataggagtattttataagaagaaaacgagaggaattcaaattccaatataaagttttagttgggcaccagcagtaacactggctgcaatccaagcactaggggagtttgaggcaggaagattatcctaaatacctgggctacactgactccatccttgttgattcctgaatcatagtggcagagcagaggggctggaaggaagagtgagcgtgcggtccccagcaccaccaatcctctccagtgtattagagaaatcctctcagtctatggagtggtccacagagaagccacggcctctcccactacacagaaccatcactgttcctaccgccctgtagtaaatccgatctccttacaatcaccccacgtaccggagtatcttcatctgcgggaagccctgcctctgaccagcagctatcttctggagaccctgagctagctagtgtcacctcatcgagctcctcctaggtttcattagaaagcctgtgagatgacattttcaactgttggagcctcacgggggacgatgctagcccacaccttgacatctcaagattcagagtacagtcactagcacctctctgtgctctgcccaccacctgcactgcacgcccaacctctccatctgtacattgcctcaccatccaaggcgccacgagagtgagcgctgcgctggggaagagaaggccaggggtaggcagagtcttttgtgtagaagtgaagaaatcttgacaacccccctgggattctttcactagtggagctgaacctgttttgaagtgtttctgggaaactgtatgggtcctgacatatcagtgggaacctgctttgatgcttaaccccgagatgcagagcaacagccgagccccaccttaacgtgtgcacatcaaatgcaagtccactgctctcaacctgtactcgatgagccaatcggaataccttcatgtcttccttgaaacctcactccaagaagtgccattctcggggctggagagatggctcagcgggtaagagcactgactgctcttctgaaggtcctgagttcaaatcccggcaaccacatggtggctcacaaccatccttaatgagatctgacgccctcttctggtgctgtctgaagacagctacagtgtacttagatttaataatgaataaatctttaggccagagtgagtggggccgaccggagtgggcaacctttgttcccagcaaccacattaaggctcacaaccatcagtacagctacagtgtactcatatacataaaataaataaatcttttaaaaaaaaaaaaaagaagtgtcattctccagacccactgagccaccaacatcagatgacaaagaaagacctctactcaagagagtacaaataaaaggcccacatgtggaaagattcttccccaattttggcaacaccaggtaacttctgaggggtggagcctaggaggctgccaggtcattagaaagttatcttcaagatttaggactacagcaggaagattgtgaacccctttaatgcaggggttctcaaccttcctaatgctgtgtccccttgacacagttcctcatgtcgttgtgacccccaactataacactactttataattgtaatctgctactgttatgaggcaaaatggaaacatctgtgctttctaacggtctcaggtaactcctgtgaaggggcaaggggtcacgactcacaggtagagaacggcttctctaatggctccctttcactttcaagccacaaagtgagactgtgcgctctgacacaccttttctaccgtaacagttacctcgccacaaatccaatggaccccactgccgctcccacactgggagccaatgtgaaatgcttctccttgtaagctgtttatctcagggagtttctcgagtcacactggctgagcacggaggtggacgtgacaccacgtgtgtactgccatcgagtgacaattctcacaaccaatcgatgcaggatgttatttttaacagaagtggagcgaggcaaccatctcaccaaccccccctagggtgtgcctacaagcatgtccaaagagggatgagctcaaccgtgctgtaggtccatgcagctcgaagggattaagaagacagcagatggtctgtgaccttgtgctggggctctctgagctttagtctacccgtgcaagagagtgactcctgaccacagagctctgtgtgagcatgctgtatagaccttccaggggcttggggagagctttcaaatctggctcagcagtagtaactaacttgcatttttgtcttaaaaattaaattttgcatgaaaaagatgagccaggattgctcatgtatttcagagatgggagatgggggggggggggtagtggtggctgagacgcacgccatggtgcaaagtgccaattagcactggggggaccaagggggcacaggatgagattaaggagcagcacaaagtcacagatccaacaagctcagcagcagcaggatagagcctttctaatcctctcctgtacccacacacaacggagtgggtggctctggaaatacatacagcatatcagggcagttgtccggcttgtccagaaggccaccctccatgacgaaacgaagaacttgctcgttggacaagccctggtagggctgctcagccagcgtggcgatctcccagaggacgaccccgaaggacctacaggtggaacagaacaggaacagctcagaaagggatgggaagggccatgtcccctccttgccagcatttcttagactctgggtcctgaaaaaaagtgtcctcaatgtgacgactgcactttgtggacatcaaaccagccagcttgctacgtgtgggacatctgttcccctttgtcactgctaaggcttcctatagggctactgcaacccagggctgaggtaattaatgttaggggcttctaacatctctcacagttttaagagcccgatctacagcggacacattcagtgtctggtcccagccattacttggacataagccaaattagctctagttcagcaaggaatgcagaggaccaggtcccagcctggccactgacctgagacagctaagcatctcaactatctcctgcagggatttctttcatgccagcttagaaatactgatgctaatggttctccacgcctccgcaagaagaatctgatcagggttcttaaaacaatgggtatcttgggctctccaaagcaattctgacctgctggttgtagggtagggagctgggcctggaatgtctcaaagttctgtaggatcttgacttgggtcaaggaagatgaaaactcgtcccctgcagaactcaggagccatggctgcagaggtggctgtgtcaggaagggaactggggtaggatacactggccagggggtgtgtcgcaataacaaagagtcccttggtctagaggctggctgaggtatgaactcatcaggcgaagcagcgaggattctactgcgtgaggtatggcctgtcagcccagaagatggaactaaaggagatccatgcatgcagacacttcagacagacaatggtcacactgtcgctcagtgtgaacacaccttctcagggagaacccttgctttcacaaaattcttcctggcctgtctgctcttatggtcaggctgtatggctagaatgaagaaggctgcccatggcaagccaacacaaggcttcggatggaaccctagcagggctgagccccacagaagagcctgaggcggccattttccacttagcactctgaggatcctggcctaagctggagcaacagcctctgactgcatcttaacttttggttcgcagcttcagtttccttctaggaacaaaagggttttgtcccaactgatgctaagcaatgtcagacaggaacagaacctcagcaatcagacccaagttccttgggaaccggcagtcactaagacagacagtgcagttacggttttcaaagacctggccctagagaagcaggtttcctgtgtgtttcaggcctgggttctgccttgcagagcctttgtggacactcggaggcattctttagaggctgcagtgctcaccctcatgtctgaatccggcctgagacaagcagtgatgtatgtggttttctctttttatcaaccggtcttgaccaggttcccttccaagccgactccccttgtctttcatgctttccctggtgcaccacaggctttcaggcattcagtcacaatcctggcccccagctcttgactgccatgtctagattgctggttacttgactctacagactcagcatgtcagctccagtgtaactcttgctggcaccctgttctcctcctggactccctgcaggccaacttcccattccatcccaccccatctcagcagccagtctgctcataggacggacaggaagatagagagtataaatcagaaagaacgaagacgtgtaagggctggccttcctgcccttgtgagggaggggacagtccctgggacatctccccagaagctcaaataacagctgtgataaatgcagatggtcagggtgtggcttgacctgaggacacctcccctcaccatgttcctaaggtctcctaccaagtcaaacaaaatccttccaacttttctttggatttttacctacttctgactgtgaattcattctaaaatgactgcaattcctctagcaagcagaggttcaggcagagctgagcatccgtttatacgttctagtgtccttgatgtccccagctgtgtgctcaaacaccccagcacttggccttttctcttcacgcattttcttattggtttgtaatggctacgtaatccaagtggaggaaacctgcaggaagcaccctgtgcgcctctggctgtggcatgcagttgttacccagagtcgggggagaatgagtctgtgctcactctttccctatcctgtaaggtgatctccaagcttccagaaaaatgaagcttagaacaaacagatggaagtgtggccctaggtgtgccctctcctgcgacctcatctacacatggaacagagttctttgattcgacagagaagatgtaacctcggagaggctaattgcagagcagacctcatcctagagtcgggaaacaaccaggccgcgctgggtcatattcaaaggctatttccaatttgcacctgtcttccaaacaagatccaaggctcttgaccgggaaaggttcacgaggctaaagaactgatgtcagtcactactgagatagtttctctgtgtggtcatccgccctttgaataaaccaaccaccgcttcttctgtgacacaacagcaacgggatctcccgaggagacagccaagacacttgctgacattcgaggagggggacgacgtgccagggctcctgcctgccaagaactccagttgtattttggaacccgatgacctccacacggtgtttccaaggaactgcagtaaacattactacagagaagtctttgcaccctctggtcagtgtgctactaccgcaagacacttattattaaggaagatgctttgggtcaggaataatggacatcgtgtgtgtgtgtgtgtgtgtgtgtgtgtgtgtgtgtgtgtgtgtgtgtgtgtgaacagatgagaccaacagaaaaccacaaccaccaagactcaagttttgggtttaaataaagcactgccttcaatctgaaggtttcgtgagctgcggcaggagcacatacatacccagtaccaactcactgagagtggggaagagagacgggggaacaggcagaggcgaaggcatttccccagtctttggaaatataatcaatatttgctaaaaatggaaggaaatgttggttctacctactccttggcttcccacagataccttttttgctttaatggactccacacaggaatatctaaaagacaaagggctaaacggcactgagagctgaactcaaggctggcagttggtcacagtgaacaaaacaaggaggagaatgagccacgagtgagaaggtccaagctggccatggagggaaactctcataccagacatcagaatgagtagtgaagacaccatccttgagggactcgggagacatccagcgcacaggcagcaaccccttcccgcctttccggtagtagtccgtctcgtagatgtctcgtgtcataccgaaatctggagggaagatgcacagttactcggctggaccaggtcttggggcagagccctgtccttccccccatattcaaggtgagacatgttcttgctccactctgagagggctctagcatcacagccacttcacaggagaatacaagacatctctggtcttcatcaggctcctttaagaaattaactcccttataacatttaaagcgagaggcagtttgcaaaatcattgagtaccatgatttttcaagaaaatatacgccgcacagggtgaggatatagttttaaagtttggtccaatacacgggaggatgaagaccccatttacttggtagtgtgtctctactgctgaggaaaaggccttttgccactccacagaggacaatgtacgaagtaagaactcaggaagataaaaggacagcaaacagctgagatgttcttcaccaggctcctggagctactacccaacaccctttcccaacacaatactctcactcagttttgaaaatgttttccattttctgagtttcatcatggtgaatggcaggcttgcttttttataatctcacattactgtgtctttatccatttttgattttccactcaaccgcaccacctttccctcacataaactcttgcattctccttccctggagggaagagctgctcaacctttaaaatgtcccctcgcacctctaagtggtgccacaaagaggtgaggtacaaaaaaggcagagcaaacccatgtagggtttgctctgcatggatgttaacccaacacaattgacttgcaccgctgtctgagctactggctgcctctcttgaactgatttagggtgtgtaatccctgagatgggggcactgggggacactgaggaatgcagaactcgcccgaggaggaaaaaggaagtgcttaaatctcatctctgaattaatccagagacatgcaaatgagaggatctgctttgacttgcataaaaggggaattctagaaaacaatctctctccctaaatgcaaaagaaaaatagaacaaccccctcttcccaaacgggcacacacaatttaattctaaaaagaaaaatcataattataatagaaaatatcctcataaatgcgccacatggttaagcagaaaatataatccttactgatttggatttaaaatggcaaaaagcacacgcacacacaaaagcatttttccaactgtaatgcaaaatgggaccaaagcagcctctcggagttcctggctgtgctgagaagggtgcacagcagggtaagatgaggacaggggcacaggcttgctgtcccagcccgcctcctcctccagctctgtcccaatccactctcactccacaggtggggaaagatggcacagggagggaaggcacctgcctcaggccactcaggagcacagtttcctctgaggactaaaatctagatccccacacataagggagggatgggaggcaggaaggaccacaaaggctggttaagaggatgcgcctcttatctagaaactctggggcggggtatcctcttaagggctgcccaccaagctaggctcccagggctccaaatatgagtgtacaaacccagagacaggctgggggtatcttcagtaactccaatgactgacatctgttctgtaacacagctgcatgccaacacccctaactcctgcaatcagcaaagaagagagcccgactttggatacctctctagagatgacacccatcgcttactaagaggccggaatggctcatgcaaaaaccacttctagggttgaggcaagcagaacatctccaggacacaagctcaagaggcggccaaaggttagtggctgatcgcttctgctacatcgtctgtccctctgcttgttctcaggatggacacttgttatgtttctgacctgctggcctatcgcagtaggaaggtaaagtatatgcaggatggttttctgagcaaccttgttactacttttttttttttttttttttgaagtaaaagacaaaaaaacaaagccaatatacaagctgtaaggctggggagaatttctagccaatgaccagagatgtaatgcaatcagtgttttaggatcacaatgtgttggcagcaacaaggtaggctgagcagctaaggtcatggcaggaagctatgcacgacagaacggccaagtgcttggagctaccactgaaggatgcacctcagtggcttaaccagcagcatacatcaggccctcggttcaattcctggcagcaggagaggaggagagaacttagagccgaccagtcctatgagtgaaaagcagtcagcagtgatctcccgcggactgacatgaccaccatatacccttcctgttacactatgcagggtttcaccataggggcacctggcctcttaggaaccccatgggcctattcagggacaatgctctttgtaagctgagaaacttgaggggtggactggatggtgacattctcctcaagccaaaaccacttcaggaagcaagctgccccgattatttctgtaacgtttgacctggggagcagggcgccaatctggactaccatgttaaggtgctctccgggttacagccaggaatagacagtcacaccgactacatacatctttgcactcaagggcgaggcactgctcctctcggacataatgaaaatctctccagtggcagactaatgactgcaaagtcccctcagcacctgtgagttcagtttagcctgtgctccaatctggaaagcaaaggacacacctccaattttgactgtgaaatcttcggctaccatgcagttcctagcagcaaggtctctgtggacgaacttgttggcattgaggtaggccatgccatctgcaatctctccagccatctggatcatcttgcttaagctcggaggaatgaggactagattattctgctgcagattcaaaacaagacaaagccatgagcaacggagacctttcccccctaacctcctgtaggtctacactaggagtcgtaaatgcatatttacttagtctgagtaccacaggtcacctgcagggagtcaggacttgctaagaaggactagcacttctagggaccagcaatctcttttcccaccccctcccatgcatgcatgtgcatggatgtgggggcctgaagcttatgttgggagctaccttctacccactggttgaggcagggtgtccctatcaaacccaacctggtcaacatagattgctctggggatcccaactcatccttccaaggctggaatcacaggtgtgctgccacaccagcccagcctttacctggcttctgacgacctgaactctggtccttatgcctgtgaccaccccctcccccctagtcttttctatttggagcaggacaaaaatgatttgaagtgaatcagcaagtttaggcattaagcattctcagctccagctagtttccatgttcctttggcttccgccatcaccaagtgtgagtaagaggaaggacagagaacccgtacaaaggccacgtgaaacagacacacttcctaagtggattaccacgagctttgcttcccgtgcaagctgccaggacttattcgtcttcattcccttcttctaaggtacaaagataggaagactaatgctttcctcttggattactgttttctacgggcactgaggataaccacagttcaggtggagttcaccttggaggggaccaaggacactgagcagcaatgaggaccaatatcatgtctgaagaggagggcaagatgctgccttaggaactgaaaagaattaacctaagaagtggtaaaatcataaacaaacaaacaaacaaacaaaataagataagatttatgtctaagaggctgatgagaatggcttagtgggaaaggtacctgctgtcaagacagggggcctatgttcatgtcccaagacaggtatggtaaaagatgaaaaccaactccctcaaggtctaagtaggtatgcacatgcatatgcatgaacacacacacacacacacacacacacacacactacacacatacaaacataaaaaattcccaaagctccgtcaccccactcccatgtgcaaaaccaaccatgacccttatgcctcagtttccctaactgaaaaccttctcatacaacactatgaagccatctcatttcctaatgtatctcaagtcacaggagagcatgaccaggcttggagatcacactatttgggctagtatataggccctttgtgctctgaccacgatgaacactcaagagcattctggctcttggccagggttgctaagttctcagcagatgccagactctaggaggccagcaacagccctctagcacgtccaggagtactctccagacgacatggcatgtgaggaccaagttgagctcatttatctatttaaccattttcactctgatccagggtctcttcctataatctaggggatgtcctcagaggatgtgccatcacaggtgcttccttgtcacgctgacctttagcaccgttagggtcttacatacacatggcaaagcaggacctctgcagttccgtgacatgtacttataggctcatgacacatactgcagtgtatgtgtgtaatgttgtgtaactgttcaacaaagagctgtcacttaacatgtgaaaaacaaccaacaatggggacttaattatgtgcaggaaagggctgggctgacattttgtcagttcatggtaatatgttgtcattcttttcttcatctcgagaaagaagaaatctttagttttgttagagcctcttaaggtgagcacacatccatacacttatctaagtaaataagcaaacagcaatggggggaggtagaatacgctgaagccggggtgtatacactgaagctgggtatatatactggagctagggtatatacactgaactgtggagtatacattgatgctaagatatatgtaagttattttttaaaaaattctgacatgagttagacaaaatttagatctgatgaatttaaagattaagttttacttccgaaagagactttcgtaatccaacctgtctctttacattgaattaaaattttgttttatgtttgtgggtgttttgcctgcatgtgtgtttgtgcactacctgcccacggaggcccatggaggccagcagaacagtgaataccgacagttatgagccgccacgtgggtgctgggaatccacccagggttctttggaagagcagccagtgctcttaactgctgagccatctctatagccctttctctctctacatgaggaagcggaggcccagaggcatctgttaactgaggactggaggtaaccaatggcaaaaacagccccgagagcacaagttacgggtttactctgggcatgtgtcaaaggcattaagcacattctgatgttaggaggtgccttaaaggaccactctccagtctccttctgtagactgggtattactggggtaggatgctgctgctgggagaaacaggcatagctttgtggctctgctatcaatgcatacaccgccttgtggctctctcctgaaacttcagaaacacacatgaggacatttgtgaacaggtactcagaactaaaatgaaggaaccatccatcacttggtagatgattacgttgtggaggaaaagaaaacgaaagcttctacagagaaggggagcaaacccagaaagggcccgagaagaggaagttccccttacagcgaatgcagagacaagctgcttagttagccacaggtatcaaagccagggccgaagtgagagcctcagagcctgaggcagccccatgaaaggcatttggtccagcaatatttctcaacacccccatcagttaactcttagtgaggacttagaagccaaacttaagccaggtgcttgtggtgcatacctttaatgccagcacttgggagacagaggcaagcgaatctctgagagttcgaggccagcctggtctacgaagcgagttccaggacagccggggctgcacagaaaaaaaaacaaaacaaaatgctgtctcaaaaaacaacaaacaaacaaacacaaaaataacaacaacaaaaaagaagtcagactcacatatgctaaaaagctggctgtgggctaacacaacagaaggggacactccacagtgactaatcaaaaaattacacaggacctactcaggttcacagtttgaaaatctcactagccttaagcagaatcatgcagctggaatggttatcatttaaaagcgcaggcacaatactggcttattgcctagcctggggagtaggttcagggtttgagaagtgtctgaagcagtgtcggggcaaggggagagcagtgtgtgccatcaaggtaaggatagcatgctcacatgtacacgtgtgtgtgtgtgtgtgtgtgtgtgtgtgtgtgtgagtgtgtttgtgagtgtgtatgcgagtgtgagagtgtgtgtgagtgtgtgtgtgtgtgtgtgtgtgtaatgggctcctgaggaaacagtggtctcgtgcacagaccgggctgcgagtcactctggccaaaacctctgacaacctgaagagtgacctttattgagtacttactgaaaaggttaccagccacggtcatgaccctgaggtaaagaagaggctcaccccgaggacctcgagagtaaacgtatgctaggcagtcaagtgtactggaaagtcagtgggggaggccagcccggcctgagtgggaagctgtgggaagggttggccatgacacgtggtagagcagctggggttggtgtaagatgtaggcaggggtagcggggatgcagaaacaagagcacagagatgctggaggttgaatgggtgggatgcagagacagaccttccaaagatcccagtctgcctacacttggacaatcctatatagagggaactgacaagaaagcatatctcaaggcactcaaggctgagaagcaaatggacttaaagacacgtttcctacaagccagctcctgaaaaaagtggaccaagcaagataacacagcgaggaacaaaactgacctccacttctggcctcagagaccggagataacttttgagatcaccgcgtgtcattagttccatgatgaccagggtgggctggccttgggataccacacccagcaaccggacctggaggaatcaagaaaaggactcagagggggaggggggcatcccacatgatgacctacgcctctcggaactgggtggtgctggggcctgtagccaggagcagagttctggtgggagggcgagggcgaggctcccacctcaagggtctgtgggtggcaagaatgcaacggggaaactacccactgggaagagggcaaggcaacaattcgtagctggccaaggacatacagggtggtcctttctgtccatactttctgtccatgtctcatcttggcttcttaccacatggtgacaattgaactccttcatcaccgaggcctcgttgagaaactcgattctttcacgcatacttgcagcctcgtttaccgtcttgatggccactctggtttcgggttcatccttgaccacacccttggccactccttcatagaccatcccaaaggacccttgtccgagctcccggttcatggtgatcttctctcgagctacctcccattcatcaggcacgtacactagtaacagacaggatggaaacggcccttagacagagattacaagatgctgttccctaagacccaccaaggagaacatcaaggtacaaacagtccagcacgggtactcaatctctgaggactccatgacactttgagggtggtactcgtagctaagaaccagaaggaaaccaagaaacctaagataggaaaaccaaatcaaaccaaacaacagtggttcctttttcatatccttggcaaaaccccaacccaacaaaataaaaaaagcaaattagccctaagagacaaagaaagtgggacttctgttttgcctgtcttcatcaagggatggaaatgtaggtcagaaggggagaacagtgggaggggaaaggaccaaagggggtcaaatctcctcctccctggatgaagccagaggtgagaaacatcctctgcatggcctgggtgccagtttacaggccacccagccccactgtaggccttcagcgcttgaagtcgtgctggaagtaagacatatactgcattttaaagacttgcttaaaacaagtaaaaaaagataacttttaaaagattttaaaatttacgtatacatgtgaaaatctctctctctctctctctctctctctctctctctctctctgtgtgtgtgtgtgtgtgtgtgtatgagcacacacacatgcacagatgtgaggcaccacccgtgtttgggtgcctgggaaacccagaagacagtgttggcttctcggacctatagttacagatggcgggaggtctcttcacagaggtgctgggaactgaattagagttctccagaaggtcagcaaggctctcaactgctgggcccatttttttttccagctttgtattccaaagacttggttaaagcaagtttaaaaagatagcttttatattaactgttggatgttgtgggctaaatgaaaattctaacagatttatctcatcagcttttacacttaaaaaaacatggctactagctgggtgtggcaacaggaaactgcagtcccaccccagcccactgggcatggaggttggagatggggtctgctgaactaaaggcatcttcagattgtgtttcaaaacaccaagacaatgtgtcagccgcctgggatgtctatcagttcacgcacttcattccacttcttccatagccatgtccgacaacaagctcaaggtggctgagagggagaaactcagtcaacactaaaggcagagactcaagagaagagtcctcagccatcaaaagaaaccgcagaaaaggagatgagaggccagcccccacccccacccccgcagagcgctgaggtaggtcttgtgcactacaagcactgtctccctatattcctccatacagctgtttaactctggaagctgcagaggttggatccagtttaacacaccctgctgcccctttcaccgagatctagtgacgatacagactgcctgccttttccacctgagaggaaggagaactcacacgttgatgaagaaaaaagcagggttgggagatgaaacccagggttcaaatcccagatgattcaaggtgctctacaggctgtaaaatgcagtttaatcccagggctgtgctgtgttgtttttcaagtgatttgacttagtggaatgcatactgttttaaaaccatcaattttgtgaatggctgttttccctgcatgcatctgcactgtacgcacatagcacctgggtgccactggcacgtgggttctgggaatcaaatcagggtccttctagaaaagcagccagtgtgtttaactgctgagccatctctctagccccggaatgcatgaaagtattaaaacttaacacaagaagccaaactcttgcctaacacaggtgaagcctaggagcccccttccaggggatctctagtgagggaagtgagcgctgggatggagggggactcagtagtgaccccacggctttggcctgcactctgcaatgccaggaacctccctgacacggacctgaacagaattccttctccagcttccatttttggttatgcttgatctggtttgagaaaaataaataaacgaatagttctcagaaggaagggggaagagaaggaaagaaaataagagaggcaagtaagaaagaccaaacacaggctccatcccataacgccagtgggtacagcgagaactgaaatcatatcaaattaatacgtaagatctggaaagagaatacctgctccatgagaaccataacctaacagagttactgacagccctgtctaacagaggatggccagcaggcatggcaagaaggaagccctagcttagagacaggcctgggaggggaatcacggctgtgtttgtttttatgcttttagatgccgagctgcagttggaggtgggagaccccaccagcaggtcatcacggttatcagtcaccctcagagcacaagcccttacttctaagtgatgtcttaagggaagggtgtttattataaattcggggctggggaccccattgacttgtattttgaagaatggaacatacatcttgtggtggtttgaataggagtggcccccacagactcacacatttgaatgcttggtcattagggagcagtgatacctgacagagattaggaggtgtggccttgttggaggaagtgtggcaccgagggtgggctttgaggtttcaaataccaggcctatgtcttgctctatttttgctgcctgatccagatgtagaactcttaacccctctccagcaccatagcaccatgtctgcctacataccatcatgtttgctaccattacaacatgggcctaaacccctgaacctgtaagctcctttaagccagccactttccatgagaagagctgctgtggtcaacggtgtctcttcacagcagcagaacactaagacacatacttatctgtctactcacttactcttacttactggctgatttggggacaggactgctctatcttgttcatacttgccttgaacttgctatgtaatccaagctagccctgaattgacaacttttctgcctcagtgtccccagtacgtggattccaggtttgtgtggccacatttggctcctgtgcatgtttttgtatttcatggaatatgtatgtctggtaatttctcaactagaattctgtcttccatgggcctcagacaacttgtgaccctgcagaggctgagcttgctgaggagagcagagggggaaaggtctttataacacctctctgtaaggatggggaccactgtctccatttattacctctaaggatccaggctggcttttaatgagaatcaactacacggcttgcttgctgcagagcatggagttaaaagcccatcacatccattccctgggaggtccctgaagttagtggtctcaaccctttttctaaactcaatccagctgccgactttagccagagtacctcagctttgccagccagagctgttcataaacttcgtacaatgttatcagcactttcctgtagggcattctcagtgcactcttgacaacagcaacatgcagattaactctgggagaaagacgtttttaaagcacctttcagatgcaatgtcactttgtagctctttgaggcaaaaggcttgtgctgctgctgtgcttgtcaaaaggccaaacaaaccaagtgcagatgaggtgacagtgttctcacctgttgacctgggtagtgacaagtttgttcttgctttgttttgaaggggggagggagactatttctctggcacctcttttctcttttataattattgacctagacacaatggctgcacggttctggctagttaggggacggaggcaaggtgttcagatcatctgaggccaggagtttgaggctggcctgtataaacaatgcatatttagtttcttgatttttgcccgtttgctttcttgtttgttttggtggtgtgaggacagaacccgttctggaacaccgtaggcaaatgctctatcactaaactacatatccaaactcacttcatttgtttgtttgtttttaggagaaagatagtctccgtaagtaactatgtaactatgctttcaagtgtggtgcttacccttctagtgtattttctatgctttataggcacaaacgtatgcacatatgcctacttaaaaaataactctgcaagaccttttgcaaacagaaataacatcattattctgcacctgattcaaattcagaagaggctgttcttgactggctttactatacacataatccagttataaatgattaaagaaaccatagtaaaaccatacgcacttacatggtgcttttaaattacaaagggttttctcttttttttttttttcccatgtgagcaggataatcaatcggactgtgtcttatctgggccgcatctacttacacttctggagcaaagaaacattcctcctgaacaaactcctgcgctgcacagacccccatctaaccagattccctacatagcccagcaacctcacagctagctcttccatacaccacatcatgaggattcaggaggaaggcttgggatgtcccttgtgcagacttcccacggctcagctgtgggagcctgaacaagtccttctcagagattctttttttgcctgcatctagatagacatgagaaagctccaagggctaatcgaggagaggacccctccccagcttacttagaaggtctgaagccaagcataagcgctaggggaaaaaaagagaccagtagctaaaacacaggttccgagtcagaacttctgagccatttaattaaatgactttcattagttactgccaaagggaatgtaaatgggtgcataggctgtatgaccctaatcaggatagaacagcagagggattccaagatcatacaacgctatatagccctcactggccagggaacgcctgcagccccaccctgagttgatacccactcaggatgcctggctggctacagcagtcctctgactagacggtagtaattagaatgctcctttcactctgatgccacacttcaagcggctaactgaactgcttagcagaccggccaccggcatctccagagcctggtttctactggggcatctgagagtcccagaatgtctgacagtcactgagtgctaaagatgttattaaaagacaagaaggaaatggtaccaaaggcagatcctgggacaaaggtatctcaaaattacgcaacgcttttctccagtcagcacctgacagctcccatattcccacaccttggccaggacgcttaccatcagctgcgctgaaatactcggggttcacagaagcatacagcactccattgcccaacctgctgttatttctgtcaaaaaaaaaaagatgaaaaccaggtcaacaaatcccatctctatacatttctgtaaataccttttcttttcctttttttctttctcacgagttatcttgagcaagggacgcaaaccatgatcactgctgacgggacccttgcctgcactcctgatccaggccccggtatctgaggacaaatctttccacttttgccctggcctcagattagagcttctaaagaaacatttgtttctttgcttcactggaaataacaaggcccactccatgggaagctaaactggcatgcttggagaagctcttacaaggcagcaatctgtgtgctagaacagccagaaagcagacgacgggtacagagaaggaagcaacccctctcctgttgctgttagtcattaggaaatgtccttttttcccctctgaagaatgtctgactaggaactctggctgtccagtctgcatggtattttcccccacagacgagctatgttcaagttcaaatcatcaagccaatgcgccacaagtactgtcttccgctggcccaggcactcacctctttctatggaagacatacagcatgataaccagccccccaacgatcagcaggatggcaaccggcagagcaatgatcagatgcatgaagttctcatacgtcgctgcagagacaaggaagaaacaggaagacgtcacgcacgaggtgaataactggctcacttcatttctgtgtatggttggaaggatgataaaaccgcctctggaatagcctttactccctcatctcacaagaggagtcttcgaggagtcttctagtgctgggacaagaccagcagacaaactgaattgtttggagaaaggtaaataaatacagactgaagcacacactcctttaaagcactgcatattctgaatacagcaagtaattctgaaggcaagcccttcctgtgtgtagttttgtggaagtttagctgaaggaaaatgagcagacactcacacacttactagttgctcacgctattcttatcttttaagaaagtcccacagacagcttgctcgccactcccgtccccccaaagcactggatgacactccctgccaatgttgctctgagtgttctacaaatatgtaaaaccaattcaagtctgaacttgctaccagtacctgggtggtaagtaccacaggatggcctccaggggttcaggtctacacctctgaaaatcctgagaattctgccagattcaagaactcagtctgtggacaggtggattaacatggatgtacgctgggcatagggggaagaagagatgggggagcgagggttgcttcccctagggaaacggcttcccgtgcagaggaatgtcttgcagagtgtattgaatgatgaaggggtttggttccagctaagtggtttccgccccattcttcagattgagaggagtcagtggtcagcagttaagcctatgaactgcagcagtgaccaggatattgagatgttcctcagtatgtaacactggcaccttgatctggggacaaccaacagctgtctaagtgcactgaaggcctgagcactgggaggaaattcttgcccgattctgtaaacctagctgactctctgtagtgtcgtggacactagagaagaacctgctactagcacttttctgaagcagtgagcttctacccacagataactagctctcaggcctcgccagagaagcctctttctgcagcagatggaaaggattagagaaaaccatgactggtcaaaatgcagggaaccactgaccatggggtacccatgcccaactgatgatagacccacaccacaacccccacaacaaatgctcatggaacactgcaaaataagtgcgtggtacgattctaagatccagaagcccagggtgtctgctgagggatgccaccttttatgtaagacaccacccgcatgcgtgaatcccaacaacgcggttacctaaacttgcgtaatgacaataccagttcacatgctttgaagtgtggggaatctctgaagccccacccctagatgagaagttaccagcagttaatggctgctgagagagaaggaggatcagtctttatcaggcataagcttgctgataggttagccaatcccacgtggtcatccctaaacacatacatgtaccagtaacactaaatgagcttggcagagcatgtgtgtatatgtatgtaggtgtatatgcatatatgtatatagacatatatgtatcaatgtgcgcacaagttgcttagcaacaataattaaagtagtcatggaactgagagaggtgggcatggaagaagctgggagagggaggagccagatggtgtaaatacagtactcatgtcagaagttctaaaaggctaaaactaaaacaatgaagaagaagcatcagtgaactcttccaaggccagactgaaaacggattcatgtactgggaagatgagtgtgtctgaggacacactgggggcctctgcacactgccaggtctagaggagttaagggaacgtaccagtctcctgtggcatgctatgcatcgctccagtcagacctcaggaagaaatccttttccataagcaagatcagagaggcgtcctaactttgccagggttgcacggataactgacaagttccaaagtcagagcctggctttcctgcagccctggctcagaacagccgcaacccagtaactgggaggctggattcccgtcatgtaccagaaagaatcagtaagactccagttaatatgggaaaacaaacaaacaaaaatgagtatgcggagaagaagaagcgaagggctgtgagctttcatctgagagcccaacggaatgcagatcagggaatgaaacggaccgaagtgcctccatgaaagcactttagatttcaggcttataaggagtctatagcaactattggacacccaagttgacatgtctacacaataaggatggcgttgtgggtaaaaacacttgctatgttaccctggcaaccagaattcaatccctggaacctgagaatcaactcttcaatgttgtcctctgacctctgcacatgtgcacaccaagtacacaccaacagtatagagatgtctccttggaaaggagctggggactcagtggagacatgttcctggaccgcgagccactcaccaatggttggtctacctctaaactctattcttaatcctgaaatactacaaaagccaaaattaagtctttttcaaaatcgatgggattcagcaccttccagtttctaaaagtactcccacgtgcgcctccctgccagagagaagctccgagctttccttttcccctaggagaattcgtggttttgtgttcctgattctacgggtcaggttgttctgtgttctttttgagtttcacatttcaacagggacacagaggaactggaacacatccagaaggacaaaactgacagaaaattgtctatgcaactggctttcaagaaagggcccaaagacctgagaatatttaatcgggagggaagaagtctgaagaggagtttaatagaattcacgcaccaagatttccttaaaaacagcaggtgatgccgagctttcaatgtccccctctgaggattagacccaagaaaactgtgctaaactgcgtcttgtcggacaccgggaaaatttcccagaagccaagagcgctagcacgcagcttcaatccccctgtgggatgtgccaacttccactggagaaaccccattagaggacctcttaagagtccccagtggcattgcagtcttagctttacaaaggcagctacaaacatgccctctcctaagaaaagccagactcacttagcttcagaagtcaagactgctccttcagagaacaccacccaatttccagaactccagtgttggcttagtagcacgacgtgctacagataagcggaaatgtaccccaaggcagagatgctcagggcttcgttgccggcctgtgacgttattgggaagtagtggcaggtaaccttaggagctgggttctagaggaagtcaggtcactggaagcaggtgtcctgtgagagaactgtgaaacccgggccctttctgccttttggaggtaaccagcttggttccatcatgtgcttcctgcttctgacattttgtctcagaacagcccaaaagtaatagaatcaacagacagagcacgacaacccccgaaactgtgggggccacccctctaatcatctctgggttttgccacatcggagagctacagtaacatcacaatggagagttagcacataagctaactgatgacgctaggctacccagaaaaaaatgagaaaattcagagtctacctgacagaactctcagtctcatcatctctcaatctagacacagtagcagcagatacagggaatcctagcttcctgcttacggttctatttacttctgtctttgtgcagtcacgatgaggattttcgaatgtcttgcgtatctttgaatatctggaactggctaggtgggctatcgaagtgtgaactgacataaagttatacatgtatacatatttaccacatacaacatatcttaaagcatgtgtacattaacccaatcagcatcagcattaagtcgcattgttaccatttctgtggtggtaacactttcttagcaatattcaataatgctttattaaccatagctagtgtgttataaagctcactcttctatctacctaaaattgtgtaccttttgagcaacaccccctaagttatgttcccctggtaacctgcaagactctgagttcacccttttagatttatttcatatatacgtgagatcacgtggtgtttccctttctgcaccttctattaacagtattccattacatatatggaccacatttttacccaggcacctgctggaggatacttcattgtttggctactgtgactagtgggccaaaaaaacaaggggggtgtagatttcaacacaactaacttccatttcttttggactgggattgctggatctgttgagaccttgtctgaatcaagaatccagaagagaccacgaagaacataagacattaggaaacaacagttccccacaccctagattctagcctcaaagctgagctgagattttggggggtggggagtgcaaaaggaattcacatttgatcctgtatttgtttggatattacatgaacacgaaataatctaaaaaaatttggcttctcataagacacgtaaaaattcactatgtattatttcaccaaaaagactcaattttttttttttttcggtcaaacaagcatgcagcccttgcatttaacatttcacaacatttacttttaaatctgaatcctaacagctcactcagtggtttctgtgaactgtgaaacattatccactagggtgggtctgtgcaaaataacgcacagagtatctttcaatgcatcctggtggatggtgaagagcacagaggaggggaagactctgactgggatgttgtcctgcagattcctgtcccggagcagtgacaccagacgatccctggggagacgctgagcgctgggtcaccctcaccgaccaggcaggagacctgatactcaactcaatgctttgcaaaatgcctccacacctgagccacgatgagaacaagatatcttctaggttgtacgatactcttctgttactcacaagagaaggctccaaggctcagcaccttcccagctctgggatgtgcgtgcgagtgcacgtgcgtgcgagtgttggggtggggggaaggtgatggtggcacagatgtgtgtccactgtgagtaaataccaagtgtcttcctttctggcgtgacacctccgttttctagatacggcttctcgctgaaagcaaaggtcaccaatggaccattctggctagctagggatctgcaaaactttgactgtcgatctcctgagcaccgaggaagcacactaccgcccctggtttccgtcaaggacttgaacttagatctttaagcaggcactttccctagtaagccacctcctcaacccagccagctctgttctaacagaaaaccagagagccccagaagctcagggcttctgagttctggcccagccagctttgaaatttaccgtgcgtctccctcaataagacttacttttggcggggacatagaagaacacaggatctgtccatgacccattcccagagagggaggtagcctgaatccgggctgtatagttccctgggtttagacggttgagtttggcccctccgtacttcctgtactcctgtctggacacacattcccgctgatcctatggaaatataaggcgttaatttcctacaagcatgggtgagatctgacatctgtcaactcaagtcatcatgctgtgactccagctgcccaacctctccttacattgtccagagtatcaacataaaggaagcaaggataccggaaaagtggttttcaggttggcacatggaaaatggtaatcaaatatgttggtaattaaatcaaatatgttggtaactccagaggaataaaagatcatcaagacgtgtctagtctgcggctggaggcctagctacagtttcattgtttgtatgaagtccagccagctccatgcacccaagttactactcaataaagatggcgttgctaggaagacctttatccttcccgactgcccatctttctcccctgtctgcttagcagtcagcagacaaccagtatgccatagcacgtgctctcagcgctgaaacacatgcagctgcaggccatggtatccctcttacctcgacttgcgacccgtatttaatttcatacattaggatcaatccgttggggttctcgggttctggccactttaaaaagatggagttttcgggtcttggctcccaggtcaccggaccagggatatcatctgctccttctgcataattaatgtaaacaagcagtcaaattgggtttaggtccccagaaagaatgataaacggtgtgctaacttgagaacaactgttcccccaatccttgtatatacaggactctggacaggggaccttgtaggagttctttcctgagatggctgctggcacagcgcctccatcccctctgactgaaaaaacctgtggccaagtacaagtgatcacgcctgaaaaacctatgtaacattctccaggtccattttcctacctaaaaagttaaaaaaaaaaaaaaagaacccttcaaacagagcggccctggggaagggcagtgagcgacagcgtgctgagcctccagctgtggcactgacacattcacagcccacaccctcacacgggagggaggaagggccagttccgaggctggcctctgaggcagctgttccggccactgtctcttccagccaccgctggcctcacttctggtggtgaaccacagctgttgggtgacagctgatgacagccgagagaggcctggggagataagctgttctctgtctcttctaggattgggaaactgaggcacgggtggctgagggctacacgggagtgccaaagccgatctcagaagctcttactgccccagttcccagcatgctttgttctccccactgactgacacctcctccacgaatgagaagtggcaaatggctggcagggtgacaggcagtatctaactgtaggaggaggcccacaggctgatctagctgaccacgaggacaacgcttcctctgcatcaagctttgagacctggagggcccctcacaggtctgtggtgtgctaaggcctttgtgctctcagtctgtagcctctggcactgagagaagacaggactttaggagattgggcttcaatgaaagaagtgatgaaatacgatgtgttgcttcattgcaagccccaaagcagtgagaggcaagtgcctgtggtctaaaactcctgaaacactgagaccaaaataaacctttccttctggtaagacaactgccttgggtattttgtaatagcaacagaaagctaactcattcagctgtgcacagaagggcacaggggtgggtgtgagcatgtgcatcgtgaaccaagccaaggagtgagagccctgaaaggggcttgtaaagagtgtctatggttaagtctggatgggagctggcactaggaatacattctctgttccttaggcacctgacggacgacgttgtgtaccaacaaccacacattcgtaggcctgaatctaagggctgtcagcctcccccgccttggctgtaagttggcggcggggctacctgccgtacctgctggcatggttctcgcaaagacgaagttggaggcgctgcagcccagcttctcagcctcgtggttgcagctgtggatatcgatgcggtacagagtgaaaggccggaggttggagatgacagtcctctccttgttatccactctgctctcaaagaaagggtactctgtctcgaactcctccgggtctgtgatattgtaggtgtcagctaccgtggtgttcctgcttcggctggacatggtcgtgttggccacttgcatgacgtctctccgcctcctttcgggcctgcaaggaggagaaagaaggtcagctgctaagttctttagttttctttgtcttttccatggctagggtagaagtaataggaaagtcctagccttgaactaaaagaaactacttaggctccttggataagaaccaacaaataaccaggattttcgccttcacaacccatcctgtactctcttgagacatcctcagagaaacatgtgcacacatggttctaggaaacagtgtgccagccatttttagccagacagtgctctgttttaccagacagtgctacttggtcaggcctgataactgagcattcaacacgacaaaccatcagcctcataagactggttgggaaaggtgaggcctaagacacacaaggtagaagagacccaaagtccaacagtagtggcttctttgatcttctctgaaatgaagatgccctagtttaagcagctagcaagtacctaaatcccagcgcttttcctgaggaacagggtctcctattgatgaatgcccccagtccctagtacacagcatggtcttggaggttcatcaaacctggctttgcatcctccttggcagtgagcttgtctgtgataggagcggagcctctgttactcactgacttgtgttagcttaagaaactacagaaaagcaacaggaacagagtctggggctccctctcttccagaaaaaacttctaacattcataaaaactgaaaaaaaaaataaataaaaatatgacatcagataagcagccctatgggctggaagcaaccctcaggttgtctgcaggtgaacagtgggcaagctgggtattgaggctgttgctggcattgcttgtgacaggacaaaggatgagaggaacaggtgggactgcactgcgtctggtaagaacatgacagagtaagacaaaagggagcctagagtacggtgctcgaggcgccttggaagccatgcccaaggcctatggatgctccctaggccaacatctgggagagtacagtagggagttgggggaaagggccgcatgctccctggtcttcagcagatggatctgctaccaaaaccatagttctttgtcgggtactgacatgccagtttccattgaaagtatatcaactgccataccagggcccatctcaaaagttaggaatggggatttgttatcataatgctggggaaagagataggaaatgagggtactattctaccatgctcacacccagcaaaccataaaccgaccaatcaacaaaccatggaaggaaagataaaggaaactactttttcaggtatgtgtgtgcgtgcatgcatgtgtgcgtgcgtgtgtgtgtgtgtgtgtgtgtgtgtgtgtgtgtgtgtggcggctaggcagatgttcaaaacattattattaaatttcctgcctccctttcaaaccagagcaacacaaacgtgtgccttcatgtaagaagttgcttgtgtttgatttattgcaaaatgatactggtccacttcttgccttgacccacccccaacaaactgggctatagcaccaagcagactcagtggcctccttacttataaacactgcactcttgcatattattcaacagttactgtggacacagagtcaggaccatgaagggaggcaatataggcatcagaaggctagggatgattcaaacagagtggtccagggggtggcaaacagcacactatcagctttccttactccataaagtatcctgatgctactggaaaaacttgtactgtttaacatctgagaatgttttagtgacagaaacatttttgaagcgtcttcaaaaatgaagatggcggccaatgaaagccatttactgataccaagtttaggaagtaactttataaatagtagcctccactctactataaactcacaggcaaagtcagacaatgcagaaagactaaatctgatatatttgccttcagcccccagataaacgtgcacctagaatgacatgaaggtaggaataggttttaggtaaactcggttgtatgatgacagagttgctcctgtcatctagggtgctcctttatgggaccttgcgagacctcgtatagatgtcagatccttgcctcggtaggaggtaaaaaacaaccccccaaaccctaatggccaggtgtggtgacccctatctgcatgctgggtctgttttaggacactgaggtgaggacacttacacgaaggaatgtgaccgagagaaaattcgtttcaaacccacagtacaagccctgcacggtcccatctgggtcaaccacagccttgccgcccaatcacttagcctagcgttacacagctgatgataacagcagtcacaaagtgatgtcatctgagtataactaaacaccaagggctttctgctatggtcggtctacccttggttctcatgctgtattgttagatgtttggtcactagtggggcagctgcaggaggaacctttagggaatggtcctgttagggggagatcctgatgtagcaaacaagctaagtgctcaacgtctgttcagcttcctgtctgccctgaaatcgctgctgtgccataagtactttggccatgcgacacacccaaagatgccttcatcaacaaaagccacactggctccttgttcttctaactttaaaactgttgagctatgcacaccactaagcatcacgcaatttgttagcaacaatgaaagaacgacctaaacctctggccatgcctacaggagtcctggatttgactgacacccagtataggggtgggtatgggaggacctctgcctggcctctttgggacgctccatacctggccactctcatggctactcttactgagtgttggttcaggtggaatgctccacatagataacccaatgaagggacggaggtttggtgttggagaatgtaggtctgagacccaagactcaaacagacagtaagttagtgaggatagaggtcactgcatccagtttttaaagcaccttttgatttgtttcctgtcttcaggtctttaaattcaaagaaacagacatgacacagaggggaacatgggtaaaagccaaaacagcagcgcctcctgtcagctgactgcaatagcagcccagcagcctgccaaccaacaggagctgctccgcgtggggcagatgggagcatgcacagcctaaggcatcaaatgtagcctttccaaagggcacactcacctgggcacaaagatggaattgtgaaggaaattctcaaagactttacggtactcagcctcctccttctcagcctgcttctcagcttcagttttagggcaagcgcagcatggccctttatcaccaccacacacttctgtcttgggattttccgtcacctcctccacgtcgatggtaccatcggcgtactttctgatgggtattttgtctgcttggggaagaccagagatgggaatgaaagccatgcaggaagtgcctgttagcattgctggtggaggggaggggtgctggtgtgcactgctgagaagcaagccctgcaacgtgacaggtgtgaccctggggctcaggttctctcagacaacagacaaggccagtggcactgcctgcctgctgcctgccagcccccagctcacctttggagcagtagttgtgccggtacaggtaaccatcctggggctgccgctgccacctcacaatgtagtaactcaagttaccattgggcagagttggaggattccacttcacaatcagctgagaggaagagtttgatgctgagaggacatctaggggaatggaagggactggaaagaagaacaaagaacatttcagagaatagaccggaatccgaggttgaggttatcaggggctcgatgcaaaccaacaggaagctgcaggcttgacctgggcacagagatagcaacggaccaaccagagcatgaggtgaaccagagctttggaaagattaccaactgggaggccagccctctctcagagcacttgctcttagataatctatgtgcaaacacacatggctcactgacctgggactgaaggggctacgagggcatagctggggttaaaggggactctaaaaaaaaacctagagttgattcagttatgacaactgtcattctccggtgtgtattaggaaatggcaaattggacggcttagtacttcacctgtgattgcaagttttaatctctgataaaaacttgataaacatgacagggagccaaaatgaagagtgggagtgggggatggacagtgtgtggggtggttatgtgcctgggtgaaaggactgtgaaacaaggagcagggactcacagatcttgtaagcaagtcatgattcaggaaagccagtacgcctgagccctgattctatgcaaagtcctgttacaagcaaatgagtttaacaaaacgacggaggcttacatactactgatgcccaggctcaaaagtagaactaacttacgtaaaaaggaaaacatcccaggaccactgctgtgtcctcaccatagcaaaagagataccacagccgaagcgcaccccatccccaacacacacacatgcacatggcaaagaatgaggcccactggttttggcagtttccaaagtgcaagagacagaaccgtggttatgacacttaagaataaggtccaaccaagctgtttttgaatctgaaaggtgaaaacaagcttgtaccggtcagtcttatgacaacttggcacaacctatctgacagaaaaaaacctcaactgagaggatggctcagtatgatcaggctgtaaggcaggcctgcagggcatcttcttaactaatggttggtggaggaaatgcccagctcattgtggtgaggaggcctctgggctggtggtcctggggtctctaagaaagcaggctgagcaagccactgggagcaagccagtgcttctccaaagttcctgtctctgctggagtttctaccctggcttccttaacggcagactgtgatcctgatgtggaagtcaaatcctttccttgccaggtcatataccaccacaccaacaggaagcgaatggacagagggctgttaggatgtatgcggtgctcaaggctctgagaaccactgagggacaacacgttggaagaccaagactgatgcagagaagacaagcacttgatgcctggtcccgtgagcgttttcaataagtatttccaaaacagtttcttactaggtatttcaatgacaagtttcccagatgaagcagaggcacatgatggactaagtacgtcactatcggtcccaagcacttttgaggtcagggaccaaatgcagtttttaatatttaggaagaagaataggatttcttaagaatctgctataatgataagaaattcctgcttagtttttttttttttcctctcagatctgctgcaatgattcactgggagtcattactcccaaactttaagatgagaaatgtattcagttgctttgctcttggaacggtgaatatgtttcattgtgttgtaagccactcaagtcgatggaattttttgttgtaagaggtcagtcattatctttacagtgaagcatatctttctacctctctcgtctttggatacaaactcacacctttgcttttccccacttctaggttctggagctggggaactagtacgtaaacacatgaagaagatgagaagggattagtcaataatccgatatcatagtactctaagcctgcgttcgcctgttgagaaagctggtggctacggaataaaggtggaaagccagcaagccactagaggcaaggggtgcaccggtcattgtcgtctcagtccctggaagtctgcactgagagagaggagaccagccacaagtggctacatcctgggcattcagagaaaccatcctactgctctggaggacaccgattcaattgcgtagagcagagatgttgacatttaagatggtaccaagtctaggaaatggctcaggagaacttaaggtgttggcagtgagaatgttaatacagtgttaagcagcaacaacaagcatctcattgctagccctccagccatactgtgcctatgtaaataaagcaagcttataaaaattcagggaacaggggttggaggttgttcctccggtgcgtggatacctgaagcattggtgcgaatgtacaagatttcacttttggccccacggatatggtcgttttccaccatggtgagggtcacagccttgacatagacagcatactgggtccagggcttcagcccatgcagtaaaatgccaggctcgccctccttgttcggaggcaggtctacatccaccatgttccagctgttggagccacaggcatcctgcccgtcatattccgtaacgtttttaaatggtctgaaagacagtcaacacaggccagagaacgttgaagggctctggcatcaaaactccccagaaccttccccaggggctgaacgtgtcacactttctatttctattgggaactccaaataaaactcatcatcaattagatttctatacaaaaataaaatcaagcatctggactcgggagctcgacagctatgaaccgcagaactgacaagcagccttagagacagcaagtccctcattcacagggactgaaaatgctgcgtctaagacatgcggatgctcattgtcttcttaagtgagagttaaaaacaacaacaacaacatcaacaaaaacccaactaatgaaaaaaaaaaaaacaaaaaacaagcagagctgtttaatgcaaacaataaactaataaaacacaacttttggacaaaactaactttcctatccccctagaactctaaaatgtaattcgataatagccatttttatttatttttatttttacttttttggtttttcgagacaaggtttctctgtgaagccctggctgtcctggaactcactctgtagaccaggctgacctcgaactgcctcccaagtgctgggatcaaagatgtgcaccaccactgcctggctcataatagctattttttttaaacaatttttttagtagatattttcttcatttacatttcaaatgctatcctgaaagtctcctataccctccccccgccctgctccccaacccacccactcccacttcctagccctggcattcccctgtactggggcatataatcttcacaagaccaagggcctctcctcccaacgatggccgactaggccatcctttgctacatatgcagctagaaacacgagctcagggggtattggttagttcatattgctgttcctcctatagggttgcagaccccttcagctccttggatactttctctagctcctccattgggggccctgtgttccatccaatagatgaatgtgagcattcacttctgtatttgccaggcactggcatagcctcacaagagacagctatatctgggtcatgtcagcaaaatcttgctggtgtatgcaatagtgtctgcgtttggtggctgagataatagctattttcgatgcttaactatctcataatattccaacactttgtttcctgattgtctcagagagtggcattggtgataccaccagttagcatcgccagtgcactggcagcactttcaaagggaagggagggctctgcacagatgcatggtaggcggggtgtctactttgacgagatgcaagggctgctagctactggcttctgaaattatgctgacatgacttgtgttcgatcacgatgggagaggggtggcccccatttacacaaacactgagtgattttgagctttctattttccctgggctataacagttcccaggaccctaggtcaagtggcagcgaagataccaggcactgccaccagcacctttgcttctggaaagatcagtgcctgggaaaacggatgaccacacagactggggaaaccacctggccacaggaggctgagtaggaaaatgggacagctcaggaagctgtgatgtggagtaagcaggatggtcctatcaggaactgctgacacctaggttcgtccctcactattctttaatgctagagggctcatggcccagagcaaagtcttggcaaggccatgcatgcatcaagtgactggaagacaatgggtcagttcagggtagccttggtgagaacagagcaccccatggggtattttgggggacagaggctaagacaatatttaaccaagtccacataatggcttatgacagtttggaggcattcagttctcttaaaattcatcccctttccccttggggtgaacttactcaaacaccatataaacaatgaaagggctgaggcgggacactctcctgactagctgctgtctcaggttgggacagtttaatatgacaactagagaggtcacatgaggatgaggtgacggtcgcctgacttgattctttataggtacaccaaagactctctattcagccaagtcacactgccacctggccgtccccaccctcaagcttgacatcagagtgagaggcccaaactcacgcctccttgtagtaaactgtgaagctgatgagatcccggtagtccggcggccggtaccggtgccacgttatgatgattcggttcttccaggtcgtggtggaggtgaaacggagaacatcactttcacctggggcacagaggcacatccatgagtgacagaaacccatccgtctctgtgatgtcactctgagagaaaaccctgactgtgcttccaaaggcaaatctatggctggcgcactcatgacctgtcaggtcaggctggctgcccctaagggagaagccctttgaaatgctccgatcacaggcttctgctatgttcttgatggggaacaggtcagggggctctgcagaaaggtgagggtgctggctgtgtacacacttaccacctctgcctaactactcttctcaagatgcctcctggatccgtagagtatcagataaatctggatactcaaaagacaaggctagccctccccccacatgtatgcacacgtctgactagctgttggtcacacagctcctgattccccactaatgctatataatgacggagggtcaaatagagctttagtatttctgtttgtttgtttttgtttccttaaaacagggttttagatgtagcccaagctggcttcaaactcacaatcctcctgcctcagcctgttaagtggtgagattataggagtgtaccatcatgcctagctccatacagtgctagaggagagactgacatccaggtctgggaccacatttggagaggggctgggttaacaggttgaactctaggctatgagatcttagggtgaaaactctcatcaccaggatggaattattactttgtccaacatgtgaaaagcattccgattcaaagacatgggctcactttgttctagaaggagcagggcagagaaaggaggccaagtgtgaggacaaggacactggacgtccacggaagtattatagtggagcatctaccctgatccttctatggcacaggagagaaaggcagagctgacctggagtggactggctcttcggtaaatattgtttgctcatattgaaaaggtatttaactgtgcaagctctaagaacctgcttggatgtttaatagactcaaatggacccttagaaaaatatatcagtatgagtgtatcaggcacccgcccactaatatgcagaggccaggcactggctgcagtggtttgacagtccatggtgaaacaaagagcccgcccacggatgccaaagactgttatctggactgtggcagtcccaggggtctgagatgctgtcactcacaggaagctcgctctccgttgttcctggtgtttatgtcccctttgctctggcgtcccttggttccggtcacttcctccatgcggtaaatttcggagacacacagcttgggattgaaagcaaagtacatctttccggacctgacggtcaggttccggtggttccagtcccacagctgctgcaagttctggttgtctaggacatagaaggagtagttcctggaagcacagaaatgcgcattagcaacgcactgagcccacagccccctcaggcgacagagagcctctctgcggccagtgtagttccataccaacaaggggtttgcttgcttactggggctttataacagcagacagtggcggtagccactggcaaaaagatgtgccaaggacaatgttttgaaatcgactggacactaattaatttcatgacctctattaccaacacacaaagcttctcccaccaaaggatgttccttgattcattccctgggaaaatgcctttggttatttctctcagagccaggtatggacaatgtgcttgaaatcagcctacaggcgaacagacatgctccgctactcaaccggaacagcttacatactagcagcaaaggctgccattatccctaggaataaatcctacccatgcccaatcagcagtgcatgatgggatgggagacagcctggctctctgtacatttgcctctcgctgcaagaagccttcatttcagtccctgagtacgtgggagaggacagacatccagtgtctccctgggctctcacatcagggtagctagatgcagcgtgtaacatggtggctttccacatttagtatgtacacagcttgttctcagtatcccctaccgctagagcttctggtgcagaggtccagagtgcaccaggaatctcgatgtctagcaggtaatgctgataaacaccagtcctgggtccacccagaaactaccaggccggccacggcactgtttccaaggcctaacttccactagaggggagatatgggcatggccaaagggaagatagctatcttagggggccaaggggagagcaaggattctacttgggtgtctgcataagttttttttttttttttaaacagttatttctaaatccacccacagaccagacaaacatgtcccaaagtcctgttctgccctaaaggctgtgtccacctaggcaccacctgctgagagtcctcacccacattagaactactgcctagggggtctgctttcaacatgaccttcacatactgagaaatgagttacagctgcagggttgccaagcctctcccgacacttcctggagaactcattagggagctcagaggccaggacccaggaccggaagagccatgaattccccacaggacaagccacaggcagtttcctcggtgctatgaagagcagcgttcatgtgcgtaggtgaagtctgaggtctcgttggttttaagattccttatgagagccagtgagatggctcatgcgtgcaagagtacttacatcacaagtctggtgatcaggaccccgagaaggaagaacagttactgtgtaaggctgtcctctgacctctgcacatacatccctctcataggtaagagtcctcacatggaggagtcggagctcaagtgtgagctaagcagttgaatggaagccccaaccctccttgagcacttctgcgcaggcttcagcgcagggtctgcacacgcacgggctcaatgagccgctagcctttggtaaggagtgttcccctaccatgcttgtttattggagtaggaaacaaagaccctgatcaaaggaatccagatcgtagtacctaagagtgtatgtcctagggaggtccttggatacctgtcagtcagaactgccacctacgggacccaccaatgttcactcttattgatatgggggtggtcaggataggcagacaactgaaggataggtgtaatggtggaaagattgggtcccattaggtgtcttgcctaaccatgggccattcttaaactgctgcctcaacgctaacctcaccggtctggctgctcccaagtcttgagagtcccaggagacaaaaatctcctgttattgaagacacctccaggccggatacaagggccaactaatcaggtcaaactcccatctccatctaggccaaggcctcttagtaaactaatttctcaagcaggaaacagaattgtactgaaaataccttccttctgagccacacattcttctccccagggagggagcatgaattagaaaccaggaatttgacaaatgaatggcaggcacatagaaactcagggtatttgactaatcactttgaaaagcaagattatcttttcacagtaaacacaaactttctctctttaaacaatgcagagaaggctgggagccagaataccaatatcaataaaatccaggcattgtggagcctacagaaggtaaagaaaaataaaaaccttcaccaaaataataatcaacctgctggggagggcccgtccacacagtgcctgcttcccaacccccacccgaggcctgcctcctcaacaccttctgcaatagtgagagttaagagatatccccaggtggagatttgtctacacaccttcaagatactaatgtccttgaatggtcattgttttctcagggagatgactagttaagggaaccactcaataaatgcgacttcataaatcgtttctaagctcactgcctttttacatccacagaatatttttaaaaaccaaactgttcattttttttcctttggctcctacacgctgtatgatctcccggagttttattgagtggtattttggctcttagttcgatccttccttctttcctaccttcctttctgcaccccccccaccccctcaagacagggtttctctgtgtagccctggctgtcctggaacttactctgtagaccaggctggcctagaactcagaaatccacctgcctctgcctcccaagtgctgggactaaagaaatgtgccaccactgcctggtagcttgtagctttttcaacattagaaaaaaaaatcccctctattggatgggtttcacctgccaatgcatagacctactaggatggttagtcaacaatttgggggccctgtcacatgataagctcacagccaacacttagctatgatctgtggctaccatttctaggcctcccttttaagtgaagactgcaggggaccgagtgtagagtcttccatgtaggaactgataaccaggcccaagcaccttctccactgcttatgaagtggcacctgaatggtttactcagcttctctagggctgctggttctcagctgcccatggagctatcttttctggtctcttcgggccccgaggccaattgagaagaactggatgctaaatataaaaggacgttataagctttgaatcacctcacaaaggcacaggatgacctgtgctactctctgaactgtcaaaagtcccccagaacaaagtcacagaaaataggtaagtcctgcaccttgatacaggcagggctgagctcaaggtctccagcctacacaggacccctctttcaagctcttactggcatcctggaagcacagacaaccaataactcatatccatttttgcaaggagacactactactgcctgtgacagctgtctctagagggcatcagaagccagtacactgaagctgtcaccagtacagtcatttataacactacttagcccatctgtagaatattggttcacttcaggtccacagttataccatctacaaaggctcagttagtatggaaatatatacccaggcaaaactaaaccaagcaccacatagattgacaaaataggccgtttgttcctgagtgctttttaaaaacagctttgctgaggtacgttttaccttctaaaattgttcccactataaatatacagctccatgggcttcaataaatttatacagatggccaatcatctttcaatcatccagattcagaacatttatatcaaactgaagcccccccccccattcttctcccatccaaggcatttcccctgtctgttcacccaggatgctacatgtaccattggctagtatcttactgtgtgttttgcgcttatactgaggaacccctagattatatctaacgcaccattttgagaaaatactatggaaaatgttgctaagagcattcacatgtgatatttgcatacacatgcttccatatctctaggaacagcctagacttgctaggcaaagtaactaattgtatgtattttaaaagtcccaagtatgtgctaaaatgtggttatgttagggtctagttccagagatgcagggcacattacaaaaccctgagtctgcaaagggtagagatgcctaaacagactaagaattctaactttatactggcccccttccagtatttttgttctgttcaagacaataatctttggcagcacctaagagcttgaacttttcatttatttgattgctcccaaaccaaagttgtccaaagcagagcattttttttttcagataatggaaatattttctctcttattcaaggagaaccacatatccctatccccagacaaaacttgattttccttcttaatcccccccatgacccccacactttgctagagatcaggtagcaagcaccacacactctaggttaagtgctctatcactacaccacaccactacttctgagaaaaccctttcctttctgtgtctgtccttgagtacacagacgtgcaggcttctggacacaggagagagacactgggcatcctcctgttactctctaccccactgtcctgaaacaggctgtctcacggagtctggctcaggtcaggcaggtcatctgactagcaagttcttgggatcagtctgtctcagctcaccacctggagctactcctgggtgcagctaggctcagtcacgccgaggattcaaacacgaggcctcatgattgccaagcaagtacacttagctagggagtcatctctccaactcctatgatcttatctgaagttgggattctacagctatgccacactacagagagagtgatggctgagaccaaagggtcctgctgctacccaagtaccgttctgtggctgtgcaccctgatgtctgctctgctgggattctgcttcacctgtcgtcggcctggaaatcagtgtacagaaatgtggcctgatgagcatgcatgacccaggtaagcaggcccagaaagaaaacacaggaactacacgccaggcaaaacacttgtgttcccacggggggctaccgagaaggccaagatcaactgcagtgctttcctggatagctaaggctgaaggataccttcatgggggtggggggtggggtggggggaaatattcctctttccatcaagacagaagtcttcagaatcctcctgagccgcacggaagcatttatgccattttcagtagccatacaaaaagctcaagaaatagattccttcaacactgagttgaggcaagattgcttaaaaataaaataaaatataaaataaaataaaaactcagaagtggcctgggactatatacatttgaaggatcacagggatagcgaagtcatttctaatctttgccatgtgccaaacatacacagaaatactcagaaagttactcgagcttatgctggcaactcccccaccccaagttttctgtctgacttggggtgaggtgaacctggtgaagttccacagggctcccctttccctgtaactgtgatgtatccacacattctgggaaacactggggggagggggaggaggctatcagtataaacgacagcaccctgaaattaaatgtcagataactctgctaatacacccctcctcaaaatctccagtgcgtcccaaggcttacctaaatgataacctctacatctgcatgcaacagtgcctcctataatcctagtactgcctacctcccccaggagctgaacacaagacaggcccaaatgtcattctgtgctctctcctcttctgtccctgtgctttctagtcccccaaacccttccaggccagctttgctcagaagcctgtgtggccttctccaccagagtcttccttttggtacccagagcaccttctgtcctctcctctggggctctctgaagcctgtgctttgcactacctgtgttcatctgcttattacaaatctttctctcctttattaaataagtgaggaagaagtgaaccagctgagccctaatactatacaacgagagacaaaggtgagttgttgctatgggtggcccctcaaagctttggatttcatcacagttaccagagaaagctactactaaagagtctcaaatgtcctggccagagatatccttgaagcaagaggctctcaacctgtgggtttagattcctttgaggtcaaatatcagacatccttcatatcatgcatttacattacaattcataacagtagtaaaattacagatatgacgtagcaatgaatagattatggttgggggtcaccactacatgaggaactgtattaaagggtggcagtactggaaggctgagaaacactgttttttaacacaagtctccaggcctcacctgccctccactcccttctcaaatgctaacagcagggctggcgtcagctttccagggaagcttccccaataccagaatccttaacaatacttggctgatatttatagtagccagaagctggaaacaacctagatgtccctcaacagaggaatggatacagaaaatgtggtacatttacacaatggagtactactcagctattaaaaacaatgacttcttgaaatttgcaggcaaatggatggatctagaaaatatcatcctgagtgaggtaactcaagtcacaaaagaacagatatggtatggactcactgataagtgggtactaggcagaaagcatggaatacccacaatacaactcatggaccacatgaagctcaagaggaagaccaaagagtggatgcttcatcctacttagaagggagaacaatataatcaagggaagtagagggtgggagggatttgggaagaagaaaggagggggaggggaaaagagggtgcagagtcaggtattgaaggagctagaggagatgtacagagggtcaggaaattgaacagaggggatggggaactggaggtagcaaccacaaagtcccagatgcgaggaaagcaagagcctcccaggaccccatggagatgacattagctgaaataacccacaaaggggaaggagaacctgtcaagaccatatccagaggttagactgccccccacccccgttgaaggatggggcaacccacccatcttcaaagttttaactcagaattgctcctgtctaaaggaaatacagggacaaagagtggagcagagactgaaggaaagtccatccagagactgtcctacctggggatccatctcacacgcagacaccaaacccagacactatttattgctgatgccaagaagtgcttgctgacaggagcctgatacagctccctcctgagaggctctgccagatcctgaccaatacagatgaagaggctggcagccaaatatcagactaagcacagggaccccaatggagaagttagaggaagaactgaaggagctgaaggggccttatctggcatcaatgggaggggaggcccttggtcctgtaaaggcttgatgccctagtgtagaggaatgctagggcagtgaggtgggactgggtgggtgggggagcacccgcatagaagcagggtaaggtgggatgggatagagggtttgcagaggggaaaccaggaaaaaggataacatttgaaatgtaaacaaataaaatactcatttaaaaaaaaaaaagacttggctggtacttcatacctaagttttgaataatgaaaaaataggctataaatattcttggaaataaaaaccagccttcaattttcagcaacatctaagtgtatgatacttttagttaaggcataatgataagataatcataccatgtaataatcctgataagaaataatcaaagcctccttttgactttctatgcctatcgcttgctcagtgtggaaaaaaggggtgggggagcttatcctccagtaactaacagatctgacagctactaatccgccaaggaaaacgtgcttcccgatgtcagggagtatagggcacagatctactggggggacataatgaaacgagatggagttacagttcactttaaaatttaatacaagcaacaatgaattaagggtgaccacggcctcatgtacgggaggccccagaatgattgcgacactctgggcagggtacctggcacatgcagaaggattcgcaaggagaaggaggtttttctgaagcttctctgctcgcaatgggaagatggacggggtgagcagctctgtttctggctgtatgtaccaggggaagacccaggccagagttggtgtgagaaaaactggaggttgtaaagaaacctcatccagacataccagcctggggacaagccttctccggatcttcccgtcactgtctttctaagggccctgattgcttagttaatacacgtataaaacttcaaagcattaacacagagctacaccagagccgtagcaaatgtctgaaggggccagcaagggcacacaggccagagtctggaaagaagctgtgtgcacatgcagtaagcaccagctttactctctgctgagaaacccacggctgctttaaaacccagcttgcaaagcatttctgtgcagaaggcacaacccctatcaatactccaaatcctgaaagctgatcaatacacagacgctgtaagctaatcaatgtacaagctctgcaagctgaaatatctctgtaataaaatgaactgtccgtactttgcaattctttctgcttctctgtttgaaaaggagatggcttgggtgtcaggtgctcaagtgcttgctgtacaagagaacccacgtaacatgtgcctgaaatctcagcacttagaaagtggacacagggacatctctgaggcttgtcagccagttagtctacaaactggtgagctccacgttcattgagagacatactcaaaagataagatagagaaaaaagacaccctgtgtctccttgaacctccagagggtgtgcacaaatatatacatgcatacaaatgcattctctctctctctcacacacacacacacacacacacacacacacacacacacaccccagagcacagtaggctgaggcaggttaagtgtcagatgaatcacaatttccctgggagttaagcaccacaggcaggctgctgtcactcctgcaggaaaggttccctgggaataccttggactgctagaactcagcgccctagcttagactcgtgtggtggtttggatgagaaatgtcacccatggggtcacacatttgaacgaacggccccaggagttggtgctacttggggaggctgtggaacctctgggaggtgctgccttgctggaggaagtgcaccaggagtgggctttgggggttcagggccttacctcatttcctgtctcatgtttgcagtttgagatgtgagctctaaccttttgtcttgcctgcttattgccatgcctctcccaccgttacggatgctccccattataagctcctccttccctaaggcattttggtcatggtgtctcatcacggcaacaaaaagtgtaactactacgatatgcaacccttgatgactgcaccttaagaatgaagacacccatttgccaaattgttagtatcctgtgacaactccacaggacttagagcaggaaggtgggtgaccacctaggcagcaggagcaggcccagggtttcaccttcattctgcgtaacaaacccaaggatatttgaaaagtacactaatcggggctgggagctggctcagcccctaagggtaagggcagctgctgtcaggtgcctacaacgagtggagttgaacccccaggagccacttgacaaaaggagagactgactcccacaagttgtattctgacacacatacacacacacacacacacacacacacacacacacacacacacacacaaacactattaagacgcagcacgccacagttctgtgaaaggagaatggagtgtttacccttccagctgctcctctcctaagatgagacgaaggttcttcaggaaggacaaggagaccaaggcatgagaatggcggatcttcacgtagccggtcaccacctcgatgagccccatgaagttctccaactccgaggcaatgttatctagagaaagaggagtccatgtcaccgagtcttcctctggtcaggtggcgcactatactcagattcacacacactggtgtggatgcgaaggggaagcaggtcttgcgtgggggaattgcagtgagaaggctggcatagcaccaaccgtccctgtctccactctccgcaagccacaccgggacaaggcctctgaggcgggaaagcagacttctaggaggctggctgctgcagttgccaagctgattaacagaacgtgaaatcagaaatgccaggctgctaattagccccagctgacaaatggaggcaacatctcttagatatgctcagtgacaggcagtcctggctccacagtgccatggcaggactgggcaaacctgtcccagcctggtaagaatggtctttgctggacctggagttccatatttaaagactccatttagggctgcaggaaaacactccaatatcacagcaatgatgcatgcaggtagttgatctatctttccccaccctggatcgagacagggtttcatgtatgctagacttaagatgaggctgaactcctgatcttcctgcttcctctccctgatccctattcctggcctgcaggcagttttgttttgttctctctctctctctctctctctctctctctctctctctctctctctaaataatttctgcatctctaagttttctatcctgctcataattacttctgtaaccagaaagttgccaggaaaagttgctaatgtaacaggaccgagagccagtgaatttcttttatcgtgcttacccacctttcacatagcaagtaacataaactttggttcatgacaaaattctaagactataataaattaggtgaatgggatttttattatatctgattcttgaaggttgatttggtgaaagtttctcctatggtttgatataaagaagaggacaaaagccttatactaagtctgagcctcacagagtcccctcacttgcaaaatacatacagtgctagttgtatgtaagggctacagggaattcaacctgaaaaaaagagtggctgcggctaagcctgtgttttgttccccaagaattgaatctataacccctgtgtgcaagacaagtgctctggccgccctgaactcactctgtagaccaggctagtctcgaattcagagatctgcctgcctctgcctcagaagcactaggatgaaaggtgtcttcactgtgtagaccaggctagcctcgagttcagagatctgcctgcctctgcctcagaagcactaggatgaaaggtgtcttcactgtgtagaccaggctagcctcgagttcagagatctgcctgcctctgcctcagaagcactaggatgaaaggtgtcttcactgtgtagaccaggctagcctcaagttcagagatctgcctgcctctgcctcagaagcactaggatgaaaggtgtcttcactgtggagaccaggctagcctcaaattcagagatctgcctgcctctgcctcagaagcactaggatgaaagttgtctgtcaccacacccagtaagtaattcttcttcatgggctgaatttcccaatcacattgatctcccatgacaggaagatataaaaacagtaaggtttatgaattttaaaaggcatgtcttgggatcaataaactcaaagaaaattaatagatctggtatgagcaaaactacaatttggttattaacataagggtcagcgttcttgtgtggggaacagggccctccattgtggaagggtagagaatgggcaataaaaactaaggacttgggggctcgagagatggctcagcagttaagagcactgactgctcttccaaaggtcctgagttcaaatcacagcaactacatggtggctcacaactatccataatgagatctgatgccctattctggggtgtctgaagacagctacagtgcacttacatataataaatcaatcaatcaatcaatcaatcaatcttaaagaaacaaacaaacaaacagacaaatgcccaagcctcaaaaactcactaccacaaaaaaccccataaagaaaaaaacaaacaaacaaaaatccaaaaccatacatacaaaaagaaaccctaaaccaaaaaaaaaaaaaaaaaaaacaacaaaaaaacccaccttgtttctattacctctacagactaagaatataattcacttatttaacaaatacttaggggcaagcaatccactataaataaagaggacactttgtctctgaagtaaagaaaaactttctttccctaaagatttaccagtaaagattaaaataatagtcttctgcctatagtattttacctcatgaaactactagtagctcaacctagaagttctagtggcaggaactgaagggcatctatctaggggactttctctgtcagtccaagactagagaagataagattacagggctctgctgccaccatgggagatgttgttcatgaatacaattctgaagcaacatcccaggagcgctgtctacaccaagtgaagaaagggagtctgcaggctcagaccacagcttgaggtgtggatggcacatctttctggctctgagaggtaaaacaggagatcactctttaatctttcctttgctcagtctcctcaacggaggcaagcaacccaaactctggccttacagaagtgtggactctgcagcagctagcatgagccagccatgggtctgagccatcagaggtgtgtgtgtgtgtgtgtgagagagagtgtgagagggtgagtgtgtgtatgtgtgtgtgtgtgagagagagtgtgagagtgtgtgtgtgtgtgagagagtgtgagagagagagagagagagagagagagagagagagagagagagagagagagagagagagaacaggcctttcctgcgtaagatgtgacttaaagaaagacgagggctgatcctctttacaggagtatattctgggctaatggctcaaacatcgactgtctttctatctggattttatgtttccgagaaactcacaggggaggcgtcacacaagaggcttacttacatcccgtgctgcttttctaagcctgtattctgcttgtgactaaagggacagagccaggaagagcatcgacgcaagtaaaccaagaagcggtccagccactggtctggtggggtggagtacttactgcctctccggatgttaataagcagattgcccttcaggatggtgcatccttggagcatttgagcagaagtcaccgaatcgatggttttcgttttcttctcttcatcgccgcagactttggggcaggggccttcgcaggggatacagtacatgctggtgaagataaagagtggaccaaaaccacaaaagtgagtttatcttcttgcattaaaacaaacaaacaaaaaagaaaacaaaacagaaaagtaaggaggctttttgtttgttattacagcagcatttcatgaaagaaccaggctgaaacatccaaacaatcacacagtataatctcctacccagggcaagccatccaagctgcaaacacacagagggaacacagactgcagaagaggaaagcatgtcgcttgtttttagacagaatacacttcccatgcaagaagcatgtgggtatcataggcttgtggctggggagccagctggtctacctgggagacacagactcacaggcactctggggctctgaataaatggagctgctcactgatgagaccctcgcctgcctgcatgccagaggaggttgggcaagctggcagcgctggggaagggacagccgcatctttgtcactaacaggagtctaattcccaaagcccagggagagattccaacaggtacctggcgcagtgcaggtccattattttcttgctaaagcacaagtgaatttatatcttgacagtgctttgcagaaacttgtaaattgatccaaatgttgttttgctgcacatacaataaaagccaggaacctccgcagctctctaatgaggtccttgtccgaatgctaccctcagtgccacacacaacctggagccagaagcagtggcatgcttcctgcttcctgcccatcatttaggaaacggcagagaagatgctaagcctggggctataccttctgagaccttagcctgtatacagaataagggccgagaagctgggtcgatttctgaatgagaaaaatgacccaatcttctggaatgggtgggacagtgttatattcaaattactgctcttaaatgagtccgtgaaacctcacgaaaagaaagaaaatcagggtaaagcaagagctgcctttactaaagtcttccatccgttcccaaaatactatctatagtctgttttgtttaaaaaccttaatagttctctgttatcttcctatttcctattaggagagtggggctgaggttttcacaggctcccatgggaagtggacctagctagccaaagtgggggacatctggcttcctataataccacatgcaacatgcttgtggtgttggaacgagaatggccccatagactcatgcatttgaaggctcagtccctagctaacagaactgcttgggaaggattaggaggcgtctggtgctgagctctcagttatttccttcactccaccatctgggatcctgccctttgaaatcacaagccaaattagacatacttctgtatacgcagctaaggtcatgacattttatcacagcactagagaagaaactaagacagctctcatttctagcctggagaggggtttccttctgaactttctttgtgctctgtttgtgtttttctcatttaactacatgacgcataaaacccagctctctctaaaaacaaacaaacaaacaaaaacatacttggaataactgcagcttcatacacagacccgtgcagacagtacagagcacccgtgtactttccacccaacgctcccaactgctgctacatccaacataacgataacacagtgacctggcacgcgtgtcaggttctgtgcatttctccatctgtctacatagacctgtaaattccctactgcaatcgagttcgaggacaacttcaacaaagtcctcctgtgtgttgcctgtttgtagctgccctctgacctgccccagacttcactatccctaactcccggtaactgccaaggtgtcttctactgccaccttgggaaatgttacagaaaggcagtcatatagcacatggcttctgaggctgatgtgcttcccaatgtacaacacccttgaagttaaaccttcctttacctctcctggtgatgttctgtggggaggcctgctttcctacttattgtaccagatatctgagttgtcctcacttcctggtattacaaacaaagaggctacagacaagtacatgaaataaacgttcagggtcaacatgactatgtttattctttccccattttttttttatccaaataggaaaacctggagccacctcccgctttgttaaaatgtctaggaatcatttagcaattgaaaacatgtctataagggtgtgttctgattatagcttaagtttcctaaaacccactcttgtatagacagagcacaaacagtattttcaaaatactttcaagcaagtgctaaataggcttcaaaggccagcagagggacagctattatttcttgatagtattttaatttaaaatgggagagactgtgagaacgggcagggagcagcagcccagtgctttaagagcacactggagggaaggagacccaactcctatgaagacacagcatcctcactgtgtgttacaggacagttgaatgcctttcagtagggagaggaaataaacttgcaggtcaaatatgatatagtgagtttgatggccagctctccctcttttttttttttaatttttattaggtattttcctcatttacatttccaatgctatctcaaaagtcccccatacccacccccccactcccctattcacccactccccctttttggccctggcgttcccctgtactggggcatataaagtttgcaagtccaatgggcctctctttccagtgatggccgactaggccatcttttgatacatatgcagctagagtcaagagctccggggtactggttagttcataatgttgttccacctatagggttgcagaccccttaagctccttgggtattttctctagctcctccattgggggccctgtgatccatccaacagctgactctgagcatccacttctgtgtttgctaggcccctggcatagtctcacaagagaccagctctccctcttaccagccagctctcccttgctgctcccagccccagaaggaaggcattgtctggactgtgggtaagcaacttcacactgccagacaaaggtctttggctcaagatgtagaggggtacatacaccccttttctatgctaaaagtgattatttagatgtaaagggcctctaaaggcttatctagtaacagcttagtcaacagctcaacaaggtagggtttactagaaggcctgtctgtccttggcacatgccaaaagcaatggacccaagagctaaaaccaacctcccccctcctcttcttcatcctcctcctcctcctcttcctcttcttcataaactgatcaccctaagtattttgttacagtaaccgagagttgactaacagagactgccaacacatgaataaaaaaatggcagacttatttttagtagactgtattatacaattctgagtcattgtagtccctggcttaccaaatcatttaaaaatatttgatcttttcctttttactgtgataatggtgcagaaattaaacaggtaggaatttacttttcatcttagtattatgaaagaaaatgtaatctttggtttgatccagcagtcagcttacagttagagacatctccaaatactatagcagcaatggaatttctttttgaagatgtactgaacatcttttatagagagtgactgtagcaaaactatacattttatatacttacagaattgttagcaatgctggagactggacctaggtccttctatgtgccagagattggatctaggaccttctacaaggtggggactggatccagggcatctcacaagtgtgggcagtattcagaacttcctagttgtattttcagaggctcagaaatgtgtatgtgtgtacagtaccggaggcagggaatgaattacaatggaaccttcccccaaagtcctctgctctgttcaggggaggaaacaacaacctcagacctctgactgtcctgtgtttccagagttataaaatcctgtcttcacgaacatcacccatgacgagtttgtggaagtacagcttgttcagtcagaaacagctctgcttccacagtgaagcctcagagtccaggaaggaaaaccagaggccacaaagatggtggaaggcttagagaaaggaggttctggcagaaaacatggcagatcagactactagttcatgcccagagcccaggcttgcaatgagacatgggagacctgtttccaggcctcatgaatgctggtgagggttgtcttcactctctctcagactgagaggcaaagctttcactggcctagagtagcaagctgggaaggagtatctgtagttaaagtctccaccgctcatgcttcagctttgcaggtgcacggggagcaaggctcgggagcacctggccagcaagctaattcttacgctctagtgctagcaaggcaccctcaaagtttagaaataaaaaagcaagctgaaacagacccaccactccaagattcgcattaaaacaagctcagccatcaccctctgacagacccatctgggtgaacccctgggaggtcctacacttaggggctatgcagcagagaaccctcctgggcctagggacaagagccgctgacctctgggtgctgttgcggatgaagcctgagggacactcctgcatgcactcatcgtcgtggataacgaagccatccgagtcactgctctcagcgttggggatgttggcgcagaaatcgcgatccacacagcgccagccctcgaacctgtaggtgccaggcgggcaggcaggcacacacacgcctttgtagtagtagtgtctgcaggccacgcaggttgtgttgtcgtccggtgtgtggcagctgcccaggcactccgggtggcagcactcgttgttctcggtgcaggctcgcttcccgcacacacttgggcacactgtgaggacagagggagagagggtggtaaggatgatgggctacacagcctcccctgggtcatccgctgccaccaccagcgtggcgtaccggagactggctagaaaagcacacctcctctgaaatctgcgtagatcttaccgtctgtgaattccacttgggtaaagacacaacccaacccaagtccattctctcctttagactcctggtttgctatgggatgagccattaacatttaacacccatggactcttcaaaatgcagtggccacacccattaaaagggagcccatggtactggtgaacagagagaaatacagacaatatgggcctctaggactatacaaggcactgtgctcctcagctctccttctgtatgagaattccattctaagaccccagtggatgcctgaaccacgcacagaacactggcctgtgcacacccccttctccctcatgcatacaaagtgatgaagagaggaggcataacaagtacggtggtttgaaggagaaatgtcctacacaggttcatggatgtgaacaccaggccggtggtggtggtctgtttgggaaggttacagagcctttagcagggagctggaggaagtcctttgctgggggtagagtttgtttggggttttacagcctggcccacttctctatttcctgtgtgtgggtggaatgcgatcactttgtgtttcctgcctgacaagactaaagccattccttcccccctccccccggccaccacaaggactctgtcccctctaggactgtacaccaagataaacctctatctctggctccatttttctgtcaaggtattttatcacagcaacaggaaagttccaaataaaacagggaactagtcaaaacctctaggttactgcagcaaatgttaaccataccctttacaaaagcactgcgtgactgtggcctctctcttctcaaactccagaaaaagaaatccaggctacggagggactactctttgccatgggcacagaagtaattatggtctctaaagaggaacaagctacaatcgaagtgcccagtgcttatagaaggtctttggtaatgcccgcatctgcaacgtctcttcacaacacaaccttcagatcacaagcaagctgctggacggtacaggatggtagtacacagggtttctgtacccaggggccacctgctataagccttgttaagttaaaggccaggtccagtaatcacagatctaaaatcacaccctgaacaggtactctgctatgcacttctgtaaatatgtcatgatgaacacacgcctatgatccttacttgtgaacaacatataaaacaaacaaacaaacaaacaaacaaacaaatctggctaacagtacatgtacccaattgggggaagggattgtttggaaatactacagaactacataaatatgtatattttaactgtgatgtaccaaagagtcaagattccatgttttattaaaaaaaaaaaaaactactctaaaaccaaaactaagccaaataagccccagtaaccacaggttaacataaagaacacacaagttcatctgcacccaaaacagccgctttgtcaacccttcatgacgtggtctttcccgggttcttcctgtgcacaggtgtatgactacacatacctgaatgttaaaccttggactacggagaaagcactggatagacttggagtggataccaccatacagcaatctcgctacgtctcaccgttacagctgtgccccagcatcaaacgcgccctccatgcccacacattactgttccatgagtacggccatctttgtgggcacctctgcacgccactgggatcagttttcagaactgactgcacggagtcagtatgtgctctggagccttacaccagtctgtctacacagctctaagtcacatccaagaattcctgtgtcactgcccttaaaaacagttaaatcagcagcatgacccagtaaggagaccggagttctcgtgacactgccaaatcgacataatcttgcatttgactctaaagtcatccctccacagaaacatcggggcatttcccctccctggttatctccccctgtaggagttttaggtgttctgctgggtatgctctttggctccaaggcctcctagcctccactgtggctagagcgtggctgctctgtgtttgaagttcgttcatcccatgggctataaagcaaaagttttactagagcttcctggaacactcctttaaaaatacagtgtgtgtctccttttttcttgctggctgaaacatagaaatgatgcacagatattcacgcagccatctcagatggaacatgaggcaacatagtgagttcaacttggcctctcaagatggctggtgtctctagaccagtggttctcagcctgtgggtcgtgatcctcttgaggggtgacatatcagacaccatgggtatcagatacgtacattatgattcataatagtaccaaagttataattatgaagtagcaacaaaataattttatggttggggtcaccacaacataaggaactgtattaaatagtcacagcatgaagaaggctgaaaccactgctctagactaacactgcagctgctgacctttgaacttcttcaggtgaagagaaaccaacttttatctatgcaactgaaccatccacacagaagacctatcttcagagccacacacaagtgaagacggaaagccttctcagctggtttatgaccttcatttattataactagcttcaggctgtttttggacacccattctagctcaggaacaagcagaagttaattatctctggtcacaccaaaaaacaaacaaacaaacaaacaaaaaacataaaacaacaacaaaaacaccaaaaatcaaaccaaacaaacaaaaacctgcaggagcccctaatatcagaattcagacactgtctaccacagcaattacctcaaagacagtaaaatggagttcactgatttaacagcctaccatgtttttagcttcacacactaccactgattcttttttcactcaaacacatcaatatatgacacagttgaagatgatagtctgatgacttaattaaacatatgcactgcacgggttacatcaaggtttggggtctaaatgagcactgaagttatttgtgttaatttttttttttttaaatctgtcaatgacagaagtcaacgatctgttggcatttacgtacccacccagaatttactggataaacgctgcacctgtgtgccaggcaactggacatcctgtacaattgtacggtggcagtccctgacctcaaggaaaggataccacaccagggaagcaggaaagaccacgcacagggtgttcctagtactgtgggttcctggaagggtcttcagagacagtgagattggcactgaaaaaggaaggccgatggggaggggcatcaggaccacccgtcacagaggtcctcagatgtaaatacaggctgagatgttgctggaaacaaaatggaccagctcagggaggtaccacaggcacaccgattgacggacagcaggaaatcaccctgctttaggcaagggttaggttctcacactagaaaggtcagcatgctgtatagcgaaggcatgataaccagagtgggcagaagggcagaagaacagccagaggagaggaatctgctactgattacactgtccatctatggctcacacagatacataaagttcaatgtaactcgctaaaggctccctggtgcagctgtcccagccatccgccaacaccattgctctgtgattcgttagctcgacgttatcagcagagattatcaatgaggactagatgccaactttggagccctttacttacttacataggaaagaaaatgatacatcacaatagatgatgaagtcaaatagctacatagtagtcaaagcccataccatataaaagcagggggaaataaccttgtacactgtcttgaatcagttaccccccaaatgcaaatgctacatctttccaggatgagaaagagaaatagaaatccttgacaaaggatttgctaaatttgagacatgttgagccctgttccaggcctgtgatgggaacttcttctcaactaagaaccataaagttaaaaacactgtctttacagatagaacggttcttttaaggcatataccagatccatgagtcccttaacactcagtgatgccaactgtgaacaaatgcgcatctgatattctgtgggtggaagaggacaaaaaaggggtatgcagagagggagagagaagatggaggaaggccgacctgcttgctaggggactgactctagagatctgtgtgtggctacagggaggaaaggggtgtggctctctctccagcacagaggattccaggaggaagcaggagaacgcagggctgggattagacagtcctggagccagcttcagaagcctgctgtaaattttgtctcatctcgatgtatataaatattattagtgttcaggactattaagggatttcttgaaaagccccgggcagtgcgtctcgccagcagatggatggctgctctccatgagtgccaaaacttaaaatttagtgcagtaaccaaatttccaattacagcaccatggggggaaaggaggggacctcccccgccactccagaggaggggcacacaggcatggttaaagggcagtccaaccttaaaaagaggaaacggaagctgtgaactagggcacgaaggaaagcaaattgattttgcccatgagaccgttctattccttgccattaatcagcgaagacaataaaaacaaaaccttattgaaacagtcagaaaagctacaacatgtcctgtgtcagccatcagaaggacaggtccagggtcagcgtgcatacggaggccacacgttttaaaactcctcttcatttctttaagagaggacaaacaagcctcgcacatcagcttggtccttgcatttagtaagttctttctaatttccctttagcccgtgattgcttctgtcagtgaatcctttcatgatcgaaagctccgagaagcgagcaagctaaagcttccgagtccaacgtgggaagaagaagtaacgagaccgcagaagagaaggatggaagcattagacaaggcagtggaaaacactggtgggaacacagaggccagtgagggaagggggaggtaacaggtgctctgggtgactgtcagagcccatggggagtaaggttccgaacatcgtattcaactgtagtccaaactctagaacagagttaaaaagccatttgctatcagtccgtaagtctctctggcaagaatcttcatattatttgaaacaccacacctaggatttccatgggcttccccagttcgaaactaagccaacaagacctgtcttcaggctctagtggcctacctccaaaggaacctttctcatttcattaacccaaccaagcactctatggaggagtgtgtggataagccttgaaatagactcgtggctaatcttgggagcatcctgcccagtacttaaacatgcagaagcctcctccgtcctgcccctcctcttctttttctttcctcctcctcctcctgtttctcctgctcctgctcctcctcctgctcctgctcctcctcctcctcctgctcctcctcctcctgctcctcctcctcctgctgctcctcctcctgctgctcctcctcctcctcctcctcctcaatttggccctttccaaggcaagaggacatgtaatactcagggccgtcacatgtatttgtttataaagatttatacattctactttatgtgtgtgtgtatgtaccacttttacgtctggtacccagaggttagaagaggatgtcagatttctgggaaactggagttacagatgattgtgaaacattatgtttgcgctgggaacctaacctgggtcccctgtaagagcaacacgtgtgaataaccactgagccacctccccggtttcctgataatgtacttgtaagactttccagacacaaccatggtaccaggagcccaagggcagagtatcaccaaggctggccatgccttctgttttgtcctcaggttaaccagcaaggaggatgctcatgatgctcccatgccttgctacctttcaggaaaggcaaagataggccagggaactcaggactcctcatagtttggatggaagttccagttttcaaagaatgagtatctctgttggcaggaactgtgcctgactgaggaggagcaggggaccaatggcaagaacaaggagaaaaagcccagctgggggtctgtgctgtgtccaccttacaagcatcacaatgctttcagtttattagtcttcaatgtacccctccactatctctcttcacatacctttccccactccaatctcctcatataaaggtactactatgacttcactgtacctctgaaacaaaaattacatgttgcaatttgactattgtcattatggtgtggtggtaagactaactaaatctaaccaaaccaaatctaatctatgttggaggtaaggcctttgggagagaattaggattgtatggtgtcactagggtgggacatcattgtggtagtagtgcctttatcttatatatagagagagacagagacagagacagacagagcaagcaggcaagctcagcaagtcatgccttggccatgccataatgcaataggaatgccctccttagagactgacactacatccttggacttctctgtctccagaactatgaaaaagtattttctttataaactaaccagcatcaggtaagataagtatacctcataaggaatagatggtgagtgttgcttcttggactcagtcagtcactgggttacatcctagcaaaatctgtggctcaactcacaaagtcaaactaaatagtgcttctcccccccgccccccccccccagggggagggatcgagggtgggagagtcagagacagagtaagaatgaggagaaaggagaatgctttctccaaaaggaagacaaggacctaaaacatacttatggtctttgcaacgtcaaaaggagaactctactgcctcactactgagcaagcatccagagtttaaaacagtgtctaagcatgtctgcctggaagactgtagccactgcatagtctgtcacacagcaaaggcaacttcacgtggcaaacagtgtcttaaaagcccaggccgaaaaatcacattggtaaaagtattctctgcctaatgccaaaaaccaaaccaaaccacaccataccaaaccaatccgaaccaaacgaaagcaatctccttttgtggcggggcggtaggcagatccagaggagacgtcattttaagccagaatccaaagctatttcagtttctgtttgaacagaaaggctgttacagatggagaacttccacagagaaaagcggaccctgagcagaacactcctggagcacagcaagctggacagagagaggacaggaccgcgtgcttttttgacctgttggcacgctagtcatgagagctcctaacttttccagcagctgatgctgtaagtcattttctccttattgactttcaaaggattttcaactgctctgggttagcttgcttcctaagaagagccctgaacagtgcacccgcacttgggcagtttcttgattcttggggagtatataaagggctataagtcacatattgttgctcccctttggctttgccctttgctttccacaccttgaaagttgcagggctgctctgcaggcagcatggtccagtcacagacggccagagtcacaggcaggtccccatcttaattccaaatacatctgcaaaacccctagacagtcttccaagcagcccaagggaagcccactgtttgcaggcggtgcccctcaatgcggagacagcactcatttggatctggtttcccaggtgaccttccggcagcattcactcctcagggctccatgaaggtctggcacatgcggggcaaggctgctctctcccacatgcttgcaaccctattctaacagcacaggatcatttgaatcagtgtggaatatgctgagactgaaagatgtggtctgccacggctagggtaacttgctcagactcagatctcaaaagtaaggacatgcccacactctgcaggctcattctgagcctctaatcactgagatctcccaggtcagcctttccacaatagctgtctgagtctctcttatataaatccaatttataccacaaaggcaaaatacaaggctgcttattaatacggagttaactttcagtgttatttcccatatgcctgatgacactttaggtgtgttacttatatgcattatttaatcctcctgataatcacaaagggtaggcatccttcacttagagatggacaaactggagacatcggagagattaaagaactcatgcaagaggatcaggctgctgtgccgttgttgggtgtcacactcagagtccccaacagatgacagcagcctctccacacatcttcccatcgtggtgtggacggcttctgagcctctctgctgtctgaaaggcatgctgcaggggagttgcttgcatgctagttttctttttaagcatgcaaccataaggaagaaataaaccaaaagccctactacccttttctactctcttgaggtatatccctaaatctagaggacaaaacaaagtaacatacacaccgaagagccccgcctcgtgcatacaagtgatactagagaaataatatgtatcaactcagtgtcctctattgagttgtagccaatggaagtctcatggacatttttctgccacaaatttaaaagactagattttttttttttttcatgagacagggtttctctgtatagccttggttgttttggaactcgatccacagaccaggctggccttgaacttagggatccacctgcctccattttcagagtgttatgattaaagatgtggaccaccctgcctggccgccaggctggatcttgttggtatctctaagagccactgcaaagttgcacactaacagagaagcttctcttctgaaagatgcacagtcaacatggcttacctgataggaaatgactgtgacatcagaccaaccagaccaacaagacccaaaagactacaggctcctcagagtcagctcggaccaaataagtcgactgaatgcagtaagcatgcttaggttttcaaatgccctgagacaatactctgagaggtgatcatagcatccttccaggaaccttgctaggtcacaactcatgcctcactcaagagctgctccttcccaagcttgtaaacccactctaacagcacagaggcattctctttagcaagccatacagttctgtaagatggaaactttgtcgtggaagaaaatctgagtcagggccagttgagatggctcagtgggtagataaaacagctatccttctgatggcctgttcgacccccacccccctcaccctggaagtcacatggaggagggacagaatcaattcctaccaagtgttttctgactgccacatacatgctgtggtgtaggtaggagtctcctcccccaataatgtttctaagttggggaacagttcagtgcgtcctccttttgggagagcacagtttgaaggctcggagaaaataaccgagtagcatcgcacagtccaggacagaaggtaagccacagagttcagctgctgaagcatctgggaacatttcattaatgagcaaaagaaacaatcgcctactgacaatgcctgtgggatcactgaggcctcgggaaccaagcagtgctgggcagagtactgagaaaacaggaaaacaaacactgcaaaaggggtaacaaagaagcctgcggagggaagttataaatagcaacattataactagaaagctgcgtggaaagagtgaggaccgaggtgcagctgacccaaagagggagagcaggaaatttcaataaatgacaaaggccggcagaacagagagggctctgcccacggtgggctaagagtttcctttcttcttcctgcacattttgtaatatcagctttacaaacacacacacacacacacacacacacacacttaaaaaagcagaaagtctggtggttttcagtgtcttcgtagagttgtgcaaatactgccacaatcgacgttagaatattttcatcatcccaaagcaaaaagcccatacatagcaaagcttcccccagctctggggaatgactaatcttactcgtctccacttctccactgaatcgcctgccaggagcattttacttagtggaatcaaacaaagggtgttctcctgtagccactgtcttccatttaaaacaatgctttcaatattcacccttggcatggtgtctactagtccttctggctttgggctgtactgattctcactgtgggaaagcgagcctttctattaatgtggagtgataacatcgtaggtattgggagtagaaatgctagaaacactgacaaactcggaaggggtaaggatgtcaaaggatggtggatgctgaggtccaggcaaggtgctgagaagggtctgtttgcatcccagactctgtatttttggctaactctatccactaacttgggccattctttgatagacagggctgtcacagtgggcataatctgtagaggaattgtcacctacagtgtggactgtgtctcccctctgctcagcaagctacctgtgccacttatggaaaaccaagtgcacacacatgagcttgagctctgtagaaatgcctgacaaatggatggctgaagctgcaacactgaagctctgctaagatggggagagagaaaaggaagggcaggagagagtcaatggaaaagatggtgacgaagggtgcagctcaaccttagagtccttttaccagcactatattgttctcacagactgtcttaattcccgtgtacttttaaatagtcgaaaacgcgattttcaacctattttaaaacccacatacaaatttgaaaaactgcaggcagattttaacacaggggagaaaacaaacaaagagagctggcatgggggtcaggacagacagggccctcaagctgatggggcatggtcagcaggggagggcagcttcatggctgcttcaaggatggagaatctctgtctcacaaaagtcacacactttgtcctcaagagttctgaaaaaaggaaaagaagcaagaagtccctgcagagggccgactcccctgcagccgttctcagcaggcttgagcacataaagatcacttgtgactgcctgtcacagatgctcaaatggacaggatggggaaaaaaaatacccttcacacacttaaaaattactctaagttattttaaatctggaactgtttgtatattccttataagtttatgaaactcacacggtcgctccatgactaactgaagcggaattgagagagtagtctctatttataacttctctgcttgtgcgtgcgtccagccctatttccattctctggtactgtacgcagagttatgtgaagccgtgtgggctctgaaatgctacttacttcctaacaaggcaggcctgtgttccataggggcaaatcagtgggagacggggtgaggggttgcaatgaccaagtgaaggccaggctcagaggggaggtctcaagcacccatcattgctcaggcagatcttttgctgttggttttgtttgtttgctttattttatttatttattttaaagacagagttgctttgtaaaccctggctgtcctgggacttcctctgtagaccaggctagcctcaaattcacagagatccatttgcctctctgcctaccgagtgtttataccacctccacccggctagactgcacctctctaaacgggtgtttgagcctgttttgggacaatcgcacacctggtgatggctgagagatctgttctgatggtgtgaattcatcaatacacacattcccaggtcagtgccttaaatctgtgtgcccgctcatgtcacctcttccacttgattttagtggaattgtgtcaccaaagggtgcaaggctttaacaggtcatgcataagcctctctccatacacaaatgtttatgtgaagataactgaaatgactgaccagagtcacccatgggacaactagtactcagagcagagcacatgaggcctaggattaagttctgttacgaaaagcaagacggttcatggagagagtaataaatgtgaaagatacacgcgcacatagactacccatggaattcggatggctacatgtacatggacacgaatcccctattcacagctgctcccatcaactgtaagaacagctgctctaaggctgcctagtcatagtttggaagaaaaaaatcaaaaccaactcctggaatcttctctaaaacaatgctctggtgactgtagttgtctcgcttcatgctgctccaagaggaggaactccccatgaaaaggaggaaaaggcagtccacagtccacgcttcctttttgggaaagatcagaaggggtaacaggcacctcacacctcaatcctgtgactaattatgaaggtggtttatttttactgcatagccttttcttttccagtttatatcacctcttaatgtctccagattcattcctttctagaatcttaaacttcattacagccatctcttatgtccatgtatgaaataaattaccactaaaaacatccctgagaaacatgtctaaggcaactcagtcactgtatgcacacagtgaagtttacaggaactaggaggactgtgacatcattatagccccgacctgaacaggatcactgatgtgactgaacctccaggatgcacagctgcgtttaaatcagtcgcagttcttgaagaagcacgcactttctaaacacagcacggatatcaacagctggtcaagtgaccttcagcctgctcctctgaggcccctgcctctctgctcaccacttccttcatcctgctgctctgctcctgtaacttggctggaaacatgacttaggtattcccaggaccatcctactacatacttttacttaccaagtagcaatcattaaaaaaaaaaaaaaagctcagctagacctttaaattatctaggagggtagaaaagcattgtccctgataattgtacatcctgaaaaatactatcactagcataaatacatggaatattcataatcagcctatctcactgttaaaaagtacctagaaaagagaccatgcaactgttgagataccatttctagaatctctctctttttctgagtgtgcatgtgcgcacacacagacttctgttggctttcattgtactttgcttgaattttgtgagcagagggggtggaaaaaggtaccaacttggctaaaacgcatcgcaagtgttgatgccccatagtgccaataggaatccgagtattctggaaatgatcacttctcagtttcaaccaggttttttcgttttgtttttaaaaatggtactgcccaccgttaaaaagttcactgaaggcattcaaagttcgagatgccacgctcttttttaaaaccattttttaaaacgattaatagttgagcacttaggtgggcagcggtggcatgcacctacaattgcagcacttgggaggcagaggcaggtggatctatgtgagcttgaggccagcctagtctacagatcaagttccaggacagaggtacatagagaaaaataaacaaacacaaaaataaaaccaaaatcaaaaccaccacaacaacaactacaaaattgggtccagactgggagccatcctaggcacagtgggtagaacgcacgccagctgactgaacagttcggatgaagatgccaacagagagaggccccttctgttttttccaattaattaggaactgtatgtctgtaaatttttatttggtctttgaactattatctcctgatctcagataatctggtttactctaaggtgctgtccaaatcatcaagtgttccttgattgctcagacctatccattatcacccttggatgtaagagactgaagcaatgagaagcatcctggacctctgaagacgagggtgaacccagggtatcctttcagacactgtgtacagggcacctgcatagtcctgtgatgtcacctgaccccacggggctgggagcagggaaggtcatacagagggagggagatacaggtagctttctgcttccttcccgtttgaacctttagcccaggtctcccttcatcacctccagacatgttctggaataccctctaggctggttttctacggttcacgggaattttcattggaagctgaaggaattaacaggtagattaactatctctataaacctgaaccccagtaagcagacagtgccagcccgtaaagcatagatgagtctggaacttggaatggtgttcaagaatcactttatccgtggtataaaaatggaagcattcagaaatgggaaagggccaaactgcactccgcctcctacaacgggacctgtgaccccacagaagtttggcaatgtcaaggacacgcgaagtttagttgctacacaaagggactggtgcctatacagcgatgcctaagatacaggaccatattttccaaatcaacttaaatgtgcaggtgtgtgtgtgcacatgtagtgcacgtggaagacaaaagggcaatgcagtatgctttccactattgctctctgcctggtcttgtgaagcaggggctctcactgaagcaggagcttactgactggccaagctggtaaccagcaggccttttctgtttcctaaggccacttagtttttacacggatgccggggatctgaactcagcttctcacacttgcagaaccatctctccattcctccaaatcaacttttaagcagagaagccttaaatgctgcgaactcccaaactatcagagtcttcctcttctaagaaagtatccccaggttttccaaattctggactcagtctgaaagcgagggtctgaaaaagccagagtcatctcttccactcgggggtgaggtggaggtgggggcaataagcactgtagcaacttccttgacattcatgtcaaaggagctgctgcctttcttcagaccccagagtttcctcatccagcaggaggaggcagggaggtcagggtgcagaagccgtggagcctgtatcctagtctgcagcgtatgtcaaggtgtgctttgtcccgatttagagacagagcgagcgttgaagtgaaaaagggttaagaggatggaacaaacagaacatactttgcaggcaatgacagtgacccaggtaaaggcagaagctgtgctcaactccctgtaatgggagccagacatgcaacttctgttatgcaatgctttgtgaaaaggggaaggcttttggtggtctccactctcccacgggggcctctctcccacggcctaggaacgcaggagagaggaggaatggccaccacaagtctgggctgcattcctctgttctgcttctccatggggcctgggagcgagcacctttgcaagagaaaactccccaagagctactggattctggagcaaaggcaactcaggaactatgaacccagaggacggacatcttcacttggcagatggagaaagtgaagttcaagcaatctaagcgaggaaggttgtagtgctcgctcactcactggaggggggttggggggagcttgttcaagcttccaaggagtgaacacagggattgggacaccagaaaagcagcttttcttcatctttgaaatcacaagccctttgaacacacgaaccgcacaccttccttaaagaaatactggggattttgaaatcaatttaagcacctctaatttccaaccctgaaacgttttactacgtgcattctctaactgcactcgtctctcaccttgtaaacaagcaagatcggctggatgagtgtcccctaattacggtttccttggtgcccgtctatccgcattggcagcacctgaactttctcctgcactgtatcttcctaaattgacattctcagcccacactggcacgctggcctggtgccattcaacagcacaaatgacacatcccaagcctgatacttccgtcctcctgccctctccacactgcgtgtccgtaatcctgccctccacccactccccaaccctttgaaatttctaaaccccagcagaggaagccctgtagggctcttaataagctcagtcatgctcgaaatatccgttttacccacagggtattgtaggcagcaattccgaccagagcacatctcggcacgagatcatggcctccaaagcccagcgttcagaactcctaagagcaccgctctcatgcagtcccactggaacacttgggaacttcaaacagtttgggggtgggaaaaaaacatcctctctgatacttccaccttccacctacactgtgggttaggaaactccttcctgttgctgtggccacggagggtacatatcgcaactctcctggctctagtgtttgtgaaacccacaggaactctgccaccacctacacagaaagtcccatgccagagacaacgacgcataaacattttatgttaagttagccacatgctaagaaaatatcatcttgatcaggtatttttctagttctgtctcacgttcaagtctcaaggcccacgtggaggggagcacggaagtacactggttgcatatcttctgccccactggaagatgcttgggatttctgaagaaaccacctgtaaatcagtctatcccttaggtaatcgagaaagagagtccatgaattgtgtttcattgatgtgagtcctataggtgcttccccttggacaaggataggtaccaagaacaccatcctgggctggggagatggctcagtaggtaaagtgcctggtgttccacaaacatagggcctgcgtttggtttcctagcagccaaataaagcacaggacttggcaacaggaccctgggatctccctggctagacgccctcgttgaaatagcaacctctgggttcagggagatgttgtagtaaagataatagtgataatcaaggtggagatggctagaggatgatggtcgacacccaccgtcaactgtggccttcacctgtgcacatgcacacacaggcagcaagtgcaccccatatactacacacacaggcagaaagtgcaccccatatactacacatacaggcagaaagcgcaccccatatactacacacataggcagcaagtgcaccccatatactacacacacaggcagcaagcgcaccccatatactacacacacaggcagcaagcacaccccatatactacacacataggcagcaagcgcacccacatatacatatacacagtttttgaaaaaaaaaaaaaagctcaaatcaatttataaaacattaagttttaagttggcccttaactcactaatattatttgagaggatcatcagtgaaattagaaacagaaagaatttaattggaacctgttgatgtgtttcaggttattcaataacaaaaacaatctgccagcccggtaacatggtcatttgctttgaatgggattgcttcattctgtggatccacattccctgaggtgggccctgaatggctaggctcatcttctgggaagagggacacaatggcacggggtagcattcctctgcacattaatcatgccaaatatcattaaaaaacaaaacaaaacaaaaaaccaaaacctaccagcccggaagccatggtggcagcagttaaagcaacaacttaagaacagacgaggcccatctgcatgccgctgtggccacatttgaaaaggcccaaggaatgtctgtctgtctgtcttctctgcctacagttttcaaaattggtttcggggagagtgaggttcatgctctgtgttagaaacatattaccgagcagctgtctggttacagaagaacacgggcttgggagaagttcacagtccagccctgtcagctgctgtaagtcaaagggactctgcctcctcagagttccaccggaggcagatgacagacaccaccccgcactgttagcccatgaacacacgcagcaaggcacacagcgacagcaagtgtctcctgcacatcggcagtgctctgtcaatatcagctgctgtctccacgtaagtctctggactggattctcaccttggctttgccaactatgtaacagtgcaactaactggtgcacaggtatagaagagatgaggaggctcctaggaccttccgccacggcacgcttggaaaaggaacgtgacgaagagcttctagttagttcccttcatgtgctgaagagctgcagatctggtttcatggaaggatggttagatgaataagaaagaaaaagagaactaaagaatgttttcaattttaaaaattatgtgaaattgtgtgtgtgtgcgtgtgtgtgtgtgtgtgtgtatgcatgtgtgcacaggccaaggtgtacacatgaagggcagaggtgtgtgtgagagtatgtgtgtgtgtgtgtgtgtgtgtgtgtgcacaggccatggtataccagtgaaaggcagagggtaagtgtgggagtcactttctctagcctttacctgctaagcctcttactggcccgtaagggcaggaatctgaagtacttaaacctcacttctgatcaaataaaacttttataaactttaaaagaccgttttcttaatttcagtactcttctcagacccttctgtcctagagtaacctctgtgtgatccacaatagacctgggctcacccagaactccctgaaggttcaatggtgatggtggtttggttttgtgcagttctaggaatgtacagagacacaggctgaccactgaggtcatgtggggattaacacttcatcaggtacaggagctgtagctcaggggcagtgtgcttgcctagcatgcttgcagccctggttccaacccccaggaacgctaaaacagagaaaacacgtaaataacatgtctgccaggtagcaagctattccaagacaaggaaggagacttctgtgtttcgaaaaatctgttttgaaacacaaagagagactgggactgtggctcagttagtgtagtgcttatctctatccgagaagtcctgggttcaattcccagcaccgtttaaatggggcatggtaatagaggcctctaatcccagtccctggagatagtggcagggggactccaagttcaaagttactcctggctatatagtgaagaatgcaaggcagactagactgggctacatgagactctgttttaaaaagggaggaagaatccttccaatacataactttactcaactttgcttcagcttacattataaacgctggtgttagcatagatacagaagctctttcctgtgtggtgatgaatcattactcacaaccaaaccaagtgaggaagcagggccccttaatattaattacgggattcagacagaggtagttagagaactgtataatggtaacacccaaaactgggttgagtaccccaaatactcaaaagaaacagacaatgctaacctgggagattagaaatgattctgcaactagttccctgctgcatgtgcagctggaggcccccacaagttggaggcttggtgggagactgagaaagcatttgctattcgttttctaggtccaaggcatttcttccaggatccatgggctggactagatggagcaagtgcaggcttaagaagcacaggtactgtccacacactcctgatcatcaagatggggagggtatccagcttgttggctggtcactagaacatccagttctccttccatttgtaccactactatctaggactcactgatcttcagtcacaggaaaaggtcatctatagccagtggccagagatagccttttatgcttgctgcacgacacagtcagggcagtattccaggaatgcacagacagcctgctcagcttctctcacttcccctctcatagcccagatcgaggaagatggattcacccatcctgtcaacaaagactgccagaatctcctgtagttgcaggtatacaattgtgagttgctatttaggttatggaagggctgtggcatgccaacagataatgtgtggctacatggaggtaggcatgggaaggggcaaatacacacacacacatatgcacacacacaaacggaacacatacatagacacgcacacacacataaacacaaacatgcacaaaggctcacacacacacatacacactcacacacataaacacaaacacacacgtgctcacatatacacacacatgcatgtacacacactcatgcatacacaaacacacataaacacacacatatataccctacttctgtacatgtgtagtcctgactcaggaccttgggacctgctgctcattcagcatatgccgctttctgttctccacagacatcctgcagatgtcccagtgacctgcctgccgcctgccttccctgtctttgaggtggtgacaactcctgccaccccaacattttatgtcacagcttcctcttcagctgacttgaaagctttattttgttcaaggtctaccgagcaaccaagaaagaagagctagtgtttcacgggtacagggtggagtgttctggggataggtcagtcatcgtttcccttttagggagacttggatcggtgttaatgaactactcacaggaactggccagaatcagagcccttgaattttatgcactggtccagggaggtcctgagagagtggatgcagagtgcactgccttttttctgcctttgcttggaatcccagaggagccagattccaggaaggaagtggggcagggaggcagcagggtataacgaacaaacagaaaaacaaaacaacaacaacaacaacaacaaccaccctcaaacctggtctggctcagcgggtaagagcactgactgcttttctgaaagccccaagttcaaatcccagcaaccacatggtggcttacaaccacccataatgagatccgatgccctcttctggtgcttctgaagtcagctacagtgtactcagtataataataaataagtctctgggccagagcaagcagagttcctaaaattcaattcccaacaaccacatgaaggttcacaaccatcagtaaatccatccatccatccatccatccatccatccatccatccatccatccatccatccatcttccaaaaaaaaaaaaaagccaaaaccaaaggcatggccacagaaacaatgtgtgcctattgctattagtaacaaggtaccttcttccatttgggctgtgtgcggcttcatggtagtttaccctgtgctgcctgtgttcatttccacagggaccccaggatggcaggaagacctgcagccttctcaccttcagaaccagagccaagaacacagagcaaatgtcatgaatggtcagagtcacacagcctgcaggaggcaaagctggaattctaagtggctgagccttgaggtctcaaagtttgtgaccaatacactgtggtggacctgccccggttccagagtcactggaaatattctcatagcaagcaggaagatctcagggacagcagacgtagttctaaatgggcatgtcttgctagaaggcacagctgatgtgccttttccagtaagcattacaagaagcccatttccatggttgaaaaatgcaaaggattattctgagccatcccgatgataaagaggacaacaatttgcctttaaaaaaaaaatggaagctgggcacagtagcactcttctgtagtctcagaacttaagatggctgaggaagaggaggacttgggaagaggagttcaaaggctggcaggggaaaacacatcaagaccttgtttcagatccacaacgtaagcaaaccagaaggtggtggcaaacctggcggcgacgcttaggacatctaaatactcaattatttctacttcaaacagtgtaattcgtacaaactggtagagcacagaagcagaagaacattaaacacaggatttaataatggatatgtgcacacacatatacactgttgaagagcttgtcgtctgaaagtttctatgaattaaatatttgttcattaactactcgagttcacttgcaaggcatgccaggcgccaagggagagcacagaagtgcacaaggccttccacacaaagaccaggaaaggagatgtgcacatagataatgagtaaggcacagacttatgtcataaagatgcgtatcaggaagccaagagcactgccagccaagaccatggcgtggaaaggcatgtgtaaagcaggtgctcccggcagggtccatgtatgcatgagaatctgctggagtgatggtgggggtgggccccttagatccatgcatttcaagggaaaactatcaattatcatctttatttatttattttatgcatgtgagtacactgtcactcttcagacacactagaagggggcatcagattcccactacaaatggctgtgagccaccatgtggttgctgggaattgaactcaggacctctggaagagcagtcagtgcttttaaccactgagccatctctccagcctaattatcatctttactgataactaaaatgccttaaaagtttaacttagtaatactctgtttaaaaggtatataactaaacacagccaatctgtttaaaacaacacagtttcctgagttgactagaagatgctagtttttcattctgtttctcctggtacaagaagatttcaaactatctacaatccctcatttaaatttaaaaaccaaccaaccaaccaaccaaccaacctccattgtgagaggtgccatgccactggtacctgcctcctgctcctcctattacaaggaaggatggcttaatggccttgctgggctgggcttcctgactgcctaacagtgtggaaggctgcacaggcagtggagggaaaaaacccccaaacctctgtcgtttgttaaaacaaacgagcattttcacattcactcatactccctgaccccatagatcctgtgaattctaaaaaggaccatgcttgatgagaatgttttttaaacattaacgcctacaaggtggagagccagaagaagggagccaagagtggatggcgaccctcttcctctcatgcttagcaatttctggtttcactcctatgcttctgagcaccccaacaggaaggcttcacagctcctgaaggtctgagctagaccagggtacacatgaatatgctgattatgtggtgctcatgcaaggagacccctaaaagatgacttccccacctaagtccgaaccgccatgactctgagataatttacccaaagttacatttctgtttcccaaagtattaacaagagtgtttggctcttttaaaaagaagcttagtctttcagggctttttttttttcccttttcttttttcattttcaacaaattgcttctatcgtctgagatctccattacggaactgtttgtattgagttttagtgaaggaagaaaaaaaaaaaaaaaagaaaacagagcggctgggaagattcaagtgtgctggagacttaacattaggatcgggcacaccccaggcttcagggttaagttaaggagggtgcttgtgtccagtgtgagaggtggggatggggatagaggaagcttcctcggaagacttgaccactccccctcttttgtgaagcctggacccctgtcacagcgagtaccagagtacagcactcacccatgagcctcaattccatgtgagcctattctttactccacgttagtctgcctcttttgcaagcacactctgcaacttttcattttaaactgtgtggtgtgctaacgagagtcagcagctctctgagtgacctaggcaagttctttcttccatgagctgcctccagcaacagagaaagggggcaatactgttgcagggtcacggcagttgggaacacttttcctatagttccagaattactggtctcctacccaaacctcagatcatcattaagacatgctaatgtgtagcgagacaggttggaaggatctttttctatataaaatactgctgactggggcgtggaacccctgtcctattgggtgctgtggccatgcaaaaagagggtcttccaaaaatttaggattaagagggcatggtgggagaggaacaacaaactttggaagccttctcaatgtactgtagaattaaaagccaaccaaggcaaggaaggtgccttccaacctagaaatccagccaccctctgcaacgagcccaggcctgctggagaccacgtgaggtggtagtgggtatgcctggaacctaacataccacttctagtaccgcccagctggcacccaatcaggagcggcacagcattctctaagcccaggttggcacctgcaatcatcttaagcatacaatggcattgatttccacttagggtgtagaaataacactcagaaattttacctatctctgctgttttgctgccaaatttcaaatactctccaagaaacgcaaacacaacagaacacggaggatgtgacacacaaattctgcacaatctcactgccaatacctcgatgttatactcagtgtattaaatggaatcagaatcctgagtcccactgtcaaacttcctcctcgtctagtcggtatgtgtgactgacagctgatgtcactgaatgcttgatgtctgaaatgcctaggattcctttttctccccccatccaaatataacagctccatttcatactccctagaatttcatttgtgactggttagcacactatcagtgagggaacacgggggctgcaggaagctcaccgtccttacagatttctgggagggagctcaagagtaagtaagtgtgtaagccttcatagctctaccaaacacatgcataaaacctgattaaacacagaggaagaacataaatattctcagtcttctaaggggcagatgaagtctgggtaagaaggactaaagaaaaagcatagagtaaaattcgctcccgctttttaataactctttaagataaggagagttgtttaatgttttcaaaataggcagagagagagagagcgagctagagagagaaagtatgtggtatagtttatgtgtgtatgtgatgtgatttatgtgtgtgtgagtgtgtgtatgtggtatggtttatatgtgtgtgtgtgtgtgtgtgtgtagcactgtgacagagtgtgtgcttggcatgtgaaatcccctggtttcaatccttccccttcagaatgtttcctttggaacaaaccaatacatctggtgacgctgacctttagtgcccctcctccactcaccctgttttctacagcaaaagtatctacaaaaaggaagccgtgcctcaggaagtcttcaccaaagcttgaagagcatggagagaagaagtaaggttccatcagatgccttgtgttgaggtaagtcctccctcccaatcaacccacaatgaatattctctgatgtcagcccctctctctgagcatcctgtctctgagaccttggaagtaagacttctgtgtctgggaccaaaactactggggctattttggcagctgagccctctgcctcctctgcagggaaaaacagtaggaacatttcaaataacattcatatttgagatattttctggctctgcgtatttttctacttcatagcacgtgtaattgtcctccctgataaaagtggacctagcactgctattttaaaagttttgtgttctacactttaaaaggatacacaaagtcctagcaggagaacgtcaattaccaagtacaaaaacaaaacaaaacaaaaataggaaatcaccatttggccaatgccacagcaatgggtgttgaatgaggaggggaagaaataatgaacagtgtgtttagagagtcccgagatctgtctcaaagtacttcttaattagagataaaaataaagatgttaagtgacgagaggaccttgcagagacagcattaaccaagtgtcacaattacctaagcatggggcatcaggtgccctggcaggctgacgtactgactcttcacagcaggaaagagcacaggaggctgtgtgtggggctgcaaggaatgacctcccatgaagtacctggatcgagctactgaagtgtgggaaaagtgtcccggaatcggagaaatcaaggaggacaggaatcagagctgggtgggcaccgctcctggatcaggaaccacaaatggcacaagagaggtcaaagggcactgggaagaaggggcagatggtggcagtacagtgcaggtgctaccttcatggttcggacagcttcatcggggttttgtaagatgctaattagaagatgtcagacacctcccacccccacgtgtgcaaaactgaagcagaaatatttcatataaaacaaagtgttttttttttaagttcctaaggtaaataaaagcaacttagtgacatatggaacacaatgtccccgacagtttgacaataaggcaacactcttccttcgggaggagagaaatctctactttgcgtagggacacatcaaggatatgcttctttggaaggtcttcaggctggcagagctggtcaccaatggggatggtgtgggggcgtttacgttctcacaattccccatctatctcatgttaccctccagtgcaacggaagtggagggtacactgagtgttcatgaagagaaagccgttaacgagatgctcaacgagaatataaccatctgtcgcacttgtcccgcatgtcccatgcccttttcctaggctgcaagcacgcaagtggtacacagatacacacccaggcaaaacatccatacaaactaaacacacacacacacaccctttttgggtcctttggagtccttgtttatgggagcagacattgttctgtcttctgtcacagatagcatcctctgagcttggaaagtttaacatgagattgatcccactctttccaagggggagagggagactaatgtgtaagcagacagctcccagtatcacaagccttagtttcctcatctatcactatgtggtggtagtgagggcagtaaaaagaccttgtcaagctttgactagaacaacctgagattcttcattctgggagtagccttaaatgtaactatattcactggcaaagaagaacaaactaaggccccaggtgaatgtaagaaggctaccatttttacttcggtgatgctgagtgttaaggcgtgtatagcacaacagaggagtactccaccgcaaagggatgcactgtctctttggagtcgaggtgccccctccaggtaatgagacagaaaggagtggatacagctggtccaggttcttggctggctccgagagctatgctaagtcctctgaatcaatggtatgacacaagtttgttctctgttgagctggtgtcttacttcatcaccagtgctcatctctaactcccgagctcaagaaatccttctgcaggattggagagatggctcagcggataagagcactgactgctctcccacaggtcctgagttgaaatggctcacaaaccatccctaatgagatccgatgccctcttctggggtgtctgaagacagctacatgtacttacatataataaataaatacatctttgggttggagcgaacggggctggggagagagacgaaaaagtgtctgaagatagctatagtgtacttatatagaaataaataaattaaaaaaaaagaaacccttctgcacagccttctgaatagctggggtttcaggcttgagccccctttagagacaggcagcagagacacaaaggcctgcccaacatgtgtaggattagatagcctgggtatggctgaagggaacaggtactcaggttggcccaggccaggagccagactctagtatattatgcaaggttgcctcctaaattataatgcacttcatttgcagagagtccaacactagactcatggtgtctggtagcccagagggtaacaggctgtgttcttcacagggaacatctgaggacagagacggaccaagagggctcttgacggtggctggtggtaaggactaggagcgcttacggcgagctgcttgggagaagccttttcaggtggttatctgagagaaggcacaggaagtcatgaaattaggacctgaggacttgtccaggataggcgcagtagtgagaactgcatccttgtgaagatgggtttgacctctgctggaggctacaatgcagctaaatgtgtctttatcttaatgactccagaaacgccatgctgtgaaagaagcaggtaggtagagggaggtcaggagagcaaaccaacctcacgtgaagggtaactcacagacttgtcgttcactttccagaacccaaccctcaaaagaacatacttctagggtgtagctgggaaccaggaagcagccagctctgagctgctgcttgcttgctctctcacacagtggtaacaagcaatgctcatctcttttcaaagcactccaagcaaataaacacctgcagaggagctgatggagacagacttctcttactggggtgagtctattaaacagaaaaggaggcataaaaatgataaacccagaccgtcagaacacagtgagtgcaagaaactgtcagcagagagcgaactgcttccttctcacgccggagggcactgctggattaagcataatacaagttatgtatgctcgctgatgttcctgagcgggtaagaaataaccagtgttcactagagtaaaaagtgcagcggttctgaatgcatgctggaaagatctccagcaagggacagtgcaggaaaacgggagcagaagtctcagccctcggtctgtgtatcacctggtcacgctttctcactgagggatattggtccccgggcatgagaggagaggttcctgctacctagtgactttctcaagactgacttaccactaacctgtctcctatagtgtcaggagcttgagtctgaaacagtcttcactaaatttttggctgtaactgaaactggactaagtaattaaaaaggtttcaatatttaagctcacatttaagttgggaatggagggatggtttgatagtgaagagcactggctgctcttccagaggacccaggctcaattcctatcacacatggaggcttagaagcatcttcttcaggagaagacctacaggggaactaatatccttttctagtgcacacatttaaaactgaggcttagtttaaaccctgttttaccaggggcaggggtgtctgtgcacaagaaagacagacagagtttagttttagttctagacttctatttgctcaccaggaaaataacattggtagactttaccaaaatctgcagcaaaaactaaatgataaatgtactctcaaaacagggctcaatatcccacaggtgatcagggtcaacttttccagggaccctggtgtcagtgggttttatacttggaatgagaacacagtaaagctgggactgccaatattcctgctaggactctttattttctttatggcttcccctagcttatttagaagacaggcttctgtccctcagggactggacttctcaagagcagaacactgctgtaggaaacagtgtcttccaagagtcaagggaaggaggaatgccaccactgagaggagatggcttgctttcctccctcaagaaaagagtgagcaaggctccatccacggcgtgttccaacaccttcccacatagctggacccagatgtcataggggacttgcagaaaggaagtttcaacatcagctagccttggcagatgctcccaagtcgttcaaaaataaaactcgggcgatgacggtaaagatgggatgacggcttaagatggagcaagaggctttccccgtggcgggcctcagcttagtgctatgcccgcttgccgtcttgttcaggaacaaaccggggcccagatgagaaacgcgctcgcgttgaccatccagaaaggcagagtgtgtgcttctgaaggcagtgttctccagcctttctaccttccattaaatggttgagtctgtccttgtgtggattgatgttatttctaaagcacacaattcatttcaagaggggagaggcaggattaagctggaaatagttttttttctattgagaaaaaattaagtgtaagtggaaacgttttcatgaaaatgtttttatatctcaacgtttcctgcgagcactctgatcccaatatatcacaggactgagctggatgagggagaatgaaggcaaaatgaatttaaataaaattgaaactgaccggctaggctttattctccaaatctcatgaaatagcaaagctgagggggacagagccacaaataaaccaaaagaaaaaaaaaaccaaacaaacaaaaaaaaaaaaaaacaagaagcaactcaacgctttcagctcaaatgagtttcaattttatttcaaaatgttttaaaatctagtttattttcatagccacaaaagcttttgaatacctatgaatcaaaagccatttggcagaaccatggagcttgtcctagttgctttctctcaaactcttccctgggcctgttaaccccttgttagtcagtcacgcacagcaggtcactctcagtacccagttcttaggggcactctacagccttagtgtgtatgagcaggcagaggctctaggctggacggcacatttttaaaatacacatttttaagtgtatgtatttttttttattagtcttaaataggatagggaaaatgatatatacagactttaactactaattactttagacaaagaacaaacagaaaacaaaaaacaaaaacaaccccctccccaaacccaaaccaaaaaaaccactaaaaaacaaaacaaaaaaaccccaatccacatcctttagagattattttgttaatctgcatcttgatctgtgctgctcaggggccacaggaggccacaggaggcctctgaaacttgaaacagttaagattaagtaaaaagtgcaacgaagagagctcaccacatcctaaatgtttctcaactactcaactcccggctaccacacgcagggcagacacaggggatctgtcgaggcacaagactacagagtaacagtgcaggtcagacagaagagcaggggtgtggtctcagtccagctctcacatagctcatgctggcctcaatttcgccatgtctctgaagatggccttgaactgatcttccggctccctttgggactgccaggtgtgtgttactctacacaggttacgtaattgtcaggcctttgtgtttgtgtgcatttccctaagcaacttcatgtacaaagctaagttgggtatgcattctctccccaggcctggccccttactctctaccgtaaaatgcttcttccctaacaacccgacttgagtggggtccctacgtacagagaaaggagatatttacagaagaaagggcaaatgactgcttttacagggaaagagcatcccaggtcaacacacgtaccagattgtagagagggcaggtgtctccatccccgtgcctctctatttcctttccaacactcagccctgtgtgagcagagagtaaggcccctctttgatgctgcacccccctattataagcacaatgcttgtaaccaaacagatggttggatgtttgggaaatgactgaatgaaacaaaagaagaatggtattctgttaaacaacagccaacatccctctagagcctggacacacgtctttatagagcctggatacaagtctttatattggatagcaacctctttactttcaaatgcaatctcagagcaacccagaagactgattcaggtagtataccacagctcagtctcttccatgctcaacaatggctccttccaaatggcattagcgttgtctacatatctgtacagtcagagctaatgtctatctgttcgtctttaaatggcccagagcttttcactttccatatactatttcacctacccttcacctggatattgtctatacaataaagaggaggaaaccaaggcttgggcgaaactgacttgccagatcacatagagttataggtatataaatggaaaagctctggtaaagattacagactatgctgattctattagctggctgtaggacagctgaatggatctctttcaaggaagggcatgatttgtaagcatcccacacgtcagtgctgagtaaggctcctaagtcctgctggcttgctgccagccagtagaccccttccagggtgagggaactgggcagagctcagcacaacccagctcacctaactgtggctgtcagaatcattggatcctgcccatcatacatccactctgtggccatatcccgcagcttcacaagcttcatcagggtcgggcctgtgtctgcaaaaagaccagcttgagaagaccaggaggtgtatatcaagtaccactcgttcattcgttcgttcattcactagtcagaacttcaaaccaataaccccagaagtaaagacaaaaactccctctgccctctggtggcttgaacttccctcttggacccaattcccacaggttagggtttccagaagctttcaaaaaaaaatcactgatgaaacactagctctttagcttagcgtattatacaaaaggctgatttccgatttatcaaagggttaccaaataggaagtataaaaaccgtatgatccttacagggaatggtcaaagagcagtgatcaagtccctgatttacataattctcagccattcttttggcagacctctacagccactaacatttccatctacggatgataaaaagataactcttcccccttcctgggtgcctgtcaggtataaacttgaagccgggcacagttaatcctcacaactccccaaggaagaggtattaccatgtttgctccagattaagaaagaagggtcaggcctgaattccaggcatcagaattctactgagagggaagagagacagacagacagctttcactctggcctagagtgcagagaaaactactactttctgtgggaagaaaagtcagtcaaccacaggaatcacagaaggatccatcacaagaggacgccactgtcccttaagcagagacaggactgagaagaacaaaggtactactgggacagaatatggagaggtcagcttgaggaagtgctctagcaaatggcatcaaggaggccatttccccaactggagagagacctcagatcctcaccacaggagccaggtagcactgtggtacgcacacataccatgtgtgtactgatgtggtgggagacacaggcagaggtggcagggtgaaatcaggtcaagaagaactgagagccaggccggggccactgctctgccctgcttatgaaagccaggggattaagtcaagacttcactcgtgattcaaaaagttatctgaagacatggcgggaagtacagctaggtctcaccacccctccccctcccccccagctctgcatgctcaactgaggacgtgccacttggcacgctggcactctcctacaggaaaacatactttggctattcagacgtgacttcctggcactcggaagctgttggcatggaaaaattaacaggctgacttggccagggaggaacggttaacacaaatcaccaggaaaagatgagccctgagcaagcgggaggtttgagggaagggtcacggaaaaaagaaggggtaagacacatttgtcaggagtcccaggctggtaccaagcccactgcatgacaaaggtcattgagccctcacagccatacacagacatcacggctttcctttcaggcagtggacaaggaggctaacggaggtgaacttacttggcctggatgaggcagctaataagggacaggagctggtacacacatcatcacaacattcttcattacggtctctttctgccttcaaccccttgcccagtttgtaacttcataaggtggggtaatgatgccaggaactttgtagccctgaggcagaactctgccccagctgtggccttcgtaacctccccttactacaagcgacacagccacctccactcaaatcctgacgacagctcagtatataacggtagcgtgttacatactaacttggtcattttggggaaagctgcttgtgtgtgtaaaggggggggggtggtttggggcttgggatcaaacaaatgagtaagtaggaaaaaagaccaaccatccactgagatcctaaccaggctttcataacttaagactggagtcttccacaaactcacacaggtagccatagaagctactcctggaaatccaatcctggccagaacatgctagggccacccacattcctacctgcagcaaccttcctttctccctcagcatcaaccctaagatcctccagggctctgtagctcagtaactttgcagggtgttaagtctagaggagcccagaggtccgtggcctcccatgtgggggttacaattcctacaaccttaaatcctgtaaagcgtgcccacaaggtccttcagcttctcgccagaacttccaaaccttgcctttacacttaacaaagcttcctactaaatatctacaccaaatttccgtacattagtttttctttctgcttgtgaaaggggtggtggtggtggtgaaactagtcaagatcggatatcctctgcaataccagcttctagaaaagagcgtttttgaatgggatgccacgtgctgtgttttgccatattgcctgcgcttcctgcttggagccagagtaacagaaaccttgggcactgggaactgttccgtgccacgcaaattccactccgtatcttccaggcaaccctgtcgcaaataattcatgtccactacgcccctaccacacagaatacaactacattgtgctgctagaaatgggaaacctgattcagatcagagaaggtagaaaaacatttagttcaacacaatactgcctttatgaaaaggggcgtgcgccgggtggtggtgtaaatcccaaacctgtaagtggggatagatttcaagacgaattttggaagaaaatgcagaatcaaaatagcatccagcttgactcagaccagtaataataataacaacaacaaaaacccagccagtccttctagagctacgcctgtttaggctgcgcggtgctcttaatggccagccagcctgttctcctgggaaatggtgctgctgctacagcgtgacttgctttgcgtgctgcaataaaaggaaaacaagcttgcacgacccaatagcttccttcagtttgtgcctagcagcggggagaaaattaaaaaaaaaaaaaaaaaaagcctgaaaaggggtcctgaggcaaggtgaatggaggacaggatcctagagaacagcactgccctgcaggatttttgtaaacagcagcctgtaatggccacggttagagaaagaaaaacagacatggagggaagcgtgtttacaactgcagcaagggcgggaggcggagggagggattccccggctctgcgctcggccctcttcactccggtggggccccggggttgggctgagcttcagctccccaccccaatccgtgctttcttggggggccccccatcttgtttatgggaaggttactcctcctcggctctggtgatcgcgtccctaatactccagctgcatccttgcgggatgccagcttgactgacaagtcctgcagccaatcgctggttgggcccttaggaaaggtggaatttcccaacaaactgattggctgagagagagggcgggccctggtgattggcgtgttattccgagtagaaagtagtaaacaaccttggtccaggggcggtggggagggcgcccggtgccagctccctctggctggcagggcggggtggggaagcacccgagacaaaaggaatcctggctgccacagcggggagggcaggaagggggtggccccaaaagtcagcacctggaggcgtcgtttggagcccccccccccccccccccgagccagaactgctgactttaagccacacatcgtttttttgttttgtgcagaacctcaaaggttttgtttccatcattttcacttcttgtatggctgacgaagtctccaatggaatgataaccgatttagtcgcttttgtccagaagccccttcctcaccgtcgaataaaaatttaaaattgtcttttacgaaagttgctttaaatcacacactaaaattgttgcactatacaatacgccattaatggcatgctggtgcaccagacacacagctacatcttttgatagcaagttttaggagctctgagcctattgggagcagctgggatggagaacatacacaaaaagaaacaacacatgacggcgttgggaccacttgataagaaataagcggttaacttgccttgcccatagtatttctggcagtctcacttgaatctcccattccctcactccagcttcaccatccttacaggaccccaggcggaggaaaccatctgctcttaggatggggggctctgctcaccccgcacataaacatgctcacaagtcgctatcctgagcaagcctcctcaatgtaaaaggggaccaggaggccagccaagccggctctctccacttcaatgcttgttgaatatacagagaccacgttgtcacttttcacaatcaagatatatcggtatcacgatatatcgataacgggtactttataggagataacagtgcaaactggccacctggcattctcctttgccattctagtccctcacctctgctatctcctatggagcctgccacaagcaaccccagtaaatgttcaccccaagtcacctttgctgtataaaacatggatttaggctttccttggtccttgcagcctggcatcagaccccaaaccagtagctactttcattgtaggcaccctccctctggacactttgttctgttcccaggtccctgcccagaccacacacatcattttactcaacgttggtttttatgctttctagactttacctctgcctgcccaggagaggccactgttctccaccgttctccctggcccagctaccccacttcttgcagaagactgaaatttgttttcttagcactaagtctctgatagggctttaatgctgtatttagactccactcctacgttaagaccctgaacttcctgaggcctggaatggtattatcttcagatttgggtcttcagtggagatgggcttctactaaagtgcccgctgcaaacttgccaaatatttctaggattcttccctagcatcctgagtatgcctacccctgaaaatctttccttacacacgtgcaattaatgtgttttctctgctgggtcaacaactctgttttctctacttggcaaatttccactctgcctaaaatcacttcctctcttggcatacctttcacaacctcccaccaactacactgtgtaccaagagcagagaggtctctttccgttcttacttctctggccagaattagctacaaaactagaactagcatggtttgttggtaaaggaagagcggagaggcaaaatttcagaagtggtatcacctgtacatctgcataaccaacccattgccatctgtaggggtgaagcagcgccatgctccgtttgtggctgtgatggtctccccctgctttctgtctgaaggcctctgtctctgaggtggattcttactcccccctccatctcaatcagagatctcctcctggctcttatgctatcaactctgcacatccccgattcctttccaccatgacactgaactgtcatctccttttctatctgaccagtcactgacttgctcacagtttagtctgtccgccatacagccatatccagtgttctaagaattgtggggttccacagctggggctcagtctctgcttacgccctcatgtgacatctgtcatcaaccctccatctttctaagcctttaatggtggaaaagagtaaacctgtagataaagagtggtaccagatttcaccttaagattacagatacttgtttgtgaatagaatttgttctggtacaggcaaatgcacggtttaaaatcaaaactaacaaaatatacagtatattagctgggaacaaatagccaactaaagcaatgtaaagctgggagtctgtattctggctcaaagcccatcacggtgaggcaaacatgatggcctgagcttcaggatgctcattaccacactgcatcctcagtcaagaagcagagagaactggttcccaggacaggggctggtgctgcctaagtcttggtcttcccgtctttaagttaacacggcccttggcaggcatgcctgaagcttgacttttcggtgattacagaccctatcaaacaccataaataataacaccacacatatattacatatataaaaactataacacagaacactcaattggctttcagtaagtgggctatttttttttctttgttgcttaatgtattctagatctgtctatgtttacaaacggtatgtcttctctactaacaaataggatttcaaattaaaaagctgaacagcaaaggagacagtgaagagacaatctgttagcttttcatctgacaagggattaatatactgaatatataaagaacaaaatttaaatgtcaaaagaacaaataatcaaaccaataaatgggcaaattaatcaaatgggcagttttcaaaagaagcacaaaagaccaatacatacatgagaaatgctcaacacccttagccaccagggaaatgcaaatgtaaaccacaccgagattcaaatctcaccccattcacaatggagcacattaagacaaccatcaacaaacactggcaaggatgcagggggaaatgtgcctgtacacattgttggtggaaataaaaatgaagtagtttagatactatggaagcctatatggccaaaaatgtgctactacctcacttcgggatgtacagatgtgatgctaagtcagcacacgacacatccacgttactatccacatcagccaagcaatgggaccaggaaaagatgaaggaaatgtgatgcacacgcacagctgtgtcagatgtcaaggataaaaccaaactatcggcgggaagttaaggagtggacaatcatctgaagtgacgctggccagaccaaagcctatctcatctgtgtattttttttttttaatgaaaataaaagggaggctttgtacagagggaggaaatgatacagaagggaaagaagggacaaacagaagataatgggtggtaatcaaagcacattttgtatatggatgaaagtattaaaacaagaaaacagccatcttatcttcacagacagcattctgctctatagataaccctgagtggtccccaaccttgcctgtgtgtttaaatcactgttacttccccagaagcggatttagttggtttagaggctatcacgtgagccttgaaatgaaacagtagaaacaaagtcccagatgattctaatgaggaggcaggtttgagagataacagtaaacctatttattatccctctactgatagaacttgtcatttcagacaacaccatgtaaagtggcctaatacacctgtcagttcaccggtgtgctggtgtgtctgtgggtgatcatcctgggagaattaccatgtcaaagggcacaggcactggtacatcagaaatgctggcaaattacattccattacacacacacacacacacacacacacacacacacacacacacacacacacacacacaccatcttccagccccctctgccaagtggggctgttttcctatagatctgtcatcagaatctgtgactgaacattttagattttcaagcatggggggctgggaggagctctgaatactcataaaaaggactttctgatgtgttctactacaacatttctggttcttttgtttgtttgttgttgtttacacctttattatgaattcacttagagtctaacaaggatggacccatgatccaacatcagttaagtaaagagcgtatttgctccaatgatataaaacgtcatcttattaaacaacgaacggccacatgttctcacatctacggctggatatcatattctgtcccttcacatactggcaccttgctagctatttaatcattaattacttaggcaccatgggatgctttaattatctggtaatttcgttgggcttgtttagcactattactattatcaccattattattgttgctattattgacgactgtagagtcagtttgtataattaaaagaaaccacatagtatttgaagtatattaactttataaacacaggaagaactgacatcttgctaacattggttagactcgcattaaaggtatggttctatcatttatttgaatttatatttggcagcattttgttcagtactatttttcttattaatttttaagcatttttctctgggaaaaacttttcttctatgatgttgtatagcaaattatttgcacactgtagcgttatcacatgactaagtctggcttatgttatttccagaagatgcttaattccatgcccttagaatctgagcatgtatgcacatgtgtgaacccacagggcaactctgggtgctgttcctccctccaccctatttcagacaggttctcttgctgggctgtagcacaccaatgaagcaaacaagtggctggccacaagccccaggggtctgtctacctctgtttctacataacggagatttcaagcttgtgccactgtgcccgtgatttttaagatagattcttcgacttgaagtcagttccccacatttgtgtggcaatcatgccattgatggagctatttgccaagttccctgtgctcttagaactctcaataaataaacgacaccaaaggtcttttcccctcactagtatgaagtttattatttttttttattggatattttctttatttacatttcaaatgttgtcccctttcctgctctcccaaaaaccctctgtcccatccctcctccccctgcttctatgagggtgttcccccacccactcactcctgcctggtggcctggcattcccctagactggggtattgagccttcacaggaccaaggtcctctcctcccattgatgcccaaccaggtcatcctctgctatgtgtgacttgagccatgggtccctccatgtattggtggtttagtccttgggagctctggggggtctggttgcttgatgtcattgttctttcaatggggttgcaaaccccttcagttccttcagtcttttttctaactcctccactggggaccctcattggggaccacatgttcagtccaatggttggctgcgagcatccacctctgtatttgtcaggctctggcagaaagtactttttggcatccacaatagtgtctgggtttggtgattgtatatgggatggatccccaggtggggcagtctttggatggcctttctttcagtctctgctccacactctgtctccacatttgtcccggtgagtactttgtaccacttactaagaaggaccaaagcactcatactttggtcttccttcttcttgagcttcatgtggcctatgaattgtatcttgggtatcctcaacttttgggctaaaataccaaggttctaatcaactcaattcaaaccaataaattttgacttgacatttacaacgtaagcaattcagatgtagggcatagcactagctggtagaggcaagactggatatggggagcttctaacccaagagtggaaagtgttgggagaacaagactgagggcttggggcagtgtgggtgaggtggaaagctaactgcgtgccttagtgcatgccttaggaaagtatctcagctaggctactatagcagctatgcatctatctataccagagaaaccccacagagctggacatacagatgagctcattcatgtctgatgagctcattcatacacgtgatagccatgatgttctgctgaacgttttatgagataaaaccaagtttcctgtttcagagctgtgtgtttaaaggtatgtccctttatgaacattaaacaaccagctgtatgtggtggtgtcaataatcccagaacaagagggctcatggaagaagggtcttaacttcagactagcttggaataaatagtgcaaccatgtctcaacagacaaacgtaactaaaatatcatcttacaaaccaagaggtcccagcataaatatcagagcagagctgcccagccttggtctcattccagacctcttaacctatgacctcttgggctgaggtcatagtcggcatagggaagtgcctggctagtaacaacaacacagtcacacatatctgtcctcaaggagctcctcgagtaaacacctttgctctgatgatgacaagcatggccacagtccctgtgcttgcagaatcttggttagaaagctcccaacaccaccatcttccactagttagcctctgctgttccccgaactgaatccctaagtaccgaatgctctcctgttccacacagaaacctctgagaagcccagtttacagaatcagagactgggccattcctcgaagcattgctaaagaactggcaggagtcactcaagttctcaggagctatgggaagataagattctccccaatgcggttgcactgaaagacagggcagtaaagaacaattatgcctaaccccgatttctctctgaatccatttattacttctcaatttaggagagtcactcttgtacctgcaccctggccactactcagaaaaacattagtgtatccaatgatccctttagtaactactggagaagcagctagctgaagcatgggggaattaaggaagaccttaggagttcaaaaccccaaagtacaaacgcactgtcagcacacggagagatctgtttttttatgcatgccttatgcaaactggcctaagctaaaaaatgttataaagcattttgaggagtaaattttgatttcagggttaaaaagaaatgtacagagtcaaagaatctttctgttggctgataacagagactccttggaaaaattgacatttcttttcgagaagcaaaaggtggagtagaaatagtgtgagcagcatatgatgtcttgtgacaatgcagagaaggcagttgggagctgccactaatgtcgagttttcattaggctcatatctccaacttttggttttgtgaggctgcaactcaattgtgtagtaaaaaaaatttgtagtccattagggtgcttttaatattcactatatctactatgaaataggagcttagatgatgatgatgacaacaacagtaatagcagcagctactttgaaacactcacaggcattgtgagttggagccctgagcactaaccactgtaatttgctatcaggaccactgggtatggcattcggaaaacggacatatgcaatcaatgcttgtgacatgggagaacaaaaccttgtcaaccaactgcaaacagatggctcatggacaacaaaaccttagagaaaaatgcaggaatatcagcggggcagtggggaggaaaagagaacactttccagactgtatgcatagaagggcctcaaaggccgccatgacagatgtgttcctgtatcggggtcaacaaagaataaagcccatagcacaattcttaggaaaagttgaaatgcctgttttgtaaataaaacacaccgacttaaatttctactacttaattttataaattccgcttaaatatgtgtctaaatacacttttttccccttgttttgagacatggtctctctacgtagccctggctatcctagaactagttatgtagaccaggctgacccctgacgcgtggggacccttttgcttcttcatctggagtgctgggattaaagacatacatcaaccagctgggcttaagtgtctagatttttaacttctttatatttattcttaccctcagtgttattgaacattgactgtgcagacttcagagtttaccaagtttgaggggctggccaaatccttctggacttactgagaaaggaacagacccaggacaccgactggagctacacggtgggcgccatggcgccaggccgagacacgtggatccttcggctttgcttctgtgtctcagctacctcagaggtttgctgtggaatcactagtattttaaacaatgcaaacgcaattacagttgaagcacattctctaggagaaggaaaaggagattggcatgtagaagcttcattaaacttttggccttttcaaagctaaacgagtcttaagaaaagccataaaaagttcccgttgattgatttaaaaacaggctttttttttttttttttttttttgcaattagcagatctaaatctacgaaagggagaaacacgtacggcacttttagttaacttcctgctttctctgtgtatttcaaatcgtaacaattaagactctctatagacacacgtacagatacacaaatacttagcaggacgtaaacttctattttggcttcatgaaaatggtagttatgtttccacacacacaatgaattctggcagtttatttacaagcagacatcccatctgcaccagggataataccctcaactatgtgacatgctacatccctcctgaacacagtagggctaggaaagcactgagaggatataaacatgttcagggcttacaatattccttcctaatgcttctttccccaacttactgtaacgatttactggaaagattggactaaattaccgtcttccctttatcgtcccaatttgtttgcatttggtatcctctcctttctcttggaatctcccttgcttttccttttgtcagtaagttaaattgcttctcctcccacatgcataaaagcattaatttataaaacacatagctgtatgtcatatctcatgtaattctattttacattttaccactagctagacttatctaacatgaacaaatagatattatttactgtagccagaatgagaattatctaatgccacttacatagccagaaattgatttttgattaatgtttaagcgaaaagcaaacaaacccatctgctctgaaaacaaacaaacaaagaggagcagagcactgagtcctcggttcgcaggcattcctgcaggcctagtgcctcagcggctgcagaatggggcccttcaccccaggggaaagcggaccactgacaagaacaggactccaggcccagactggagtttcttctccagcaagtgttgagtaaaagattttttacttattgggggggggggggagacgggacgggactggggaccacaaacaacaacaaatctctaaaccctctcaggatcagcagagaatttcatcttgggcgcccactggaagtcaaggaagttgttaatggtcatgttttccttcttccctcttgttcactaggtcagttaaaagacaaagaaaaaggctgggcatggtggtgcatgcctttagtcctagcacttagaaggcagaggcagaggttctcttggagtttgaggccagcctggtctacagagttagttttaggagagctagccttcacggtcatctgcccacacacacacacacacacacacacacacacacacacacacacaaactgacaaaatgtaaatcaagaaagaaacaaaaacaaagaaaaacagccctgtgccatttattcttataatctctgcggtttcctcagtatgaagaacagagtgcagtttatagttcttgtccactccatttatattgaatatataaattattttctaagatacttatatacataaccaaatttgtttttaacaaatttaatgaaaacattcagcatgaaactaaatagcagtaagactctcagatggtgtcttaggttaaaaacaagaggagcactccaaccttttctttgacaaggtctcttgtatcccaggctaacccgtatgtagccgaagatgacttttaacagctgacccagtcccttccaccgcttctgttgtactggatcccagggcctccacgcaccccagagaagcactctaacaactcactgccagctaagctacagcccggcctcctttggagcactttcaacagccaaaactgacacaatacatacgcatcaacaagcaagctgaacacccaccaccggtgtgctgagagaagagactcaaactcacaactttcaaagtcccataccaagcctgatgatgcaaacacaacttgaagtcttattggcgacgctccggatactaccagttgctaaaggaacagagtgggactgcgactgcattctgaaacattttctacaaatactcatcctgcacgaagaagtaaggtgcaagtgcctttatgactctatttccactctagacgctcaggctcaaggcagagcgcttagatcctgctgcacttccataaaaacaagcctgatttatgctgaaagactttaatgacaaagttgtcctgatgtgcgtgcgtgcgtgtgtgcacgcgcacacacacagacacacagagagagagagagagagagagagagagagagagagagagacagagagggagagagagagagagagagagagagagagagagagagagagagagagaggacctagagaacaagggcctttgtcttgctctgccgaagcacagtgagcatttcctgagccctgatttagcctttaactgatggttctggtttgtgcttcaacagaaataattgcatgactgaccaaaaaaggaaaggccaaacaagtcttaaataatgattgttggcgtaccaactgcaccaaccttttctaattacaactctccattttattattgtcaggcaaagtgagtaccttaacttgaaaggctaagctgagggggaaaagcctttttgtctctactttacaggtttgtttttataaacactccctcagtccccccaaagcaacaggaggcaggcacaatgaccctactgaggagtacagcttttacctccatgggacaatagattgcttttctgttagcccctgggagtaaaaccagctttaatatggatgccctttccttatcaaagacagcagggtaatacggggagggctgccaagcttgggttgtgcggacttggcaggtatgaggcctacctcgcttctacaccacgtcactcaaggaacagagcagaagaaacacgttctggaactttccagagaggccatccttgttcatgtattctagtccagagttctacttctctctacagagatggatgaaacaaaagcaaaacaacacagagaaaaaccccttctaagggagctggagggaggagataatctagagagaggatgccaaaagtggcttgtttccaagtgacaaaatgcacctttgctggggaagctagcaaggtagaagcccctgttcagaacttttcaaggagcagatgaagacaaaaaaatgccacgcaatgggggacggaatcctctagatgtcgagaaaaagcagcggccatgcccagggagcaggactagagagaggcaagccaactcctgctcatggcaaggcggcttgcctctccagcgcactctgcagatgtttccgcacgttgtttctatagtctgaagacactccgctggcagtgtattgttggatgctggcagaatctctagtcaagactagcaactccacctccttcctgacatcagcaagggggaacacaccaggagccaggagacaaggtgtaggtgcaggagacaaacaccgtgtgtggtccagagctgaacttgctgctggagactgtcctgtcaggaaaagtaaaaaacaaaccaacacaaaaatccaggaggtgcagaggtagaggcagaggcaggcgggtccctgtgaatttgaggccagtctgctttacacagtgagttccaggacagcaggggctgtatagagagaccctgcctcaaaaaacaacacaaacaaacaaaaacaaaagaagggggtgaagaagagagggagaaggtggggcgggggccaggggttgggtatggggagggaaacttaccctggtggagacagtgaaactgttgtttgccactgcctctcttaggtagggtctaactgtactcctgactaatcttcaacttgctgtattaatcaagttgggttccaactactcttggaactacaggtgcaacccaccacaccatcgaagtcttggcgcacagctctagggggcataggaaatggctttccccgtgccctgtagaaatggaactgaaagctcatcatggcggtaagactgttttttagctcacactctcacactgcaaatgacctaatgacacagcagggcctccggtaccatcctcactcccacgctcccactccatcctaatgttagctcacttactcagtctaccacaggcttaggtgacatctgaaggaatagcatactataaaaacctgacttggccaccacagcatgcaagctctaaggcaggtagaacatactgggtttttgttgttgttgtttgtttttgtttgtttgtttttcttttccagtttctcccctcaaagatcttcacatcatgagttatgatactcgttcattacatcctcctagaatggctccagtcaagaagagaaggctcaatgacccaccccaactgcaaaagcaaaattatcttagaagaagaagaaaaggaatggaccatgtaattaaaattacgaggggcagtgacttacagaggtaaaaagttaatcacaaggactatctcaaagcctagtgaaaaatggggttttcagaatcagacctgtcaaaggtaaccacgagatgcaataatgtggtttggaagtctgggtgacgggcagcagcaggaacaggtcagggaaggggggagtaaattgtttgcaaaatgaaatgcagaatgaaagaccatttgagcacacaggcatctactctaaaacctgcaaatacctcagcctcagtcctctcacggaggactggggagtgtgcactcttcttgttgctttagaggaggtctctgctgcagaagcagggtgggtgggtagaggttcctactgaaagctactttgggtatccagcaccccggggttggtagcaaggagtccctttaatgaataaatgaagctggagctttccagatgtttcattaatggggcacccagaaccaatgctgctgcacccgtgccccctacaactactaccggcatctgggagcacttagctctaaagtaggttagagctggagtgtaactgtatctatgtgaagactgagtttgtatatgggaagctcagcatacagcaaggccctacgtgctggctatcactgcaatccaggcaaaggcttcctggaaggctaacgaactataacttggctgataatgccgttcgttcatggcttcgtgagcgctccagtcccggtggccttggcgagagttggagttgagtggagactgcagggacggaaacttttccagcaagaaagagtccctcttctcccacggtcactaaggaggatacttgtatttttccaggatcagctgagtcgtcacaagaggagagtggaggggcaggaggagaccgcctcaggcggcgacaagggagcccagcacctcccctgaagttgcagccagctgcttcttggctggaaacttatttaagttgtcctctgtcccacttctgaggctctacagggatgctctgggcactgggatccatgaacaaacagggatgagcggagcgaagggaacactctgctcagatgagtctgcctctcaacgccctttgccctcactgtgctggtggggagtgggggtgggggaatctgatgggcaaaagcaaggggcggagatgtgactgtcacgcagaaggtgtgcccagctcttagaaaagtgaagaattcaagaaagcaaacacaagcatctctcctgcttctactccctagttatcccaatcccaatgaatcttggaataattttagttactgacactccagatagcatcttcaacagtgaagccagaagcagagcaaagggatcttagtagcatgtgttaattttcatgcaggatgcaaaacaggcatttcaactttcatctttctggatgcagctacagtggccaccatccctgaccaggactctgtcttcatctcatccttctaacagttatgcatcaagagtgtatcagtaaccatatcaccatgcaggatgtgtaacttgcctcttctctcccgtttcaggacagccagccactggaaagtctgaggggcccggctaactgaaatctgcagatacgcttaggaatccctcccaaccagccgggttaacctccatatgtttctaatctctaacttgtaccccttgtgcagcctcaaagagatacatgtcacttcccgcactgatctgacaaccctctctctgagaggctcataagcaccctttcccaaggatggtcacttacagaaagctgcaggcaaagtaaatccaagtctgggcagcatataataaatggctagtggcagataatgaaaaggctgaaatcacaaaatcaaaattctttccaataaaggtgaatcccacacctattcattaacagtcccctactgtctaagatgtttgtgtaagatcaatgaataagccatgtcagaagacttgccgactatggaaactatttggataatgctccaaggggctcaggaataggacagatccaatttagaactgggggggggggggagggggacactctgtctccaggccaggtgatgaagacatacccagggtcattctttcaccatgacaaagacatctccattgtttcaaagtcactttcagtctcctacaaagttatctggagaatagtgtgccatgtgaatgaatgttcaaacttaagcaagggtaccaccttcctttcagaaacaaccagagaaatacgaaactgcctctggggcatcgcaataagtagctggggctggtaagctccaagtcttacctctgaagtcatcagtaagcatgggacagtgaaggctcagacataacatctcatccaagtaatcaagagtaacgccacccatcacagatgtactggtgccaccctgggcacagtgacacaccgagggcactgtatacccttcctgaaggagttaatcactcaaatgcacaaacctaatgtcacgagagaccccagtttaagagcaggttatggggtcataggatgagtgctcgttaaaagtgacaacaccatgcaaaatcagcaagacaagcagacccatcacagcctcgagaagaccacacagataagacacatccacacgaccaaagacttgggaatgaaagaaagcacgttagtgggaaaactggtggaatttaaataaagcctgctaagaatattgtgccaatgttagtgtcttggttgtgataactggattatgattatgtaagatgttaacatgaggagagctgggtgacatttcaggaactctctgtgccgttttcaactttttctaaggtttttttttttttttttaaatgaagctgaggaggaagcagagagcctgggaggctggcttactctttagtttgctttgtcactttgtccctggtaagtagggtcattatactacagagctggcttgtgttacagctcttccgattttgctccctccatgtcttcataacttttggggaatcggatgttactcaggtgagacagaagatgagtgaagtcacaatgtcattggagattagagagagaaagagagagagacagacagatagacagagagagacagagaacaaacttcaaatttataaacacattattcttccacaaaggtccacatcacacactccccccatttgtgcaccacccttgacaacacagctgtgtggttctctagaagatgtagttggaaccaacttttagcgtcctaaggaatgatgtcatcctttgctctataggagctggttccagaggttttgtggaacagctttctttccagaaagcagccaggtctgaagctgctcagacttgctctagacttcttctgtctacagagtacggcaacatctcgtctacagggactgcgactgttccggctctttgatcatcctgttccctaaacttaacttccttaagttgcaaaacatttactcttggttgtttgggtccttcctcggtatagaaattcatattctctgtttctctctctctctcaacggggctgtggactgaacttacgtgaacttcttgtgtttgcctggctttcactgaccctacaccttcagtccttttcctttgtacggagaaagaaatcattaaagagaaaattgttccttaaaatcatccctttccccatcctccaagccttctgtagaaagtgcagtaatgaaaaaaagatgtgtactcacactttgcttgtatcagctatggatgggttcctacctatcttattgccaaactgaaaaactacttggaaaatgttccaatgggctcggtagaataatagctccaagtcagaactggggtgcatggggtgtgtatagtctccagagtggggaaggctggctacttgtcgggtcctgccctatcagcctaccagttactacgctgctcttccctggcagctcagtcagtgtgcggcgtggtctctcctcctggccatctaaaatgtctgcagttattgtaacacaggatgtgatttttagatcaatataaacctggtgaacagatcaatcctgtccctcataaacacaggcaagtatctgggtatctagacggtcatgacctctaacctctgaaccaaattctcaacccaaaccatcattagcagattgagcccaagtaaatgcatgtgtgcagaagtagaggttgactccttcttaactgctaggtttataccagctaccttatggggtgccccaaataccatctccaactaaaaccctttcagtttaaagggcaaagggcccttaggtaaatggttcatttggtgaggcctcctctctttgagcagtcacatgaatccacaatggagaaagagactctaactcctgggatttctggtaacctaaagagttccatgtctttcctacacaattggtccccagacggctcaagtcttattgaaccgatatatcaattctggtggagttaaaatgctgagtaagagaaaataaagctgaagtaagcgtaaagacaagagttgttttgttttagctggaacatgattctttgatgagaaaactctcataaacaggaaaaagcacattattgaagctgaagtgtttgatggcttccattttcctaagagcatttcgccttagacacaggtgataactgtcaagagcagccagagtgattcctctggatatttctaccataaatatttctgaggtcctactgtgtgccagggattgtagtgatagtggggatacaaagataggagttgaggctatatacactcttttcaaaggcaaagtctcgagctctaaacgcacgactcaaacacaaaaccatgatatgcaaaggaaacaaatggtttgtttcttggggacaggttgaacagtaagtatggtgacaggttttaattagaagacttcagaagagcagggctgggccatgtaaatcaaatcctatatcagggtctgtactaaactcagaaagactaactcaggcagttactagagttatcatgatcctacacagaagttcatataaggaccctaactctgaaagcagagatttgttgttttgttttttttggctgcctctagctagcacatttatgaaaaacaggttaagttactggctccaaattactccttaataaaaaaaacaaacaaacaaaacagcataagcttttaagattttcttttttaaaacctagaaaacagtctccctgtcaagagcaggcatgattaatgacttctgtgctgtaagcttgggtatccgccgtgttactagtgtgtgatactcctaacagcttgtaattgccccgaggatacttttggaactgaaaggtgtggggcggggcctaccttcttaatatctccataattttttgggggggggttagactacatttatcaatgggacattgaaaatacagaaacattgtcaagtcatccacatgaaattatgaatgcattatcatctgaagagtcatttccataaaacggcagatgacaaatagtcagctctcagtatccatgggggatgggttcaagatccctccctcagacatgaaaatctgagatgagcaagtttcttattatagatggtaaatctgcatatgatttaagtttttcttcctgcatactagaaatcagagctagatggtttgccatacttgagagcgcacaaacgctgcgtaaacagtcaaccgtacggcactgcttagagcagtggttctcaaccttcctaacgctgcttctcaaatgctgggaatggaaggtgtgtggcaccatgctcagctagaatcccattgtaagtatggcactggctggacctacatctgcagaactccagggtactgaagggctgtactgcatcctagcagcttcctaggtcttaggaagctaccaacctgcaaactccttctgaggtgaaaccacaacagggcatccaccaaactcaaccactggaacccaatgaaccagtcaagttaacagaacactgagccaccttttgtgctccatgacagccctgacagacaacataggcaagctctatcacactgtatggctcaaccagccatattttccatagaggggaaatggagtttgctgctttgtttggcagatgcaaagagtaaagtagaggggaaccaaatgagcttagagcacttgggaaggtaggctgacttcctagtgtgttccactgtaccacgcaatcccactattagctccccacttcctctggtcaacaccagatctgaacgaaacctacaggaaggtttgaaggtacctggccttctaaattgtggcttacataataacatcaggccctctggtagaccctgtaggtcaacaacctaggaagccaattcctatagtccagcttaaataacaaccttgtgtttcaagggaaaataggaaggtgtggaaagcctccattgacaccccaactaacttgcctttgcttttctagagacaactatgcacttaagaaaggcaatcgagatctgcagttaatttacagtcactgagatgactcaacatacgttcatactagtagtatcttcagcttctccagttttttttttttaaagtagaaacaaaatgtccggcctgacattttaaaaacctgacctatgaggaacttgaaatggtcagttttacaaattttccttcctttcctctctttcttctaagtcattcactgaattatgtaactgactcctagaacacactgcagaccccatatggaataagacagcacaactcctgcatgcatgatcactgctgggctgcaggaatgtgttaagacacatgtctgtccctcagtagtgtagacacaatccctgtcttgccctttagaagtttataagatccctgggcacacagaacttgtgcatcaagttaaagactaaagggccagagtggggttgacaagagcaatttcttaagagtctgaagaaagggagccagaaacatggagaaggcagtgaagactattttcagcagagagaagagagaatgttattgagatgaaaatgtggatggcagattccagtgcaaggggtgcagatctcatagaaggtgggtcagggatgtggggtttgaaaatggctaacagcgagctccattatgtcacagcagagatgacgcaaggagctgtctcccagcaggtgagatgatgctgggtaggcaggaggtagacacaggaagtggatttaggaacccatagcaaccatgagggagaatgagggtggaacttaaataagggagcatttacgtagaaaagagttggcagaattaactccatgggaaacttagtggaactgaggacaacgtgtcaactacaaagaccagaaagcagggctagttagatggctgagtgaggaagggtgcttgctctgacttcaatcttcaggagtcacattccaggctctatctccacacgtgtgctatggcacgtccacacatagagcaaccaaataaataaacataacattgaaagataccagaatgcatgctgtctttgaaaaccctattcattgtcttaagtataagatgacagggccatttcccaaaaccatacatcttgtggagacacaggtggggcaaaaccaggtcaagttcagtggacagcaggtaccaaatggacttatggctatacctggtaaggcaagccatgaagatgatgagttctggctccctcgttcttcgggaacctgaaatctggagaagggggaggggggaggaggaggaggaggagaaggggaagaggtagtggtggtcactggtggacagggaagaacatctaggaggaaagaagctacagaagttactgacggcaggagagggtccttccaggccatggaaacactgggcctcaaaggacaggctgctccagtctgatgctgcatcagaaacccacagtcaatatattgaaggtggaaatctgaaaagcacttatttttataattaaatacttttatgatagagaaaaaagcaaacagagaaaaaaaagcaaaagcatcaggtttcacccaaacacagcaatggtcagtttgggttcatctgagatgaccaacaaatgccaatattagagttacagtttttcaaggccataattgctacatttaaaacatcaagagtcacagactgtccccaaacagtcagagccctcatgacctgcctgtcccaggatctgagagctacagtgttctgcttctatttagagcattatgaatgctaaatagaaatgttcaaaatattttacaaaatgaaaagtagacatcagtccactgcaggtctaaggctgttctccaactttaaaatacattagttcttttttttcctttcccctttttcttttctggagacaaggtctcaggtagcccaggctggcctagtattttatgtgtgactgagaatgaccttgagctcccgattctcctgcctccatggaccaacgagtgcagttctttccccttaagagtcacatagttgaaccaggaaggatggctcatgcctgcaatcccagcactcaaaggggaaaggcaggagggctgtcttgagttcgaagccagcagggactataaagtgagctccaagataggctgcgttgcagaatcacaacacgtctcaaacaaacaacgtagcagacatatctaacccttaagcatgacaacgacacatttgtatgatacctgaataataagaagggggtgcatcattataattattattattttacagttgtaaacatgggaaagtgctgagcagttcaatgagcttagggctacagtcactcaactgcatgctagctcagatgaggataatgcagaaacgcctgccgggaggaaggaaggagcaaacacttctgccactccgtgaaatgtcactcaaataacaaaacaaacccaccgaccgacctctcaggataaggaagtgtagcggagtgggaacacactcttgactggtgtgtgaggggccctgggattctgtacctagcatcacacacaaaaactgcaaacaaattctttaagccatgcttggtaaaaatgactcattttgaacctagtgtgaattggagattatttttcaattaatgaagtttttctagttggaaggacttacaacaagggccactatcccaaaagcccttagggacttgtctaaatgagtaaaaatattgtgcaattaggccaacaactgaaactaagtgcgtctgagagcttaaaagtagtcgttctcaagtactagttcgaagagcatcaaatcatataaaaggttcaaaggaatttaagtgaactgggttgacttttcaaggcactgaggtaattacaaaatacactaaatagcaaatattaggcataccctaggtttagacaaacaaaaccaccctaaaaccgccagatcatatagacacacacacacacacacacactcattttctctctctctctaacacacacacacacaaaacacattagggtaaatatgtattagaaaaacctttgcctaagatttcctttaaacagtgggtattattcatttttgtttttttctttacagaacattaatttctttgcaaaatgtttggggtacagagattgcctgacaagagtggccaccatgaagcttatgggcacgtgagaggagaggtgaagaagcctcaatctttctgagcccatttcatctcccctattattctccacgattttcaaaagccactctactacaaacctgagaaacacaggaggtcagattcacaaatgaaatctctttgtctcccttctgtatgcacccagcatctgctccacctagctgaccgttacaggggcagggccagtgttaacatgtcaagtgggcagagggctctctgcatctcctagtacatttctggctacaggtgaactaaatgacaccaaagaactgctctcaaacgtgcatcccaaggtagagctacaaaggttcaatataaccccagtattttgagagatcttaaaacgcagtcaaagttaactgtcatgtccatagcaaggaggaactcactccttaggatacactgggctggtcttccagtgggcatttcagagagccctgctttcccatatcagctccatcctgtatcctcaccgttgcttccaacagggaaccgtccacatctgactgcccaatcagatttcagcaggtggcatgggggatccagaaagcatttctctgcaccttttaactctgaactgcatgcagaacccgtggtaagaaatgcttctctgatcgctcctagcacttttagtggccatgttaaatcagctaagtaagaatgactcttccatctgtgttaagtggccttttaaagctaattcacactatctccaaataccctgaattaccaagtctgagggctgcctagattcctgcttgtaagcaatagaatgacactgaggactctcggctctcttctctctcccatatcctctctggttcactgagaaccaaacatcatctaaacagaacaaaaacatcagaactgaaggaatcgccaaagtatatacacgtgactcccgtctgaatgcattcagatccgaccagtgactccaagctttctgcatgtttcccatgagctatttgccctgaatcaactcctcaccacaactctcgaaatgacatggacctctgctgttgcacttattattccttctctgatggggagtggttatgactgaaaattttcgcgcaaccagcctctaaacagaaatgcagtttattctgagtactcaaattcaaatctcagtaacacagcaattacattttaaaataaattctctccatgttcttgcttctaagaagagggtttagtgggatatgaagagtctaatcctaggcaaccatatggccaggccaattataaaccccatatatgttcacacatcaagcagggcaaactgctctgggaacaacccttaatattttattgcctgcccaggtgcctgagcacttctgagatacacctgtcacaggaaaatacagcacttccttccatctttaccccatagttgccagacaattgggcagagctaaagtgggtaatgttgcagtcaacacttccctaaaacatttatacatgccactgataaacaatagaaatttaaaggagatacaaatgtactccccaaaccctaaatcttcctcccacggaggcctaaaaacttaaaaatcttcatctacctggcacagaagaaggaatacaaattccaaggggagcctagatttatggcctaaatatgtcttcctctagaatatttaatacacttacgctttagtcattccatacattttgaaaatactctagcacgataaattacagtagctgcatctttcatggttgttcacataaatatcatttggctttcaggaaaccaattacggagcggtaaatcatttttacattgcttttcaaaatctttaatttaacttgcaacacggacagggcacacacaggctcccaggaagaacgcagctcacacattgtatcattccctcttgtccgagtttagcaacaacccctgagtaagccacaacaatattttcatgttctaaaatatttgggttatatctatatatttcacacaggtattaggctcagcaagtatttcctaacctcctgtagtatttagttttcaagacttggggtaggattgggaccacattgatttcagggtgctgactgtacttctgtcaccatcagctagtttaaggacaatgactgtcctggcagacaccgcaaacacagagcctctatggtgttcttgagaaatcaagaccatcatctcattggaaaggcggtgggaaacaaggtgcagggccattgaggccacacaatcaacaacaggatttgctccagcctcagagacacagagtggactggcatactatatcaatttggaatcccctcggctatgacaagtttgtgaaatgaaagaacaaaacaatacagtacaagatcaagttttagtagagagaatgcagacaaccacaaccgagtacttagggagaaaaaaaaaaaacccagctcttagctggggagtgacagtcaaggggggaaatcacgcgaaggaatttctttcaggtgcaagaaggcatgagagtgggagacaaggattgagcatcttggcagaaggatcagaaacccagtccaaggtctacacttccgtggagcagccgcagacaagcttaggtatccggtggggataacctacagttaagacatcacattacagacactagggagccattgtgaattacagccaacaggtgagaatcataacttagcggggcggacacaacaaccaagccgtgatctagagcaggaagcgggggatagacagaaagacaggttgtaatggaggagaccaagcagagggctgcattaaggcagaggcttctgtgctgaccaagtgactaacacaaagggcccttgcagaagaccagagatgtcaggggggagaattccttctgagggggcctagttacactagccacttgatattcaccgggaaggaggcagagaagctaggagggtgggcacaaaggcagtggctctcttctacctgaggagagctggcagctctatcaatacggactactcctaaatgggctgcagtcagcccagaaaaaaaaaaaatcactcaatactctcaggttacctaccctggctcccaaagctggttatgcaagaaatattttgttgttgttttgcttttaaaaggagacaactgatgcttgttttagtattgtttgaactacatcactcacaaatgagagactgataaagagattgacaagtctgactctagaagcaaacacttccagtctgctctcatctgccctcccccacttcctccattctcctcggcctctaccccacaatatgcttgaaaatagtaggtaactgagtttgttttgtacggtggttgtcttgttatttcaagcagggtggtccagagagaaaaaaacattttctggaaagattttaactactggatcataaacaagcgtgtggtggccacgcagcaatctttgtaataacgaatccaagacgtcctggttgtgaagcccagtgaatgtaatgttagctgaaaagcaccgcctgacacacctggaaatgtgccacgggccagctttgtagactgcgacttcaaagggaaactggagtctacttccgcttttcagtgagttagaaatgattttggctgtattggtagggcagaaccagaaaataagttttgaatttgtcataataatttacaaaaaccaaccaacttgttaagatgcatcctccaagcacaactaagctggctatagaggctattctgcattctgcatctggtcccctggactgctcaggcttagggtgagggaagaaacatgacttaagccattacccttattctgtgggcatttgaagtggctgccaggcacgtgacaccaactgttccaattggttcacaatgacatcactttattattcaactctgtttgcatttcagtctcctcctttttcgggggacttgaggccaatcctggccaagtcactttagaagagaaagaagccagtcttaggtagtcatgaagtgccaagtgtcttttgtgtctcctagcctagtttgtaagccgggaactggtgacataaagggctctgtcaacagcgcccgggtgtgctgcttccctcgcagcgggagagggagattgggaacccagtagaaggtggcggttcggccttgggtaggtcccaactgaccaagttcaataaaatccctgagagtttgttttcaattgggtaagaaaactataaaatggaaaccgcatgcgagcttacgctggaacctcatttaaagcacttgtacttccaaaaaaaaaaaaaaaaatgcagaaaacatttctaatttagtcaagcgcactcttctttcctcgagcaattagtcttaattcatgtcacctaactactatttctgcattaagcaacgtgacctctagaacctacagtaaccccctttctgatggtgaaagtcaattattctgatttttttcatcttcacacaataagttaccagagaataaactaacttcaaaaaagcattacagtgtcatgtgtgttcgcctgttgatatgtgcacacattcatggcacctgtgggtctgtatccagctcttctactgtgcctgtctctcaattttgcagctcaacctagagatcaccaagtggccagcgagctccatggatcagcccgtttctgccctcctctgtactaagattacacgcaatcatgctgagcctctaagaacatggccgctcatggtctgaactcggatcctccacctagcatggcaaacatgttaggaacggggccatttcctcaacacaaaggaaaaaggcttcctcatgggagagaatgactggatgggcgagtagacagatgaaaatggacttttcacaccaactggttagagaccctgttgcttccaggcacaaagcacaccagtttctagtatcagtgggttgaaatgcatgtttaagctctctcatccttacttggctcttgacaggaaacttttatggttgaactttattctgtagaaatgcagtggcattaaccaagcacacaaagccaactcaagcctaaagtgtgcccatgactccaaccctacactcattggttcttgccagaggcaacacgaaacagtgtatagactcagaactctgcccaggcctgtgaggcagggccagggctacacaccttgcaaacacacagcaggctctgcccaaggagcgttcactaggtaactatcagggtggctcaagggctgtacgacagacagaccctgcacaggcagggaaagtttcctggagacctaccatctattttcttgtaatctcaactgatgtgttctgccaagtcaactattatttatcatttataaagcagctcctattggcagcatactctgcatatgtttcagcacttcataaaccctccacggtataatcgcttgtggaggagactgggttgcctggagctggaatacagacccacacgcatgtgagcccgtgtaaatgcacacatgaccgttccctaccgctctttcacgagtcatgtcttggctctgtaggccgagtatctataactaaagctctctacaggtgtcaaatcaagcaatcttggtcctctcagatgtcagctaatggcttagagccccccccccctaacagttctatttgcattgctaaccaaggctatccaccttagtatcatcatcagtgcaagagaagccaaactgcacacgaagacctggagagcacttcagagcacacatccgacctcttcctcatccgagtacctagcaagccagttttccttcctctcctgtctccagaagaagttccaactccacctcggagcaaagcagcagctgcagtgactagcagtgactatgcctttgtgactgcgtatcactcatccaattcacagactacttagcatcgacttgtatacactttgcccaagaataaatatttatattcccgaaacacagtcgatcgagagcagctgatacagggctggagatgacagaggcagcaaacacaaacaagtaagacctccaggagtgtgagggacagggctagagacaaagtaaacagaggccacaagatcctaggcgctaggtgggggctactggtacgtaagggctgctggaaagaagagtcctccggggactggctcccacaaaggctgcccaatctcaagtggtcagctctgggcgcttgtctagatggcaatgctgcgtggactcagtgggttatgtatgcgtgtgtacatgcacatgctaggataataattaaaaataagaggtcatgagtttgggaggttggagtttgctcatgggagcaactggaagtggggggcagagaaagggctgggagtgacacaggtgtagttcttgtgtatggtctaaagattaaattagagttagggaaaaaaaaaccccacaggaattgggaagagtgattcccaagccactgacacagcacagcactgcttgcctgtggaggtgctcctgtgccatcctccctaatctttatgttgcacatcttaagagtggtctgtttgtgtatcctctcatgcatgtatctcctcagcgttctcatccacgaatgcttacaggtgacgttagcatcagcagttggagaacaagatacactggaaagaaaacattgtaccctgatttgcaccgggaagaggagaaagggaacacgctaagcatataaacatgttaacggtcaagtagagtccaaatcagatcttgccactcttcaaactgaaggtctgtgatacttaaaagggggaaaaaacccattgaaatgcagaaggcagagaccatcactcaaagatactcatcgcttggaggtcagtttatatttggaacagtgacttgggattggccaaccatcactcatgccattaaagtaatatttcagtttcaactgttttacatgcatttagcatgtagacgtattcaatttatagttgtaaacggtctacaagtgtaagaagccgatacccaagcttcgaatttgtaggtgtttaagtctaagtcccaattcataccggcactacaggtcagcaagtgtatggtttttaactttaagagggcgctcaataaactcggatagaaaatctgggagcaccaggccttgttgtgaagctactttcaaggcgatgatcctcatcgccaggaggaagttggtttatacccatcatagttcctttaacacaagcaatgcagaatggccttcacaagtgtctcctctaaaatgactgtttaaattgtgttgtttcaaaaagaggacatccacgagccgcatgtttccattatcccttggaactggagctacaggcagtcaagggcttccttacttgggtgctgggacacaactctggccctcatggtagaacagcaagcccagcaacctttctagttccaggattaggtcttaactttcgtggtctcttgatggcacacagaaattgtctagaagtcagaggctatgtacgggtggatgatgtacacccagtcgccctccaagcaagccaaatatgaaagcaattttttttttcaaaaataaaatagcaccacactgctctcccaccaggcgtgtagagattttccccaacaaaatatgacacacatcatatctatgattgcttctaaatgaagaagtctttaaaagttttaacttcttaagggccaaatatcaatggatatattccatataaataaaaattcatcagatttgtcttcacctattttggttgctgtaaccaaatccatcaaccaaccaactaaccaaccaatcaaccaaccaaccaaccaaccaaccaaccaaccaaccaaccaggtagtctctgtcttgttccagtcccattttcctggtgcacaaaaggactgattttacttgttagaaggttatcgggcaattctctgtgtctctcttacaagggtactaagcattctcaggagggttcaatctttatggcccaatcactccccttcgccccaaaacaatgtcttgatgaccttccaacacagtcatggcagattatggtctgtaggctttactggtgccaagggctcctgagaatacaacaccaaagtgatctcatcccaagaaggatgcttcagacagacagtcccacatgctttcctctactcctggctcatcccctgtattccatacatctatatcccccgtccctcttagctctcctctgagtggcaaagaggcatctccgacttaacgagatgcaaggcagagttctagctccatctgccatccccaccacacacctcccaaacaaacctactcccttcctcctgccagctcgggaacgggcaacttcacttctcgtttcatctaagccaaatgcctgaatcatcctttatccctctttgccccacctgcaagatttcacaagtccctaaatattcctgaatctatctactcttagttactgtaactgatgggtgcagttctcaacaaggaactcttctgtcccggcactacctgcatccagccatgtctagatacatttttcaattgtgtctgcgtagacaaggggtagatgtaccaccagtatctagcaggaagatgcatagacaaggggtagatgtaccaccagtatctagcaggaagaggccagggatattgatagacattctataatgcacacagcagctcctatcatcaaagactgtccagctcaaaatgtcaatggcattgagaaactgagctctattccaagccagtagaatctctccaaaaaaaccttctcaaaacaacttcctaactgttttccctgatttttcactcttaattgctctgctcgatgaaaacaactaaagttatcagcagaaatacagagtgtgacccactagagtgctttgtcttgtgtcatggcctgtaggcccttgcctactccactggcccccggccagctcctacattctcatcccaccaaattcctacgtcagagccctaagccctcaggtgactctgctagagacagactctacggtagtaactgaaggttaagtgagcccgtaaggaggaactgagccttgtaatggttggtgccttcatgaaagacacacggaagcttgctctctcatccactctgctgtgaagcttgctctcccatccactctgctgtgagaagatggccatccacacagaaaaaggacctcactggaatttgaccttgctggaactctgatcttggcctcatagttctaggactttgagaaaagaaacgtcagcagcttaagccacccaggctgtggtactttgttgtgaaagcttggactaatacattcaaccttgctaccttgccaagttccagttcactggccatcagtccacttacaccacagcccttgccctccctcttctgaactgccattagctgacccctaataacccctagtcaaaatgccaccctccccaggagggactgtgttactccatccttcctggattttgaggacagccccatggttggcattaaccaagctgcctaccctctttgcttactggtctccttgacccagtgcagaaacctctcagtcttgccccttgtcgaatcaggataagataatctgtgtggcttatatctcttaaactcaaaacaaggaaaggtagggggtctgtgaacatgctttgccccatgacccagttccctctgccgcttctccaaagagagctaccatacttatgcctaccttagtttcccaagagcccatccagggcaggagggaatcattacatgtaacatctagaatgagtacttccttatattctttcactttagggagcagcaaaggttcaaggttacggtaaagtacaggtatgggggcatcaccctgatatgatgaagcaccaaggaactccccaatgttaactatctagccacagagcaggtaagggtatgctccagttagttttacgaggtcaactctgaacagaggaaacaaaagcaagcagtaaaggactatgaggcacgtgggtttgtgctgtgtacagccgagcagtcacagatgcctcactaaggaggtggcatggggactaagagtgactggagttagccatgggaaattcaggcaaaaagcaaggaaggtctctctgaactggtgaggaacagcagtcgtcagtcagtattactgtccgttatttctctacactggccccaggcaggtgcttttgaatgactggctgtttgcaaagggaagtccagatacagatggccgaatgaggggccacgagcctgtgcaggacatacagataatggaacaggaaccttgcaatgctgagacctactacgtgacgggtgcttaacagcttttgaggttaacggtgggaatttctgagagaatcccaaagagagctagctggttgcttgaggccaacaagggcaaggcttataagtcaggggcaccccagaaaggaggcacaccccagaaaggaggcagcttctaagaaagaggaaaagagagaaagcaggcaagcctgagaaatcaggcttgggtggcctgaaaaggcagaccatgatgtgtgtggtgggaaagcagggcaggactcctcctccttctggaggaatacatgtgctggcactgggacatccagcttcaaccccaggagcccaggcccagcatgacctcctaacgcactgtttagtagacagggtagagcctttaagaatggtggctcgatggaggccctgtcccatcctccaagatgtctaaagggggtgggactggctaagtccctccttctcttcctcaccccctccacatccaacctcctccaaaaacatcaaatattttcttccataaataattgcaacaggatgccattgctttcctcaaagaaacaatggcctgattgtgccctgtccgacacgggcccagccccagcacacacaaagggtggctttcagaagccctgctgtagagaatgggggtacacctcctttcttctcttctgtaccaagtccactgccctccaccaggaaaggacatcaggaaggggctgctaccacaggaataattcccatcagaggacagctatcattcacctgcctttagattgccctttggccacctctagaaagttcttcgttctggtgaacagaagagcacctattggctctaaaatccaacacacacacacatacagttttaggctggagatatctataatttttttttctaattttcaaacttatttgaaattgtcattaattaacttttaaaatatttgaaatcctagcattcaggaggcagaggctggacaattgccccacatttgaggccagtctggtatacactgcaagtttaggtcagccaggcctatgtagtgagctcctgtctcaaaaaaaaaatacataagtaaataaaaattataaatagataataaaatagtcccattttacgtgtacatcttcagtttcacaatttgagataaagtgaatacagatattttcattcagtatctgtggtagtgccaacactggatgctcacacaggctcatgcatgtgaacatctggtccccgtggctggcacagtttgggaaggttatggaacctttaggaggtggagtcttgctggaagaagtgagctaagccactgggtgcagactcggaggctgatagctgggccccacttcctgtccgctctcctgtttcttcaccccactccacttcctaagtacaggaagcgcttcatctagcttcctgcccctgccaccacagcatcccagattaattccatggaaactctaagtcaaaataagccctttattacttaagttgtttttgtaacactaatcaagtgtgtttcttggttaaaaaggacaggctggactaaaggattcaatttcagcacttaaggggctggagagatgagagagcctgctgctggggctgtgggctgggataagttgaggcccgagtttagttctcagcacccatgtttggttcccaacattccatgttcaccccaggcctccaagggcctccacacacatagatatacccaccctgagatattcgcatgcatataaataaacataaaataaatatttaaagaacccatcactttgcagccatggcttgacaaacaggctctttggtgtcttcctcacataggtgcaagaggaactcaggcaggggtgttgagtttttggattttgcacttatttatttatttattggtggtactctctgtttattgggggagttgtggtgggttgccatttgtaacatttccattaaagatgttaaaaggctgccttcccattttcaaagactcagactgatcactctcccagccagaggcttctgtcagctactaaagggttcctttcccttctattttatggcctttctcccatccacttctgctttgttcttttaattaaaatgaaatgtaggccctccatggctgagatgctaataaggcccctgaaagtcatctgtgagcacatccactcttgtttaaagagccatgtaaacagaccacagacggctacagagagcaacgctgcacagggcaaggaaaagggaatctggctgcaccgtacagtgctacgttgaagtgatttatcacttatgccaggaggaggaactgaaacacaaacgcaatttcaagtttaagcatggcgtctccacaggatcttccatagcaagcactctgcactcgggggctccctgtcaggaaactcctggacaacggttggtttgccgtctgatccacttcagttacctcaccaaacattcaagaggtactttctgatgtgtgtgggctttagcgctaggttgatacaaaacagtacctggatctgtactggaagtcaactatatttgctgccaaatccattgtaactggctctgcatggagccatccgtgtgagagaggctgatgtgtctgccaccactctgcagacactggaaacagcgcaggaccaatctgtatccatcgttcctacctgtttctcagtgggtaacattctcaaagcattttcagatgaagaatttagtcagacagacagacagatggagacacggatcactggaagactgctgtatggggccacatggggaaacactgggttagtcaggaagacacggcggtttctacagggaaagtctgggtgtccctttggagttcttgtgtgagaaagttgtccccaaggtatcagtgctgggagatacaagttttttgggtttttttgtttgtttgtttgcttgtttgtttgtttgtttcaagacagggtttctctgtgtagctctggctgtcctggaactcactttgtagaccaggctggcctcgaactcagaaatccgcctgtctctgcctcctgagtgctgggattaaaggcgtgcgccaccacgcccaggtgggagatacaagttttaagaggtgggcctggtgataggttcttaggccactgggtaccctgtcctcagagattaaggtaattctcacaagagggctagagaaacgggccaagtgtgacctctctccacatctgcttcttatgtttacttgctctcaggttcctgtcactgactcgagcaagagcaacccttaccatagctgcactatgatgtttggacttcaaaaccaaactctggacattaactaagatttctctttttaaaatcagtaatacactgttgactaacaatgaatgaatgaagcagagtaagtaggctgaggatggactggttcaagttatcttggcaggcagtgaagttgggtagtgtacggcttccctggcatgtggccctagggagaccaggacagaagaatattgacgcagagtgtgagaggccaatcaaggaagtgtgggggctgagatatgtgcttctttactacctaggaatctacgagaatctcttcaggctcagtcgaggccccccaaacggcaagcctgtctaagtacaggaaacaggcaagattaatatggaatagtggcgcaaaccaattagctcaaaaatttccacaaccccatgtattagccctctgctcagctcagggagataatgagaacacataacggaagctcccagttcaccaatgcaactggggagcggggggggatgggtacctcaaccactcttaatgagtctgtagactaagataacagaatggcagtcttccatcaacatgaggtaaagctagcatggcacctcagcctttcaaagttactgttcactgaaatgttattagaaaaattgccaattgtgataaagcctatggatcaaacactactcccattgcaaggaacattcataaacagaaagctgttgtcagtccccgaaacttaaactgtatagagcagagaaaacctggaagagggaagcgccataatacggatgagaaaacagggctaggggctcgtaaatggcagatcaggaaggaatcccatccgccttggagttggtgatcttgtaatgttcagagaatggactcatttcaactcacaggttcggtgtgacccacgtgcttgctgaagcaccgtgcttactgtatggtgctacacaagtgaggtctctcataagctgcagagacacagaaaagtctcaagggacaggtgggtgcatgtgtccaagacctggtcgagcagttaatacgtccaggttgtggcttgtgctctgcttcattagtgtgttgccagcgactaggaatgtgacggctaccaccttgctgtttggactcatttcatggggtaactgcagcaggcacagggaatgagctaagacagaccctgggtgagggaggcagctcatcttcaatgcccggagagcacaacctatctgatctgatatactctccagtttacaaactactgacaatcacatcaagtgaagggcagctgcagccacagctctcccttgttcacagtttgctttccttggttctagttacctgaaggcaactatgacataacaatattaaatggaaaattacagaaataaagtagttccttggcctcaagttgcctgccactggaagttacaggaagccatcttgttgtcctgctctgtaagggtcctccctttctgcaacggatccaggctgtatacccaaccatcggctagcgacgtcatagagatctgagtagaacaagtgctgttgttcaagtacccctgactttacttaacagtgacccccaaacgggaaaaagagtagtgttggcaacttattacagccaaaaacatgttattacaatgtatttttatgactgctgtattgatactgttaattgctggttataattaagcttcgttatagatacgtatttataggaattagcagcatacatcaggtttggtggtatttatggtttcaggaatctactacagatctttgaaagtatctccctcacagggagagagactactgtaccattgctcttcaatggtgactcctaatactggcatatagacagctctgctgaccttttagaagtgccagagaacaggcactctggacacagcgctgctcttggcctcgagggatctgcaagaaggaatggagggaaggatatgtctctggggaccagcttctcctttgaacatgcaggaactaatgagacgctttttgaaggagtccaggtgagtgacacctattaaaggtgcttagttaatgctcatccccataaacgcccattacggcgacaggttcccactactcagccggaacgtttgagctctagatgctcctctgaagaaatggctgaaaaatcagggagagatgctaaacccaaacacagtagcagtgctggcaggagatgagccaggctggtgagctgttcgtaaaccagctgtccccgcccccaagactggagagtacagagaattccatgcacccagggtcgtcttaagacatgaacatgctttcagagctgcatccctaagaaactcctacaaacctgctccagaccatgaccctgcaggctctgtctcacccctgcccttccactcagctgctcctgagttacagcctggctaaaatagcaatgctacattttctgccatttgggggccactgaggcaaatgatcatgttgaaggggtaactggctattcactcaaacctctgcattcctcagtgagaggacagtctcagaggatcccaccaggatgtgctggctctgcatggtgggtgtcagaactacattcgatttgcaggtaccatatggtgtttgcggggctggaaccctgcttgtgggtattgaaaatcacgtactgggagtcaggagcgttgtgagtacaaacatttcagagtgttgagagtgcttggtgggggtttaggaacaaggcaggctctgacttaccagaactgcagcttttattctgaagtttcttttaagaaccaagattaggggctggagagatggctcagaggttaagagcacccgactgctcttccgaaggtcctgagttcaaatcccagcaaccacatggtggctcacaaccatctgtaatgagatctgactccctcttctggggtgtctgaagacagctacggcgtacttacatagaataaataaataaataaatctttaaaaaaagaaaaaaaggaaccaagattaggcatttgattcattaaacaggacaggggatcaaatacctccgttgggctctcgagaacatgacagggtggggtgtgcagataagaaggctctagctagggtagctggaatccctctttcaacgatggcgaagcactggcacccgtagaaagaaggaatgatacgaatcagcccactattctgaactatgtccctgcccagacacagaggcacatgctgggcctgcagcaaatagccctggagccacactcgtggttggcgtgcagaggaggaaccaacccatcagaactctgtccgtgtgggtgtgtgtaggtgcctgtggtgaaggtcagaggtcaactcaggtgtccaatccaggctccttttgaacttctagcttagtttgtcaacactggcagctgaattgggtttgtttgtttgttttttgagatgtgatcttgatctgcagcccaggctgggcttgaattc

SEQ ID NO: 7 - DNA, 5293 bp, Sus scrofa
  misc_feature (3177)..(3178): n is a, c, g, or t
aaagggaatttcatcccaaataaaaggaatgaagtctggctccggaggagggtccccgacctcgctgtgggggctcctgtttttctccgccgcgctctcgctctggccgacgagtggagaaatctgcgggccgggcattgacatccgcaacgactaccagcagctgaagcgcctggagaactgcacggtgatcgagggctacctccacatcctgctcatctccaaggctgaggactaccgcagctaccgctttcccaagctcacggtcatcaccgagtacctgctgctgttccgtgtggctggccttgagagccttggcgacctcttccccaatctcacggtcatccgcggctggaagctcttctacaactacgccctggtcatcttcgagatgaccaacctcaaggacatcgggctctacaacctgaggaatattacccggggggccatcaggatcgagaagaatgcygacctctgctacctctctacggtggactggtccctgatcctggatgccgtgtccaataactacatcgtgggcaacaagccccctaaggaatgcggggacttgtgcccagggacgctggaggagaagccgctgtgtgagaagacgaccatcaacaacgagtacaactatcgctgctggaccacgaaccgctgccagaaaatgtgcccgagcgtgtgcgggaagcgggcgtgcacagagaaccacgagtgctgccaccccgagtgcctgggcagctgcagcgcacccgacaacgacacrgcctgtgtggcctgccgccactactactacgccggagtctgcgtgcccagctgcccacccaacacctaccgcttcgagggctggcgctgcgtggaccgcgacttttgcgccaacatccccagtgcggagagcagtgactccgagggctttgtgatccatgatggsgagtgcatgcaggagtgcccttcaggcttcatccgcaatggcagccagagcatgtactgtatcccttgcgaaggcccttgccccaaagtctgtgaggaagagaagaagacaaagaccattgattctgtcacttctgctcagatgctccaaggatgcaccatcttcaagggcaacctgctcattaacatccgacgcggcaacaacattgcctcggagctagagaacttcatggggctcatcgaggtggtgacgggctacgtgaagatccgccactcccatgccttggtctccttgtccttcctgaaaaatcttcgccagatcctaggggaggagcagctggaggggaattactccttctacgtccttgacaaccagaacttgcagcagctgtgggactgggaccatcgcaacctgaccatcaaagctgggaaaatgtactttgctttcaatcccaaattgtgtgtctccgagatttaccgcatggaggaagtgacagggactaaagggcgccagagcaaaggggacataaacaccaggaacaatggagagagagcctcctgtgaaagtgacgtcctkcacttcacctccaccaccacgtggaagaaccgcatcatcatcacctggcaccgataccggccccctgactacagggacctcatcagcttcactgtctactacaaggaagcgccctttaaaaacgtcacggagtatgatgggcaggatgcctgtggctccaacagctggaacatggtggacgtggacctccctcccaataaggacgtcgagcctggcattctactgcatgggctgaagccctggacacagtacgcygtttacgtcraggccgtgaccctcaccatggtggagaatgaccacatccgtggggccaagagtgagatcttgtacattcgcaccaatgcttcagttccttccattcccctggatgtcctctcagcatcaaactcctcttctcagctgatagtgaagtggaacccgccctccctgcccaatggcaacctgagctactacatcgtgcggtggcagcgccagcctcaggacagctatctctaccggcataattactgctccaaaggtgagggggacatgggacacctgtggctctgactcccgagccctatgctacgcattcagcatcaggctgctgctgtgtgcagccttggccatggtcacaggtgctaaccgyggtgtcytcccgtaccggttgggtaacttggctctccttgggctgctcttttccaagcagacaaaatccccatcaggaagtatgctgatggcaccattgatgtcgaggaggctacagagaaccccaagactgaagtgtgtggcggggagaagggaccatgctgtgcttgccccaaaacagaagctgagaagcaggcagagaaggaggaggctgagtaccgcaaggtcttcgagaacttcctgcacaatgccatctttgtgcccagacctgaacggaagcggagggaggtcatgcaggtcgccaayaccaccatgtccagccggagcagaaacaccacagtggtggacacctacaatgtcacggacccagaggagcttgagacagagtaccctttttttgagagcagagtggataacaaggagagaactgtcatttccaacctccggccttttactttgtaccgaattgacatccacagctgtaaccatgaggctgagaagctgggctgcagtgcctccaactttgtctttgcaagaaccatgcctgcaggtatggtgtgatccagtcactccattcctaattcactcacagccttgatttgtacagatggaggctgacacccagggcaatgacccttactctcagtcctgcttattaaagaatgagtggtcttttgtaggcagctcactagtccagcagcccaggaatggagttcattcatagcaaccccactctctccccagtctctccatacagagtgcttgagagcagggaactcaattcagggtctacactttccctctccttggttcatgttgtgcattgccgtgggcccttttttttgcccatttatgcaaagggaaatttgccaggtctctcctgagttctcagagattctgtgaaatcctcagttggctgccctgaatcaaagtgagtgtagctcctgaaggtcagctcccagagcagcttcgcgtcctgcttgtttggtcctcaccttcctcttcttttagaagctacctgtggcccagacagcaagccatcnnaaaaaaaaaaaagaaacctactgtgtatgtgaggtcaggtcaggagaaatgaacgcaggtaccgtctgcagtcctgtatgcatggttgaagagggtattcttaggtttaatccagcattttttgtcttttttgggggacttgagtctaacttcattgcttgtttcaattgttgcacagaaggagcagatgacattcctgggccagtgacctgggagccacggcctgagaactccatctttttaaagtggccagaacctgagaatcctaatggattgattctaatgtatgaaataaaatacggatcacaagtcgaggatcagcgggaatgtgtgtccagacaggagtacaggaagtatggaggggccaagctgaacaggctcaacccggggaactacacggcccggattcaggccacctctctctccgggaatgggtcatggacggaacctgtgttcttctacgtccaggccaaaacgacatatgaaaacttcatccatctgatcatcgccctaccagtggccgttctgttgatagtaggagggttggttataatgttgtacgtcttccacagaaagagaaataacagcaggctggggaatggagtgctgtatgcttccgtgaacccggagtacttcagcgcagctgatgtgtacgtgcccgacgagtgggaggtcgcccgggagaagatcaccatgagccgggagctgggacagggctccttcgggatggtttatgaaggcgtcgccaasggygtggtcaaagatgagcctgagaccagggtggccattaagacagtgaatgaggccgccagcatgcgygagaggattgagtttctcaacgaggcctccgtgatgaaggagttcaactgtcaccacgtggtgagggaagggccccaagggttgggrccacccttatgggtttgcgactcatcctgtctctgtaggatccctstcactgctcaagaaggttgggggggatgtgcaggtccagggagtctggtgccacatcatccagggttgctcagggtggtcctggtggggttggggggctgcgggggctcaccccctgtctccttctctttccccctcacaggtgcggctgctgggtgtggtgtcccagggccagccaacgctggtcatcatggaactgatgacccggggggacctcaaaagttatctccggtctctgagaccagaaatggagaataatccagtcctagcacctccaagcctaagcaaaatgatccagatggctggagagatygcagatggcatggcatacctcaacgccaataagtttgtccacagagacctcgctgcccggaactgyatggtggccgaagacttcacagtcaaaatcggagattttgggatgacgagagacatctatgagacagactattaccgcaagggagggaaagggctgctgcctgtgcgctggatgtcccctgagtccctcaaggacggagtcttcaccacgcactctgatgtctggtccttcggggtcgtcctctgggagattgccacgctggccgagcagccataccagggcttgtccaacgagcaagtccttcgcttcgtcatggagggcggccttctggacaagccggacaactgtcccgacatgctgtttgavctgatgcgcatgtgctggcagtacaaccccaagatgaggccttccttcctggagatcatcagcagcatcaaggaygagatggagcctggcttccgggaggtctccttctactacagtgaggagaacaagccgcctgagccggaggagctggacctggagccggagaacatggagagcgtccccctggacccctcggcctcctcgtcctccctgccgctgcccgacagacactcaggacacaaggctgagaacggcccaggccctggcgtgctggtgctgcgagccagcttcgacgagagacagccttacgcgcacatgaacgggggccgcaagaacgagcgggccctgcctctgccccagtcctcgacctgctgatcctggatcccgatcccgtgcaaacagtaccgtgcgcacgcgggcgggcggggggagagttttaacaatctattcacaagcctcctgtacctcagtggatcttc

SEQ ID NO: 8 - DNA, 22 bp, Mus musculus
ggagatcatcggcagcatcaag

SEQ ID NO: 9 - DNA, 21 bp, Mus musculus
gccattctcagccttgtgtcc

SEQ ID NO: 10 - DNA, 21 bp, Mus musculus
gcatgtgctggcagtataacc

SEQ ID NO: 11 - DNA, 21 bp, Mus musculus
cagaggcccatgtcagttaag

SEQ ID NO: 12 - DNA, 21 bp, Mus musculus
agagtggccatcaagacggta

SEQ ID NO: 13 - DNA, 19 bp, Mus musculus
ggcctcagagaccggagat

SEQ ID NO: 14 - DNA, 20 bp, Sus scrofa
cctccgtgatgaaggagttc

SEQ ID NO: 15 - DNA, 20 bp, Sus scrofa
tcagttccatgatgaccagc

SEQ ID NO: 16 - DNA, 20 bp, Sus scrofa
cctccgtgatgaaggagttc

SEQ ID NO: 17 - DNA, 20 bp, Sus scrofa
tcagttccatgatgaccagc

SEQ ID NO: 18 - DNA, 19 bp, Sus scrofa
ggagtatgatgggcaggat

SEQ ID NO: 19 - DNA, 20 bp, Sus scrofa
gaagcattggtgcgaatgta

SEQ ID NO: 20 - DNA, 12 bp, Mus musculus
tggagatggagc

SEQ ID NO: 21 - DNA, 17 bp, Sus scrofa
aagatgaggccttcctt

SEQ ID NO: 22 - DNA, 20 bp, Sus scrofa
gatcagcaggtcgaggactg